SEQUENCE LISTING (42 sequences)

SEQ ID NO: 1 - DNA, 18162 bp, Cedar virus
accagacaaaggaagtctagtctccggattaaatcatattcgtatgattaatcttaggatcccggtatctagaatctggatctggattcggtttaattgaattgcgatcgtttataaattagaaaggagatttactactcaaaatgtctgacattttcaatgagactcaatcatttagaaactatcagtccaacttaggcagagatggcagggccagtgcagcaacgactactttgacaactaaagtgaggatctttgttccagcgaataataatccaaacctcagatggcgtttaacactattcttgatggatgtcgtgaggtcacctgcctccgcagagtctatgaaagtgggtgctgggatatccttggtatctatgtatgctgaaaaacccggggctcttgtgagagcattattgaatgacccagatgttgaagcgataatcatagatgtttatggctttgatgaaggtattcctataatggaacgaagaggtgataaagctacagatgacatggattccctaagaaagattgttaaagctgcacatgatttcagcagaggaaggagtttatttgttgatcaaagggtccaggatattgttatgtcagatatggggtcatttgtgaatgctattacttccatagagacgcagatatggattttgatcgcaaaggctgtaactgccccagatacagcagaagagagcgaaggaagaagatgggcaaaatatgttcagcaaaagagggttaatcctttgttcttgatttctccacaatggatcaatgacatgagatccctgattgcggcaagtctttcgcttcgtaaattcatggttgaactactgatggaagctaagaaaggacgggggacaaaaggaagaataatggagattgtatccgatatcggaaattacgttgaagagacaggaatggcagggttcttcgctacaataaagttcggtcttgagaccaaattccctgctttggcacttaatgagctccagagtgacttgaacacaatgaaaagtctcatgatactgtacagaagcataggaccaaaggccccctttatggtgttgttggaagattcaattcagaccaaatttgctccaggaagctatccacttctttggagttttgcgatgggtgtaggcacaactattgacagagctatgggtgccttgaacattaacagaagttatcttgaacctgtctattttaggctagggcaacaatcagctaaacatcaagcaggaaatgttgacaaagaaatggcagaaaagttaggattgacagaagaccagatcgtgcacctatcagctaatgtgaaggatgcaagtcaaggtagagatgacaatcaaatcaacatccgagaagggaagttcacaaatgttgttgatgacatccaggatcatgcccagagttcctctgaggattacaatcctagtaaaaagagtttctcaatattgacgagcatcacatccaccgtagatagtgctgacagtaggtctgcaatgaatgagtcaatgacaacaacatccttgctgaaattgagacagaggctggcagagaagaaaggagactccaagaacagtcaagacacacctccaaaaccacccagagcaaaagatcaacccactgatgaggtctccttcatggattccaatatatgatcagaatgatggttaaaatcaaccaactaagggcgcgtagagtaccttcagatagaacactacattaatcgggtgaaacaatagatttatgggtttggtgcttaatttttatttaatcttacttgcaaaacaggcagctgctacactcgtaaccactcctcacagtaagggcaacacgggtcatagaacttatgcctatagattacctctatctgtatatctagctatgattaaaatgtatacttctgctgaccggttttctagcaacagtccacattattactttatgggtattttttaatcaaccttttataatcaaatatattacaaaaaacttaggatccaagtggtccaaactttttttgatcaagagtcatattggctactttaggaggacactttaaacacaaattgttacaagaggatattcatcagatggacaaactacaattgattgaagatggcctctctactatcaattttatacaggaaaataaggaaaaattacagcattcttacggaagatcctccatcagagagccacccacaagtgtcagggttgaagagtgggagaaatttattcgaaagatcgcttctggacctgaacaagttcaagggggaggatctgagactgagatcacaggcgataatggagatagaggcaattttaccaatcctgatcagggaggcggagtcacaggacaattcgaagaaaggtatcaaaaatgggggtcacaagattcagaattacaactggacccaatggttgtacacgatttcttctatgacgagagaagggagaatcccgacaatggaaaatatgaccgcagctctaaaaaacgggataatatcagagaaggaacacgacaggataagtacaataatcagtctactgatgaattactgtcctgcctacaaccatcttctaagaacgatgtcatcaagaatgaaagtacatcagtgtcaaatttgcatgttacaggaaataaactgaatcctgacgcaaaaccctttgaacccacctcccagtcgaaagagcacccaaccaccacacagcacaacaaaaatgaccatcagaccgatgatgattataagaatagaagatccagtgaaaacaatgtgatctctgatcatgccaccacaatggaagacaacaacaattttatcccggcgaccaaaagaaagaatgcattgagcgaacccatatacgtccaggtattgccctcaaacacagagggtttctcgggaaaagattatccactcctcaaggacaactctgtcaagaagcgtgcagagccagtcatcctagaaactgccaaccaccctgcaggctctgccgaccaagacacaaatcagattgaagaaaacatgcagttcaaccttccaaaactgctcacagaagatacagacgatgaaccagaggataacaatgattccatgcctcttgaggaagacattagagagatcggttccatgctaaaagatggaaccaaagatatcaagacaaggatgaatgagatagatgacgcaatcaagaagataaataagaaatcaaaaaatagaagtctggatctagaatcagacggtaaagatcaggggagaagagatccatcagtagacctcgggattaaaaaaagaaaggaagggctaaaggccgcaatgcaaaagacaaaagagcaattgtctataaaagtggagagagagattggattgaacgacaggatatgtcaaaattcgaagatgagtacagaaaagaaattgatatatgctgggatggaaatggagtatggacaaacgagtactgggtcaggaggtccacaaggatcaaaggatgggacttctgatgatgtccaggtagacgaagactacgatgaaggggaagactatgaggctatgccgtcagataggttttatacaacattatcaggtgaacaaaaggatagatttgatctagatgctaaccaaatgtctcagtatgacctcgaggcccaggtggatgaattaaccagaatgaatctcatactctattctagattagaaactactaataagttgcttattgacatattagatctagctaaagaaatgccaaagttagttagaaaagtggataatcttgagagacagatgggtaacttgaatatgttaacctctacccttgagggtcacctatcttctgtaatgattatgatacccggtaaggataagagcgaaaaggaaatccctaaaaatccggacctgagaccaatactggggagaagcaacacgtcgttaactgatgttatcgacctagaccattaccctgataaaggctccaaaggtatcaaaccaagtggatctggagacagacagtacatcggctctctagagagcaaattttctataaatgatgagtacaattttgctccataccctatcagggacgaactcctattgccaggtttaagagatgacaaaaccaatgcttcatcgttcatcccagatgacacggacaggtctccaatggtgctcaaaataataattcgacagaacatccatgatgaagaagtgaaggatgagctactgtccatactagaacaacataacactgtggaggaattgaatgaaatatggaatactgtgaatgattacctcgatggcaacatctgattaacagatattgagattgatcctattctaaacaagtaatctctgataatgatagtatggaataagaatactaatcacactattgtactcttgtagaatcttaacgagtgtctaatgtcagattttagcaacacatactaataacttgtaatccatttctccttattccatttaatctcacattagaaaaaacttaggatcccagatttgcaaagtcaaaacgggatctactatcaggtgttggagctaacaatagcggagtctgcataacaaatagcgttcaaagaagtttgaaaaccatcatagaatatggatccgtcagatttgaggaggattataatggaggatgataagagtctggtcaacaatgatgatagtacagaaactgattttctcgagaaaacttggagagaagggagtaagattgacaagatcacaccagaggttgatgaaaacgggaatatggtccccaagtacgttgtcttcaacccggggaaaaatgagaggaaaacatccggatatcaatatatgatttgttatggtttcattgaggatggacctatcaatggctcaccaagagtcaaaggtaatatcagaaccaccgcttcttttcctttgggtgttggaaaaacttactcgtctccagaagagatcttacaagagctgacaacactcaagatcactgtcagaaggacagccggatcaaatgagaagttggtgtatggaataacagggcctttaaatcacctttacccgtggtataaagttttgacaggtggctccatttttagtgcggtgaaggtctgtaggaatgtggatcaaatactattagacagaccccaaatacttagagtattctttctaagtataactaaattaacagataaaggtgtgtatatgatacccaaaagtgttctcgacttcagatcggataattcgatggccttcaatctgcttgtgtatctcaagatagacactgacatcaccaaagcaggcatcagagggattgtcaacaaagaaggggagaggataacgtcattcatgttacacatcggtaactttacaagaagaggaggaaaacattactcagtggagtattgcaaaaggaaaattgacaaaatgaagctcacattcgccttaggcactataggcggtctaagcttacatatcaggatcgatggaaggataagtaaaaggctccaagcacaagttggctttcagagaaacatttgctactcactaatggacacaaacccatggttgaataaattaacgtggaacaatagttgtgaaatacacaaagtcaccgctgtcattcagccatctgtgccaaaggacttcatgttgtatgaggacatcttaatagataatacaggcaagatcttaaaataaagtaggagagtcagtcattacccagtatattgaatactaatgacaactttattaatccaattctatctccagttactagaatttctaaaacaattctactgctcagcaacgcatctcaaacattgtgatcttcaattatgatcgacgcattgtaatctatatagcttttagttcatgaaatactaaaaagggcttaatcttgtaagttctcagcaaatactccaatgcaaaagagcgcctcaacatctcaagcagcaccaaaataaaccacaatcaatgtgcaacaagagcaatcgtctaaagtgtgaaaaccaaaatcacagatcagaaagggcacatatttcagtcctgtaaaaataccaagtgggattaataaaagaggatcaatccttatcattttaagaaaaacttaggatcccagagatcctaaagagccaattcctttatattttgatcttgaagggctagaagtcaggctgaaacacagaggtggaggaacacaggaactaaaattgatgaaatcaaccttagctcaacatctaatcaatcaagcttaagtcatcctaatactgtatacaaccagcagcgtagagagtggatttgatttcggcacccttgcgaagtgaaggctattactgcctgtcctttcaatcagaaaattacatttacccataaagtaatctcaacatgtctaacaagaggacaacagtattgatcataataagctatacgttattttatttgaataatgcagcaattgtagggtttgattttgataaattgaataaaataggtgtggtgcaagggagagtcctaaattataaaattaaaggagatccaatgacaaaagaccttgtcttgaaatttatccctaacatagtgaatatcactgaatgtgtgagagagcccttgagtaggtacaatgagaccgtgaggagattgcttttacctatacacaacatgcttgggttatacttgaataacacaaatgctaaaatgactgggttgatgatcgcgggtgtgatcatgggtgggatagcaataggtatagccacagcagctcagatcacagcaggttttgctctttatgaggcaaaaaagaacacagaaaatattcagaaattaacagacagcatcatgaaaacacaggactcgattgataaacttactgacagtgtggggacaagcatacttatattgaataagctacagacatacatcaacaatcaactggtaccaaatctagagcttctatcctgccgacaaaacaaaattgagtttgatctaatgttaaccaagtatttggtggatcttatgactgttattggtcctaatatcaataatcctgttaataaagatatgactattcaatctttgtcacttctttttgatggcaattatgatataatgatgtcagaacttggttatacacctcaggatttcttagatttgatagagagtaagagtataacagggcaaataatttatgttgatatggaaaacttgtacgttgtgatcaggacatatctacctaccctaattgaagtacctgatgcccaaatatatgagttcaacaaaataactatgagtagcaatggaggagaatacttgtcaaccatacctaatttcatattaataagaggtaattatatgtctaatatagatgttgcaacatgttatatgaccaaagcaagcgtaatttgtaatcaagattattcactcccgatgagccaaaacttaagaagctgttatcaaggtgagacagaatactgtcctgttgaggcagtcatcgcgtcacactctccaagatttgctcttacaaatggagttattttcgccaattgtataaatacaatttgtaggtgtcaagacaatggtaagactatcactcaaaacataaaccaattcgtaagcatgatcgacaacagtacttgtaatgatgtcatggtagataagtttactatcaaggtaggaaaatatatggggagaaaagatatcaataatattaatatccagataggaccgcagatcataattgataaggttgacttgtctaatgaaataaacaagatgaatcaatctttaaaagatagtattttctacctgagagaagccaagagaattttagactcagtaaatatcagtcttatatctccaagcgttcaattgtttctaataataatatcagtcctctcatttattatattattgattatcatagtatacttgtactgtaaatcaaaacattcatataaatataacaaatttatagatgatcctgattattacaatgattacaaaagagaacgtattaatggcaaagccagtaagagtaacaatatatattatgtaggtgattaacaatcgataatctaaaggattacctcactatcactaccaaggtaacttccatgtaagatcggaccttccccgaagacattaaataaaacttaggatcccagagtatccctctaagtgatccttctagattggttactgatatatatacatatttatcctctttccgtcgttgtttattgatcattaataatgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtctgcacgatatcaagttttgtatttattgtatattctcactgctaattatcattacaataatcaatataatcacaatatcaattgttataactcgtctgaaagtacatgaagagaataatggcatggaatctcctaatttacaatctattcaagatagtctctcatctcttactaacatgatcaatacagagataactcctagaatagggattttagttacagccacttctgttactctctcttcatctatcaattatgtcgggactaagacaaatcaactggtcaatgaattaaaagattatataaccaaaagttgtggctttaaggtccctgaattaaagttacatgaatgcaacataagttgtgctgatccaaaaattagcaaatctgcaatgtacagcaccaatgcctatgccgagcttgctggtccacctaagatattttgtaaaagtgtatccaaagaccccgactttagactgaagcagatagattatgtaataccagtgcagcaagatcggtctatttgtatgaacaaccctttattggatatttctgatgggttttttacctacatacattatgaaggaataaatagctgtaaaaaatcagattcatttaaagtgctgctgtcacatggtgaaatagttgacaggggtgattatcgaccatcattatatctattatcaagtcattaccatccttattcaatgcaggtaataaactgtgtacctgtgacttgtaaccagtcatcctttgtattctgtcatatctccaacaacactaaaacattggacaattcagattactcgtcagacgagtactacataacatatttcaatggcatagatcgtcccaaaaccaagaagattcccattaacaatatgacagcagacaatcgttatatccattttacattctcaggtgggggaggtgtatgtttaggtgaagaatttattattcctgttaccacagtcatcaatactgatgtattcacgcatgattattgtgagagtttcaactgttcagtccaaaccggtaaaagtctaaaggagatatgctctgagtcattaagatctccaacgaactcatcgcgatacaatttaaacggaatcatgattataagtcaaaacaacatgacagattttaagattcagttgaatggtataacttataacaaactgtcattcggaagtcctggaagactgagcaagacactgggccaggtcctttattaccaatcttcaatgagttgggatacttatctaaaggcaggatttgtcgagaaatggaaaccctttaccccgaattggatgaacaatactgtgatatccagacctaaccaaggtaattgtccaaggtatcataaatgccccgagatatgttatggagggacatacaatgatattgctcctttagatctaggaaaagacatgtatgttagcgttattctagattcagatcagcttgcagagaatccagagattacagtatttaactctactactatactttataaggagagagtatccaaagatgaactaaacacaagaagtactacaacgagctgttttcttttcctagatgaaccttggtgtatatcagtattagaaacaaacagatttaacggcaaatctattaggcccgagatttattcatacaaaattcctaagtattgttaatttgatgagcttattcctcatacttcaatcaaatttaatataactaatatcaaattgttgcactcagctattattaaaactggatcatcagacaataaagatgtatacaaagatatatcgaagagggtattaaagaaaacttaggatcccagatccttcaataaggcagagccttgattgtatcagcgtcatttacaattgaatctcaattaacaacactgattaataacttaagcagaatactcctattacagtgtttaattgacttaattttaattgaggattttataatcctataattggagcagatctaaactctcaccgattcagttctaatcctttattaactaaagaacaaattctaaataattggatgacgtcacaggagacaagctggaaacaatttagttagaaggaagaaaccttttaccagatatggaaagtgactttgatatatctgttagcgacgtactgtacccagaatgtcatttggacagtcctatagtcggcggtaagctcattacttctcttgagtatgcgaatttgactcataaccaacctcatgaagatcagacattgctgactaatataaatgtcaataaaaagaagaagataaaaagtcctctaatatcccaacaatctttatttggaaatgaggttaataaggagattttcgatcttaaaaattattaccatgtcccctatccagaatgtaacagagatttattcttaatctctgatgacaaaatagcattcaaactcagtaaaatcatggataattctaataaactgtttgatggtttagagaggaaactgagtcgcttaatttcgaatgtagataatcaactattaaatgcaacctctcttcataataattctgagatggatcggaagggaaaagaacatccttgcttcccagaaaagagcacaattgatgatgtaagacagcagagacagacacgagattttccaaagaattcaactagagagggaagatctccaaaacaccctgatgccggtcctacacctgaaaacagtgccaaaaacgatttgcatagagacaacacagacaatatgccaacaggccatagttcgacatctatgaaaaaacctaaaatatctggagaagaatatcttagtatgtggctagactcagaggatttgggttctaaacgaatttctgcacaattagggaaggatgtatcatgtaaaggccatctgcacacgacagaagacaaaccgataatagttcctgacactcgatatatccaaaatcatgaatctaataacgatattttccccaaaaaagagaaaaaattctgcaaacttccaccgtcatcggataatttaaccaaaatcatggtgaattcaaaatggtacaatcctttccttttttggtttactgtcaagactgaacttagagcctgccagaaggagaactacaaaaggaaaaacagaaaattgggaattatcacatcgattaaaggttcatgctataagttgatactcaaccagaatctagtagcaatattcgaggaagacagcagtggatactcagatcataaaaaaagaaaaaaacgatgctactatctaactcccgaaatggtccttatgttctccgatgtaactgaaggaagattgatgattgatgttgcaatgagatttgacaaaaagtacaaaactctagagaaaaaggctttgaaattatggtttcttatagacgagttatttccttctatgggaaatagagtgtataatattatatccatgcttgagcctttgactctcgcgatattacaggttaaggatgagtcaaggttgttgagaggtgcattcatgcatcattgtttaggtgacctcttcgaagaacttcgagagtccaagaactacccggaagatgagatcaagagatttgccaacgacctaataaatgtcatgacctgtcgggacattcatttagtagcagaattcttctcattctttaggactttcggacatccaatattgaacgctcaaactgcagccaggaaagttagagagtacatgttagcagataaaatccttgagtacgaacctatcatgaaaggtcatgcgattttctgtgctataatcataaatggatttagagatagacatggaggagtttggcctcctcttgatcttccaaaacattgttcaaagaacataatatctctcaaaaatacaggtgaaggggtaacttatgaagtagcaataaacaattggagatcatttgtcgggttaaagttcaaatgttttatgggtctcaatttagacaatgatctcagcatgtacatgaaagataaagcattatcacctttaagggatctttgggattcaatctattcacgtgaagtaatgtcctaccaaccacctagaaacaaaaaatcaagaagattggttgaggttttcgttgatgatcaggactttgatcccgttgatatgataaattatgttctgaccggagaatatctcagagatgatgatttcaatgcttcttatagtttaaaagagaaagagaccaaacaagttggcaggttgtttgctaagatgacttataaaatgagggcctgtcaagttattgctgagaatttaattgcacatgggattgggagatatttccatgaaaacgggatggttaaggatgagcatgagctcagcaaatcactgtttcaattgtctatatcaggaataccaagagggaacaaaaacaacaaatcgacgaacgacacaatccacgaaagcaagatcgagaataaccattcctttaaaaacatccagaatcgatcatttcgaaagacggataacccatacaatagatttaacattgataacccaactttcttatccccaaactgtaaccccaagtataaccgtaagaattcagagacaataggtatattctctcgtgcagaaaccaaaagcatgattagagaacagaaaagtcacagagaagtcaaaataaataagctagatatcggcagtgataatgaagagcaaggaaaagagatagatgccgccaagtacaaaatcacggacaacccaaatccacacataaatcctcaagatcaacccggaatctgtcaagaagacaaaggcaaagaaggagcaaagtcagatctcacagaaggcatgagttttctggagatgcacacactctttaacccgagtaagagcgatatcagaacaaatctcgaattggaaaagagttcactttcaaaccctggatttatatcacaaaaagagaaaagaggcaaaacttataatgaatcccattcactgggaaagttctctaaagaggatgaagaaagatacgatgtcatcagtgcattcctgacaacagatttacgaaaattctgcttaaattggagacatgaatcaatcggcatttttgcaagaaggatggacgaaatctatggtttgcctggtttctttaattggatgcacagaagactagagcgatctgtgttatatgttgcggaccctcattgcccgccgtctatcaatgaacatatcgatctaaacgattcacccgaaagagacatatttatacatcatccgaaagggggtatagaaggatacagccaaaaactgtggacaatagcgactatcccttttctattcctcagtgctcatgagacaaacacccggatagcggcagttgtacaaggtgacaatcaatcaattgcaattacacataaggtccaccctcatttgccttacaaaatgaagaaagaactctctgcaatgcaggcaaaaaaatatttttcaaggttacggcacaacatgaaggcattagggcatgaattgaaggcgaccgagactatcattagtactcatttcttcatttattccaagaaaatccactatgacggggctgttttatcacaatctctgaaatcaatggcaaggtgtgtattttggtcagaaacccttgttgatgaaactagagcagcatgcagtaatatcagcacaacaattgcaaaggctattgagaatggttatagcaggagatctggctatctgataaatgttcttaaaaccatccaacaaattaatatatcattgagttttaatataaatgaatgcatgacagatgacataatcagaccgtttagagataatccaaactggatcaaacatgccgcattaatccccgccagcttgggaggactcaactatatgaacatgtctcgattgtatgtgaggaatataggggatccagtcacagcatcgatagcagatgttaagagaatgattctcggtggtgtactacccattggaatactccacaatatcatgttgcaagaacccggtgatgccacttatttggactggtgtagtgatccatactccatcaacctaaagcagactcaaagtatcacaaaagttataaagaacataacggcaagagtgatactaaggaattcggtcaatccactgctcaaaggtctatttcatgaaggtgcttatgaggaggacactgaattagcaacattcattttggacaggagagtcatcttaccacgagtcggtcacgagatcttaaacaactccatcacaggagcaagagaagagatctcgggcttactggataccacaaaaggattgataagaattggcatagcaaagggaggattaactcagagaacattatctcgaatttccaattatgattatgaacaatttttgaacctaatgaatatgttgaagaacaaagaacaaaacagtgtcatttccctgtcagcttgctctgttgactttgctatagctttaagaagcaggatgtggaggaaattggcaaaaggaagattaatatatggtttagaagtccctgatccaatagaagcaatgattggctttctcattcttgggagtgaaaattgtctactctgtgattcaggaagcaaaaactatacctggtttttcataccaaaggatgtacagttggataagattgataaagatcacgcatcaataagggtaccctatgtcggatcaactaccgaagaaagatcagagataaagttaggatccgtgaaaaatccaagcaaatccctgaaatctgctataagactcgcaactgtgtacacttgggcatttggcacaagtgatgctgaatggtgggaggcttggtacttgtctaatcaacgagcaaatatacccttagatgttctcaaaacgataacacctatatctacttcaacgaatattgctcatagattacgagaccgatcaacacaggttaaatacgccagtacatctcttaacagagtatcgcggcatgtaacaattagtaacgataacatgaattttgaatttgacggggttaaaatggataccaacttgatttatcaacaagtcatgctgttagggctttcatgcttggagagtttattccgaaataggaaaatgacaaatagttacaatatcgtgtaccatttacacgttcaagaacattgttgtgtaaaggctctgaatgatttaccttatacaccgtcaacacatccagtgccaaattatacagaagttagagataataggttaatttacgatcctcaacctatattagaatttgatgagctaagattagcaattcagcaaacaaagaaagtagatttggaattttcattgtgggatacaaaagaacttcatgagaatttagctcaaagtttagcgattacagtaacggatattatgacaaaatctgataaagatcatattaaagaccaaagaagtatagatgttgatgataatattaagacactaataactgagtttttattagtagaccctgaaatgtttgccgtaaatttaggattgcatatatcaataaaatggtcatttgatattcactttaaaagaccaagaggacgctatagcatgatagaatacttgactgatcttttggataatacttcttctcatgtttatcgaatccttactaatgtattatctcatcccagagttatgagaaaattcactaatgccgggctactagtaccgaaatacggtccctaccttacaagtcaagatttcaaaaagatggcggtagatttcataataacagcgtataccacatttttgaccaattggtgtaataataacaagttttcaattctaatacctgaacaagaccctgatatacttgaattaagaaaagacatcactcatgcaaggcatttatgtatgatctcggatctttactgctactctttcaagcaaccttggataaaggagcttacaccacaagagaagatctgcgtcatggaggacttcatagccaattgtgttgctaatgatcaaacaagtgcgggctggaacataacgcccttaagagtttacaatctccctgcatcgaccacatacatcaggagagggataataaaacaattaagaatccgtcaaagcaatgagcctattgatctggaagatattaggattggtcagaaccccgattttgtgaataaacctattgagttttgtagcagtgaattcggtatcacaatttataaccttgaagaaattcttcaatcaaatgtgcatctcagtgtaaatatgaacattgactcctcaacaagtaacaatactgaaaatcatttatttagaagggtaggcttgaactctacttcatcttataaagcactatctttaacacctgttattaaaagatatcatcaacagaacactaataggctgtttataggagaaggatcagggtctatgatgtatctttaccagaaaaccttgggggagacaatatgcttctttaattcgggagttcagtacaatgaggatctgggtcaaagggaacaatcattatacccgagtgaatacagtatctgtgaacaaggagtaaaaaaagaaaaccctctcaccgggcatgttataccactattcaatggaagaccagaaaccacatgggtaggcaatgatgattctttcaagtatatattggaacatactataaatagagacatcgggcttgttcactccgatatggaaacaggaatagggaaggataattatactatcttaaatgaacatgcacatcttatagcactgagccttacagtaatgattgatgatggaatcttggtgtctaaggtagcttatgcccctgggttttgcatctcttcattattgaatatgtaccggacatttttttcattagttctatgtgcgtttccaccgtatagcaattttgaatcaactgaattttacctgatttgcttgcaaaaaagtatacccggacctatcacaccagctagagccatccaacaaacgacgaagcaatctagagaagaggataatagtataactaataatatcctcaaaatcaaaaatcttgttcagaaagaatttatcaaaacagtaaagaaaaaatacgaaatccatccttcgtttaactgtcctatcaacttcacaaaggatgataaatatttaatgagtgttgggtttcaagccaatggtcctgatatgatacgtaaagagacgggctatgacataggtagcaatgtagagaatctccgagatgtcttaatcaagttgtttgcagatgcagtcaccttctatgatgatgtcacaaataaaaagaactttttaaatccttatccagtctacacaagaactcagtataaaattctgatggataaaatatgcaagaaagtcaccttatacaccttaatcatatcatgtaaaggatccaatcaatattgctgggaaattaaatcccaaataagaaagcattgtctcatacttgatttgaaaagtaaggtttttacaaaacttattccaaagggattaagagaaaggggtgactcaaaagggatgaagagcatatggttcactaaactaaccagtcaagaggtgaaaagatggtggaagatgatatcttacatcgtgataataagcaatccataaccacatccaacttgtcagttaaacacttaaatcacaataaacttgtcatcagattaaagaaaacttataattcccttttttaggt

SEQ ID NO: 2 - DNA, 18168 bp, Cedar virus
accagaaaaaggaagtctagtctccggattaaatcatattcgtatgattaatcttaggatcccggtatctagaatctggatctggattcggtttaattgaattgcgatcgtttataaattagaaaggagatttactactcaaaatgtctgacattttcaatgagactcaatcatttagaaactatcagtccaacttaggcagagatggcagggccagtgcagcaacgactactttgacaactaaagtgaggatctttgttccagcgaataataatccaaacctcagatggcgtttaacactattcttgatggatgtcgtgaggtcacctgcctccgcagagtctatgaaagtgggtgctgggatatccttggtatctatgtatgctgaaaaaccaggggctcttgtgagagcattattgaatgacccagatgttgaagcgataatcatagatgtttatggctttgatgaaggtattcctataatggaacgaagaggtgataaagctacagatgacatggattccctaagaaagattgttaaagctgcacatgatttcagcagaggaaggagtttatttgttgatcaaagggtccaggatattgttatgtcagatatggggtcatttgtgaatgctattacttccatagagacgcagatatggattttgatcgcaaaggctgtaactgccccagatacagcagaagagagcgaaggaagaagatgggcaaaatatgttcagcaaaagagggttaatcctttgttcttgatttctccacaatggatcaatgacatgagatccctgattgcggcaagtctttcgcttcgtaaattcatggttgaactactgatggaagctaagaaaggacgggggacaaaaggaagaataatggagattgtatccgatatcggaaattacgttgaagagacaggaatggcagggttcttcgctacaataaagttcggtcttgagaccaaattccctgctttggcacttaatgagctccagagtgacttgaacacaatgaaaagtctcatgatactgtacagaagcataggaccaaaggccccctttatggtgttgttggaagattcaattcagaccaaatttgctccaggaagctatccacttctttggagttttgcgatgggtgtaggcacaactattgacagagctatgggtgccttgaacattaacagaagttatcttgaacctgtctattttaggctagggcaacaatcagctaaacatcaagcaggaaatgttgacaaagaaatggcagaaaagttaggattgacagaagaccagatcgtgcacctatcagctaatgtgaaggatgcaagtcaaggtagagatgacaatcaaatcaacatccgagaagggaagttcacaaatgttgttgatgacatccaggatcatgcccagagttcctctgaggattacaatcctagtaaaaagagtttctcaatattgacgagcatcacatccaccgtagatagtgctgacagtaggtctgcaatgaatgagtcaatgacaacaacatccttgctgaaattgagacagaggctggcagagaagaaaggagactccaagaacagtcaagacacacctccaaaaccacccagagcaaaagatcaacccactgatgaggtctccttcatggattccaatatatgatcagaatgatggttaaaatcaaccaactaagggcgcgtagagtaccttcagatagaacactacattaatcgggtgaaacaatagatttatgggtttggtgcttaatttttatttaatcttacttgcaaaacaggcagctgctacactcgtaaccactcctcacagtaagggcaacacgggtcatagaacttatgcctatagattacctctatctgtatatctagctatgattaaaatgtatacttctgctgaccggttttctagcaacagtccacattattactttatgggtattttttaatcaaccttttataatcaaatatattacaaaaaacttaggatccaagtggtccaaactttttttgatcaagagtcatattggctactttaggaggacactttaaacacaaattgttacaagaggatattcatcagatggacaaactacaattgattgaagatggcctctctactatcaattttatacaggaaaataaggaaaaattacagcattcttacggaagatcctccatcagagagccacccacaagtgtcagggttgaagagtgggagaaatttattcgaaagatcgcttctggacctgaacaagttcaagggggaggatctgagactgagatcacaggcgataatggagatagaggcaattttaccaatcctgatcagggaggcggagtcacaggacaattcgaagaaaggtatcaaaaatgggggtcacaagattcagaattacaactggacccaatggttgtacacgatttcttctatgacgagagaagggagaatcccgacaatggaaaatatgaccgcagctctaaaaaacgggataatatcagagaaggaacacgacaggataagtacaataatcagtctactgatgaattactgtcctgcctacaaccatcttctaagaacgatgtcatcaagaatgaaagtacatcagtgtcaaatttgcatgttacaggaaataaactgaatcctgacgcaaaaccctttgaacccacctcccagtcgaaagagcacccaaccaccacacagcacaacaaaaatgaccatcagaccgatgatgattataagaatagaagatccagtgaaaacaatgtgatctctgatcatgccaccacaatggaagacaacaacaattttatcccggcgaccaaaagaaagaatgcattgagcgaacccatatacgtccaggtattgccctcaaacacagagggtttctcgggaaaagattatccactcctcaaggacaactctgtcaagaagcgtgcagagccagtcatcctagaaactgccaaccaccctgcaggctctgccgaccaagacacaaatcagattgaagaaaacatgcagttcaaccttccaaaactgctcacagaagatacagacgatgaaccagaggataacaatgattccatgcctcttgaggaagacattagagagatcggttccatgctaaaagatggaaccaaagatatcaagacaaggatgaatgagatagatgacgcaatcaagaagataaataagaaatcaaaaaatagaagtctggatctagaatcagacggtaaagatcaggggagaagagatccatcagtagacctcgggattaaaaaaagaaaggaagggctaaaggccgcaatgcaaaagacaaaagagcaattgtctataaaagtggagagagagattggattgaacgacaggatatgtcaaaattcgaagatgagtacagaaaagaaattgatatatgctgggatggaaatggagtatggacaaacgagtactgggtcaggaggtccacaaggatcaaaggatgggacttctgatgatgtccaggtagacgaagactacgatgaaggggaagactatgaggctatgccgtcagataggttttatacaacattatcaggtgaacaaaaggatagatttgatctagatgctaaccaaatgtctcagtatgacctcgaggcccaggtggatgaattaaccagaatgaatctcatactctattctagattagaaactactaataagttgcttattgacatattagatctagctaaagaaatgccaaagttagttagaaaagtggataatcttgagagacagatgggtaacttgaatatgttaacctctacccttgagggtcacctatcttctgtaatgattatgatacccggtaaggataagagcgaaaaggaaatccctaaaaatccggacctgagaccaatactggggagaagcaacacgtcgttaactgatgttatcgacctagaccattaccctgataaaggctccaaaggtatcaaaccaagtggatctggagacagacagtacatcggctctctagagagcaaattttctataaatgatgagtacaattttgctccataccctatcagggacgaactcctattgccaggtttaagagatgacaaaaccaatgcttcatcgttcatcccagatgacacggacaggtctccaatggtgctcaaaataataattcgacagaacatccatgatgaagaagtgaaggatgagctactgtccatactagaacaacataacactgtggaggaattgaatgaaatatggaatactgtgaatgattacctcgatggcaacatctgattaacagatattgagattgatcctattctaaacaagtaatctctgataatgatagtatggaataagaatactaatcacactattgtactcttgtagaatcttaacgagtgtctaatgtcagattttagcaacacatactaataacttgtaatccatttctccttattccatttaatctcacattagaaaaaacttaggatcccagacgcgtatttgcaaagtcaaaacgggatctactatcaggtgttggagctaacaatagcggagtctgcataacaaatagcgttcaaagaagtttgaaaaccatcatagaatatggatccgtcagatttgaggaggattataatggaggatgataagagtctggtcaacaatgatgatagtacagaaactgattttctcgagaaaacttggagagaagggagtaagattgacaagatcacaccagaggttgatgaaaacgggaatatggtccccaagtacgttgtcttcaacccagggaaaaatgagaggaaaacatccggatatcaatatatgatttgttatggtttcattgaggatggacctatcaatggctcaccaagagtcaaaggtaatatcagaaccaccgcttcttttcctttgggtgttggaaaaacttactcgtctccagaagagatcttacaagagctgacaacactcaagatcactgtcagaaggacagccggatcaaatgagaagttggtgtatggaataacagggcctttaaatcacctttacccgtggtataaagttttgacaggtggctccatttttagtgcggtgaaggtctgtaggaatgtggatcaaatactattagacagaccccaaatacttagagtattctttctaagtataactaaattaacagataaaggtgtgtatatgatacccaaaagtgttctcgacttcagatcggataattcgatggccttcaatctgcttgtgtatctcaagatagacactgacatcaccaaagcaggcatcagagggattgtcaacaaagaaggggagaggataacgtcattcatgttacacatcggtaactttacaagaagaggaggaaaacattactcagtggagtattgcaaaaggaaaattgacaaaatgaagctcacattcgccttaggcactataggcggtctaagcttacatatcaggatcgatggaaggataagtaaaaggctccaagcacaagttggctttcagagaaacatttgctactcactaatggacacaaacccatggttgaataaattaacgtggaacaatagttgtgaaatacacaaagtcaccgctgtcattcagccatctgtgccaaaggacttcatgttgtatgaggacatcttaatagataatacaggcaagatcttaaaataaagtaggagagtcagtcattacccagtatattgaatactaatgacaactttattaatccaattctatctccagttactagaatttctaaaacaattctactgctcagcaacgcatctcaaacattgtgatcttcaattatgatcgacgcattgtaatctatatagcttttagttcatgaaatactaaaaagggcttaatcttgtaagttctcagcaaatactccaatgcaaaagagcgcctcaacatctcaagcagcaccaaaataaaccacaatcaatgtgcaacaagagcaatcgtctaaagtgtgaaaaccaaaatcacagatcagaaagggcacatatttcagtcctgtaaaaataccaagtgggattaataaaagaggatcaatccttatcattttaagaaaaacttaggatcccagagatcctaaagagccaattcctttatattttgatcttgaagggctagaagtcaggctgaaacacagaggtggaggaacacaggaactaaaattgatgaaatcaaccttagctcaacatctaatcaatcaagcttaagtcatcctaatactgtatacaaccagcagcgtagagagtggatttgatttcggcacccttgcgaagtgaaggctattactgcctgtcctttcaatcagaaaattacatttacccataaagtaatctcaacatgtctaacaagaggacaacagtattgatcataataagctatacgttattttatttgaataatgcagcaattgtagggtttgattttgataaattgaataaaataggtgtggtgcaagggagagtcctaaattataaaattaaaggagatccaatgacaaaagaccttgtcttgaaatttatccctaacatagtgaatatcactgaatgtgtgagagagcccttgagtaggtacaatgagaccgtgaggagattgcttttacctatacacaacatgcttgggttatacttgaataacacaaatgctaaaatgactgggttgatgatcgcgggtgtgatcatgggtgggatagcaataggtatagccacagcagctcagatcacagcaggttttgctctttatgaggcaaaaaagaacacagaaaatattcagaaattaacagacagcatcatgaaaacacaggactcgattgataaacttactgacagtgtggggacaagcatacttatattgaataagctacagacatacatcaacaatcaactggtaccaaatctagagcttctatcctgccgacaaaacaaaattgagtttgatctaatgttaaccaagtatttggtggatcttatgactgttattggtcctaatatcaataatcctgttaataaagatatgactattcaatctttgtcacttctttttgatggcaattatgatataatgatgtcagaacttggttatacacctcaggatttcttagatttgatagagagtaagagtataacagggcaaataatttatgttgatatggaaaacttgtacgttgtgatcaggacatatctacctaccctaattgaagtacctgatgcccaaatatatgagttcaacaaaataactatgagtagcaatggaggagaatacttgtcaaccatacctaatttcatattaataagaggtaattatatgtctaatatagatgttgcaacatgttatatgaccaaagcaagcgtaatttgtaatcaagattattcactcccgatgagccaaaacttaagaagctgttatcaaggtgagacagaatactgtcctgttgaggcagtcatcgcgtcacactctccaagatttgctcttacaaatggagttattttcgccaattgtataaatacaatttgtaggtgtcaagacaatggtaagactatcactcaaaacataaaccaattcgtaagcatgatcgacaacagtacttgtaatgatgtcatggtagataagtttactatcaaggtaggaaaatatatggggagaaaagatatcaataatattaatatccagataggaccgcagatcataattgataaggttgacttgtctaatgaaataaacaagatgaatcaatctttaaaagatagtattttctacctgagagaagccaagagaattttagactcagtaaatatcagtcttatatctccaagcgttcaattgtttctaataataatatcagtcctctcatttattatattattgattatcatagtatacttgtactgtaaatcaaaacattcatataaatataacaaatttatagatgatcctgattattacaatgattacaaaagagaacgtattaatggcaaagccagtaagagtaacaatatatattatgtaggtgattaacaatcgataatctaaaggattacctcactatcactaccaaggtaacttccatgtaagatcggaccttccccgaagacattaaataaaacttaggatcccagagtatccctctaagtgatccttctagattggttactgatatatatacatatttatcctctttccgtcgttgtttattgatcattaataatgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtctgcacgatatcaagttttgtatttattgtatattctcactgctaattatcattacaataatcaatataatcacaatatcaattgttataactcgtctgaaagtacatgaagagaataatggcatggaatctcctaatttacaatctattcaagatagtctctcatctcttactaacatgatcaatacagagataactcctagaatagggattttagttacagccacttctgttactctctcttcatctatcaattatgtcgggactaagacaaatcaactggtcaatgaattaaaagattatataaccaaaagttgtggctttaaggtccctgaattaaagttacatgaatgcaacataagttgtgctgatccaaaaattagcaaatctgcaatgtacagcaccaatgcctatgccgagcttgctggtccacctaagatattttgtaaaagtgtatccaaagaccccgactttagactgaagcagatagattatgtaataccagtgcagcaagatcggtctatttgtatgaacaaccctttattggatatttctgatgggttttttacctacatacattatgaaggaataaatagctgtaaaaaatcagattcatttaaagtgctgctgtcacatggtgaaatagttgacaggggtgattatcgaccatcattatatctattatcaagtcattaccatccttattcaatgcaggtaataaactgtgtacctgtgacttgtaaccagtcatcctttgtattctgtcatatctccaacaacactaaaacattggacaattcagattactcgtcagacgagtactacataacatatttcaatggcatagatcgtcccaaaaccaagaagattcccattaacaatatgacagcagacaatcgttatatccattttacattctcaggtgggggaggtgtatgtttaggtgaagaatttattattcctgttaccacagtcatcaatactgatgtattcacgcatgattattgtgagagtttcaactgttcagtccaaaccggtaaaagtctaaaggagatatgctctgagtcattaagatctccaacgaactcatcgcgatacaatttaaacggaatcatgattataagtcaaaacaacatgacagattttaagattcagttgaatggtataacttataacaaactgtcattcggaagtcctggaagactgagcaagacactgggccaggtcctttattaccaatcttcaatgagttgggatacttatctaaaggcaggatttgtcgagaaatggaaaccctttaccccgaattggatgaacaatactgtgatatccagacctaaccaaggtaattgtccaaggtatcataaatgccccgagatatgttatggagggacatacaatgatattgctcctttagatctaggaaaagacatgtatgttagcgttattctagattcagatcagcttgcagagaatccagagattacagtatttaactctactactatactttataaggagagagtatccaaagatgaactaaacacaagaagtactacaacgagctgttttcttttcctagatgaaccttggtgtatatcagtattagaaacaaacagatttaacggcaaatctattaggcccgagatttattcatacaaaattcctaagtattgttaatttgatgagcttattcctcatacttcaatcaaatttaatataactaatatcaaattgttgcactcagctattattaaaactggatcatcagacaataaagatgtatacaaagatatatcgaagagggtattaaagaaaacttaggatcccagatccttcaataaggcagagccttgattgtatcagcgtcatttacaattgaatctcaattaacaacactgattaataacttaagcagaatactcctattacagtgtttaattgacttaattttaattgaggattttataatcctataattggagcagatctaaactctcaccgattcagttctaatcctttattaactaaagaacaaattctaaataattggatgacgtcacaggagacaagctggaaacaatttagttagaaggaagaaaccttttaccagatatggaaagtgactttgatatatctgttagcgacgtactgtacccagaatgtcatttggacagtcctatagtcggcggtaagctcattacttctcttgagtatgcgaatttgactcataaccaacctcatgaagatcagacattgctgactaatataaatgtcaataaaaagaagaagataaaaagtcctctaatatcccaacaatctttatttggaaatgaggttaataaggagattttcgatcttaaaaattattaccatgtcccctatccagaatgtaacagagatttattcttaatctctgatgacaaaatagcattcaaactcagtaaaatcatggataattctaataaactgtttgatggtttagagaggaaactgagtcgcttaatttcgaatgtagataatcaactattaaatgcaacctctcttcataataattctgagatggatcggaagggaaaagaacatccttgcttcccagaaaagagcacaattgatgatgtaagacagcagagacagacacgagattttccaaagaattcaactagagagggaagatctccaaaacaccctgatgccggtcctacacctgaaaacagtgccaaaaacgatttgcatagagacaacacagacaatatgccaacaggccatagttcgacatctatgaaaaaacctaaaatatctggagaagaatatcttagtatgtggctagactcagaggatttgggttctaaacgaatttctgcacaattagggaaggatgtatcatgtaaaggccatctgcacacgacagaagacaaaccgataatagttcctgacactcgatatatccaaaatcatgaatctaataacgatattttccccaaaaaagagaaaaaattctgcaaacttccaccgtcatcggataatttaaccaaaatcatggtgaattcaaaatggtacaatcctttccttttttggtttactgtcaagactgaacttagagcctgccagaaggagaactacaaaaggaaaaacagaaaattgggaattatcacatcgattaaaggttcatgctataagttgatactcaaccagaatctagtagcaatattcgaggaagacagcagtggatactcagatcataaaaaaagaaaaaaacgatgctactatctaactcccgaaatggtccttatgttctccgatgtaactgaaggaagattgatgattgatgttgcaatgagatttgacaaaaagtacaaaactctagagaaaaaggctttgaaattatggtttcttatagacgagttatttccttctatgggaaatagagtgtataatattatatccatgcttgagcctttgactctcgcgatattacaggttaaggatgagtcaaggttgttgagaggtgcattcatgcatcattgtttaggtgacctcttcgaagaacttcgagagtccaagaactacccggaagatgagatcaagagatttgccaacgacctaataaatgtcatgacctgtcgggacattcatttagtagcagaattcttctcattctttaggactttcggacatccaatattgaacgctcaaactgcagccaggaaagttagagagtacatgttagcagataaaatccttgagtacgaacctatcatgaaaggtcatgcgattttctgtgctataatcataaatggatttagagatagacatggaggagtttggcctcctcttgatcttccaaaacattgttcaaagaacataatatctctcaaaaatacaggtgaaggggtaacttatgaagtagcaataaacaattggagatcatttgtcgggttaaagttcaaatgttttatgggtctcaatttagacaatgatctcagcatgtacatgaaagataaagcattatcacctttaagggatctttgggattcaatctattcacgtgaagtaatgtcctaccaaccacctagaaacaaaaaatcaagaagattggttgaggttttcgttgatgatcaggactttgatcccgttgatatgataaattatgttctgaccggagaatatctcagagatgatgatttcaatgcttcttatagtttaaaagagaaagagaccaaacaagttggcaggttgtttgctaagatgacttataaaatgagggcctgtcaagttattgctgagaatttaattgcacatgggattgggagatatttccatgaaaacgggatggttaaggatgagcatgagctcagcaaatcactgtttcaattgtctatatcaggaataccaagagggaacaaaaacaacaaatcgacgaacgacacaatccacgaaagcaagatcgagaataaccattcctttaaaaacatccagaatcgatcatttcgaaagacggataacccatacaatagatttaacattgataacccaactttcttatccccaaactgtaaccccaagtataaccgtaagaattcagagacaataggtatattctctcgtgcagaaaccaaaagcatgattagagaacagaaaagtcacagagaagtcaaaataaataagctagatatcggcagtgataatgaagagcaaggaaaagagatagatgccgccaagtacaaaatcacggacaacccaaatccacacataaatcctcaagatcaacccggaatctgtcaagaagacaaaggcaaagaaggagcaaagtcagatctcacagaaggcatgagttttctggagatgcacacactctttaacccgagtaagagcgatatcagaacaaatctcgaattggaaaagagttcactttcaaaccctggatttatatcacaaaaagagaaaagaggcaaaacttataatgaatcccattcactgggaaagttctctaaagaggatgaagaaagatacgatgtcatcagtgcattcctgacaacagatttacgaaaattctgcttaaattggagacatgaatcaatcggcatttttgcaagaaggatggacgaaatctatggtttgcctggtttctttaattggatgcacagaagactagagcgatctgtgttatatgttgcggaccctcattgcccgccgtctatcaatgaacatatcgatctaaacgattcacccgaaagagacatatttatacatcatccgaaagggggtatagaaggatacagccaaaaactgtggacaatagcgactatcccttttctattcctcagtgctcatgagacaaacacccggatagcggcagttgtacaaggtgacaatcaatcaattgcaattacacataaggtccaccctcatttgccttacaaaatgaagaaagaactctctgcaatgcaggcaaaaaaatatttttcaaggttacggcacaacatgaaggcattagggcatgaattgaaggcgaccgagactatcattagtactcatttcttcatttattccaagaaaatccactatgacggggctgttttatcacaatctctgaaatcaatggcaaggtgtgtattttggtcagaaacccttgttgatgaaactagagcagcatgcagtaatatcagcacaacaattgcaaaggctattgagaatggttatagcaggagatctggctatctgataaatgttcttaaaaccatccaacaaattaatatatcattgagttttaatataaatgaatgcatgacagatgacataatcagaccgtttagagataatccaaactggatcaaacatgccgcattaatccccgccagcttgggaggactcaactatatgaacatgtctcgattgtatgtgaggaatataggggatccagtcacagcatcgatagcagatgttaagagaatgattctcggtggtgtactacccattggaatactccacaatatcatgttgcaagaacccggtgatgccacttatttggactggtgtagtgatccatactccatcaacctaaagcagactcaaagtatcacaaaagttataaagaacataacggcaagagtgatactaaggaattcggtcaatccactgctcaaaggtctatttcatgaaggtgcttatgaggaggacactgaattagcaacattcattttggacaggagagtcatcttaccacgagtcggtcacgagatcttaaacaactccatcacaggagcaagagaagagatctcgggcttactggataccacaaaaggattgataagaattggcatagcaaagggaggattaactcagagaacattatctcgaatttccaattatgattatgaacaatttttgaacctaatgaatatgttgaagaacaaagaacaaaacagtgtcatttccctgtcagcttgctctgttgactttgctatagctttaagaagcaggatgtggaggaaattggcaaaaggaagattaatatatggtttagaagtccctgatccaatagaagcaatgattggctttctcattcttgggagtgaaaattgtctactctgtgattcaggaagcaaaaactatacctggtttttcataccaaaggatgtacagttggataagattgataaagatcacgcatcaataagggtaccctatgtcggatcaactaccgaagaaagatcagagataaagttaggatccgtgaaaaatccaagcaaatccctgaaatctgctataagactcgcaactgtgtacacttgggcatttggcacaagtgatgctgaatggtgggaggcttggtacttgtctaatcaacgagcaaatatacccttagatgttctcaaaacgataacacctatatctacttcaacgaatattgctcatagattacgagaccgatcaacacaggttaaatacgccagtacatctcttaacagagtatcgcggcatgtaacaattagtaacgataacatgaattttgaatttgacggggttaaaatggataccaacttgatttatcaacaagtcatgctgttagggctttcatgcttggagagtttattccgaaataggaaaatgacaaatagttacaatatcgtgtaccatttacacgttcaagaacattgttgtgtaaaggctctgaatgatttaccttatacaccgtcaacacatccagtgccaaattatacagaagttagagataataggttaatttacgatcctcaacctatattagaatttgatgagctaagattagcaattcagcaaacaaagaaagtagatttggaattttcattgtgggatacaaaagaacttcatgagaatttagctcaaagtttagcgattacagtaacggatattatgacaaaatctgataaagatcatattaaagaccaaagaagtatagatgttgatgataatattaagacactaataactgagtttttattagtagaccctgaaatgtttgccgtaaatttaggattgcatatatcaataaaatggtcatttgatattcactttaaaagaccaagaggacgctatagcatgatagaatacttgactgatcttttggataatacttcttctcatgtttatcgaatccttactaatgtattatctcatcccagagttatgagaaaattcactaatgccgggctactagtaccgaaatacggtccctaccttacaagtcaagatttcaaaaagatggcggtagatttcataataacagcgtataccacatttttgaccaattggtgtaataataacaagttttcaattctaatacctgaacaagaccctgatatacttgaattaagaaaagacatcactcatgcaaggcatttatgtatgatctcggatctttactgctactctttcaagcaaccttggataaaggagcttacaccacaagagaagatctgcgtcatggaggacttcatagccaattgtgttgctaatgatcaaacaagtgcgggctggaacataacgcccttaagagtttacaatctccctgcatcgaccacatacatcaggagagggataataaaacaattaagaatccgtcaaagcaatgagcctattgatctggaagatattaggattggtcagaaccccgattttgtgaataaacctattgagttttgtagcagtgaattcggtatcacaatttataaccttgaagaaattcttcaatcaaatgtgcatctcagtgtaaatatgaacattgactcctcaacaagtaacaatactgaaaatcatttatttagaagggtaggcttgaactctacttcatcttataaagcactatctttaacacctgttattaaaagatatcatcaacagaacactaataggctgtttataggagaaggatcagggtctatgatgtatctttaccagaaaaccttgggggagacaatatgcttctttaattcgggagttcagtacaatgaggatctgggtcaaagggaacaatcattatacccgagtgaatacagtatctgtgaacaaggagtaaaaaaagaaaaccctctcaccgggcatgttataccactattcaatggaagaccagaaaccacatgggtaggcaatgatgattctttcaagtatatattggaacatactataaatagagacatcgggcttgttcactccgatatggaaacaggaatagggaaggataattatactatcttaaatgaacatgcacatcttatagcactgagccttacagtaatgattgatgatggaatcttggtgtctaaggtagcttatgcccctgggttttgcatctcttcattattgaatatgtaccggacatttttttcattagttctatgtgcgtttccaccgtatagcaattttgaatcaactgaattttacctgatttgcttgcaaaaaagtatacccggacctatcacaccagctagagccatccaacaaacgacgaagcaatctagagaagaggataatagtataactaataatatcctcaaaatcaaaaatcttgttcagaaagaatttatcaaaacagtaaagaaaaaatacgaaatccatccttcgtttaactgtcctatcaacttcacaaaggatgataaatatttaatgagtgttgggtttcaagccaatggtcctgatatgatacgtaaagagacgggctatgacataggtagcaatgtagagaatctccgagatgtcttaatcaagttgtttgcagatgcagtcaccttctatgatgatgtcacaaataaaaagaactttttaaatccttatccagtctacacaagaactcagtataaaattctgatggataaaatatgcaagaaagtcaccttatacaccttaatcatatcatgtaaaggatccaatcaatattgctgggaaattaaatcccaaataagaaagcattgtctcatacttgatttgaaaagtaaggtttttacaaaacttattccaaagggattaagagaaaggggtgactcaaaagggatgaagagcatatggttcactaaactaaccagtcaagaggtgaaaagatggtggaagatgatatcttacatcgtgataataagcaatccataaccacatccaacttgtcagttaaacacttaaatcacaataaacttgtcatcagattaaagaaaacttataattcccttttttaggt

SEQ ID NO: 3 - DNA, 1641 bp, Henipavirus nipahense
atggcagttatacttaacaagagatattattctaatctcttaatactgattttgatgatctcggagtgcagtgtcgggattttgcattatgagaaattgagtaagattgggcttgtcaaaggaataacaagaaaatacaagatcaaaagcaatcctctcacaaaagacattgttattaaaatgattccgaatgtgtcaaacatgtctcaatgcacggggagtgtcatggaaaactataaaacacgattaaacggtatcctaacgcctataaagggggcattagagatttacaagaacaacactcatgaccttgtcggtgatgtaagactggccggagttataatggcaggagttgctattggaattgcaaccgcagctcaaattactgcaggtgtagcattatatgaggcaatgaaaaatgctgacaacatcaacaaactcaaaagcagcatagaatcaactaatgaagctgttgttaagcttcaagagactgcagaaaagacagtctatgtactgaccgctttgcaggattacattaatactaacttggtaccgacaattgacaagataagctgcaaacagacggaactctcattagatctagcactatcaaagtacctctctgatttgctttttgtatttggtcccaaccttcaagacccagtttctaattcaatgactatacaggctatatctcaggcattcggtggaaattatgaaacactgctaagaacgttgggttacgctacagaagactttgatgatcttctagaaagtgacagcataacgggtcaaattatctacgttgatttaagtggctactacataattgtcagggtttattttcctatcctgactgaaatccaacaggcctatatccaagaattgttgccagtgagctttaacaatgacaattcagaatggatcagcattgtcccaaatttcatattggtaaggaacacattaatatcaaatatagagattggattttgcctaattacaaagaggagtgtgatctgcaaccaagattatgcaacacccatgacaaacaatatgagggaatgtttgacggggtcgactgagaagtgtcctcgagagctggtggtttcatcacacgttcccagatttgcactatctaacggggttttgtttgctaattgcataagcgtcacatgccagtgtcaaacaacaggtagggcaatctcacagtcaggagaacaaactctgctgatgattgataacaccacctgtcctacagccgtactcggtaatgtgatcatcagcttgggaaaatatcttgggtcagtaaattataactctgaaggcattgctattggtcctcctgtctttactgataaagttgacatatcaagtcaaatatctagcatgaatcagtccttacaacaatctaaggactatatcaaagaagctcaacgactccttgatactgttaacccgtcattaataagcatgttgtctatgatcatactgtatgtactatcaattgcatcattgtgtataggattgattacatttatcagttttatcattgttgagaaaaaaagaaacacctatagcagattagaggacaggagagtcagacctacaagtagtggggatctctattacattgggacgtag

SEQ ID NO: 4 - DNA, 1809 bp, Henipavirus nipahense
atgccgacagaaagcaagaaagttagattcgaaaatactgcttcagacaaggggaaaaatcctagtaaagttattaagagctactacggaaccatggacattaagaaaataaatgaagggttattggacagcaagatattaagtgctttcaacacagtgatagcactgcttggatccattgtaatcatagtgatgaatataatgatcatccaaaactacacaagatcaacagataatcaggccatgatcaaagatgcattgcagagtatccagcagcagatcaaggggcttgccgacaaaattggcacagagatagggccgaaagtatcactgattgatacatccagtactatcactattccagctaatattgggctgttaggttcaaagatcagccagtcaactgcaagtataaatgagaatgtgaatgaaaaatgcaaatttacactgcctcccttgaaaatccacgaatgtaacatttcttgtcctaacccactcccttttagagagtataagccgcagacagaaggagtgagcaatctggtaggattacctaataatatctgtctgcaaaagacatctaatcagatactgaaaccaaagctgatttcatacaccttacccgtagtcggtcaaagtggcacctgtatcacagacccactgctggctatggatgagggctactttgcatatagccacctggaaaaaatcggatcatgttcaagaggggtctccaaacaaagaataataggagttggagaggtactagacagaggtgacgaagtaccttctttgtttatgactaacgtctggaccccatcaaatccaaacaccgtttaccattgcagtgccgtgtacaacaatgaattctattatgtgctttgtgcagtgtcagttgttggagaccctattctgaatagcacctactggtccggatcactaatgatgactcgtctagctgtaaaacctaagaataatggtgagagttacaatcaacatcaatttgccttacggaatattgagaaagggaagtatgataaagttatgccatatggaccctcaggcatcaaacaaggtgacaccctgtactttcctgctgtaggatttttggtcaggacagagttcacatacaatgattcaaattgtcccatcgcagagtgtcaatacagcaaacctgaaaactgcaggctatctatggggattagaccaaacagtcattatatccttcgatctggactactaaaatacaatctatcggatgaggagaactctaaaattgtattcattgaaatatctgatcaaagactatctattggatctcctagcaaaatctatgattctttgggtcaacctgttttctaccaagcatctttttcatgggacactatgattaaatttggagatgtccaaacagttaaccctttagttgtaaattggcgtgacaacacggtaatctcaagacctgggcaatcacaatgccctagattcaacaagtgcccagaggtttgctgggaaggggtttataatgatgctttcctgattgatagaatcaattggataagcgcgggtgtattccttgacagcaaccagaccgcagagaatcctgtttttactgtattcaaagataatgaagtactttacagagcacaactagcttccgaggacaccaatgcacaaaaaacaataactaattgcttccttttgaagaataagatctggtgtatatcactggttgagatatacgacacaggagacaatgttataagacctaaactattcgcagttaagataccagagcaatgtacataa

SEQ ID NO: 5 - DNA, 1641 bp, Henipavirus hendraense
atggctacacaagaggttaggctaaagtgtttgctctgtgggatcatagttctggttttgtcattagaagggctaggaatactacattatgagaaacttagtaagatagggctggttaaaggtattacaagaaagtacaagattaagagtaaccctttgaccaaggatattgtaatcaaaatgatccctaatgtttcgaatgtctcaaagtgcaccgggactgttatggagaattacaaaagcagactcacagggattctctcaccaatcaaaggcgccatcgaactgtacaataataacacgcatgacctagttggtgatgtcaagcttgcaggtgtggtgatggcagggattgcaatcgggatagctactgctgcacaaatcacagcaggtgttgccttatatgaggcaatgaagaacgcagacaatatcaataaactcaagagcagcatagagtctacaaatgaggctgttgtcaaattacaggaaacagctgagaaaacagtctacgtccttactgctcttcaagattacatcaacactaaccttgttcctacaatagatcaaattagctgcaagcaaacagaactcgcattagacttggcgttgtctaagtatctgtctgatctgctctttgttttcggacctaacttacaggatccagtctctaattccatgactatccaagcaatatctcaagcatttgggggcaattacgaaaccttactgagaacgcttggttacgcgaccgaggatttcgacgaccttttagaaagtgatagcataacaggccagatagtctatgtagatctcagtagctattacataatagtaagggtgtattttcctatactaacagagatccaacaggcttatgtgcaggagttgcttccagtgagttttaataacgataattcagaatggatcagcattgtcccgaatttcgtgctgattaggaacacgctgatttcaaatatagaagttaagtactgcttaatcaccaagaaaagtgtgatttgtaatcaagactatgctacacccatgacggctagcgtgagagaatgcttgacaggatccacagataagtgcccaagggagttagtagtctcatcccatgttccaagatttgccctctcaggaggagtcttgtttgcaaattgtataagtgtgacatgtcagtgtcagactactgggagggcaatatctcaatcaggggaacagacactactgatgattgacaatactacctgcacaacagttgttctaggaaacataatcataagccttggaaaatatttgggatcaataaattacaattccgagagcattgctgttgggccaccagtctatacagacaaagttgatatctcaagtcagatatctagtatgaatcaatcactacaacaatctaaggattacattaaagaagctcaaaagatcttggacactgtgaatccgtcgttgataagtatgctatcaatgatcatcctttatgttttgtccattgcggcactgtgcattggtctgatcactttcataagctttgtaatagttgagaaaaagagagggaattacagcaggctagatgataggcaggtgcgaccggtcagtaatggtgatctgtattatattggaacataa

SEQ ID NO: 6 - DNA, 1641 bp, Henipavirus hendraense
atggctacacaagaggtcaggctaaagtgtttgctctgtgggatcatagttctggttttgtcattagaagggctagggatactacattatgagaaacttagtaagatagggctggttaaaggtattacaagaaagtacaagattaagagtaaccctttgaccaaggatattgtgatcaaaatgatccctaatgtctcgaatgtctcaaagtgcaccgggactgttatggagaattacaaaagcagactcacagggattctctcaccaatcaaaggcgccatcgaactgtacaataataacacgcatgacctagttggtgatgtcaagcttgcaggtgtggtgatggcagggattgcaatcgggatagctactgctgcacaaatcacagcaggtgttgccttatatgaggcaatgaagaacgcagacaatatcaataaactcaagagcagcatagagtctacaaatgaggctgttgtcaaattacaggaaacagctgagaaaacagtctacgtccttactgctcttcaagattacatcaacactaaccttgttcctacaatagatcaaattagctgcaagcaaacagagctcgcattagacttggcgttgtctaagtatctgtctgatctgctctttgttttcggacctaacttacaggatccagtctctaattccatgactatccaagcaatatctcaagcatttgggggcaattacgaaaccttactgagaacgcttggttacgcgaccgaggacttcgacgaccttttagaaagtgatagcataacaggccagatagtctatgtagatctcagtagctattacataatagtaagggtgtattttcccatactaacagagatccaacaggcttatgtgcaggagttgcttccagtgagttttaataacgataattcagaatggatcagcattgtcccgaatttcgtgctgattaggaacacgctgatttcaaatatagaagtcaagtactgcttaatcaccaagaaaagtgtgatttgtaatcaggactatgctacacccatgacggctagcgtgagagaatgcttgacaggatccacagataagtgcccaagggagttagtagtctcatcccatgttccaagatttgccctctcaggaggagtcttgtttgcaaattgtataagtgtgacatgtcagtgtcagactactgggagggcaatatctcaatcaggggaacagacactactgatgattgacaatactacctgcacaacagttgttctaggaaacataatcataagccttggaaaatatttgggatcaataaattacaattctgagagcattgctgttgggccaccagtctatacagacaaagttgatatctcaagtcagatatctagtatgaatcaatcactacaacaatctaaggattacattaaagaagctcaaaagatcttggacactgtgaatccgtcgttgataagtatgctatcaatgatcatcctttatgttttgtccattgcagcactgtgcattggtctgatcactttcataagctttgtaatagttgagaaaaagagagggaattacagcaggctagatgataggcaagtgcgaccggtcagtaatggtgatctgtattatattggaacataa

SEQ ID NO: 7 - DNA, 1815 bp, Henipavirus hendraense
atgatggctgattccaaattggtaagcctgaacaataatctatctggtaaaatcaaggatcaaggtaaagttatcaagaattattacggcacaatggacatcaagaaaattaacgatgggttattagatagtaagatacttggggcgtttaacacagtgatagctttgttgggatcaatcatcatcattgtgatgaatatcatgataattcaaaattacaccagaacgactgataatcaggcactaatcaaagagtcactccagagtgtacagcaacaaatcaaagctttaacagacaaaatcgggacagagataggccccaaagtctcactgattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccaaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcttggacagagtcactgtctctgattcgtcttgctgtaagaccaaaaagtgatagtggagactacaatcagaaatacatcgctataactaaagttgaaagagggaagtacgataaggtaatgccttacggtccatcaggtatcaagcaaggggatacattgtactttccggccgtcggtttcttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctgtctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagatcggctaaactgggttagtgctggtgtttatttaaacagtaaccaaactgcagagaaccctgtgtttgccgtattcaaggataacgagatcctttaccaagttccactggctgaggatgacacaaatgcacaaaaaaccatcacagattgcttcttgctggagaatgtcatatggtgtatatcactagtagaaatatacgataccggagacagtgtgataagaccaaaactgtttgcagtcaagatacctgcccaatgttcagagagttga

SEQ ID NO: 8 - DNA, 1815 bp, Henipavirus hendraense
atgatggctgattccaaattggtaagcctgaacaataatctatctggtaaaatcaaggatcaaggtaaagttatcaagaattattacggcacaatggacatcaagaaaattaacgatgggttattagatagtaagatacttggggcgtttaacacagtgatagctttgttgggatcaatcatcatcattgtgatgaatatcatgataattcaaaattacaccagaacgactgataatcaggcactaatcaaagagtcactccagagtgtacagcaacaaatcaaagctttaacagacaaaatcgggacagagataggccccaaagtctcactaattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccgaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcctggacagagtcactgtctctgattcgtcttgctgtaagaccaaaaagtgatagtggagactacaatcagaaatacatcgctataactaaagttgaaagagggaagtacgataaggtgatgccttacggtccatcaggtatcaagcaaggggatacattgtactttccggccgtcggttttttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctatctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagaccggctaaactgggttagtgctggtgtttatttaaacagtaaccaaactgcagagaaccctgtgtttgccgtattcaaggataacgagatcctttaccaagttccactggctgaagatgacacaaatgcacaaaaaaccatcacagattgcttcttgctggagaatgtcatatggtgtatatcactagtagaaatatacgatacaggagacagtgtgataaggccaaaactatttgcagtcaagatacctgcccaatgttcagagagttga

SEQ ID NO: 9 - DNA, 1638 bp, Mojiang virus
atggcactaaataaaaatatgttcagttcactgttccttggttatctattagtgtacgctacgactgttcagtctagtatacactatgactccttatctaaggtcggtgtcattaagggtctgacatacaactataagatcaagggttcgccatctacaaagctaatggtggtcaaattgatacctaacattgatagtgttaaaaactgtactcagaaacagtatgatgaatacaagaacttagtaaggaaagccttagaaccggtcaaaatggctattgacaccatgctcaataatgttaagtcgggtaataacaagtacagatttgcaggtgcaattatggctggagttgccctcggtgttgcaacagcagccactgttacagcagggatagctctccatagatcaaatgaaaatgcacaggcaattgcaaacatgaagagtgctattcaaaatacaaatgaggcagtaaagcaattgcaattggccaataaacaaacactagctgtgattgacaccataagaggagagatcaataacaatataatacccgttataaatcaattgagctgtgacacaattgggctcagtgtaggtataagactcactcagtactactctgaaataataactgcatttgggccagctttgcagaatccagtaaatacaaggattaccattcaagcaatatctagtgtgtttaatggcaactttgatgaactgctgaagattatggggtatacaagtggtgatctttatgaaattctacatagtgaattaattagaggcaacattatagacgttgatgtagatgcaggatacatagctctagaaatagaattccccaatctaacattggtacctaatgctgtagtacaggagttaatgcctatcagttataacatagacggggatgagtgggtcacacttgtgccaaggtttgtacttacaaggactacactgttatcaaatattgatacgagtagatgtacaatcacagatagtagtgtcatatgtgacaacgactacgccttgcctatgtcacacgagcttattggctgcttacagggagatacatctaagtgtgctagagagaaggtagtctcaagttatgtccctaaatttgcgttgtctgatgggttagtgtatgcaaattgcctcaatactatctgccgatgtatggatacagatactccaatctcacaaagtctcggagccactgtatcattactagacaacaagaggtgttcagtatatcaggtaggagatgtcttgatttctgtcggatcatatctaggagatggagaatataatgctgataatgtagagcttggcccacctatagttatagataagattgacataggaaatcagctggcaggtattaatcaaaccttacaagaggcagaagattacattgagaagtcagaagagttcttaaaaggggttaacccttcaattatcactcttggttccatggttgtcctttatatatttatgatattaatagccattgtgtcagtaatagcactagtattgtcaattaaattaacagtaaaaggtaacgtggtaaggcagcagttcacgtatactcagcatgttcctagcatggagaatatcaattatgtaagtcattaa

SEQ ID NO: 10 - DNA, 1878 bp, Mojiang virus
atggcaacgaatagagacaataccatcacatcagcagaggtctcccaagaagataaggttaaaaaatattatggagtggaaactgctgagaaggttgccgacagcataagtggtaataaggtattcatattgatgaatacacttctgatactgacaggtgctattattacaataacactaaatatcaccaacctgacagcggctaaaagtcaacagaatatgctgaaaataatccaagatgacgtgaatgccaaattagaaatgttcgtgaatcttgatcaattggtgaagggtgaaattaagccaaaagtgtcactcataaatacagcagtgagcgtcagcatacccggtcagatctcaaacctccagaccaaattcctgcaaaaatatgtttacttagaagaatctattactaagcagtgcacttgcaaccctttatctgggatatttccaacatcaggcccaacctaccctccaactgataaaccagacgatgataccacagatgatgacaaagtggacaccacgattaagcctattgagtaccccaagccggatgggtgcaatagaactggcgaccatttcacgatggagcccggagctaacttttatactgtccctaacctaggaccggcaagttctaattctgacgagtgttacacaaacccctctttttcaattgggtcctccatctatatgttttctcaagagattagaaaaacggactgcacagcaggagagatattatcaattcagatcgtcttaggccgaatagtagacaagggtcagcagggtcctcaagcatcacccttattagtatgggccgtcccaaatccaaagatcataaactcgtgtgctgtcgcagctggagacgagatgggatgggtgttatgctcagtgacattaactgcagcatcaggggagcccatacctcacatgtttgatgggttctggttgtataagttagaacctgacaccgaagttgtatcctatagaatcacaggctatgcttatctcttagataaacaatatgactctgtctttataggtaagggcggtggtattcagaaaggtaacgatctatactttcagatgtatggattgtccagaaataggcaaagttttaaggcactgtgtgaacatggatcatgcctcggcactggaggtggagggtatcaagtgttgtgtgacagggctgtgatgtctttcgggagtgaagaatcactaattacaaatgcatatctgaaggtgaatgatctggcaagtgggaaacctgtgataataggacagacatttccgccctcagattcttataaaggctcaaatggtcggatgtacactataggtgataaatatggtctgtatcttgctccgtcatcctggaacagatatcttagatttgggataacaccagatatttctgtaagatcaacaacctggttgaaaagtcaagatccgataatgaagattttgtcaacatgcacgaacactgatagagatatgtgtcctgaaatttgcaatactagaggttatcaagatatttttccattgtcggaggattcagagtattatacatacataggcataactcctaataatggtggaactaaaaactttgtggccgtacgtgactcagatggtcatatagcatccattgatattttacaaaattattatagtatcacctcagctactataagctgcttcatgtacaaagatgagatttggtgtattgcaatcacagaagggaaaaaacagaaagacaatcctcaacggatatatgcacattcttacaaaattaggcaaatgtgttataatatgaagtctgccacagtgactgtgggtaatgccaaaaatatcacaatcaggaggtattaa

SEQ ID NO: 11 - DNA, 1989 bp, Ghana virus
atgaagaaaaagacggacaatcccacaatatcaaagaggggtcacaaccattctcgaggaatcaaatctagagcgctactcagagagacagataattattccaatgggctaatagttgagaatttagttagaaactgtcatcatccaagtaagaacaatctaaactatactaagacacaaaaaagagattctacaatcccttatcgtgtggaagagagaaaaggacattatccaaagattaaacatcttattgataaatcttacaagcatataaaaagagggaagagaagaaatggtcataatgggaacattataactataattctgttgttgattttaattttgaagacacagatgagtgaaggtgctatccattacgagactctaagtaagatcggattaataaagggaatcaccagagagtacaaagtcaaaggaactccgtcaagtaaagacatagtcatcaaattgattccgaatgtcaccggtcttaacaagtgcacgaacatatcaatggaaaactataaagaacaacttgacaaaatactaattcctattaacaacataattgaattgtatgcaaactcaactaaatcagcccctgggaatgcacgttttgctggcgttataattgcaggagtggcattaggtgttgcagcggcagcccaaataactgccggcattgcactgcatgaagctcgacagaatgcagagagaattaatctcttaaaggatagcatttctgccactaacaacgcagtagcagaactccaggaagcaactggtggaatagtaaatgtcattacaggaatgcaagattacatcaatacaaatctagtcccgcagattgacaaactgcaatgtagtcagatcaaaacggcattagacatatctctctcccaatactattcagaaatattaacagtgttcggtccaaaccttcaaaatccagtaactacttccatgtcaatacaagccatatcacaatcctttgggggaaatatagatttgctcttaaacctactaggttacactgcaaacgacttattggatttgctcgaaagtaaaagtataacaggccaaataacatacataaatcttgaacattacttcatggtaatcagagtatattatcctataatgacaacaatcagcaatgcttatgtccaggaattgatcaaaattagcttcaatgtcgatggcagtgaatgggtatctcttgtaccctcgtatatattgattagaaactcatatctctcaaacatagacatatcagaatgtctcataaccaaaaattcagtgatatgtcgtcatgactttgcaatgccaatgagttacaccttaaaggaatgcctaactggagacactgaaaagtgtccgagagaggctgttgtaacctcatatgtcccaagatttgctatctccgggggagtgatttatgctaattgtctaagtacaacatgtcaatgctatcaaactggcaaagtaattgctcaagacggcagccaaacattgatgatgatcgataatcaaacatgttcaatagtaagaattgaagaaatcctcatatcaacagggaaatatctgggaagtcaggagtacaatacgatgcatgtgtcagtcggcaatcctgtcttcactgacaagctggacataacaagtcaaatttccaacatcaaccaatccattgaacaatccaaattttatctagataagtctaaggctatacttgacaagataaatctcaacttaattggctctgtaccgatatcaatacttttcataattgcgatcttatcattgattctctctattataacttttgtgattgtgatgataattgtcagaagatataacaaatacactcctcttataaactctgatccatccagtaggaggagtactatacaggacgtatatatcatcccgaaccccggagaacattcgattagatcagctgctcgatcaattgacagagatcgagattga

SEQ ID NO: 12 - DNA, 1899 bp, Ghana virus
atgccgcagaagactgtggaattcattaacatgaattcccctctagaaagaggggtcagcactctttcagataagaagaccctcaatcaatctaaaatcaccaagcaggggtattttgggttaggatcccacagtgagagaaattggaagaagcagaagaatcaaaatgatcattacatgactgtttcaaccatgattcttgagatattagttgtcctgggcatcatgtttaatctcatagttttaactatggtgtattatcagaatgacaacatcaatcaaaggatggcagaacttacaagcaatatcacagtcctgaatttaaatcttaatcaattgacaaacaaaattcaaagagaaattattcctaggatcactcttattgacacagcaaccaccattacaattcctagtgccattacttacatattagcaactctgacaaccagaatctcggaattattgccgtcaatcaaccaaaagtgtgagttcaagacaccgacacttgtcctgaatgactgcagaataaactgtaccccaccactaaacccgtctgatggagtgaaaatgagttctcttgccactaacttggttgcacatgggccctctccctgtagaaacttttcatccgtacctacaatttactattatcggattccaggattatacaatagaacagcattggacgaaagatgtatactaaacccgagattgacaataagcagtacaaaatttgcttatgtccactctgaatatgataaaaattgcaccagaggattcaaatactatgaattgatgacatttggagaaatactggagggtccggaaaaagaacccagaatgttttctaggtcattttattcgcccacaaatgctgtgaactatcattcttgtacgccgatcgtgactgtcaatgaaggatattttctttgccttgaatgcacctcctcagatcccttgtacaaagcaaatctatctaatagcacattccatttggtgatactgaggcataacaaggatgagaaaatagtttcaatgcctagctttaacctttctactgatcaagagtatgttcagataatccctgcagaaggtggcggcacagcagagagtggcaatctttacttcccttgtattggaaggctcttacacaaacgagtcacccatcctttatgcaaaaagtcaaattgttcgcgaactgatgatgaatcttgcctgaaaagttattacaatcaagggtcgcctcagcaccaagtagtcaactgtctgataaggatcagaaatgcacagagagataatccaacctgggatgttatcacagttgatctgactaatacatacccaggatcaaggagcaggatctttggaagcttctccaaaccgatgctttatcaatcatcagtatcatggcatactcttcttcaggtagcagagataacagacctagataagtatcaattggactggttggatacaccctatatatctcgtcctggaggatctgagtgccctttcggaaattattgtccaacggtatgctgggaagggacatataatgatgtctatagcttaactccaaataacgatctttttgtcactgtgtatttgaagagtgaacaagttgcagagaacccttatttcgcaatcttctcccgggatcaaatcttgaaagaattccctcttgatgcatggataagcagtgcacgaactacgacaatatcgtgcttcatgttcaacaatgaaatttggtgtatagctgcattagagatcacaagattgaatgatgacatcataagaccaatttattactctttctggctgcctactgattgccggacaccatatccccacaccggtaagatgaccagggttcccttgcgctccacatataactactaa

SEQ ID NO: 13 - DNA, 1638 bp, Artificial Sequence
  Description of Artificial Sequence Synthetic polynucleotide
atggcagttatacttaacaagagatattattctaatctcttaatactgattttgatgatctcggagtgcagtgtcgggattttgcattatgagaaattgagtaagattgggcttgtcaaaggaataacaagaaaatacaagatcaaaagcaatcctctcacaaaagacattgttattaaaatgattccgaatgtgtcaaacatgtctcaatgcacggggagtgtcatggaaaactataaaacacgattaaacggtatcctaacgcctataaagggggcattagagatttacaagaacaacactcatgaccttgtcggtgatgtaagactggccggagttataatggcaggagttgctattggaattgcaaccgcagctcaaattactgcaggtgtagcattatatgaggcaatgaaaaatgctgacaacatcaacaaactcaaaagcagcatagaatcaactaatgaagctgttgttaagcttcaagagactgcagaaaagacagtctatgtactgaccgctttgcaggattacattaatactaacttggtaccgacaattgacaagataagctgcaaacagacggaactctcattagatctagcactatcaaagtacctctctgatttgctttttgtatttggtcccaaccttcaagacccagtttctaattcaatgactatacaggctatatctcaggcattcggtggaaattatgaaacactgctaagaacgttgggttacgctacagaagactttgatgatcttctagaaagtgacagcataacgggtcaaattatctacgttgatttaagtggctactacataattgtcagggtttattttcctatcctgactgaaatccaacaggcctatatccaagaattgttgccagtgagctttaacaatgacaattcagaatggatcagcattgtcccaaatttcatattggtaaggaacacattaatatcaaatatagagattggattttgcctaattacaaagaggagtgtgatctgcaaccaagattatgcaacacccatgacaaacaatatgagggaatgtttgacggggtcgactgagaagtgtcctcgagagctggtggtttcatcacacgttcccagatttgcactatctaacggggttttgtttgctaattgcataagcgtcacatgccagtgtcaaacaacaggtagggcaatctcacagtcaggagaacaaactctgctgatgattgataacaccacctgtcctacagccgtactcggtaatgtgatcatcagcttgggaaaatatcttgggtcagtaaattataactctgaaggcattgctattggtcctcctgtctttactgataaagttgacatatcaagtcaaatatctagcatgaatcagtccttacaacaatctaaggactatatcaaagaagctcaacgactccttgatactgttaacccgtcattaataagcatgttgtctatgatcatactgtatgtactatcaattgcatcattgtgtataggattgattacatttatcagttttatcattgttgagaaaaaaagaaacacctatagcagattagaggacaggagagtcagacctacaagtagtggggatctctattacattgggtag

SEQ ID NO: 14 - DNA, 1644 bp, Artificial Sequence
  Description of Artificial Sequence Synthetic polynucleotide
atggctacacaagaggtcaggctaaagtgtttgctctgtgggatcatagttctggttttgtcattagaagggctagggatactacattatgagaaacttagtaagatagggctggttaaaggtattacaagaaagtacaagattaagagtaaccctttgaccaaggatattgtgatcaaaatgatccctaatgtctcgaatgtctcaaagtgcaccgggactgttatggagaattacaaaagcagactcacagggattctctcaccaatcaaaggcgccatcgaactgtacaataataacacgcatgacctagttggtgatgtcaagcttgcaggtgtggtgatggcagggattgcaatcgggatagctactgctgcacaaatcacagcaggtgttgccttatatgaggcaatgaagaacgcagacaatatcaataaactcaagagcagcatagagtctacaaatgaggctgttgtcaaattacaggaaacagctgagaaaacagtctacgtccttactgctcttcaagattacatcaacactaaccttgttcctacaatagatcaaattagctgcaagcaaacagagctcgcattagacttggcgttgtctaagtatctgtctgatctgctctttgttttcggacctaacttacaggatccagtctctaattccatgactatccaagcaatatctcaagcatttgggggcaattacgaaaccttactgagaacgcttggttacgcgaccgaggacttcgacgaccttttagaaagtgatagcataacaggccagatagtctatgtagatctcagtagctattacataatagtaagggtgtattttcccatactaacagagatccaacaggcttatgtgcaggagttgcttccagtgagttttaataacgataattcagaatggatcagcattgtcccgaatttcgtgctgattaggaacacgctgatttcaaatatagaagtcaagtactgcttaatcaccaagaaaagtgtgatttgtaatcaggactatgctacacccatgacggctagcgtgagagaatgcttgacaggatccacagataagtgcccaagggagttagtagtctcatcccatgttccaagatttgccctctcaggaggagtcttgtttgcaaattgtataagtgtgacatgtcagtgtcagactactgggagggcaatatctcaatcaggggaacagacactactgatgattgacaatactacctgcacaacagttgttctaggaaacataatcataagccttggaaaatatttgggatcaataaattacaattctgagagcattgctgttgggccaccagtctatacagacaaagttgatatctcaagtcagatatctagtatgaatcaatcactacaacaatctaaggattacattaaagaagctcaaaagatcttggacactgtgaatccgtcgttgataagtatgctatcaatgatcatcctttatgttttgtccattgcagcactgtgcattggtctgatcactttcataagctttgtaatagttgagaaaaagagagggaattacagcaggctagatgataggcaagtgcgaccggtcagtaatggtgatctgtattatattggaacataataa

SEQ ID NO: 15 - DNA, 1815 bp, Artificial Sequence
  Description of Artificial Sequence Synthetic polynucleotide
atgatggctgattccaaattggtaagcctgaacaataatctatctggtaaaatcaaggatcaaggtaaagttatcaagaattattacggcacaatggacatcaagaaaattaacgatgggttattagatagtaagatacttggggcgtttaacacagtgatagctttgttgggatcaatcatcatcattgtgatgaatatcatgataattcaaaattacaccagaacgactgataatcaggcactaatcaaagagtcactccagagtgtacagcaacaaatcaaagctttaacagacaaaatcgggacagagataggccccaaagtctcactaattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccgaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcctggacagagtcactgtctctgattcgtcttgctgtaagaccaaaaagtgatagtggagactacaatcagaaatacatcgctataactaaagttgaaagagggaagtacgataaggtgatgccttacggtccatcaggtatcaagcaaggggatacattgtactttccggccgtcggttttttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctatctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagaccggctaaactgggttagtgctggtgtttatttaaacagtaaccaaacagcagagaaccctgtgtttgccgtattcaaggataacgagatcctttaccaagttccactggctgaagatgacacaaatgcacaaaaaaccatcacagattgcttcttgctggagaatgtcatatggtgtatatcactagtagaaatatacgatacaggagacagtgtgataaggccaaaactatttgcagtcaagatacctgcccaatgttcagagagttga

SEQ ID NO: 16 - DNA, 1641 bp, Artificial Sequence
  Description of Artificial Sequence Synthetic polynucleotide
atggcactaaataaaaatatgttcagttcactgttccttggttatctattagtgtacgctacgactgttcagtctagtatacactatgactccttatctaaggtcggtgtcattaagggtctgacatacaactataagatcaagggttcgccatctacaaagctaatggtggtcaaattgatacctaacattgatagtgttaaaaactgtactcagaaacagtatgatgaatacaagaacttagtaaggaaagccttagaaccggtcaaaatggctattgacaccatgctcaataatgttaagtcgggtaataacaagtacagatttgcaggtgcaattatggctggagttgccctcggtgttgcaacagcagccactgttacagcagggatagctctccatagatcaaatgaaaatgcacaggcaattgcaaacatgaagagtgctattcaaaatacaaatgaggcagtaaagcaattgcaattggccaataaacaaacactagctgtgattgacaccataagaggagagatcaataacaatataatacccgttataaatcaattgagctgtgacacaattgggctcagtgtaggtataagactcactcagtactactctgaaataataactgcatttgggccagctttgcagaatccagtaaatacaaggattaccattcaagcaatatctagtgtgtttaatggcaactttgatgaactgctgaagattatggggtatacaagtggtgatctttatgaaattctacatagtgaattaattagaggcaacattatagacgttgatgtagatgcaggatacatagctctagaaatagaattccccaatctaacattggtacctaatgctgtagtacaggagttaatgcctatcagttataacatagacggggatgagtgggtaacacttgtgccaaggtttgtacttacaaggactacactgttatcaaatattgatacgagtagatgtacaatcacagatagtagtgtcatatgtgacaacgactacgccttgcctatgtcacacgagcttattggctgcttacagggagatacatctaagtgtgctagagagaaggtagtctcaagttatgtccctaaatttgcgttgtctgatgggttagtgtatgcaaattgcctcaatactatctgccgatgtatggatacagatactccaatctcacaaagtctcggagccactgtatcattactagacaacaagaggtgttcagtatatcaggtaggagatgtcttgatttctgtcggatcatatctaggagatggagaatataatgctgataatgtagagcttggcccacctatagttatagataagattgacataggaaatcagctggcaggtattaatcaaaccttacaagaggcagaagattacattgagaagtcagaagagttcttaaaaggggttaacccttcaattatcactcttggttccatggttgtcctttatatatttatgatattaatagccattgtgtcagtaatagcactagtattgtcaattaaattaacagtaaaaggtaacgtggtaaggcagcagttcacgtatactcagcatgttcctagcatggagaatatcaattatgtaagtcattaataa

SEQ ID NO: 17 - DNA, 1992 bp, Artificial Sequence
  Description of Artificial Sequence Synthetic polynucleotide
atgaagaaaaagacggacaatcccacaatatcaaagaggggtcacaaccattctcgaggaatcaaatctagagcgctactcagagagacagataattattccaatgggctaatagttgagaatttagttagaaactgtcatcatccaagtaagaacaatctaaactatactaagacacaaaaaagagattctacaatcccttatcgtgtggaagagagaaaaggacattatccaaagattaaacatcttattgataaatcttacaagcatataaaaagagggaagagaagaaatggtcataatgggaacattataactataattctgttgttgattttaattttgaagacacagatgagtgaaggtgctatccattacgagactctaagtaagatcggattaataaagggaatcaccagagagtacaaagtcaaaggaactccgtcaagtaaagacatagtcatcaaattgattccgaatgtcaccggtcttaacaagtgcacgaacatatcaatggaaaactataaagaacaacttgacaaaatactaattcctattaacaacataattgaattgtatgcaaactcaactaaatcagcccctgggaatgcacgttttgctggcgttataattgcaggagtggcattaggtgttgcagcggcagcccaaataactgccggcattgcactgcatgaagctcgacagaatgcagagagaattaatctcttaaaggatagcatttctgccactaacaacgcagtagcagaactccaggaagcaactggtggaatagtaaatgtcattacaggaatgcaagattacatcaatacaaatctagtcccgcagattgacaaactgcaatgtagtcagatcaaaacggcattagacatatctctctcccaatactattcagaaatattaacagtgttcggtccaaaccttcaaaatccagtaactacttccatgtcaatacaagccatatcacaatcctttgggggaaatatagatttgctcttaaacctactaggttacactgcaaacgacttattggatttgctcgaaagtaaaagtataacaggccaaataacatacataaatcttgaacattacttcatggtaatcagagtatattatcctataatgacaacaatcagcaatgcttatgtccaggaattgatcaaaattagcttcaatgtcgatggcagtgaatgggtatctcttgtaccctcgtatatattgattagaaactcatatctctcaaacatagacatatcagaatgtctcataaccaaaaattcagtgatatgtcgtcatgactttgcaatgccaatgagttacaccttaaaggaatgcctaactggagacactgaaaagtgtccgagagaggctgttgtaacctcatatgtcccaagatttgctatctccgggggagtgatttatgctaattgtctaagtacaacatgtcaatgctatcaaactggcaaagtaattgctcaagacggcagccaaacattgatgatgatcgataatcaaacatgttcaatagtaagaattgaagaaatcctcatatcaacagggaaatatctgggaagtcaggagtacaatacgatgcatgtgtcagtcggcaatcctgtcttcactgacaagctggacataacaagtcaaatttccaacatcaaccaatccattgaacaatccaaattttatctagataagtctaaggctatacttgacaagataaatctcaacttaattggctctgtaccgatatcaatacttttcataattgcgatcttatcattgattctctctattataacttttgtgattgtgatgataattgtcagaagatataacaaatacactcctcttataaactctgatccatccagtaggaggagtactatacaggacgtatatatcatcccgaaccccggagaacattcgattagatcagctgctcgatcaattgacagagatcgagattgatga

SEQ ID NO: 18 - DNA, 1899 bp, Artificial Sequence
  Description of Artificial Sequence Synthetic polynucleotide
atgccgcagaagactgtggaattcattaacatgaattcccctctagaaagaggggtcagcactctttcagataagaagaccctcaatcaatctaaaatcaccaagcaggggtattttgggttaggatcccacagtgagagaaattggaagaagcagaagaatcaaaatgatcattacatgactgtttcaaccatgattcttgagatattagttgtcctgggcatcatgtttaatctcatagttttaactatggtgtattatcagaatgacaacatcaatcaaaggatggcagaacttacaagcaatatcacagtcctgaacttaaatcttaatcaattgacaaacaaaattcaaagagaaattattcctaggatcactcttattgacacagcaaccaccattacaattcctagtgccattacttacatattagcaactctgacaaccagaatctcggaattattgccgtcaatcaaccaaaagtgtgagttcaagacaccgacacttgtcctgaatgactgcagaataaactgtaccccaccactaaacccgtctgatggagtgaaaatgagttctcttgccactaacttggttgcacatgggccctctccctgtagaaacttttcatccgtacctacaatttactattatcggattccaggattatacaatagaacagcattggacgaaagatgtatactaaacccgagattgacaataagcagtacaaaatttgcttatgtccactctgaatatgataaaaattgcaccagaggattcaaatactatgaattgatgacatttggagaaatactggagggtccggaaaaagaacccagaatgttttctaggtcattttattcgcccacaaatgctgtgaactatcattcttgtacgccgatcgtgactgtcaatgaaggatattttctttgccttgaatgcacctcctcagatcccttgtacaaagcaaatctatctaatagcacattccatttggtgatactgaggcataacaaggatgagaaaatagtttcaatgcctagctttaacctttctactgatcaagagtatgttcagataatccctgcagaaggtggcggcacagcagagagtggcaatctttacttcccttgtattggaaggctcttacacaaacgagtcacccatcctttatgcaaaaagtcaaattgttcgcgaactgatgatgaatcttgcctgaaaagttattacaatcaagggtcgcctcagcaccaagtagtcaactgtctgataaggatcagaaatgcacagagagataatccaacctgggatgttatcacagttgatctgactaatacatacccaggatcaaggagcaggatctttggaagcttctccaaaccgatgctttatcaatcatcagtatcatggcatactcttcttcaggtagcagagataacagacctagataagtatcaattggactggttggatacaccctatatatctcgtcctggaggatctgagtgccctttcggaaattattgtccaacggtatgctgggaagggacatataatgatgtctatagcttaactccaaataacgatctttttgtcactgtgtatttgaagagtgaacaagttgcagagaacccttatttcgcaatcttctcacgggatcaaatcttgaaagaattccctcttgatgcatggataagcagtgcacgaactacgacaatatcgtgcttcatgttcaacaatgaaatttggtgtatagctgcattagagatcacaagattgaatgatgacatcataagaccaatttattactctttctggctgcctactgattgccggacaccatatccccacaccggtaagatgaccagggttcccttgcgctccacatataactactaa

SEQ ID NO: 19 - DNA, 1566 bp, Artificial Sequence
  Description of Artificial Sequence Synthetic polynucleotide
atggtggtgatcctggacaagaggtgctactgcaacctgctgatcctgatcctgatgatcagcgagtgcagcgtgggcatcctgcactacgagaagctgtccaagatcggcctggtgaagggcgtgaccaggaagtacaagatcaagagcaaccccctgaccaaggacatcgtgatcaagatgatccccaacgtgagcgacatgagccagtgcaccggcagcgtgatggagaactacaagaccaggctgaacggcatcctgacccccatcaagggcgccctggagatctacaagaacaacacccacgacctggtgggcgacgtgagactggccggcgtgatcatggccggcgtggccatcggcatcgctacagccgcccagatcacagccggagtggccctgtacgaggccatgaagaacgccgacaacatcaacaagctgaagagcagcatcgagagcaccaacgaggccgtggtgaagctgcaggagaccgccgaaaagaccgtgtacgtgctgaccgccctgcaggactacatcaacaccaacctggtgcccaccatcgacaagatcagctgcaagcagaccgagctgtccctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtcagcttcaacaacgacgacagcgagtggatcagcatcgtgcccaacttcatcctggtgcggaacaccctgatcagcaacatcgagatcggcttttgcctgatcaccaagagaagcgtgatctgcaaccaggactacgccacccccatgaccaacaacatgagagagtgcctgaccggcagcaccgagaagtgccctcgggaactggtggtgtccagccacgtgcccaggttcgccctgtccaacggcgtgctgttcgccaactgcatcagcgtgacctgccagtgccagaccaccggcagggccatctcccagagcggcgagcagacactgctgatgatcgacaacaccacctgccccaccgccgtgctgggcaacgtgatcatcagcctgggaaagtacctgggcagcgtgaactacaacagcgagggcatcgccatcggccctcccgtgttcaccgacaaggtggacatcagcagccagatcagcagcatgaaccagagcctgcagcagagcaaggattacatcaaggaggcccagaggctgctggacaccgtgaaccccagcatgaagcagatcgaggacaagatcgaggagatcctgagcaagatctaccacatcgagaacgagatcgccaggatcaagaagctgatcggcgaggcccctggcggc

SEQ ID NO: 20 - DNA, 1566 bp, Artificial Sequence
  Description of Artificial Sequence Synthetic polynucleotide
atggccacccaggaggtgcggctgaagtgcctgctgtgcggcatcatcgtgctggtgctgtccctggagggcctgggcatcctgcactacgagaagctgtccaagatcggcctggtgaagggcatcaccaggaagtacaagatcaagagcaaccccctgaccaaggacatcgtgatcaagatgatccccaacgtgagcaacgtgtccaagtgcaccggcaccgtgatggagaactacaagagcaggctgaccggcatcctgagccccatcaagggcgccatcgagctgtacaacaacaacacccacgacctggtgggcgacgtgaagctggccggcgtggtgatggccggcatcgccatcggaatcgccacagccgcccagatcacagccggcgtggccctgtacgaggccatgaagaacgccgacaacatcaacaagctgaagagcagcatcgagagcaccaacgaggccgtggtgaagctgcaggagaccgccgaaaagaccgtgtacgtgctgaccgccctgcaggactacatcaacaccaacctggtgcccaccatcgaccagatcagctgcaagcagaccgagctggccctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacggcctgctggagagcgacagcatcaccggccagatcgtgtacgtggacctgagcagctactacatcatcgtgagggtgtacttccccatcctgaccgagatccagcaggcctacgtgcaggagctgctgcccgtcagcttcaacaacgacaacagcgagtggatcagcatcgtgcccaacttcgtgctgatcaggaacaccctgatcagcaacatcgaggtgaagtactgcctgatcaccaagaaaagcgtgatctgcaaccaggactacgccacccccatgaccgccagcgtgagagagtgcctgaccggcagcaccgacaagtgccctcgggaactggtggtgtccagccacgtgcccaggttcgccctgagcggcggagtgctgttcgccaactgcatcagcgtgacctgccagtgccagaccaccggcagggccatctcccagagcggcgagcagacactgctgatgatcgacaacaccacctgcaccaccgtggtgctgggcaacatcatcatcagcctgggaaagtacctgggcagcatcaactacaactccgagagcatcgccgtgggaccccccgtgtacaccgacaaggtggacatcagcagccagatcagcagcatgaaccagagcctgcagcagagcaaggattacatcaaggaggcccagaaaatcctggacaccgtgaaccccagcatgaagcagatcgaggacaagatcgaggagatcctgagcaagatctaccacatcgagaacgagatcgccaggatcaagaagctgatcggcgaggcccctggcggc

SEQ ID NO: 21 - DNA, 1656 bp, Henipavirus nipahense
atggaaaccgacactctgctgctgtgggtcctgctgctgtgggtccctggctcaactggcgacgcagcatcaacagataatcaggccatgatcaaagatgcattgcagagtatccagcagcagatcaaggggcttgccgacaaaattggcacagagatagggccgaaagtatcactgattgatacatccagtactatcactattccagctaatattgggctgttaggttcaaagatcagccagtcaactgcaagtataaatgagaatgtgaatgaaaaatgcaaatttacactgcctcccttgaaaatccacgaatgtaacatttcttgtcctaacccactcccttttagagagtataagccgcagacagaaggagtgagcaatctggtaggattacctaataatatctgtctgcaaaagacatctaatcagatactgaaaccaaagctgatttcatacaccttacccgtagtcggtcaaagtggcacctgtatcacagacccactgctggctatggatgagggctactttgcatatagccacctggaaaaaatcggatcatgttcaagaggggtctccaaacaaagaataataggagttggagaggtactagacagaggtgacgaagtaccttctttgtttatgactaacgtctggaccccatcaaatccaaacaccgtttaccattgcagtgccgtgtacaacaatgaattctattatgtgctttgtgcagtgtcagttgttggagaccctattctgaatagcacctactggtccggatcactaatgatgactcgtctagctgtaaaacctaagaataatggtgagagttacaatcaacatcaatttgccttacggaatattgagaaagggaagtatgataaagttatgccatatggaccctcaggcatcaaacaaggtgacaccctgtactttcctgctgtaggatttttggtcaggacagagttcacatacaatgattcaaattgtcccatcgcagagtgtcaatacagcaaacctgaaaactgcaggctatctatggggattagaccaaacagtcattatatccttcgatctggactactaaaatacaatctatcggatgaggagaactctaaaattgtattcattgaaatatctgatcaaagactatctattggatctcctagcaaaatctatgattctttgggtcaacctgttttctaccaagcatctttttcatgggacactatgattaaatttggagatgtccaaacagttaaccctttagttgtaaattggcgtgacaacacggtaatctcaagacctgggcaatcacaatgccctagattcaacaagtgcccagaggtttgctgggaaggggtttataatgatgctttcctgattgatagaatcaattggataagcgcgggtgtattccttgacagcaaccagaccgcagagaatcctgtttttactgtattcaaagataatgaagtactttacagagcacaactagcttccgaggacaccaatgcacaaaaaacaataactaattgcttccttttgaagaataagatctggtgtatatcactggttgagatatacgacacaggagacaatgttataagacctaaactattcgcagttaagataccagagcaatgtacataataa

SEQ ID NO: 22 - DNA, 1662 bp, Henipavirus hendraense
atggaaaccgacactctgctgctgtgggtcctgctgctgtgggtccctggctcaactggcgacgcagcaagaacgactgataatcaggcactaatcaaagagtcactccagagtgtacagcaacaaatcaaagctttaacagacaaaatcgggacagagataggccccaaagtctcactaattgacacatccagcaccatcacaattcctgctaacatagggttactgggatccaagataagtcagtctaccagcagtattaatgagaatgttaacgataaatgcaaatttactcttcctcctttaaagattcatgagtgtaatatctcttgtccgaatcctttgcctttcagagaataccgaccaatctcacaaggggtgagtgatcttgtaggactgccgaaccagatctgtctacagaagacaacatcaacaatcttaaagcccaggctgatatcctatactctaccaattaataccagagaaggggtttgcatcactgacccacttttggctgttgataatggcttcttcgcctatagccatcttgaaaagatcggatcatgtactagaggaattgcaaaacaaaggataataggggtgggtgaggtattggataggggtgataaggtgccatcaatgtttatgaccaatgtttggacaccacccaatccaagcaccatccatcattgcagctcaacttaccatgaagatttttattacacattgtgcgcagtgtcccatgtgggagatcctatccttaacagtacttcctggacagagtcactgtctctgattcgtcttgctgtaagaccaaaaagtgatagtggagactacaatcagaaatacatcgctataactaaagttgaaagagggaagtacgataaggtgatgccttacggtccatcaggtatcaagcaaggggatacattgtactttccggccgtcggttttttgccaaggaccgaatttcaatataatgactctaattgtcccataattcattgcaagtacagcaaagcagaaaactgtaggctttcaatgggtgtcaactccaaaagtcattatattttgagatcaggactattgaagtataatctatctcttggaggagacatcatactccaatttatcgagattgctgacaatagattgaccatcggttctcctagtaagatatacaattccctaggtcaacccgttttctaccaggcatcatattcttgggatacgatgattaaattaggcgatgttgataccgttgaccctctaagagtacagtggagaaataacagtgtgatttctagacctggacagtcacagtgtcctcgatttaatgtctgtcccgaggtatgctgggaagggacatataatgatgcttttctaatagaccggctaaactgggttagtgctggtgtttatttaaacagtaaccaaacagcagagaaccctgtgtttgccgtattcaaggataacgagatcctttaccaagttccactggctgaagatgacacaaatgcacaaaaaaccatcacagattgcttcttgctggagaatgtcatatggtgtatatcactagtagaaatatacgatacaggagacagtgtgataaggccaaaactatttgcagtcaagatacctgcccaatgttcagagagttga

SEQ ID NO: 23 - DNA, 1668 bp, Mojiang virus
atggcactaaataaaaatatgttcagttcactgttccttggttatctattagtgtacgctacgactgttcagtctagtatacactatgactccttatctaaggtcggtgtcattaagggtctgacatacaactataagatcaagggttcgccatctacaaagctaatggtggtcaaattgatacctaacattgatagtgttaaaaactgtactcagaaacagtatgatgaatacaagaacttagtaaggaaagccttagaaccggtcaaaatggctattgacaccatgctcaataatgttaagtcgggtaataacaagtacagatttgcaggtgcaattatggctggagttgccctcggtgttgcaacagcagccactgttacagcagggatagctctccatagatcaaatgaaaatgcacaggcaattgcaaacatgaagagtgctattcaaaatacaaatgaggcagtaaagcaattgcaattggccaataaacaaacactagctgtgattgacaccataagaggagagatcaataacaatataatacccgttataaatcaattgagctgtgacacaattgggctcagtgtaggtataagactcactcagtactactctgaaataataactgcatttgggccagctttgcagaatccagtaaatacaaggattaccattcaagcaatatctagtgtgtttaatggcaactttgatgaactgctgaagattatggggtatacaagtggtgatctttatgaaattctacatagtgaattaattagaggcaacattatagacgttgatgtagatgcaggatacatagctctagaaatagaattccccaatctaacattggtacctaatgctgtagtacaggagttaatgcctatcagttataacatagacggggatgagtgggtaacacttgtgccaaggtttgtacttacaaggactacactgttatcaaatattgatacgagtagatgtacaatcacagatagtagtgtcatatgtgacaacgactacgccttgcctatgtcacacgagcttattggctgcttacagggagatacatctaagtgtgctagagagaaggtagtctcaagttatgtccctaaatttgcgttgtctgatgggttagtgtatgcaaattgcctcaatactatctgccgatgtatggatacagatactccaatctcacaaagtctcggagccactgtatcattactagacaacaagaggtgttcagtatatcaggtaggagatgtcttgatttctgtcggatcatatctaggagatggagaatataatgctgataatgtagagcttggcccacctatagttatagataagattgacataggaaatcagctggcaggtattaatcaaaccttacaagaggcagaagattacattgagaagtcagaagagttcttaaaaggggttaacccttcaattatcactcttggttccatggttgtcctttatatatttatgatattaatagccattgtgtcagtaatagcactagtattgtcaattaaattaacagtaaaaaaatataacaaatttatagatgatcctgattattacaatgattacaaaagagaacgtattaatggcaaagccagtaagagtaacaatatatattatgtaggtgattga

SEQ ID NO: 24 - DNA, 1953 bp, Ghana virus
atgaagaaaaagacggacaatcccacaatatcaaagaggggtcacaaccattctcgaggaatcaaatctagagcgctactcagagagacagataattattccaatgggctaatagttgagaatttagttagaaactgtcatcatccaagtaagaacaatctaaactatactaagacacaaaaaagagattctacaatcccttatcgtgtggaagagagaaaaggacattatccaaagattaaacatcttattgataaatcttacaagcatataaaaagagggaagagaagaaatggtcataatgggaacattataactataattctgttgttgattttaattttgaagacacagatgagtgaaggtgctatccattacgagactctaagtaagatcggattaataaagggaatcaccagagagtacaaagtcaaaggaactccgtcaagtaaagacatagtcatcaaattgattccgaatgtcaccggtcttaacaagtgcacgaacatatcaatggaaaactataaagaacaacttgacaaaatactaattcctattaacaacataattgaattgtatgcaaactcaactaaatcagcccctgggaatgcacgttttgctggcgttataattgcaggagtggcattaggtgttgcagcggcagcccaaataactgccggcattgcactgcatgaagctcgacagaatgcagagagaattaatctcttaaaggatagcatttctgccactaacaacgcagtagcagaactccaggaagcaactggtggaatagtaaatgtcattacaggaatgcaagattacatcaatacaaatctagtcccgcagattgacaaactgcaatgtagtcagatcaaaacggcattagacatatctctctcccaatactattcagaaatattaacagtgttcggtccaaaccttcaaaatccagtaactacttccatgtcaatacaagccatatcacaatcctttgggggaaatatagatttgctcttaaacctactaggttacactgcaaacgacttattggatttgctcgaaagtaaaagtataacaggccaaataacatacataaatcttgaacattacttcatggtaatcagagtatattatcctataatgacaacaatcagcaatgcttatgtccaggaattgatcaaaattagcttcaatgtcgatggcagtgaatgggtatctcttgtaccctcgtatatattgattagaaactcatatctctcaaacatagacatatcagaatgtctcataaccaaaaattcagtgatatgtcgtcatgactttgcaatgccaatgagttacaccttaaaggaatgcctaactggagacactgaaaagtgtccgagagaggctgttgtaacctcatatgtcccaagatttgctatctccgggggagtgatttatgctaattgtctaagtacaacatgtcaatgctatcaaactggcaaagtaattgctcaagacggcagccaaacattgatgatgatcgataatcaaacatgttcaatagtaagaattgaagaaatcctcatatcaacagggaaatatctgggaagtcaggagtacaatacgatgcatgtgtcagtcggcaatcctgtcttcactgacaagctggacataacaagtcaaatttccaacatcaaccaatccattgaacaatccaaattttatctagataagtctaaggctatacttgacaagataaatctcaacttaattggctctgtaccgatatcaatacttttcataattgcgatcttatcattgattctctctattataacttttgtgattgtgatgataattaaatataacaaatttatagatgatcctgattattacaatgattacaaaagagaacgtattaatggcaaagccagtaagagtaacaatatatattatgtaggtgattgatga

SEQ ID NO: 25 - DNA, 1959 bp, Mojiang virus
atgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtagtggtaataaggtattcatattgatgaatacacttctgatactgacaggtgctattattacaataacactaaatatcaccaacctgacagcggctaaaagtcaacagaatatgctgaaaataatccaagatgacgtgaatgccaaattagaaatgttcgtgaatcttgatcaattggtgaagggtgaaattaagccaaaagtgtcactcataaatacagcagtgagcgtcagcatacccggtcagatctcaaacctccagaccaaattcctgcaaaaatatgtttacttagaagaatctattactaagcagtgcacttgcaaccctttatctgggatatttccaacatcaggcccaacctaccctccaactgataaaccagacgatgataccacagatgatgacaaagtggacaccacgattaagcctattgagtaccccaagccggatgggtgcaatagaactggcgaccatttcacgatggagcccggagctaacttttatactgtccctaacctaggaccggcaagttctaattctgacgagtgttacacaaacccctctttttcaattgggtcctccatctatatgttttctcaagagattagaaaaacggactgcacagcaggagagatattatcaattcagatcgtcttaggccgaatagtagacaagggtcagcagggtcctcaagcatcacccttattagtatgggccgtcccaaatccaaagatcataaactcgtgtgctgtcgcagctggagacgagatgggatgggtgttatgctcagtgacattaactgcagcatcaggggagcccatacctcacatgtttgatgggttctggttgtataagttagaacctgacaccgaagttgtatcctatagaatcacaggctatgcttatctcttagataaacaatatgactctgtctttataggtaagggcggtggtattcagaaaggtaacgatctatactttcagatgtatggattgtccagaaataggcaaagttttaaggcactgtgtgaacatggatcatgcctcggcactggaggtggagggtatcaagtgttgtgtgacagggctgtgatgtctttcgggagtgaagaatcactaattacaaatgcatatctgaaggtgaatgatctggcaagtgggaaacctgtgataataggacagacatttccgccctcagattcttataaaggctcaaatggtcggatgtacactataggtgataaatatggtctgtatcttgctccgtcatcctggaacagatatcttagatttgggataacaccagatatttctgtaagatcaacaacctggttgaaaagtcaagatccgataatgaagattttgtcaacatgcacgaacactgatagagatatgtgtcctgaaatttgcaatactagaggttatcaagatatttttccattgtcggaggattcagagtattatacatacataggcataactcctaataatggtggaactaaaaactttgtggccgtacgtgactcagatggtcatatagcatccattgatattttacaaaattattatagtatcacctcagctactataagctgcttcatgtacaaagatgagatttggtgtattgcaatcacagaagggaaaaaacagaaagacaatcctcaacggatatatgcacattcttacaaaattaggcaaatgtgttataatatgaagtctgccacagtgactgtgggtaatgccaaaaatatcacaatcaggaggtattaa

SEQ ID NO: 26 - DNA, 1926 bp, Ghana virus
atgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtcaaaatgatcattacatgactgtttcaaccatgattcttgagatattagttgtcctgggcatcatgtttaatctcatagttttaactatggtgtattatcagaatgacaacatcaatcaaaggatggcagaacttacaagcaatatcacagtcctgaacttaaatcttaatcaattgacaaacaaaattcaaagagaaattattcctaggatcactcttattgacacagcaaccaccattacaattcctagtgccattacttacatattagcaactctgacaaccagaatctcggaattattgccgtcaatcaaccaaaagtgtgagttcaagacaccgacacttgtcctgaatgactgcagaataaactgtaccccaccactaaacccgtctgatggagtgaaaatgagttctcttgccactaacttggttgcacatgggccctctccctgtagaaacttttcatccgtacctacaatttactattatcggattccaggattatacaatagaacagcattggacgaaagatgtatactaaacccgagattgacaataagcagtacaaaatttgcttatgtccactctgaatatgataaaaattgcaccagaggattcaaatactatgaattgatgacatttggagaaatactggagggtccggaaaaagaacccagaatgttttctaggtcattttattcgcccacaaatgctgtgaactatcattcttgtacgccgatcgtgactgtcaatgaaggatattttctttgccttgaatgcacctcctcagatcccttgtacaaagcaaatctatctaatagcacattccatttggtgatactgaggcataacaaggatgagaaaatagtttcaatgcctagctttaacctttctactgatcaagagtatgttcagataatccctgcagaaggtggcggcacagcagagagtggcaatctttacttcccttgtattggaaggctcttacacaaacgagtcacccatcctttatgcaaaaagtcaaattgttcgcgaactgatgatgaatcttgcctgaaaagttattacaatcaagggtcgcctcagcaccaagtagtcaactgtctgataaggatcagaaatgcacagagagataatccaacctgggatgttatcacagttgatctgactaatacatacccaggatcaaggagcaggatctttggaagcttctccaaaccgatgctttatcaatcatcagtatcatggcatactcttcttcaggtagcagagataacagacctagataagtatcaattggactggttggatacaccctatatatctcgtcctggaggatctgagtgccctttcggaaattattgtccaacggtatgctgggaagggacatataatgatgtctatagcttaactccaaataacgatctttttgtcactgtgtatttgaagagtgaacaagttgcagagaacccttatttcgcaatcttctcacgggatcaaatcttgaaagaattccctcttgatgcatggataagcagtgcacgaactacgacaatatcgtgcttcatgttcaacaatgaaatttggtgtatagctgcattagagatcacaagattgaatgatgacatcataagaccaatttattactctttctggctgcctactgattgccggacaccatatccccacaccggtaagatgaccagggttcccttgcgctccacatataactactaa

SEQ ID NO: 27 - DNA, 6 bp, Artificial Sequence
  Description of Artificial Sequence Synthetic oligonucleotide
acgcgt

SEQ ID NO: 28 - DNA, 1674 bp, Cedar virus
atgtctaacaagaggacaacagtattgatcataataagctatacgttattttatttgaataatgcagcaattgtagggtttgattttgataaattgaataaaataggtgtggtgcaagggagagtcctaaattataaaattaaaggagatccaatgacaaaagaccttgtcttgaaatttatccctaacatagtgaatatcactgaatgtgtgagagagcccttgagtaggtacaatgagaccgtgaggagattgcttttacctatacacaacatgcttgggttatacttgaataacacaaatgctaaaatgactgggttgatgatcgcgggtgtgatcatgggtgggatagcaataggtatagccacagcagctcagatcacagcaggttttgctctttatgaggcaaaaaagaacacagaaaatattcagaaattaacagacagcatcatgaaaacacaggactcgattgataaacttactgacagtgtggggacaagcatacttatattgaataagctacagacatacatcaacaatcaactggtaccaaatctagagcttctatcctgccgacaaaacaaaattgagtttgatctaatgttaaccaagtatttggtggatcttatgactgttattggtcctaatatcaataatcctgttaataaagatatgactattcaatctttgtcacttctttttgatggcaattatgatataatgatgtcagaacttggttatacacctcaggatttcttagatttgatagagagtaagagtataacagggcaaataatttatgttgatatggaaaacttgtacgttgtgatcaggacatatctacctaccctaattgaagtacctgatgcccaaatatatgagttcaacaaaataactatgagtagcaatggaggagaatacttgtcaaccatacctaatttcatattaataagaggtaattatatgtctaatatagatgttgcaacatgttatatgaccaaagcaagcgtaatttgtaatcaagattattcactcccgatgagccaaaacttaagaagctgttatcaaggtgagacagaatactgtcctgttgaggcagtcatcgcgtcacactctccaagatttgctcttacaaatggagttattttcgccaattgtataaatacaatttgtaggtgtcaagacaatggtaagactatcactcaaaacataaaccaattcgtaagcatgatcgacaacagtacttgtaatgatgtcatggtagataagtttactatcaaggtaggaaaatatatggggagaaaagatatcaataatattaatatccagataggaccgcagatcataattgataaggttgacttgtctaatgaaataaacaagatgaatcaatctttaaaagatagtattttctacctgagagaagccaagagaattttagactcagtaaatatcagtcttatatctccaagcgttcaattgtttctaataataatatcagtcctctcatttattatattattgattatcatagtatacttgtactgtaaatcaaaacattcatataaatataacaaatttatagatgatcctgattattacaatgattacaaaagagaacgtattaatggcaaagccagtaagagtaacaatatatattatgtaggtgattaa

SEQ ID NO: 29 - DNA, 1869 bp, Cedar virus
atgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtctgcacgatatcaagttttgtatttattgtatattctcactgctaattatcattacaataatcaatataatcacaatatcaattgttataactcgtctgaaagtacatgaagagaataatggcatggaatctcctaatttacaatctattcaagatagtctctcatctcttactaacatgatcaatacagagataactcctagaatagggattttagttacagccacttctgttactctctcttcatctatcaattatgtcgggactaagacaaatcaactggtcaatgaattaaaagattatataaccaaaagttgtggctttaaggtccctgaattaaagttacatgaatgcaacataagttgtgctgatccaaaaattagcaaatctgcaatgtacagcaccaatgcctatgccgagcttgctggtccacctaagatattttgtaaaagtgtatccaaagaccccgactttagactgaagcagatagattatgtaataccagtgcagcaagatcggtctatttgtatgaacaaccctttattggatatttctgatgggttttttacctacatacattatgaaggaataaatagctgtaaaaaatcagattcatttaaagtgctgctgtcacatggtgaaatagttgacaggggtgattatcgaccatcattatatctattatcaagtcattaccatccttattcaatgcaggtaataaactgtgtacctgtgacttgtaaccagtcatcctttgtattctgtcatatctccaacaacactaaaacattggacaattcagattactcgtcagacgagtactacataacatatttcaatggcatagatcgtcccaaaaccaagaagattcccattaacaatatgacagcagacaatcgttatatccattttacattctcaggtgggggaggtgtatgtttaggtgaagaatttattattcctgttaccacagtcatcaatactgatgtattcacgcatgattattgtgagagtttcaactgttcagtccaaaccggtaaaagtctaaaggagatatgctctgagtcattaagatctccaacgaactcatcgcgatacaatttaaacggaatcatgattataagtcaaaacaacatgacagattttaagattcagttgaatggtataacttataacaaactgtcattcggaagtcctggaagactgagcaagacactgggccaggtcctttattaccaatcttcaatgagttgggatacttatctaaaggcaggatttgtcgagaaatggaaaccctttaccccgaattggatgaacaatactgtgatatccagacctaaccaaggtaattgtccaaggtatcataaatgccccgagatatgttatggagggacatacaatgatattgctcctttagatctaggaaaagacatgtatgttagcgttattctagattcagatcagcttgcagagaatccagagattacagtatttaactctactactatactttataaggagagagtatccaaagatgaactaaacacaagaagtactacaacgagctgttttcttttcctagatgaaccttggtgtatatcagtattagaaacaaacagatttaacggcaaatctattaggcccgagatttattcatacaaaattcctaagtattgttaa

SEQ ID NO: 30 - DNA, 1566 bp, Henipavirus nipahense
atggcagttatacttaacaagagatattattctaatctcttaatactgattttgatgatctcggagtgcagtgtcgggattttgcattatgagaaattgagtaagattgggcttgtcaaaggaataacaagaaaatacaagatcaaaagcaatcctctcacaaaagacattgttattaaaatgattccgaatgtgtcaaacatgtctcaatgcacggggagtgtcatggaaaactataaaacacgattaaacggtatcctaacgcctataaagggggcattagagatttacaagaacaacactcatgaccttgtcggtgatgtaagactggccggagttataatggcaggagttgctattggaattgcaaccgcagctcaaattactgcaggtgtagcattatatgaggcaatgaaaaatgctgacaacatcaacaaactcaaaagcagcatagaatcaactaatgaagctgttgttaagcttcaagagactgcagaaaagacagtctatgtactgaccgctttgcaggattacattaatactaacttggtaccgacaattgacaagataagctgcaaacagacggaactctcattagatctagcactatcaaagtacctctctgatttgctttttgtatttggtcccaaccttcaagacccagtttctaattcaatgactatacaggctatatctcaggcattcggtggaaattatgaaacactgctaagaacgttgggttacgctacagaagactttgatgatcttctagaaagtgacagcataacgggtcaaattatctacgttgatttaagtggctactacataattgtcagggtttattttcctatcctgactgaaatccaacaggcctatatccaagaattgttgccagtgagctttaacaatgacaattcagaatggatcagcattgtcccaaatttcatattggtaaggaacacattaatatcaaatatagagattggattttgcctaattacaaagaggagtgtgatctgcaaccaagattatgcaacacccatgacaaacaatatgagggaatgtttgacggggtcgactgagaagtgtcctcgagagctggtggtttcatcacacgttcccagatttgcactatctaacggggttttgtttgctaattgcataagcgtcacatgccagtgtcaaacaacaggtagggcaatctcacagtcaggagaacaaactctgctgatgattgataacaccacctgtcctacagccgtactcggtaatgtgatcatcagcttgggaaaatatcttgggtcagtaaattataactctgaaggcattgctattggtcctcctgtctttactgataaagttgacatatcaagtcaaatatctagcatgaatcagtccttacaacaatctaaggactatatcaaagaagctcaacgactccttgatactgttaacccgtcaatgaagcagatcgaggacaagatcgaggagatcctgagcaagatctaccacatcgagaacgagatcgccaggatcaagaagctgatcggcgaggcccctggcggc

SEQ ID NO: 31 - DNA, 1746 bp, Henipavirus nipahense
atggaaaccgacactctgctgctgtgggtcctgctgctgtgggtccctggctcaactggcgacgtcgacatgaagcagatcgaggacaagctggaggagatcgagagcaagctgaagaagatcgagaacgagctggccaggatcaagaaggtcgactacacctcaacagataatcaggccatgatcaaagatgcattgcagagtatccagcagcagatcaaggggcttgccgacaaaattggcacagagatagggccgaaagtatcactgattgatacatccagtactatcactattccagctaatattgggctgttaggttcaaagatcagccagtcaactgcaagtataaatgagaatgtgaatgaaaaatgcaaatttacactgcctcccttgaaaatccacgaatgtaacatttcttgtcctaacccactcccttttagagagtataagccgcagacagaaggagtgagcaatctggtaggattacctaataatatctgtctgcaaaagacatctaatcagatactgaaaccaaagctgatttcatacaccttacccgtagtcggtcaaagtggcacctgtatcacagacccactgctggctatggatgagggctactttgcatatagccacctggaaaaaatcggatcatgttcaagaggggtctccaaacaaagaataataggagttggagaggtactagacagaggtgacgaagtaccttctttgtttatgactaacgtctggaccccatcaaatccaaacaccgtttaccattgcagtgccgtgtacaacaatgaattctattatgtgctttgtgcagtgtcagttgttggagaccctattctgaatagcacctactggtccggatcactaatgatgactcgtctagctgtaaaacctaagaataatggtgagagttacaatcaacatcaatttgccttacggaatattgagaaagggaagtatgataaagttatgccatatggaccctcaggcatcaaacaaggtgacaccctgtactttcctgctgtaggatttttggtcaggacagagttcacatacaatgattcaaattgtcccatcgcagagtgtcaatacagcaaacctgaaaactgcaggctatctatggggattagaccaaacagtcattatatccttcgatctggactactaaaatacaatctatcggatgaggagaactctaaaattgtattcattgaaatatctgatcaaagactatctattggatctcctagcaaaatctatgattctttgggtcaacctgttttctaccaagcatctttttcatgggacactatgattaaatttggagatgtccaaacagttaaccctttagttgtaaattggcgtgacaacacggtaatctcaagacctgggcaatcacaatgccctagattcaacaagtgcccagaggtttgctgggaaggggtttataatgatgctttcctgattgatagaatcaattggataagcgcgggtgtattccttgacagcaaccagaccgcagagaatcctgtttttactgtattcaaagataatgaagtactttacagagcacaactagcttccgaggacaccaatgcacaaaaaacaataactaattgcttccttttgaagaataagatctggtgtatatcactggttgagatatacgacacaggagacaatgttataagacctaaactattcgcagttaagataccagagcaatgtacataa

SEQ ID NO: 32 - DNA, 1758 bp, Henipavirus hendraense
atggaaaccgacactctgctgctgtgggtcctgctgctgtgggtccctggctcaactggcgacgtcgacatgaagcagatcgaggacaagctggaggagatcgagagcaagctgaagaagatcgagaacgagctggccaggatcaagaaggtcgactacacccggaccaccgacaaccaggccctgatcaaagagtccctgcagagcgtccagcagcagatcaaggccctgaccgacaagatcggcaccgagatcggccccaaagtgtccctgatcgacaccagcagcaccatcaccatccccgccaacatcgggctgctgggctccaagatcagccagagcaccagctccatcaacgagaacgtgaacgacaagtgcaagttcaccctgccccccctgaagatccacgagtgcaacatcagctgccccaaccccctgcccttccgggagtaccggcccatcagccagggcgtgagcgacctggtgggcctgcccaaccagatctgcctgcagaaaaccacctccaccatcctgaagccccggctgatcagctacaccctgcccatcaacacccgggagggcgtgtgcatcaccgaccctctgctggccgtggacaacggcttcttcgcctacagccacctggaaaagatcggcagctgcacccggggcattgccaagcagcggatcatcggcgtgggcgaggtgctggaccggggcgacaaggtgcccagcatgttcatgaccaacgtgtggaccccccccaaccccagcacaatccaccactgcagcagcacctaccacgaggacttctactacaccctgtgcgccgtgagccacgtgggcgaccccatcctgaacagcaccagctggaccgagagcctgagcctgatccggctggccgtgcggcccaagagcgacagcggcgactacaaccagaagtatatcgccatcaccaaggtggagcggggcaagtacgacaaagtgatgccctacggccccagcggcatcaagcagggcgacacactgtacttccccgccgtgggcttcctgccccggaccgagttccagtacaacgacagcaactgccccatcatccactgcaagtacagcaaggccgagaactgcagactgagcatgggcgtgaacagcaagagccactacatcctgcggagcggcctgctgaagtacaacctgtccctgggcggcgacatcatcctgcagttcatcgagatcgccgacaaccggctgaccatcggcagccccagcaagatctacaacagcctgggccagcccgtgttctaccaggccagctacagctgggacaccatgatcaagctgggggacgtggacaccgtggaccccctgcgggtgcagtggcggaacaacagcgtgatcagcagacccggccagagccagtgcccccggttcaacgtgtgccccgaagtgtgctgggagggcacctacaacgacgcctttctgatcgaccggctgaactgggtgtccgccggagtgtacctgaactccaaccagaccgccgagaaccccgtgttcgccgtgttcaaggacaacgagatcctgtaccaggtgcccctggccgaggacgacaccaacgcccagaaaaccatcaccgactgctttctgctggaaaacgtgatctggtgcatcagcctggtggagatctacgacaccggcgactccgtgatccggcccaagctgtttgccgtgaagatccccgcccagtgcagcgagagctgatga

SEQ ID NO: 33 - DNA, 1677 bp, Mojiang virus
atggcactaaataaaaatatgttcagttcactgttccttggttatctattagtgtacgctacgactgttcagtctagtatacactatgactccttatctaaggtcggtgtcattaagggtctgacatacaactataagatcaagggttcgccatctacaaagctaatggtggtcaaattgatacctaacattgatagtgttaaaaactgtactcagaaacagtatgatgaatacaagaacttagtaaggaaagccttagaaccggtcaaaatggctattgacaccatgctcaataatgttaagtcgggtaataacaagtacagatttgcaggtgcaattatggctggagttgccctcggtgttgcaacagcagccactgttacagcagggatagctctccatagatcaaatgaaaatgcacaggcaattgcaaacatgaagagtgctattcaaaatacaaatgaggcagtaaagcaattgcaattggccaataaacaaacactagctgtgattgacaccataagaggagagatcaataacaatataatacccgttataaatcaattgagctgtgacacaattgggctcagtgtaggtataagactcactcagtactactctgaaataataactgcatttgggccagctttgcagaatccagtaaatacaaggattaccattcaagcaatatctagtgtgtttaatggcaactttgatgaactgctgaagattatggggtatacaagtggtgatctttatgaaattctacatagtgaattaattagaggcaacattatagacgttgatgtagatgcaggatacatagctctagaaatagaattccccaatctaacattggtacctaatgctgtagtacaggagttaatgcctatcagttataacatagacggggatgagtgggtaacacttgtgccaaggtttgtacttacaaggactacactgttatcaaatattgatacgagtagatgtacaatcacagatagtagtgtcatatgtgacaacgactacgccttgcctatgtcacacgagcttattggctgcttacagggagatacatctaagtgtgctagagagaaggtagtctcaagttatgtccctaaatttgcgttgtctgatgggttagtgtatgcaaattgcctcaatactatctgccgatgtatggatacagatactccaatctcacaaagtctcggagccactgtatcattactagacaacaagaggtgttcagtatatcaggtaggagatgtcttgatttctgtcggatcatatctaggagatggagaatataatgctgataatgtagagcttggcccacctatagttatagataagattgacataggaaatcagctggcaggtattaatcaaaccttacaagaggcagaagattacattgagaagtcagaagagttcttaaaaggggttaacccttcaattatcactccaagcgttcaattgtttctaataataatatcagtcctctcatttattatattattgattatcatagtatacttgtactgtaaatcaaaacattcatataaatataacaaatttatagatgatcctgattattacaatgattacaaaagagaacgtattaatggcaaagccagtaagagtaacaatatatattatgtaggtgattaataa

SEQ ID NO: 34 - DNA, 1962 bp, Mojiang virus
atgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtctgcacgatatcaagttttgtatttattgtatattctcactgctaattatcattacaataatcaatataatcacaatatcaattgttataactcgtctgaaagtacaacagaatatgctgaaaataatccaagatgacgtgaatgccaaattagaaatgttcgtgaatcttgatcaattggtgaagggtgaaattaagccaaaagtgtcactcataaatacagcagtgagcgtcagcatacccggtcagatctcaaacctccagaccaaattcctgcaaaaatatgtttacttagaagaatctattactaagcagtgcacttgcaaccctttatctgggatatttccaacatcaggcccaacctaccctccaactgataaaccagacgatgataccacagatgatgacaaagtggacaccacgattaagcctattgagtaccccaagccggatgggtgcaatagaactggcgaccatttcacgatggagcccggagctaacttttatactgtccctaacctaggaccggcaagttctaattctgacgagtgttacacaaacccctctttttcaattgggtcctccatctatatgttttctcaagagattagaaaaacggactgcacagcaggagagatattatcaattcagatcgtcttaggccgaatagtagacaagggtcagcagggtcctcaagcatcacccttattagtatgggccgtcccaaatccaaagatcataaactcgtgtgctgtcgcagctggagacgagatgggatgggtgttatgctcagtgacattaactgcagcatcaggggagcccatacctcacatgtttgatgggttctggttgtataagttagaacctgacaccgaagttgtatcctatagaatcacaggctatgcttatctcttagataaacaatatgactctgtctttataggtaagggcggtggtattcagaaaggtaacgatctatactttcagatgtatggattgtccagaaataggcaaagttttaaggcactgtgtgaacatggatcatgcctcggcactggaggtggagggtatcaagtgttgtgtgacagggctgtgatgtctttcgggagtgaagaatcactaattacaaatgcatatctgaaggtgaatgatctggcaagtgggaaacctgtgataataggacagacatttccgccctcagattcttataaaggctcaaatggtcggatgtacactataggtgataaatatggtctgtatcttgctccgtcatcctggaacagatatcttagatttgggataacaccagatatttctgtaagatcaacaacctggttgaaaagtcaagatccgataatgaagattttgtcaacatgcacgaacactgatagagatatgtgtcctgaaatttgcaatactagaggttatcaagatatttttccattgtcggaggattcagagtattatacatacataggcataactcctaataatggtggaactaaaaactttgtggccgtacgtgactcagatggtcatatagcatccattgatattttacaaaattattatagtatcacctcagctactataagctgcttcatgtacaaagatgagatttggtgtattgcaatcacagaagggaaaaaacagaaagacaatcctcaacggatatatgcacattcttacaaaattaggcaaatgtgttataatatgaagtctgccacagtgactgtgggtaatgccaaaaatatcacaatcaggaggtattaa

SEQ ID NO: 35 - DNA, 1968 bp, Ghana virus
atgaagaaaaagacggacaatcccacaatatcaaagaggggtcacaaccattctcgaggaatcaaatctagagcgctactcagagagacagataattattccaatgggctaatagttgagaatttagttagaaactgtcatcatccaagtaagaacaatctaaactatactaagacacaaaaaagagattctacaatcccttatcgtgtggaagagagaaaaggacattatccaaagattaaacatcttattgataaatcttacaagcatataaaaagagggaagagaagaaatggtcataatgggaacattataactataattctgttgttgattttaattttgaagacacagatgagtgaaggtgctatccattacgagactctaagtaagatcggattaataaagggaatcaccagagagtacaaagtcaaaggaactccgtcaagtaaagacatagtcatcaaattgattccgaatgtcaccggtcttaacaagtgcacgaacatatcaatggaaaactataaagaacaacttgacaaaatactaattcctattaacaacataattgaattgtatgcaaactcaactaaatcagcccctgggaatgcacgttttgctggcgttataattgcaggagtggcattaggtgttgcagcggcagcccaaataactgccggcattgcactgcatgaagctcgacagaatgcagagagaattaatctcttaaaggatagcatttctgccactaacaacgcagtagcagaactccaggaagcaactggtggaatagtaaatgtcattacaggaatgcaagattacatcaatacaaatctagtcccgcagattgacaaactgcaatgtagtcagatcaaaacggcattagacatatctctctcccaatactattcagaaatattaacagtgttcggtccaaaccttcaaaatccagtaactacttccatgtcaatacaagccatatcacaatcctttgggggaaatatagatttgctcttaaacctactaggttacactgcaaacgacttattggatttgctcgaaagtaaaagtataacaggccaaataacatacataaatcttgaacattacttcatggtaatcagagtatattatcctataatgacaacaatcagcaatgcttatgtccaggaattgatcaaaattagcttcaatgtcgatggcagtgaatgggtatctcttgtaccctcgtatatattgattagaaactcatatctctcaaacatagacatatcagaatgtctcataaccaaaaattcagtgatatgtcgtcatgactttgcaatgccaatgagttacaccttaaaggaatgcctaactggagacactgaaaagtgtccgagagaggctgttgtaacctcatatgtcccaagatttgctatctccgggggagtgatttatgctaattgtctaagtacaacatgtcaatgctatcaaactggcaaagtaattgctcaagacggcagccaaacattgatgatgatcgataatcaaacatgttcaatagtaagaattgaagaaatcctcatatcaacagggaaatatctgggaagtcaggagtacaatacgatgcatgtgtcagtcggcaatcctgtcttcactgacaagctggacataacaagtcaaatttccaacatcaaccaatccattgaacaatccaaattttatctagataagtctaaggctatacttgacaagataaatctcaacttaattggcccaagcgttcaattgtttctaataataatatcagtcctctcatttattatattattgattatcatagtatacttgtactgtaaatcaaaacattcatataaatataacaaatttatagatgatcctgattattacaatgattacaaaagagaacgtattaatggcaaagccagtaagagtaacaatatatattatgtaggtgattgatga

SEQ ID NO: 36 - DNA, 1929 bp, Ghana virus
atgctttctcagctccaaaaaaattacttagacaactcaaaccaacaaggtgataaaatgaacaacccagataagaaattaagtgtcaacttcaaccctttagaattagataaaggtcaaaaagatctcaataagtcttattatgttaaaaacaagaattataacgtttcaaatctattaaatgaaagtctgcacgatatcaagttttgtatttattgtatattctcactgctaattatcattacaataatcaatataatcacaatatcaattgttataactcgtctgaaagtaaatgacaacatcaatcaaaggatggcagaacttacaagcaatatcacagtcctgaacttaaatcttaatcaattgacaaacaaaattcaaagagaaattattcctaggatcactcttattgacacagcaaccaccattacaattcctagtgccattacttacatattagcaactctgacaaccagaatctcggaattattgccgtcaatcaaccaaaagtgtgagttcaagacaccgacacttgtcctgaatgactgcagaataaactgtaccccaccactaaacccgtctgatggagtgaaaatgagttctcttgccactaacttggttgcacatgggccctctccctgtagaaacttttcatccgtacctacaatttactattatcggattccaggattatacaatagaacagcattggacgaaagatgtatactaaacccgagattgacaataagcagtacaaaatttgcttatgtccactctgaatatgataaaaattgcaccagaggattcaaatactatgaattgatgacatttggagaaatactggagggtccggaaaaagaacccagaatgttttctaggtcattttattcgcccacaaatgctgtgaactatcattcttgtacgccgatcgtgactgtcaatgaaggatattttctttgccttgaatgcacctcctcagatcccttgtacaaagcaaatctatctaatagcacattccatttggtgatactgaggcataacaaggatgagaaaatagtttcaatgcctagctttaacctttctactgatcaagagtatgttcagataatccctgcagaaggtggcggcacagcagagagtggcaatctttacttcccttgtattggaaggctcttacacaaacgagtcacccatcctttatgcaaaaagtcaaattgttcgcgaactgatgatgaatcttgcctgaaaagttattacaatcaagggtcgcctcagcaccaagtagtcaactgtctgataaggatcagaaatgcacagagagataatccaacctgggatgttatcacagttgatctgactaatacatacccaggatcaaggagcaggatctttggaagcttctccaaaccgatgctttatcaatcatcagtatcatggcatactcttcttcaggtagcagagataacagacctagataagtatcaattggactggttggatacaccctatatatctcgtcctggaggatctgagtgccctttcggaaattattgtccaacggtatgctgggaagggacatataatgatgtctatagcttaactccaaataacgatctttttgtcactgtgtatttgaagagtgaacaagttgcagagaacccttatttcgcaatcttctcacgggatcaaatcttgaaagaattccctcttgatgcatggataagcagtgcacgaactacgacaatatcgtgcttcatgttcaacaatgaaatttggtgtatagctgcattagagatcacaagattgaatgatgacatcataagaccaatttattactctttctggctgcctactgattgccggacaccatatccccacaccggtaagatgaccagggttcccttgcgctccacatataactactaa

SEQ ID NO: 37 - DNA, 12 bp, Artificial Sequence
  Description of Artificial Sequence Synthetic oligonucleotide
gtcgactacacc

SEQ ID NO: 38 - DNA, 23 bp, Artificial Sequence
  Description of Artificial Sequence Synthetic oligonucleotide
taatacgactcactatagggaga

SEQ ID NO: 39 - DNA, 52 bp, Artificial Sequence
  Description of Artificial Sequence Synthetic oligonucleotide
gggagattggtctgatgagtccgtgaggacgaaacggagtctagactccgtc

SEQ ID NO: 40 - protein, 21 aa, Artificial Sequence
  Description of Artificial Sequence Synthetic peptide
METDTLLLWVLLLWVPGSTGD

SEQ ID NO: 41 - protein, 27 aa, Artificial Sequence
  Description of Artificial Sequence Synthetic peptide
MKQIEDKLEEIESKLKKIENELARIKK

SEQ ID NO: 42 - protein, 4 aa, Artificial Sequence
  Description of Artificial Sequence Synthetic peptide
VDYT

(19) United States
(12) Patent Application Publication
(10) Pub. No.: US 2025/0263442 A1
(43) Pub. Date: Aug. 21, 2025

(54) RECOMBINANT CEDAR VIRUS CHIMERAS

(71) Applicant: THE HENRY M. JACKSON FOUNDATION FOR THE ADVANCEMENT OF MILITARY MEDICINE, INC., Bethesda, MD (US)

(72) Inventors: Christopher C. Broder, Bethesda, MD (US); Moushimi Amaya, Bethesda, MD (US)

(73) Assignee: THE HENRY M. JACKSON FOUNDATION FOR THE ADVANCEMENT OF MILITARY MEDICINE, INC., Rockville, MD (US)

(21) Appl. No.: 18/557,247

(22) PCT Filed: Apr. 27, 2022
(86) PCT No.: PCT/US2022/026456

§ 371 Date: Oct. 25, 2023

Related U.S. Application Data
(60) Provisional application No. 63/180,516, filed on Apr. 27, 2021.

(51) Int. Cl. C07K 14/005; A61K 39/00; C12N 15/86

(52) U.S. Cl. CPC C07K 14/005; C12N 15/86; A61K 39/00; A61K 2039/5254; A61K 2039/70; C12N 2760/18222; C12N 2760/18234; C12N 2760/18243; C12N 2760/18244

(57) ABSTRACT

Described herein are replication-competent recombinant Cedar virus chimeras are described that are engineered to express antigenic surface or soluble proteins/polypeptides of a non-CedV henipavirus, such as of a pathogenic henipavirus, such as Nipah virus or Hendra virus. Vaccine compositions containing the recombinant Cedar virus chimeras are also described, as are therapeutic methods and uses for protecting against pathogenic henipavirus infection.

### CROSS-REFERENCE STATEMENT

This application is the U.S. National Stage of International Application No. PCT/US2022/026456, filed Apr. 27, 2022, and claims priority under 35 U.S.C. § 119(e) to U.S. Provisional Application 63/180,516 filed Apr. 27, 2021. The entire contents of each application is incorporated herein by reference.

### STATEMENT OF GOVERNMENT SUPPORT

This invention was made with federal support under U19 AI142764 01 awarded by the National Institutes of Health. The United States government has certain rights in the invention.

### SEQUENCE LISTING

The instant application contains a Sequence Listing which has been submitted electronically in ASCII format and is hereby incorporated by reference in its entirety. Said ASCII copy, created on Jun. 18, 2024, is named “103783-0347_SL.txt” and is 130,244 bytes in size.

### FIELD

The present disclosure relates to recombinant virus chimeras. More specifically, the disclosure relates to recombinant henipavirus chimeras.

### BACKGROUND

The following discussion is provided to aid the reader in understanding the disclosure and is not admitted to describe or constitute prior art thereto.

Henipaviruses are unique members of the Paramyxoviridae family (6). The prototypical henipavirus species, Hendra henipavirus, Hendra virus (HeV) and Nipah henipavirus, Nipah virus (NiV), are highly pathogenic Biological Safety Level-4 (BSL-4) select agents that emerged in the 1990s in Australia and peninsular Malaysia, respectively. They possess a broad host range spanning six mammalian orders (7) and cause infections that can result in severe respiratory illnesses and/or encephalitis with associated high fatality rates in humans (40-100%) (8) and other mammals, such as horses and pigs (2).

Presently, the well-characterized and well-accepted animal models of NiV (both the Malaysian (NiV-M) and Bangladesh (NiV-B) strains) and HeV infection and pathogenesis that replicate features of human henipavirus disease are the hamster, ferret, and African green monkey (7). There are no licensed NiV and HeV therapeutics or vaccines approved for human use currently available. A licensed equine vaccine for use in Australia (Equivac® HeV) was launched by Zoetis, Inc., in November 2012 on a minor use permit by the regulatory authority, the Australian Pesticides and Veterinary Medicines Authority (APVMA). All vaccinated horses are microchipped, and a database is maintained, and Equivac® HeV received full registration by the APVMA in 2015 (37, 40).

The genus Henipavirus also includes three additional species, two of which include viruses that were detected in, or isolated from, individual bats. The species, Ghanaian bat henipavirus, includes Ghana virus (GhV), which was identified by targeted RNA sequencing of fecal samples collected from straw-colored fruit bats (Eidolon helvum) (9). Cedar virus (CedV) (Cedar henipavirus), a non-pathogenic virus, was isolated from fruit bat urine samples in Australia (1). The third additional Henipavirus species, Mojiang virus (MojV) (Mojiang henipavirus), was discovered in 2012 specimens collected from yellow-breasted rats (Rattus flavipectus) in the Tongguan mine in Mojiang, Yunnan, China, where three miners had died of pneumonia of unknown etiology (10). No viral isolates of GhV and MojV have been recovered to date. GhV and MojV are known only from genetic sequence data and the pathogenic potential of either of these henipaviruses in animals or humans remains unknown.

Products of the P gene of NiV and HeV inhibit both double-stranded RNA signaling and interferon signaling. The P, V, W and C proteins all antagonize the host interferon response, and have now been demonstrated to play roles in the modulation of henipavirus pathogenicity (11). The NiV W protein is the most potent interferon antagonist and P protein the least (12). Infectious virus studies have shown that interferon signaling remains functional during henipavirus infection of human cell lines while interferon production was inhibited (13). NiV has been central to understanding the V, W, P and C protein's roles in antagonizing the innate immune responses via a diverse set of mechanisms. Notably, recombinant NiV lacking either the V or C protein suppressed the interferon response to similar levels as observed by wild-type NiV, but were significantly less pathogenic in hamsters suggesting that their roles in pathogenicity can also be independent of their interferon antagonist activity (14). Studies with recombinant NiV variants in the ferret model further detailed the relative importance of the V, W, C and P proteins in pathogenesis, revealing that their absence (with the exception of V) or disruption in their STAT1-binding capacity leads to an altered, but still lethal, pathological outcome in comparison to wild-type NiV, whereas only a recombinant NiV lacking the V protein resulted in a non-lethal productive infection in ferrets (15-17). Thus, the inhibition of viral recognition and innate immune signaling induction may be the major role of NiV P, V, and W proteins in NiV-mediated disease, and the inhibition of IFN signaling is less important (15).

In contrast, CedV does not cause pathogenesis following infection in ferrets, guinea pigs or hamsters (1, 18), or African green monkeys (Geisbert, T. W. and Broder, C. C., unpublished). Unlike HeV and NiV, CedV does not possess the RNA editing site within the phosphoprotein gene (P) that results in the expression of the V and W proteins (1). CedV infection of human cells elicits an interferon-β response (1). In contrast to the BSL-4 requirements for NiV and HeV (and potentially for additional henipaviruses, such as GhV and MojV), CedV can be used and manipulated at a lower biocontainment level (BSL-2) (4, 5). Cedar virus can thus serve as a novel, recombinant, henipavirus platform using BSL-2 containment for countermeasure developments against pathogenic henipaviruses.

CedV is a novel virus platform that has allowed the development of henipavirus targeted technologies for use in antiviral drug discovery, assays and diagnostics development, and live-attenuated vaccine development strategies. A reverse genetics system was previously developed to manipulate and generate novel recombinant CedV (rCedV) that has been used in antiviral discovery programs (e.g., anti-henipavirus therapies) (4,5). The genomic sequence of CedV is disclosed in U.S. Pat. No. 10,227,664.

The henipavirus virion bears surface projections composed of the F (fusion) and G (attachment) glycoproteins that are anchored in the viral membrane and are the major structural protein targets of neutralizing antibodies and the antigens employed in various vaccine strategies (7). Within the paramyxovirus family the attachment glycoproteins are also known as the receptor-binding protein (RBP). Infection of host cells by henipaviruses is mediated by the F and G glycoproteins (41). The F glycoprotein facilitates membrane fusion between the virus and host cell. The G glycoprotein consists of a characteristic stalk with a globular head that engages entry receptors on host cells leading to the fusion activation of F and virus infection. The native structure of G is a tetramer, while F is a trimer, and together they are the key determinants of infection and tropism (42). NiV and HeV utilize the host cell proteins ephrin-B2 and ephrin-B3 for entry (43-46). Ephrin-B2 and -B3 are members of a large family of ligands that bind to Eph receptors and are highly sequence conserved among mammals (47). In contrast, CedV has a uniquely broad ephrin tropism and can utilize mouse ephrin-A1, as well as human ephrin-A2, -A5, -B1 and -B2 as entry receptors (4).

Currently existing technology that has been extensively used as a surrogate assay or pseudovirus system or vaccine platform for henipaviruses are the recombinant Vesicular Stomatitis Virus (VSV) (a rhabdovirus) platform that has been used as a replication-incompetent pseudovirus whereby VSV with a deletion of its G glycoprotein is prepared by production in cell culture in which the F (fusion) and G (attachment) glycoproteins of NiV or HeV are transiently expressed in the cells so that progeny virions produced bud from the cells and acquire the F and G glycoproteins that are expressed on the cell surfaces (26-28). This is a tedious method of production, difficult to produce in large quantities, and although is suitable to measure antibody activity or neutralizing titers in sera against NiV and HeV, the measured titers are often quite different to those measured titers or neutralizing antibody activity when compared to assays conducted with authentic live NiV and HeV. Also, a replication competent VSV whereby its G glycoprotein is genetically replaced with NiV F and G, had enhanced pathogenicity in animals (29). Pseudoviruses using retroviruses have also been produced as surrogate neutralization assay systems for NiV and HeV, and these also have the same limitations described for VSV (30).

In regards to vaccines, recombinant VSV vectors have also been a widely used vaccine platform for NiV (31-36). Other viral vectors have also been used, but none are based on an authentic henipavirus that is naturally attenuated (reviewed in (37)).

Thus, there remains a need for constructs useful in vaccines against henipaviruses, and useful in antiviral drug discovery, assays and diagnostics development.

### SUMMARY

Described herein are novel chimeric forms (“chimeras”) of Cedar virus that express one or more pathogenic henipavirus proteins (e.g., F protein, G protein, or a combination thereof) and methods and uses of the same for treating, reducing the risk of, or preventing henipavirus infections or stimulating an immune response to henipaviruses. Additionally, the disclosed Cedar virus chimeras can be used to safely and effectively study or test vaccines and therapeutic agents against pathogenic henipaviruses.

In a first aspect, the present disclosure provides replication-competent recombinant Cedar virus (rCedV) chimeras wherein one or both of the F and G envelope glycoprotein genes of CedV are replaced with one or both of the F and G envelope glycoprotein genes, respectively, of a non-CedV henipavirus. The non-CedV henipavirus can be a pathogenic henipavirus, such as Hendra virus (HeV), Nipah virus (NiV), Ghana virus (GhV), or Mojiang virus (MojV). The non-CedV henipavirus can be the Malaysian strain of NiV (NiV-M) or the Bangladesh strain of NiV (NiV-B). In some embodiments, one or both of the F and G envelope glycoprotein genes of CedV are replaced with one or both of the F and G envelope glycoprotein genes of HeV (rCedV-HeV). In some embodiments, one or both of the F and G envelope glycoprotein genes of CedV are replaced with one or both of the F and G envelope glycoprotein genes of NiV (rCedV-NiV). In some embodiments, one or both of the F and G envelope glycoprotein genes of CedV are replaced with one or both of the F and G envelope glycoprotein genes of NiV-M (rCedV-NiV-M). In some embodiments, one or both of the F and G envelope glycoprotein genes of CedV are replaced with one or both of the F and G envelope glycoprotein genes of NiV-B (rCedV-NiV-B). The rCedV chimera can further comprise a reporter sequence, such as a reporter sequence that encodes a green fluorescent protein (GFP) or a luciferase protein (Luc).

In a second aspect, the present disclosure provides replication-competent recombinant Cedar virus (rCedV) chimeras, comprising the F and G envelope glycoprotein genes of CedV, and further comprising a coding sequence for one or both of (i) a soluble F envelope glycoprotein (sF) of a non-CedV henipavirus and (ii) a soluble G envelope glycoprotein (sG) of a non-CedV henipavirus. The sF and sG coding sequences can be coding sequences of any pathogenic henipavirus, such as NiV, HeV, GhV, or MojV.

In a third aspect, the present disclosure provides replication-competent recombinant Cedar virus (rCedV) chimeras, comprising one or both of (i) a gene encoding a henipavirus F envelope protein fusion protein, and (ii) a gene encoding a henipavirus G envelope protein fusion protein, wherein the fusion protein comprises the ectodomain and transmembrane domain of a non-CedV henipavirus F envelope protein or G envelope protein, respectively, fused to the cytoplasmic tail domain of CedV F envelope protein or G envelope protein, respectively, or (iii) a gene encoding a henipavirus G envelope protein fusion protein, wherein the fusion protein comprises the ectodomain of a non-CedV henipavirus F envelope protein or G envelope protein, respectively, fused to the cytoplasmic tail and transmembrane domains of CedV F envelope protein or G envelope protein, respectively. The non-CedV henipavirus can be a pathogenic henipavirus, such as HeV, NiV, GhV, or MojV. In some embodiments, the non-CedV henipavirus is Hendra virus (HeV) or Nipah virus (NiV). In some embodiments, the non-CedV henipavirus is the Malaysian strain of NiV (NiV-M) or the Bangladesh strain of NiV (NiV-B).

In a fourth aspect, the present disclosure provides vaccine compositions, comprising a rCedV chimera as disclosed herein and a pharmaceutically acceptable carrier. Such a vaccine composition may optionally further comprise an adjuvant. A vaccine as disclosed herein may further comprise one or both of (i) a soluble F envelope glycoprotein (sF) of a non-CedV henipavirus and (ii) a soluble G envelope glycoprotein (sG) of a non-CedV henipavirus. The non-CedV henipavirus sF and sG can be those of any pathogenic henipavirus, such as NiV, HeV, GhV, or MojV.

In a fifth aspect, the present disclosure provides methods of treating, reducing the risk of, or preventing henipavirus infection in a subject in need thereof, comprising administering to the subject an effective amount of a vaccine composition as disclosed herein.

In a sixth aspect, the present disclosure provides methods of inducing an immune response against a pathogenic henipavirus in a subject in need thereof, comprising administering to the subject an effective amount of a vaccine composition as disclosed herein.

In a seventh aspect, the present disclosure provides uses of a vaccine composition as disclosed herein for treating, reducing the risk of, or preventing henipavirus infection in a subject.

In an eighth aspect, the present disclosure provides uses of a vaccine composition as disclosed herein for inducing an immune response against a pathogenic henipavirus in a subject.

In a ninth aspect, the present disclosure provides vaccine compositions as disclosed herein for treating, reducing the risk of, or preventing henipavirus infection in a subject.

In a tenth aspect, the present disclosure provides vaccine compositions as disclosed herein for inducing an immune response against a pathogenic henipavirus in a subject.

In the context of the disclosed methods and uses, the target henipavirus may be any pathogenic henipavirus, including HeV, NiV (including NiV-M or NiV-B), GhV, or MojV. In the context of the disclosed methods and uses, the subject can be a human or a non-human mammal, including but not limited to livestock.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

The present invention may be better understood by reference to the drawings, which are incorporated in and constitute a part of this specification. The drawings are included to provide a further understanding of the disclosure and are merely exemplary to illustrate certain features that may be present or used singularly or in combination with other features. The present invention is not limited to the embodiments shown.

FIGS. 1A-B show a schematic representation of the rCedV chimeric antigenome plasmids described herein. FIG. 1A discloses SEQ ID NOS: 38 and 39, in order of appearance.

FIGS. 2A-B show western blots demonstrating intracellular expression of NiV-B (FIG. 2A) and HeV (FIG. 2B) envelope glycoproteins in infected Vero E6 cells as described herein.

FIG. 3A-B shows representative syncytia formation (giant cells) by rCedV chimeras rCedV-NiV-B-GFP and rCedV-HeV-GFP, rCedV-NiV-B-Luc and rCedV-HeV-Luc, and no reporter gene containing rCedV-NiV-B and rCedV-HeV (Scale bar, 50 m).

FIGS. 4A-B show a comparison of rCedV-NiV-B (FIG. 4A) and rCedV-HeV (FIG. 4B) progeny virus production.

FIGS. 5A-B show ephrin-B2 and ephrin-B3 are recognized as entry receptors by rCedV-NiV-B and rCedV-HeV and comparison to rCedV controls (Scale bar, 50 m).

FIGS. 6A-C show a comparison of rCedV chimeras described herein and live NiV-B and HeV by plaque reduction neutralization test (PRNT).

FIGS. 7A-H show correlation analysis of neutralization values shown in FIGs 6A-C.

FIG. 8 shows neutralization of rCedV-NiV-B-GFP and rCedV-HeV-GFP by NiV and HeV cross-reactive monoclonal antibodies by fluorescence reduction neutralization test (FRNT).

FIGS. 9A-H show correlation analysis of neutralization assays using the GFP expressing rCedV chimeric viruses from PRNT (y-axes) and FRNT (x-axes).

FIG. 10 show the analysis of sera from Rhesus macaques immunized with a mixture of NiV-B and NiV-M sG using the rCedV chimeras described herein by fluorescence reduction neutralization assay (FRNT).

FIG. 11A-B are a schematic representation of recombinant Cedar virus chimeric antigenome plasmids described herein comprising coding sequences for soluble G (sG) proteins of HeV or NiV-B. FIG. 11A discloses SEQ ID NOS: 40. FIG. 11B discloses SEQ ID NOs: 40, 41, and 42, in order of appearance.

FIG. 12 shows syncytia induced by rCedV expressing HeV sG glycoprotein.

FIG. 13 shows western blot data demonstrating the intracellular expression of the HeV sG glycoprotein in cell infected with rCedV-HeV sG.

FIG. 14 shows western blot data demonstrating that rCedV does not affect expression and secretion of HeV sG or new versions of HeV sG tetrameric (tet) constructs.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that specific details disclosed may not be required to practice the invention. Descriptions of specific applications are provided only as representative examples. The present invention is not limited to the embodiments shown, but is to be accorded the widest possible scope consistent with the principles and features disclosed herein.

Described herein are novel rCedV chimeras that are far superior constructs for any heretofore described assays or vaccine systems for henipaviruses, at least because CedV is non-pathogenic and is an authentic henipavirus that is closely related, genetically and structurally, to the other henipavirus species within the genus, including other pathogenic henipaviruses, such as HeV and NiV. As noted above, in some aspects, there are provided replication-competent rCedV chimeras wherein one or both of the F and G envelope glycoprotein genes of CedV are replaced with the F and G envelope glycoprotein genes, respectively, of a non-CedV henipavirus. As noted above, in other aspects, there are provided replication-competent rCedV chimeras comprising the F and G envelope glycoprotein genes of CedV, and further comprising a coding sequence for one or both of a soluble F envelope glycoprotein (sF) and a soluble G envelope glycoprotein (sG) from a non-CedV henipavirus. As noted above, in yet other aspects, there are provided rCedV chimeras comprising a gene encoding a chimeric fusion henipavirus F or G envelope glycoproteins, wherein, in the fusion protein, the ectodomain and transmembrane domain, of a non-CedV henipavirus F or G envelope glycoprotein is fused to the cytoplasmic tail of a CedV F or G envelope glycoprotein, respectively. There are also provided rCedV chimeras comprising a gene encoding a chimeric fusion henipavirus F or G envelope glycoproteins, wherein, in the fusion protein, the ectodomain of a non-CedV henipavirus F or G envelope glycoprotein is fused to the transmembrane domain and cytoplasmic tail of a CedV F or G envelope glycoprotein, respectively.

### I. Definitions

In order that the present invention may be more readily understood, certain terms are first defined. Additional definitions are set forth throughout the detailed description.

Technical and scientific terms used herein have the meanings commonly understood by one of ordinary skill in the art to which the present disclosure pertains, unless otherwise defined.

As used herein, the singular forms “a,” “an,” and “the” designate both the singular and the plural, unless expressly stated to designate the singular only. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, the term “henipavirus” refers to all viruses and strains thereof in the genus Henipavirus, which is a genus of negative-strand RNA viruses in the family Paramyxoviridae, including but not limited to Nipah virus (NiV), Hendra virus (HeV), Cedar virus (CedV), Ghana virus (GhV) and Mojiang virus (MojV). Further, as used herein, the terms “Nipah virus,” “NiV,” “Hendra virus,” “HeV,” “Cedar virus,” “CedV,” “Ghana virus,” “GhV,” “Mojiang virus,” and “MojV” are inclusive of any strains or sub-types of the respective virus species.

As used herein, the term “recombinant,” when used in reference to a virus, generally refers to a virus that is produced using genetic engineering techniques and is distinct from a naturally occurring virus.

As used herein, the term “chimera,” when used in reference to a virus, generally refers to a virus that encodes a gene expression product that the corresponding naturally occurring, or wild-type, virus does not express.

As used herein, the term “vaccine” refers to a preparation of a chimera as described herein, that, upon administration to a subject, stimulates one or both of antibody production and cellular immunity against a target virus, but is incapable of causing severe infection.

As used herein, the term “treat,” and variations thereof, refers to a reduction of the level of infection or viral load, including reduction to an undetectable level or elimination of infection, by administration of a chimera, composition, or vaccine as described herein. However, “treatment” does not require the achievement of a complete elimination of infection.

As used herein, the terms “reduce the risk of,” “prevent,” and variations thereof, refers to elimination or reduction of the incidence or onset or progression of infection by administration of a chimera, composition, or vaccine as described herein, as compared to that which would occur in the absence of the measure taken.

Alternatively stated, the treatments disclosed herein slow, delay, control, or decrease the likelihood or probability of infection or progression of infection in the subject, as compared to that which would occur in the absence of the measure taken.

As used herein, the term “immune response” generally refers to innate and acquired immune responses including, but not limited to, both humoral immune responses (mediated by B lymphocytes) and cellular immune responses (mediated by T lymphocytes).

As used herein, a “therapeutically effective” or “effective amount” designates a dose that causes a specific pharmacological effect for which the chimera, composition, or vaccine described herein is administered to a subject in need of such treatment, e.g., to reduce the risk of, prevent, or reduce infection, as may optionally be assessed through clinical testing and evaluation, patient observation, and/or the like. “Therapeutically effective amount” or “effective amount” can further designate a dose that causes a detectable change in biological or chemical activity. The detectable changes optionally may be detected and/or further quantified by one skilled in the art for the relevant mechanism or process. Moreover, “therapeutically effective amount” or “effective amount” can designate an amount that maintains a desired physiological state, i.e., reduces viral load, or prevents significant increase in viral load, and/or promotes improvement in the condition of interest (e.g., infection status). As is generally understood in the art, the dosage will vary depending on the administration routes, symptoms and body weight of the patient, but also depending upon the compound (e.g., chimera) or composition (e.g., vaccine) administered.

As used herein, the term “adjuvant” refers to a substance that can enhance or increase an immune response to an antigen. In general, an adjuvant as described herein will be pharmaceutically acceptable for use in the subject to which it is administered by the intended route of administration.

As used herein, the term “subject” refers to any animal in whom protection from pathogenic henipaviruses is intended, including but not limited to, vertebrates, such as mammals and birds. As used herein, the term “mammal” refers to any animal classified as a mammal, including humans and non-human primates, domestic/pet and farm/livestock animals (such as dogs, cats, horses, cows, pigs, sheep, goats, etc.), weasels, rodents, bats, and zoo or sports animals, etc. The terms “individual,” “subject,” and “patient” are used interchangeably herein.

### II. Cedar Virus Chimeras

Cedar virus (CedV) is a non-pathogenic henipavirus that can function as a platform for the development of pathogenic henipavirus-targeted technologies for use in antiviral drug discovery, assay and diagnostics development, and vaccine development strategies. Described herein are novel recombinant Cedar virus (rCedV) chimeras based on the non-pathogenic CedV, and engineered to express antigenic surface or soluble proteins/polypeptides of a non-CedV henipaviruses including pathogenic henipaviruses, such as Hendra virus (HeV), Nipah virus (NiV), Ghana virus (GhV), and Mojiang virus (MojV). The rCedV chimeras described herein have properties that make them advantageous as assay reagents and immunogenic agents (e.g., vaccines) because they are based on an authentic, non-pathogenic henipavirus (rCedV) that is selectively altered with chosen proteins from another henipavirus, such as a pathogenic henipavirus.

In general, the rCedV chimeras of the present disclosure are designed to either: (1) express a full length F envelope glycoprotein from a non-CedV henipavirus, a full length G envelope glycoprotein from a non-CedV henipavirus, or both, in place of the naturally occurring F, G, or both glycoproteins of CedV; (2) express a soluble form of a F envelope glycoprotein (sF) from a non-CedV henipavirus, a soluble form of a G envelope glycoprotein (sG) from a non-CedV henipavirus, or both, in addition to the naturally occurring F and G glycoproteins of CedV; or (3) express a henipavirus F envelope protein fusion protein, a henipavirus G envelope protein fusion protein, or both, in place of the naturally occurring F or G, or both, glycoproteins of CedV (respectively), wherein the fusion proteins comprise the ectodomain and transmembrane domain of a non-CedV henipavirus and the cytoplasmic domain of CedV, or the ectodomain of a non-CedV henipavirus and the transmembrane domain and cytoplasmic tail domain of CedV. Each of these designs provides a non-pathogenic chimeric virus that is replication-competent. The chimeric viruses can be used for study of the non-CedV henipavirus of which the F/G proteins are expressed. Additionally or alternatively, the chimeric viruses can be used for eliciting an immune response against henipaviruses, including against the non-CedV henipavirus of which the F/G proteins are expressed (e.g., in the form of a vaccine).

The non-CedV henipavirus can be any henipavirus, including any pathogenic henipavirus. For example, the non-CedV henipavirus can be Hendra virus (HeV), Nipah virus (NiV), Ghana virus (GhV), Mojiang virus (MojV), or any other henipavirus.

A chimera as disclosed herein may further comprise a reporter sequence, such as a reporter sequence that encodes green fluorescent protein (GFP) or luciferase protein (Luc). Such a reporter sequence may facilitate study of the chimera, be useful in assays using the chimera, or permit tracking of the chimera after administration.

The genome sequences of a CedV isolate and an exemplary recombinant CedV are provided in Table 9 at the end of the specification. Certain modifications including a C7A substitution, a C395A substitution, a C4816A substitution, and incorporations of a MluI restriction site (ACGCGT; SEQ ID NO: 27) are shown in bold and underlined in Table 9 (SEQ ID NO: 2). The C395A and C4816A substitutions remove internal SmaI restriction sites. The MluI restriction site can be inserted between the P and M genes at nucleotide position 4531, after the M transcriptional start sequence, to facilitate insertion of a modified turbo Green Fluorescent Protein (GFP) gene or a firefly luciferase protein (Luc) gene, or the soluble F or soluble G gene sequences from a non-CedV henipavirus. The genome sequences of HeV, NiV, GhV, and MojV are published and available on Genbank. Each of the foregoing chimera designs are described in more detail below.

### A. Chimeras Expressing Full-Length Non-CedV Proteins

One aspect of the present disclosure is directed to replication-competent, recombinant Cedar virus (rCedV) chimeras wherein one or both of the F and G envelope glycoprotein genes of CedV are replaced with one or both of the F and G envelope glycoprotein genes, respectively, of a non-CedV henipavirus. While not wanting to be bound by theory, it is believed that in order for this type of rCedV chimera to remain replication-competent, the virus can comprise only one gene encoding a full-length F protein and one gene encoding a full-length G protein. Thus, in these embodiments, if the rCedV chimera expresses a non-CedV F protein, then the gene encoding the CedV F protein is deleted, and if the rCedV chimera expresses a non-CedV G protein, then the gene encoding the CedV G protein is deleted. If the rCedV expresses both F and G non-CedV proteins, then the genes encoding the CedV F and G proteins are deleted.

For example, the gene encoding the F envelope glycoprotein of CedV may be replaced by a gene encoding a F envelope glycoprotein of a non-CedV henipavirus (e.g., HeV, NiV, GhV, or MojV) while the G envelope glycoprotein of the rCedV remains unaltered; the gene encoding the G envelope glycoprotein of CedV may be replaced by a gene encoding a G envelope glycoprotein of a non-CedV henipavirus (e.g., HeV, NiV, GhV, or MojV) while the F envelope glycoprotein of the rCedV remains unaltered; or the genes encoding both the F envelope glycoprotein and the G envelope glycoprotein of CedV may be replaced, respectively, by a gene encoding a F envelope glycoprotein and a gene encoding a G envelope glycoprotein of a non-CedV henipavirus (e.g., HeV, NiV, GhV, or MojV). In some embodiments, when both the F and G envelope glycoprotein genes are replaced, both replacement genes are from the same non-CedV henipavirus (e.g., HeV, NiV, GhV, or MojV). In other embodiments, when both the F and G envelope glycoprotein genes are replaced, each replacement gene is from a different non-CedV henipavirus.

Coding sequences for exemplary F and G proteins of various Henipaviruses (SEQ ID NOs: 3-12) that can replace the F and G proteins of CedV (SEQ ID NOs: 28 and 29) are shown in Table 1 below.

TABLE 1
F and G Protein Coding Sequences for Henipaviruses

| | SEQ | |
|---|---|---|
| | ID | |
| Name | NO: | Sequence |
| NiV-B | 3 | ATGGCAGTTATACTTAACAAGAGATATTATTCTAATCTCTTAATACTGATTTTGATGATC |
| F coding | | TCGGAGTGCAGTGTCGGGATTTTGCATTATGAGAAATTGAGTAAGATTGGGCTTGTCAA |
| sequence | | AGGAATAACAAGAAAATACAAGATCAAAAGCAATCCTCTCACAAAAGACATTGTTATTA |
| GenBank: | | AAATGATTCCGAATGTGTCAAACATGTCTCAATGCACGGGGAGTGTCATGGAAAACTAT |
| JN808864.1 | | AAAACACGATTAAACGGTATCCTAACGCCTATAAAGGGGGCATTAGAGATTTACAAGAA |
| Predicted | | CAACACTCATGACCTTGTCGGTGATGTAAGACTGGCCGGAGTTATAATGGCAGGAGTTG |
| TM domain | | CTATTGGAATTGCAACCGCAGCTCAAATTACTGCAGGTGTAGCATTATATGAGGCAATG |
| underlined | | AAAAATGCTGACAACATCAACAAACTCAAAAGCAGCATAGAATCAACTAATGAAGCTG |
| | | TTGTTAAGCTTCAAGAGACTGCAGAAAAGACAGTCTATGTACTGACCGCTTTGCAGGAT |
| | | TACATTAATACTAACTTGGTACCGACAATTGACAAGATAAGCTGCAAACAGACGGAACT |
| | | CTCATTAGATCTAGCACTATCAAAGTACCTCTCTGATTTGCTTTTTGTATTTGGTCCCAAC |
| | | CTTCAAGACCCAGTTTCTAATTCAATGACTATACAGGCTATATCTCAGGCATTCGGTGGA |
| | | AATTATGAAACACTGCTAAGAACGTTGGGTTACGCTACAGAAGACTTTGATGATCTTCT |
| | | AGAAAGTGACAGCATAACGGGTCAAATTATCTACGTTGATTTAAGTGGCTACTACATAA |
| | | TTGTCAGGGTTTATTTTCCTATCCTGACTGAAATCCAACAGGCCTATATCCAAGAATTGT |
| | | TGCCAGTGAGCTTTAACAATGACAATTCAGAATGGATCAGCATTGTCCCAAATTTCATAT |
| | | TGGTAAGGAACACATTAATATCAAATATAGAGATTGGATTTTGCCTAATTACAAAGAGG |
| | | AGTGTGATCTGCAACCAAGATTATGCAACACCCATGACAAACAATATGAGGGAATGTTT |
| | | GACGGGGTCGACTGAGAAGTGTCCTCGAGAGCTGGTGGTTTCATCACACGTTCCCAGAT |
| | | TTGCACTATCTAACGGGGTTTTGTTTGCTAATTGCATAAGCGTCACATGCCAGTGTCAAA |
| | | CAACAGGTAGGGCAATCTCACAGTCAGGAGAACAAACTCTGCTGATGATTGATAACACC |
| | | ACCTGTCCTACAGCCGTACTCGGTAATGTGATCATCAGCTTGGGAAAATATCTTGGGTCA |
| | | GTAAATTATAACTCTGAAGGCATTGCTATTGGTCCTCCTGTCTTTACTGATAAAGTTGAC |
| | | ATATCAAGTCAAATATCTAGCATGAATCAGTCCTTACAACAATCTAAGGACTATATCAA |
| | | AGAAGCTCAACGACTCCTTGATACTGTTAACCCGTCATTAATAAGCATGTTGTCTATGAT |
| | | CATACTGTATGTACTATCAATTGCATCATTGTGTATAGGATTGATTACATTTATCAGTTTT |
| | | ATCATTGTTGAGAAAAAAAGAAACACCTATAGCAGATTAGAGGACAGGAGAGTCAGAC |
| | | CTACAAGTAGTGGGGATCTCTATTACATTGGGACGTAG |
| NIV-B | 4 | ATGCCGACAGAAAGCAAGAAAGTTAGATTCGAAAATACTGCTTCAGACAAGGGGAAAA |
| G coding | | ATCCTAGTAAAGTTATTAAGAGCTACTACGGAACCATGGACATTAAGAAAATAAATGAA |
| sequence | | GGGTTATTGGACAGCAAGATATTAAGTGCTTTCAACACAGTGATAGCACTGCTTGGATC |
| GenBank: | | CATTGTAATCATAGTGATGAATATAATGATCATCCAAAACTACACAAGATCAACAGATA |
| JN808864.1 | | ATCAGGCCATGATCAAAGATGCATTGCAGAGTATCCAGCAGCAGATCAAGGGGCTTGCC |
| Predicted | | GACAAAATTGGCACAGAGATAGGGCCGAAAGTATCACTGATTGATACATCCAGTACTAT |
| TM domain | | CACTATTCCAGCTAATATTGGGCTGTTAGGTTCAAAGATCAGCCAGTCAACTGCAAGTAT |
| underlined | | AAATGAGAATGTGAATGAAAAATGCAAATTTACACTGCCTCCCTTGAAAATCCACGAAT |
| | | GTAACATTTCTTGTCCTAACCCACTCCCTTTTAGAGAGTATAAGCCGCAGACAGAAGGA |
| | | GTGAGCAATCTGGTAGGATTACCTAATAATATCTGTCTGCAAAAGACATCTAATCAGAT |
| | | ACTGAAACCAAAGCTGATTTCATACACCTTACCCGTAGTCGGTCAAAGTGGCACCTGTA |
| | | TCACAGACCCACTGCTGGCTATGGATGAGGGCTACTTTGCATATAGCCACCTGGAAAAA |
| | | ATCGGATCATGTTCAAGAGGGGTCTCCAAACAAAGAATAATAGGAGTTGGAGAGGTACT |
| | | AGACAGAGGTGACGAAGTACCTTCTTTGTTTATGACTAACGTCTGGACCCCATCAAATCC |
| | | AAACACCGTTTACCATTGCAGTGCCGTGTACAACAATGAATTCTATTATGTGCTTTGTGC |
| | | AGTGTCAGTTGTTGGAGACCCTATTCTGAATAGCACCTACTGGTCCGGATCACTAATGAT |
| | | GACTCGTCTAGCTGTAAAACCTAAGAATAATGGTGAGAGTTACAATCAACATCAATTTG |
| | | CCTTACGGAATATTGAGAAAGGGAAGTATGATAAAGTTATGCCATATGGACCCTCAGGC |
| | | ATCAAACAAGGTGACACCCTGTACTTTCCTGCTGTAGGATTTTTGGTCAGGACAGAGTTC |
| | | ACATACAATGATTCAAATTGTCCCATCGCAGAGTGTCAATACAGCAAACCTGAAAACTG |
| | | CAGGCTATCTATGGGGATTAGACCAAACAGTCATTATATCCTTCGATCTGGACTACTAAA |
| | | ATACAATCTATCGGATGAGGAGAACTCTAAAATTGTATTCATTGAAATATCTGATCAAA |
| | | GACTATCTATTGGATCTCCTAGCAAAATCTATGATTCTTTGGGTCAACCTGTTTTCTACCA |
| | | AGCATCTTTTTCATGGGACACTATGATTAAATTTGGAGATGTCCAAACAGTTAACCCTTT |
| | | AGTTGTAAATTGGCGTGACAACACGGTAATCTCAAGACCTGGGCAATCACAATGCCCTA |
| | | GATTCAACAAGTGCCCAGAGGTTTGCTGGGAAGGGGTTTATAATGATGCTTTCCTGATTG |
| | | ATAGAATCAATTGGATAAGCGCGGGTGTATTCCTTGACAGCAACCAGACCGCAGAGAAT |
| | | CCTGTTTTTACTGTATTCAAAGATAATGAAGTACTTTACAGAGCACAACTAGCTTCCGAG |
| | | GACACCAATGCACAAAAAACAATAACTAATTGCTTCCTTTTGAAGAATAAGATCTGGTG |
| | | TATATCACTGGTTGAGATATACGACACAGGAGACAATGTTATAAGACCTAAACTATTCG |
| | | CAGTTAAGATACCAGAGCAATGTACATAA |
| HeV | 5 | ATGGCTACACAAGAGGTTAGGCTAAAGTGTTTGCTCTGTGGGATCATAGTTCTGGTTTTG |
| F coding | | TCATTAGAAGGGCTAGGAATACTACATTATGAGAAACTTAGTAAGATAGGGCTGGTTAA |
| sequence | | AGGTATTACAAGAAAGTACAAGATTAAGAGTAACCCTTTGACCAAGGATATTGTAATCA |
| GenBank: | | AAATGATCCCTAATGTTTCGAATGTCTCAAAGTGCACCGGGACTGTTATGGAGAATTAC |
| JN255805.1 | | AAAAGCAGACTCACAGGGATTCTCTCACCAATCAAAGGCGCCATCGAACTGTACAATAA |
| Predicted | | TAACACGCATGACCTAGTTGGTGATGTCAAGCTTGCAGGTGTGGTGATGGCAGGGATTG |
| TM domain | | CAATCGGGATAGCTACTGCTGCACAAATCACAGCAGGTGTTGCCTTATATGAGGCAATG |
| underlined | | AAGAACGCAGACAATATCAATAAACTCAAGAGCAGCATAGAGTCTACAAATGAGGCTG |
| | | TTGTCAAATTACAGGAAACAGCTGAGAAAACAGTCTACGTCCTTACTGCTCTTCAAGATT |
| | | ACATCAACACTAACCTTGTTCCTACAATAGATCAAATTAGCTGCAAGCAAACAGAACTC |
| | | GCATTAGACTTGGCGTTGTCTAAGTATCTGTCTGATCTGCTCTTTGTTTTCGGACCTAACT |
| | | TACAGGATCCAGTCTCTAATTCCATGACTATCCAAGCAATATCTCAAGCATTTGGGGGCA |
| | | ATTACGAAACCTTACTGAGAACGCTTGGTTACGCGACCGAGGATTTCGACGACCTTTTA |
| | | GAAAGTGATAGCATAACAGGCCAGATAGTCTATGTAGATCTCAGTAGCTATTACATAAT |
| | | AGTAAGGGTGTATTTTCCTATACTAACAGAGATCCAACAGGCTTATGTGCAGGAGTTGC |
| | | TTCCAGTGAGTTTTAATAACGATAATTCAGAATGGATCAGCATTGTCCCGAATTTCGTGC |
| | | TGATTAGGAACACGCTGATTTCAAATATAGAAGTTAAGTACTGCTTAATCACCAAGAAA |
| | | AGTGTGATTTGTAATCAAGACTATGCTACACCCATGACGGCTAGCGTGAGAGAATGCTT |
| | | GACAGGATCCACAGATAAGTGCCCAAGGGAGTTAGTAGTCTCATCCCATGTTCCAAGAT |
| | | TTGCCCTCTCAGGAGGAGTCTTGTTTGCAAATTGTATAAGTGTGACATGTCAGTGTCAGA |
| | | CTACTGGGAGGGCAATATCTCAATCAGGGGAACAGACACTACTGATGATTGACAATACT |
| | | ACCTGCACAACAGTTGTTCTAGGAAACATAATCATAAGCCTTGGAAAATATTTGGGATC |
| | | AATAAATTACAATTCCGAGAGCATTGCTGTTGGGCCACCAGTCTATACAGACAAAGTTG |
| | | ATATCTCAAGTCAGATATCTAGTATGAATCAATCACTACAACAATCTAAGGATTACATTA |
| | | AAGAAGCTCAAAAGATCTTGGACACTGTGAATCCGTCGTTGATAAGTATGCTATCAATG |
| | | ATCATCCTTTATGTTTTGTCCATTGCGGCACTGTGCATTGGTCTGATCACTTTCATAAGCT |
| | | TTGTAATAGTTGAGAAAAAGAGAGGGAATTACAGCAGGCTAGATGATAGGCAGGTGCG |
| | | ACCGGTCAGTAATGGTGATCTGTATTATATTGGAACATAA |
| HeV | 6 | ATGGCTACACAAGAGGTCAGGCTAAAGTGTTTGCTCTGTGGGATCATAGTTCTGGTTTTG |
| F coding | | TCATTAGAAGGGCTAGGGATACTACATTATGAGAAACTTAGTAAGATAGGGCTGGTTAA |
| sequence | | AGGTATTACAAGAAAGTACAAGATTAAGAGTAACCCTTTGACCAAGGATATTGTGATCA |
| GenBank: | | AAATGATCCCTAATGTCTCGAATGTCTCAAAGTGCACCGGGACTGTTATGGAGAATTAC |
| MN062017.1 | | AAAAGCAGACTCACAGGGATTCTCTCACCAATCAAAGGCGCCATCGAACTGTACAATAA |
| Predicted | | TAACACGCATGACCTAGTTGGTGATGTCAAGCTTGCAGGTGTGGTGATGGCAGGGATTG |
| TM domain | | CAATCGGGATAGCTACTGCTGCACAAATCACAGCAGGTGTTGCCTTATATGAGGCAATG |
| underlined | | AAGAACGCAGACAATATCAATAAACTCAAGAGCAGCATAGAGTCTACAAATGAGGCTG |
| | | TTGTCAAATTACAGGAAACAGCTGAGAAAACAGTCTACGTCCTTACTGCTCTTCAAGATT |
| | | ACATCAACACTAACCTTGTTCCTACAATAGATCAAATTAGCTGCAAGCAAACAGAGCTC |
| | | GCATTAGACTTGGCGTTGTCTAAGTATCTGTCTGATCTGCTCTTTGTTTTCGGACCTAACT |
| | | TACAGGATCCAGTCTCTAATTCCATGACTATCCAAGCAATATCTCAAGCATTTGGGGGCA |
| | | ATTACGAAACCTTACTGAGAACGCTTGGTTACGCGACCGAGGACTTCGACGACCTTTTA |
| | | GAAAGTGATAGCATAACAGGCCAGATAGTCTATGTAGATCTCAGTAGCTATTACATAAT |
| | | AGTAAGGGTGTATTTTCCCATACTAACAGAGATCCAACAGGCTTATGTGCAGGAGTTGC |
| | | TTCCAGTGAGTTTTAATAACGATAATTCAGAATGGATCAGCATTGTCCCGAATTTCGTGC |
| | | TGATTAGGAACACGCTGATTTCAAATATAGAAGTCAAGTACTGCTTAATCACCAAGAAA |
| | | AGTGTGATTTGTAATCAGGACTATGCTACACCCATGACGGCTAGCGTGAGAGAATGCTT |
| | | GACAGGATCCACAGATAAGTGCCCAAGGGAGTTAGTAGTCTCATCCCATGTTCCAAGAT |
| | | TTGCCCTCTCAGGAGGAGTCTTGTTTGCAAATTGTATAAGTGTGACATGTCAGTGTCAGA |
| | | CTACTGGGAGGGCAATATCTCAATCAGGGGAACAGACACTACTGATGATTGACAATACT |
| | | ACCTGCACAACAGTTGTTCTAGGAAACATAATCATAAGCCTTGGAAAATATTTGGGATC |
| | | AATAAATTACAATTCTGAGAGCATTGCTGTTGGGCCACCAGTCTATACAGACAAAGTTG |
| | | ATATCTCAAGTCAGATATCTAGTATGAATCAATCACTACAACAATCTAAGGATTACATTA |
| | | AAGAAGCTCAAAAGATCTTGGACACTGTGAATCCGTCGTTGATAAGTATGCTATCAATG |
| | | ATCATCCTTTATGTTTTGTCCATTGCAGCACTGTGCATTGGTCTGATCACTTTCATAAGCT |
| | | TTGTAATAGTTGAGAAAAAGAGAGGGAATTACAGCAGGCTAGATGATAGGCAAGTGCG |
| | | ACCGGTCAGTAATGGTGATCTGTATTATATTGGAACATAA |
| HeV | 7 | ATGATGGCTGATTCCAAATTGGTAAGCCTGAACAATAATCTATCTGGTAAAATCAAGGA |
| G coding | | TCAAGGTAAAGTTATCAAGAATTATTACGGCACAATGGACATCAAGAAAATTAACGATG |
| sequence | | GGTTATTAGATAGTAAGATACTTGGGGCGTTTAACACAGTGATAGCTTTGTTGGGATCA |
| GenBank: | | ATCATCATCATTGTGATGAATATCATGATAATTCAAAATTACACCAGAACGACTGATAA |
| JN255805.1 | | TCAGGCACTAATCAAAGAGTCACTCCAGAGTGTACAGCAACAAATCAAAGCTTTAACAG |
| Predicted | | ACAAAATCGGGACAGAGATAGGCCCCAAAGTCTCACTGATTGACACATCCAGCACCATC |
| TM domain | | ACAATTCCTGCTAACATAGGGTTACTGGGATCCAAGATAAGTCAGTCTACCAGCAGTAT |
| underlined | | TAATGAGAATGTTAACGATAAATGCAAATTTACTCTTCCTCCTTTAAAGATTCATGAGTG |
| | | TAATATCTCTTGTCCGAATCCTTTGCCTTTCAGAGAATACCGACCAATCTCACAAGGGGT |
| | | GAGTGATCTTGTAGGACTGCCAAACCAGATCTGTCTACAGAAGACAACATCAACAATCT |
| | | TAAAGCCCAGGCTGATATCCTATACTCTACCAATTAATACCAGAGAAGGGGTTTGCATC |
| | | ACTGACCCACTTTTGGCTGTTGATAATGGCTTCTTCGCCTATAGCCATCTTGAAAAGATC |
| | | GGATCATGTACTAGAGGAATTGCAAAACAAAGGATAATAGGGGTGGGTGAGGTATTGG |
| | | ATAGGGGTGATAAGGTGCCATCAATGTTTATGACCAATGTTTGGACACCACCCAATCCA |
| | | AGCACCATCCATCATTGCAGCTCAACTTACCATGAAGATTTTTATTACACATTGTGCGCA |
| | | GTGTCCCATGTGGGAGATCCTATCCTTAACAGTACTTCTTGGACAGAGTCACTGTCTCTG |
| | | ATTCGTCTTGCTGTAAGACCAAAAAGTGATAGTGGAGACTACAATCAGAAATACATCGC |
| | | TATAACTAAAGTTGAAAGAGGGAAGTACGATAAGGTAATGCCTTACGGTCCATCAGGTA |
| | | TCAAGCAAGGGGATACATTGTACTTTCCGGCCGTCGGTTTCTTGCCAAGGACCGAATTTC |
| | | AATATAATGACTCTAATTGTCCCATAATTCATTGCAAGTACAGCAAAGCAGAAAACTGT |
| | | AGGCTTTCAATGGGTGTCAACTCCAAAAGTCATTATATTTTGAGATCAGGACTATTGAAG |
| | | TATAATCTGTCTCTTGGAGGAGACATCATACTCCAATTTATCGAGATTGCTGACAATAGA |
| | | TTGACCATCGGTTCTCCTAGTAAGATATACAATTCCCTAGGTCAACCCGTTTTCTACCAG |
| | | GCATCATATTCTTGGGATACGATGATTAAATTAGGCGATGTTGATACCGTTGACCCTCTA |
| | | AGAGTACAGTGGAGAAATAACAGTGTGATTTCTAGACCTGGACAGTCACAGTGTCCTCG |
| | | ATTTAATGTCTGTCCCGAGGTATGCTGGGAAGGGACATATAATGATGCTTTTCTAATAGA |
| | | TCGGCTAAACTGGGTTAGTGCTGGTGTTTATTTAAACAGTAACCAAACTGCAGAGAACC |
| | | CTGTGTTTGCCGTATTCAAGGATAACGAGATCCTTTACCAAGTTCCACTGGCTGAGGATG |
| | | ACACAAATGCACAAAAAACCATCACAGATTGCTTCTTGCTGGAGAATGTCATATGGTGT |
| | | ATATCACTAGTAGAAATATACGATACCGGAGACAGTGTGATAAGACCAAAACTGTTTGC |
| | | AGTCAAGATACCTGCCCAATGTTCAGAGAGTTGA |
| HeV | 8 | ATGATGGCTGATTCCAAATTGGTAAGCCTGAACAATAATCTATCTGGTAAAATCAAGGA |
| G coding | | TCAAGGTAAAGTTATCAAGAATTATTACGGCACAATGGACATCAAGAAAATTAACGATG |
| sequence | | GGTTATTAGATAGTAAGATACTTGGGGCGTTTAACACAGTGATAGCTTTGTTGGGATCA |
| GenBank: | | ATCATCATCATTGTGATGAATATCATGATAATTCAAAATTACACCAGAACGACTGATAA |
| MN062017.1 | | TCAGGCACTAATCAAAGAGTCACTCCAGAGTGTACAGCAACAAATCAAAGCTTTAACAG |
| Predicted | | ACAAAATCGGGACAGAGATAGGCCCCAAAGTCTCACTAATTGACACATCCAGCACCATC |
| TM domain | | ACAATTCCTGCTAACATAGGGTTACTGGGATCCAAGATAAGTCAGTCTACCAGCAGTAT |
| underlined | | TAATGAGAATGTTAACGATAAATGCAAATTTACTCTTCCTCCTTTAAAGATTCATGAGTG |
| | | TAATATCTCTTGTCCGAATCCTTTGCCTTTCAGAGAATACCGACCAATCTCACAAGGGGT |
| | | GAGTGATCTTGTAGGACTGCCGAACCAGATCTGTCTACAGAAGACAACATCAACAATCT |
| | | TAAAGCCCAGGCTGATATCCTATACTCTACCAATTAATACCAGAGAAGGGGTTTGCATC |
| | | ACTGACCCACTTTTGGCTGTTGATAATGGCTTCTTCGCCTATAGCCATCTTGAAAAGATC |
| | | GGATCATGTACTAGAGGAATTGCAAAACAAAGGATAATAGGGGTGGGTGAGGTATTGG |
| | | ATAGGGGTGATAAGGTGCCATCAATGTTTATGACCAATGTTTGGACACCACCCAATCCA |
| | | AGCACCATCCATCATTGCAGCTCAACTTACCATGAAGATTTTTATTACACATTGTGCGCA |
| | | GTGTCCCATGTGGGAGATCCTATCCTTAACAGTACTTCCTGGACAGAGTCACTGTCTCTG |
| | | ATTCGTCTTGCTGTAAGACCAAAAAGTGATAGTGGAGACTACAATCAGAAATACATCGC |
| | | TATAACTAAAGTTGAAAGAGGGAAGTACGATAAGGTGATGCCTTACGGTCCATCAGGTA |
| | | TCAAGCAAGGGGATACATTGTACTTTCCGGCCGTCGGTTTTTTGCCAAGGACCGAATTTC |
| | | AATATAATGACTCTAATTGTCCCATAATTCATTGCAAGTACAGCAAAGCAGAAAACTGT |
| | | AGGCTTTCAATGGGTGTCAACTCCAAAAGTCATTATATTTTGAGATCAGGACTATTGAAG |
| | | TATAATCTATCTCTTGGAGGAGACATCATACTCCAATTTATCGAGATTGCTGACAATAGA |
| | | TTGACCATCGGTTCTCCTAGTAAGATATACAATTCCCTAGGTCAACCCGTTTTCTACCAG |
| | | GCATCATATTCTTGGGATACGATGATTAAATTAGGCGATGTTGATACCGTTGACCCTCTA |
| | | AGAGTACAGTGGAGAAATAACAGTGTGATTTCTAGACCTGGACAGTCACAGTGTCCTCG |
| | | ATTTAATGTCTGTCCCGAGGTATGCTGGGAAGGGACATATAATGATGCTTTTCTAATAGA |
| | | CCGGCTAAACTGGGTTAGTGCTGGTGTTTATTTAAACAGTAACCAAACTGCAGAGAACC |
| | | CTGTGTTTGCCGTATTCAAGGATAACGAGATCCTTTACCAAGTTCCACTGGCTGAAGATG |
| | | ACACAAATGCACAAAAAACCATCACAGATTGCTTCTTGCTGGAGAATGTCATATGGTGT |
| | | ATATCACTAGTAGAAATATACGATACAGGAGACAGTGTGATAAGGCCAAAACTATTTGC |
| | | AGTCAAGATACCTGCCCAATGTTCAGAGAGTTGA |
| MojV | 9 | ATGGCACTAAATAAAAATATGTTCAGTTCACTGTTCCTTGGTTATCTATTAGTGTACGCT |
| F coding | | ACGACTGTTCAGTCTAGTATACACTATGACTCCTTATCTAAGGTCGGTGTCATTAAGGGT |
| sequence | | CTGACATACAACTATAAGATCAAGGGTTCGCCATCTACAAAGCTAATGGTGGTCAAATT |
| GenBank: | | GATACCTAACATTGATAGTGTTAAAAACTGTACTCAGAAACAGTATGATGAATACAAGA |
| KF278639.1 | | ACTTAGTAAGGAAAGCCTTAGAACCGGTCAAAATGGCTATTGACACCATGCTCAATAAT |
| Predicted | | GTTAAGTCGGGTAATAACAAGTACAGATTTGCAGGTGCAATTATGGCTGGAGTTGCCCT |
| TM domain | | CGGTGTTGCAACAGCAGCCACTGTTACAGCAGGGATAGCTCTCCATAGATCAAATGAAA |
| underlined | | ATGCACAGGCAATTGCAAACATGAAGAGTGCTATTCAAAATACAAATGAGGCAGTAAA |
| | | GCAATTGCAATTGGCCAATAAACAAACACTAGCTGTGATTGACACCATAAGAGGAGAG |
| | | ATCAATAACAATATAATACCCGTTATAAATCAATTGAGCTGTGACACAATTGGGCTCAG |
| | | TGTAGGTATAAGACTCACTCAGTACTACTCTGAAATAATAACTGCATTTGGGCCAGCTTT |
| | | GCAGAATCCAGTAAATACAAGGATTACCATTCAAGCAATATCTAGTGTGTTTAATGGCA |
| | | ACTTTGATGAACTGCTGAAGATTATGGGGTATACAAGTGGTGATCTTTATGAAATTCTAC |
| | | ATAGTGAATTAATTAGAGGCAACATTATAGACGTTGATGTAGATGCAGGATACATAGCT |
| | | CTAGAAATAGAATTCCCCAATCTAACATTGGTACCTAATGCTGTAGTACAGGAGTTAAT |
| | | GCCTATCAGTTATAACATAGACGGGGATGAGTGGGTCACACTTGTGCCAAGGTTTGTAC |
| | | TTACAAGGACTACACTGTTATCAAATATTGATACGAGTAGATGTACAATCACAGATAGT |
| | | AGTGTCATATGTGACAACGACTACGCCTTGCCTATGTCACACGAGCTTATTGGCTGCTTA |
| | | CAGGGAGATACATCTAAGTGTGCTAGAGAGAAGGTAGTCTCAAGTTATGTCCCTAAATT |
| | | TGCGTTGTCTGATGGGTTAGTGTATGCAAATTGCCTCAATACTATCTGCCGATGTATGGA |
| | | TACAGATACTCCAATCTCACAAAGTCTCGGAGCCACTGTATCATTACTAGACAACAAGA |
| | | GGTGTTCAGTATATCAGGTAGGAGATGTCTTGATTTCTGTCGGATCATATCTAGGAGATG |
| | | GAGAATATAATGCTGATAATGTAGAGCTTGGCCCACCTATAGTTATAGATAAGATTGAC |
| | | ATAGGAAATCAGCTGGCAGGTATTAATCAAACCTTACAAGAGGCAGAAGATTACATTGA |
| | | GAAGTCAGAAGAGTTCTTAAAAGGGGTTAACCCTTCAATTATCACTCTTGGTTCCATGGT |
| | | TGTCCTTTATATATTTATGATATTAATAGCCATTGTGTCAGTAATAGCACTAGTATTGTCA |
| | | ATTAAATTAACAGTAAAAGGTAACGTGGTAAGGCAGCAGTTCACGTATACTCAGCATGT |
| | | TCCTAGCATGGAGAATATCAATTATGTAAGTCATTAA |
| MojV | 10 | ATGGCAACGAATAGAGACAATACCATCACATCAGCAGAGGTCTCCCAAGAAGATAAGG |
| G coding | | TTAAAAAATATTATGGAGTGGAAACTGCTGAGAAGGTTGCCGACAGCATAAGTGGTAAT |
| sequence | | AAGGTATTCATATTGATGAATACACTTCTGATACTGACAGGTGCTATTATTACAATAACA |
| GenBank: | | CTAAATATCACCAACCTGACAGCGGCTAAAAGTCAACAGAATATGCTGAAAATAATCCA |
| KF278639.1 | | AGATGACGTGAATGCCAAATTAGAAATGTTCGTGAATCTTGATCAATTGGTGAAGGGTG |
| Predicted | | AAATTAAGCCAAAAGTGTCACTCATAAATACAGCAGTGAGCGTCAGCATACCCGGTCAG |
| TM domain | | ATCTCAAACCTCCAGACCAAATTCCTGCAAAAATATGTTTACTTAGAAGAATCTATTACT |
| underlined | | AAGCAGTGCACTTGCAACCCTTTATCTGGGATATTTCCAACATCAGGCCCAACCTACCCT |
| | | CCAACTGATAAACCAGACGATGATACCACAGATGATGACAAAGTGGACACCACGATTA |
| | | AGCCTATTGAGTACCCCAAGCCGGATGGGTGCAATAGAACTGGCGACCATTTCACGATG |
| | | GAGCCCGGAGCTAACTTTTATACTGTCCCTAACCTAGGACCGGCAAGTTCTAATTCTGAC |
| | | GAGTGTTACACAAACCCCTCTTTTTCAATTGGGTCCTCCATCTATATGTTTTCTCAAGAG |
| | | ATTAGAAAAACGGACTGCACAGCAGGAGAGATATTATCAATTCAGATCGTCTTAGGCCG |
| | | AATAGTAGACAAGGGTCAGCAGGGTCCTCAAGCATCACCCTTATTAGTATGGGCCGTCC |
| | | CAAATCCAAAGATCATAAACTCGTGTGCTGTCGCAGCTGGAGACGAGATGGGATGGGTG |
| | | TTATGCTCAGTGACATTAACTGCAGCATCAGGGGAGCCCATACCTCACATGTTTGATGG |
| | | GTTCTGGTTGTATAAGTTAGAACCTGACACCGAAGTTGTATCCTATAGAATCACAGGCTA |
| | | TGCTTATCTCTTAGATAAACAATATGACTCTGTCTTTATAGGTAAGGGCGGTGGTATTCA |
| | | GAAAGGTAACGATCTATACTTTCAGATGTATGGATTGTCCAGAAATAGGCAAAGTTTTA |
| | | AGGCACTGTGTGAACATGGATCATGCCTCGGCACTGGAGGTGGAGGGTATCAAGTGTTG |
| | | TGTGACAGGGCTGTGATGTCTTTCGGGAGTGAAGAATCACTAATTACAAATGCATATCT |
| | | GAAGGTGAATGATCTGGCAAGTGGGAAACCTGTGATAATAGGACAGACATTTCCGCCCT |
| | | CAGATTCTTATAAAGGCTCAAATGGTCGGATGTACACTATAGGTGATAAATATGGTCTG |
| | | TATCTTGCTCCGTCATCCTGGAACAGATATCTTAGATTTGGGATAACACCAGATATTTCT |
| | | GTAAGATCAACAACCTGGTTGAAAAGTCAAGATCCGATAATGAAGATTTTGTCAACATG |
| | | CACGAACACTGATAGAGATATGTGTCCTGAAATTTGCAATACTAGAGGTTATCAAGATA |
| | | TTTTTCCATTGTCGGAGGATTCAGAGTATTATACATACATAGGCATAACTCCTAATAATG |
| | | GTGGAACTAAAAACTTTGTGGCCGTACGTGACTCAGATGGTCATATAGCATCCATTGAT |
| | | ATTTTACAAAATTATTATAGTATCACCTCAGCTACTATAAGCTGCTTCATGTACAAAGAT |
| | | GAGATTTGGTGTATTGCAATCACAGAAGGGAAAAAACAGAAAGACAATCCTCAACGGA |
| | | TATATGCACATTCTTACAAAATTAGGCAAATGTGTTATAATATGAAGTCTGCCACAGTGA |
| | | CTGTGGGTAATGCCAAAAATATCACAATCAGGAGGTATTAA |
| GhV | 11 | ATGAAGAAAAAGACGGACAATCCCACAATATCAAAGAGGGGTCACAACCATTCTCGAG |
| F coding | | GAATCAAATCTAGAGCGCTACTCAGAGAGACAGATAATTATTCCAATGGGCTAATAGTT |
| sequence | | GAGAATTTAGTTAGAAACTGTCATCATCCAAGTAAGAACAATCTAAACTATACTAAGAC |
| GenBank: | | ACAAAAAAGAGATTCTACAATCCCTTATCGTGTGGAAGAGAGAAAAGGACATTATCCAA |
| HQ660129.1 | | AGATTAAACATCTTATTGATAAATCTTACAAGCATATAAAAAGAGGGAAGAGAAGAAA |
| Predicted | | TGGTCATAATGGGAACATTATAACTATAATTCTGTTGTTGATTTTAATTTTGAAGACACA |
| TM domain | | GATGAGTGAAGGTGCTATCCATTACGAGACTCTAAGTAAGATCGGATTAATAAAGGGAA |
| underlined | | TCACCAGAGAGTACAAAGTCAAAGGAACTCCGTCAAGTAAAGACATAGTCATCAAATTG |
| | | ATTCCGAATGTCACCGGTCTTAACAAGTGCACGAACATATCAATGGAAAACTATAAAGA |
| | | ACAACTTGACAAAATACTAATTCCTATTAACAACATAATTGAATTGTATGCAAACTCAA |
| | | CTAAATCAGCCCCTGGGAATGCACGTTTTGCTGGCGTTATAATTGCAGGAGTGGCATTA |
| | | GGTGTTGCAGCGGCAGCCCAAATAACTGCCGGCATTGCACTGCATGAAGCTCGACAGAA |
| | | TGCAGAGAGAATTAATCTCTTAAAGGATAGCATTTCTGCCACTAACAACGCAGTAGCAG |
| | | AACTCCAGGAAGCAACTGGTGGAATAGTAAATGTCATTACAGGAATGCAAGATTACATC |
| | | AATACAAATCTAGTCCCGCAGATTGACAAACTGCAATGTAGTCAGATCAAAACGGCATT |
| | | AGACATATCTCTCTCCCAATACTATTCAGAAATATTAACAGTGTTCGGTCCAAACCTTCA |
| | | AAATCCAGTAACTACTTCCATGTCAATACAAGCCATATCACAATCCTTTGGGGGAAATA |
| | | TAGATTTGCTCTTAAACCTACTAGGTTACACTGCAAACGACTTATTGGATTTGCTCGAAA |
| | | GTAAAAGTATAACAGGCCAAATAACATACATAAATCTTGAACATTACTTCATGGTAATC |
| | | AGAGTATATTATCCTATAATGACAACAATCAGCAATGCTTATGTCCAGGAATTGATCAA |
| | | AATTAGCTTCAATGTCGATGGCAGTGAATGGGTATCTCTTGTACCCTCGTATATATTGAT |
| | | TAGAAACTCATATCTCTCAAACATAGACATATCAGAATGTCTCATAACCAAAAATTCAG |
| | | TGATATGTCGTCATGACTTTGCAATGCCAATGAGTTACACCTTAAAGGAATGCCTAACTG |
| | | GAGACACTGAAAAGTGTCCGAGAGAGGCTGTTGTAACCTCATATGTCCCAAGATTTGCT |
| | | ATCTCCGGGGGAGTGATTTATGCTAATTGTCTAAGTACAACATGTCAATGCTATCAAACT |
| | | GGCAAAGTAATTGCTCAAGACGGCAGCCAAACATTGATGATGATCGATAATCAAACATG |
| | | TTCAATAGTAAGAATTGAAGAAATCCTCATATCAACAGGGAAATATCTGGGAAGTCAGG |
| | | AGTACAATACGATGCATGTGTCAGTCGGCAATCCTGTCTTCACTGACAAGCTGGACATA |
| | | ACAAGTCAAATTTCCAACATCAACCAATCCATTGAACAATCCAAATTTTATCTAGATAA |
| | | GTCTAAGGCTATACTTGACAAGATAAATCTCAACTTAATTGGCTCTGTACCGATATCAAT |
| | | ACTTTTCATAATTGCGATCTTATCATTGATTCTCTCTATTATAACTTTTGTGATTGTGATG |
| | | ATAATTGTCAGAAGATATAACAAATACACTCCTCTTATAAACTCTGATCCATCCAGTAGG |
| | | AGGAGTACTATACAGGACGTATATATCATCCCGAACCCCGGAGAACATTCGATTAGATC |
| | | AGCTGCTCGATCAATTGACAGAGATCGAGATTGA |
| GhV | 12 | ATGCCGCAGAAGACTGTGGAATTCATTAACATGAATTCCCCTCTAGAAAGAGGGGTCAG |
| G coding | | CACTCTTTCAGATAAGAAGACCCTCAATCAATCTAAAATCACCAAGCAGGGGTATTTTG |
| sequence | | GGTTAGGATCCCACAGTGAGAGAAATTGGAAGAAGCAGAAGAATCAAAATGATCATTA |
| GenBank: | | CATGACTGTTTCAACCATGATTCTTGAGATATTAGTTGTCCTGGGCATCATGTTTAATCTC |
| HQ660129.1 | | ATAGTTTTAACTATGGTGTATTATCAGAATGACAACATCAATCAAAGGATGGCAGAACT |
| Predicted | | TACAAGCAATATCACAGTCCTGAATTTAAATCTTAATCAATTGACAAACAAAATTCAAA |
| TM domain | | GAGAAATTATTCCTAGGATCACTCTTATTGACACAGCAACCACCATTACAATTCCTAGTG |
| underlined | | CCATTACTTACATATTAGCAACTCTGACAACCAGAATCTCGGAATTATTGCCGTCAATCA |
| | | ACCAAAAGTGTGAGTTCAAGACACCGACACTTGTCCTGAATGACTGCAGAATAAACTGT |
| | | ACCCCACCACTAAACCCGTCTGATGGAGTGAAAATGAGTTCTCTTGCCACTAACTTGGTT |
| | | GCACATGGGCCCTCTCCCTGTAGAAACTTTTCATCCGTACCTACAATTTACTATTATCGG |
| | | ATTCCAGGATTATACAATAGAACAGCATTGGACGAAAGATGTATACTAAACCCGAGATT |
| | | GACAATAAGCAGTACAAAATTTGCTTATGTCCACTCTGAATATGATAAAAATTGCACCA |
| | | GAGGATTCAAATACTATGAATTGATGACATTTGGAGAAATACTGGAGGGTCCGGAAAAA |
| | | GAACCCAGAATGTTTTCTAGGTCATTTTATTCGCCCACAAATGCTGTGAACTATCATTCT |
| | | TGTACGCCGATCGTGACTGTCAATGAAGGATATTTTCTTTGCCTTGAATGCACCTCCTCA |
| | | GATCCCTTGTACAAAGCAAATCTATCTAATAGCACATTCCATTTGGTGATACTGAGGCAT |
| | | AACAAGGATGAGAAAATAGTTTCAATGCCTAGCTTTAACCTTTCTACTGATCAAGAGTA |
| | | TGTTCAGATAATCCCTGCAGAAGGTGGCGGCACAGCAGAGAGTGGCAATCTTTACTTCC |
| | | CTTGTATTGGAAGGCTCTTACACAAACGAGTCACCCATCCTTTATGCAAAAAGTCAAATT |
| | | GTTCGCGAACTGATGATGAATCTTGCCTGAAAAGTTATTACAATCAAGGGTCGCCTCAG |
| | | CACCAAGTAGTCAACTGTCTGATAAGGATCAGAAATGCACAGAGAGATAATCCAACCTG |
| | | GGATGTTATCACAGTTGATCTGACTAATACATACCCAGGATCAAGGAGCAGGATCTTTG |
| | | GAAGCTTCTCCAAACCGATGCTTTATCAATCATCAGTATCATGGCATACTCTTCTTCAGG |
| | | TAGCAGAGATAACAGACCTAGATAAGTATCAATTGGACTGGTTGGATACACCCTATATA |
| | | TCTCGTCCTGGAGGATCTGAGTGCCCTTTCGGAAATTATTGTCCAACGGTATGCTGGGAA |
| | | GGGACATATAATGATGTCTATAGCTTAACTCCAAATAACGATCTTTTTGTCACTGTGTAT |
| | | TTGAAGAGTGAACAAGTTGCAGAGAACCCTTATTTCGCAATCTTCTCCCGGGATCAAAT |
| | | CTTGAAAGAATTCCCTCTTGATGCATGGATAAGCAGTGCACGAACTACGACAATATCGT |
| | | GCTTCATGTTCAACAATGAAATTTGGTGTATAGCTGCATTAGAGATCACAAGATTGAAT |
| | | GATGACATCATAAGACCAATTTATTACTCTTTCTGGCTGCCTACTGATTGCCGGACACCA |
| | | TATCCCCACACCGGTAAGATGACCAGGGTTCCCTTGCGCTCCACATATAACTACTAA |
| CedV | 28 | ATGTCTAACAAGAGGACAACAGTATTGATCATAATAAGCTATACGTTATTTTATTTGAAT |
| F coding | | AATGCAGCAATTGTAGGGTTTGATTTTGATAAATTGAATAAAATAGGTGTGGTGCAAGG |
| sequence | | GAGAGTCCTAAATTATAAAATTAAAGGAGATCCAATGACAAAAGACCTTGTCTTGAAAT |
| GenBank: | | TTATCCCTAACATAGTGAATATCACTGAATGTGTGAGAGAGCCCTTGAGTAGGTACAAT |
| JQ001776.1 | | GAGACCGTGAGGAGATTGCTTTTACCTATACACAACATGCTTGGGTTATACTTGAATAAC |
| Predicted | | ACAAATGCTAAAATGACTGGGTTGATGATCGCGGGTGTGATCATGGGTGGGATAGCAAT |
| TM domain | | AGGTATAGCCACAGCAGCTCAGATCACAGCAGGTTTTGCTCTTTATGAGGCAAAAAAGA |
| underlined | | ACACAGAAAATATTCAGAAATTAACAGACAGCATCATGAAAACACAGGACTCGATTGA |
| | | TAAACTTACTGACAGTGTGGGGACAAGCATACTTATATTGAATAAGCTACAGACATACA |
| | | TCAACAATCAACTGGTACCAAATCTAGAGCTTCTATCCTGCCGACAAAACAAAATTGAG |
| | | TTTGATCTAATGTTAACCAAGTATTTGGTGGATCTTATGACTGTTATTGGTCCTAATATCA |
| | | ATAATCCTGTTAATAAAGATATGACTATTCAATCTTTGTCACTTCTTTTTGATGGCAATTA |
| | | TGATATAATGATGTCAGAACTTGGTTATACACCTCAGGATTTCTTAGATTTGATAGAGAG |
| | | TAAGAGTATAACAGGGCAAATAATTTATGTTGATATGGAAAACTTGTACGTTGTGATCA |
| | | GGACATATCTACCTACCCTAATTGAAGTACCTGATGCCCAAATATATGAGTTCAACAAA |
| | | ATAACTATGAGTAGCAATGGAGGAGAATACTTGTCAACCATACCTAATTTCATATTAAT |
| | | AAGAGGTAATTATATGTCTAATATAGATGTTGCAACATGTTATATGACCAAAGCAAGCG |
| | | TAATTTGTAATCAAGATTATTCACTCCCGATGAGCCAAAACTTAAGAAGCTGTTATCAAG |
| | | GTGAGACAGAATACTGTCCTGTTGAGGCAGTCATCGCGTCACACTCTCCAAGATTTGCTC |
| | | TTACAAATGGAGTTATTTTCGCCAATTGTATAAATACAATTTGTAGGTGTCAAGACAATG |
| | | GTAAGACTATCACTCAAAACATAAACCAATTCGTAAGCATGATCGACAACAGTACTTGT |
| | | AATGATGTCATGGTAGATAAGTTTACTATCAAGGTAGGAAAATATATGGGGAGAAAAG |
| | | ATATCAATAATATTAATATCCAGATAGGACCGCAGATCATAATTGATAAGGTTGACTTG |
| | | TCTAATGAAATAAACAAGATGAATCAATCTTTAAAAGATAGTATTTTCTACCTGAGAGA |
| | | AGCCAAGAGAATTTTAGACTCAGTAAATATCAGTCTTATATCTCCAAGCGTTCAATTGTT |
| | | TCTAATAATAATATCAGTCCTCTCATTTATTATATTATTGATTATCATAGTATACTTGTAC |
| | | TGTAAATCAAAACATTCATATAAATATAACAAATTTATAGATGATCCTGATTATTACAAT |
| | | GATTACAAAAGAGAACGTATTAATGGCAAAGCCAGTAAGAGTAACAATATATATTATGT |
| | | AGGTGATTAA |
| CedV | 29 | ATGCTTTCTCAGCTCCAAAAAAATTACTTAGACAACTCAAACCAACAAGGTGATAAAAT |
| G coding | | GAACAACCCAGATAAGAAATTAAGTGTCAACTTCAACCCTTTAGAATTAGATAAAGGTC |
| sequence | | AAAAAGATCTCAATAAGTCTTATTATGTTAAAAACAAGAATTATAACGTTTCAAATCTAT |
| GenBank: | | TAAATGAAAGTCTGCACGATATCAAGTTTTGTATTTATTGTATATTCTCACTGCTAATTAT |
| JQ001776.1 | | CATTACAATAATCAATATAATCACAATATCAATTGTTATAACTCGTCTGAAAGTACATGA |
| Predicted | | AGAGAATAATGGCATGGAATCTCCTAATTTACAATCTATTCAAGATAGTCTCTCATCTCT |
| TM domain | | TACTAACATGATCAATACAGAGATAACTCCTAGAATAGGGATTTTAGTTACAGCCACTT |
| underlined | | CTGTTACTCTCTCTTCATCTATCAATTATGTCGGGACTAAGACAAATCAACTGGTCAATG |
| | | AATTAAAAGATTATATAACCAAAAGTTGTGGCTTTAAGGTCCCTGAATTAAAGTTACAT |
| | | GAATGCAACATAAGTTGTGCTGATCCAAAAATTAGCAAATCTGCAATGTACAGCACCAA |
| | | TGCCTATGCCGAGCTTGCTGGTCCACCTAAGATATTTTGTAAAAGTGTATCCAAAGACCC |
| | | CGACTTTAGACTGAAGCAGATAGATTATGTAATACCAGTGCAGCAAGATCGGTCTATTT |
| | | GTATGAACAACCCTTTATTGGATATTTCTGATGGGTTTTTTACCTACATACATTATGAAG |
| | | GAATAAATAGCTGTAAAAAATCAGATTCATTTAAAGTGCTGCTGTCACATGGTGAAATA |
| | | GTTGACAGGGGTGATTATCGACCATCATTATATCTATTATCAAGTCATTACCATCCTTAT |
| | | TCAATGCAGGTAATAAACTGTGTACCTGTGACTTGTAACCAGTCATCCTTTGTATTCTGT |
| | | CATATCTCCAACAACACTAAAACATTGGACAATTCAGATTACTCGTCAGACGAGTACTA |
| | | CATAACATATTTCAATGGCATAGATCGTCCCAAAACCAAGAAGATTCCCATTAACAATA |
| | | TGACAGCAGACAATCGTTATATCCATTTTACATTCTCAGGTGGGGGAGGTGTATGTTTAG |
| | | GTGAAGAATTTATTATTCCTGTTACCACAGTCATCAATACTGATGTATTCACGCATGATT |
| | | ATTGTGAGAGTTTCAACTGTTCAGTCCAAACCGGTAAAAGTCTAAAGGAGATATGCTCT |
| | | GAGTCATTAAGATCTCCAACGAACTCATCGCGATACAATTTAAACGGAATCATGATTAT |
| | | AAGTCAAAACAACATGACAGATTTTAAGATTCAGTTGAATGGTATAACTTATAACAAAC |
| | | TGTCATTCGGAAGTCCTGGAAGACTGAGCAAGACACTGGGCCAGGTCCTTTATTACCAA |
| | | TCTTCAATGAGTTGGGATACTTATCTAAAGGCAGGATTTGTCGAGAAATGGAAACCCTTT |
| | | ACCCCGAATTGGATGAACAATACTGTGATATCCAGACCTAACCAAGGTAATTGTCCAAG |
| | | GTATCATAAATGCCCCGAGATATGTTATGGAGGGACATACAATGATATTGCTCCTTTAG |
| | | ATCTAGGAAAAGACATGTATGTTAGCGTTATTCTAGATTCAGATCAGCTTGCAGAGAAT |
| | | CCAGAGATTACAGTATTTAACTCTACTACTATACTTTATAAGGAGAGAGTATCCAAAGA |
| | | TGAACTAAACACAAGAAGTACTACAACGAGCTGTTTTCTTTTCCTAGATGAACCTTGGTG |
| | | TATATCAGTATTAGAAACAAACAGATTTAACGGCAAATCTATTAGGCCCGAGATTTATT |
| | | CATACAAAATTCCTAAGTATTGTTAA |

The non-CedV henipavirus that can be chimerized with CedV (i.e., the henipavirus whose F and/or G proteins are expressed) may be selected from Hendra virus (HeV), Nipah virus (NiV), Ghana virus (GhV), and Mojiang virus (MojV). In specific embodiments, the non-CedV henipavirus may be a strain of NiV selected from the Malaysian strain of NiV (NiV-M) and the Bangladesh strain of NiV (NiV-B). Other henipaviruses may similarly be chimerized with CedV.

In some embodiments, the rCedV chimera comprises one or both of the F and G envelope glycoprotein genes of a non-CedV henipavirus (e.g., HeV, NiV, GhV, MojV, or any other pathogenic henipavirus). In some embodiments, the rCedV chimera comprises the F and G envelope glycoprotein genes of a non-CedV henipavirus (e.g., HeV, NiV, GhV, MojV, or any other pathogenic henipavirus). For instance, none limiting examples include a rCedV chimera may comprise a MojV F envelope glycoprotein gene and a GhV G envelope glycoprotein gene or a GhV F envelope glycoprotein gene and a MojV G envelope glycoprotein gene, or any other combination of non-henipavirus F and G envelope glycoprotein genes.

In some embodiments, the rCedV chimera comprises one or both of the F and G envelope glycoprotein genes of HeV. In some embodiments, the rCedV chimera comprises both F and G envelope glycoprotein genes of HeV (designated herein as chimera rCedV-HeV). In some embodiments, the rCedV chimera comprises a CedV F envelope glycoprotein gene and a HeV G envelope glycoprotein gene. In some embodiments, the rCedV comprises a CedV G envelope glycoprotein gene and a HeV F envelope glycoprotein gene.

In some embodiments, the rCedV chimera comprises one or both of the F and G envelope glycoprotein genes of NiV. In some embodiments, the rCedV chimera comprises both F and G envelope glycoprotein genes of NiV (designated herein as chimera rCedV-NiV). In some embodiments, the rCedV chimera comprises a CedV F envelope glycoprotein gene and a NiV G envelope glycoprotein gene. In some embodiments, the rCedV comprises a CedV G envelope glycoprotein gene and a NiV F envelope glycoprotein gene.

In some embodiments, the rCedV chimera comprises one or both of the F and G envelope glycoprotein genes of NiV-M. In some embodiments, the rCedV chimera comprises both F and G envelope glycoprotein genes of NiV-M (designated herein as chimera rCedV-NiV-M). In some embodiments, the rCedV chimera comprises a CedV F envelope glycoprotein gene and a NiV-M G envelope glycoprotein gene. In some embodiments, the rCedV comprises a CedV G envelope glycoprotein gene and a NiV-M F envelope glycoprotein gene.

In some embodiments, the rCedV chimera comprises one or both of the F and G envelope glycoprotein genes of NiV-B. In some embodiments, the rCedV chimera comprises both F and G envelope glycoprotein genes of NiV-B (designated herein as chimera rCedV-NiV-B). In some embodiments, the rCedV chimera comprises a CedV F envelope glycoprotein gene and a NiV-B G envelope glycoprotein gene. In some embodiments, the rCedV comprises a CedV G envelope glycoprotein gene and a F envelope glycoprotein gene.

In some embodiments, the rCedV chimera comprises one or both of the F and G envelope glycoprotein genes of GhV. In some embodiments, the rCedV chimera comprises both F and G envelope glycoprotein genes of GhV. In some embodiments, the rCedV chimera comprises a CedV F envelope glycoprotein gene and a GhV G envelope glycoprotein gene. In some embodiments, the rCedV comprises a CedV G envelope glycoprotein gene and a GhV F envelope glycoprotein gene.

In some embodiments, the rCedV chimera comprises one or both of the F and G envelope glycoprotein genes of MojV. In some embodiments, the rCedV chimera comprises both F and G envelope glycoprotein genes of MojV. In some embodiments, the rCedV chimera comprises a CedV F envelope glycoprotein gene and a MojV G envelope glycoprotein gene. In some embodiments, the rCedV comprises a CedV G envelope glycoprotein gene and a MojV F envelope glycoprotein gene.

In some embodiments, the rCedV comprises a modified version of a gene encoding a non-CedV henipavirus F or G protein. For example, to maintain the “rule of 6,” a feature of henipaviruses to promote replication initiation, the F coding region of NiV-B can be modified. For example, the last 3 nucleotides (ACG) before the stop codon (TAG) of the NiV-B F coding region may be deleted in the chimeric design, as deletion of this amino acid (Threonine) does not interfere with endocytosis, trafficking, or fusion, and therefore would not impede the successful rescue of the chimeric virus nor interfere with the functionality of the NiV-B F protein. An example of a modified NiV-B F protein coding sequence is set forth below:

Modified NiV-B F Protein Coding Sequence
(SEQ ID NO: 13)
ATGGCAGTTATACTTAACAAGAGATATTATTCTAATCTCTTAATACTGATTTTGATGA
TCTCGGAGTGCAGTGTCGGGATTTTGCATTATGAGAAATTGAGTAAGATTGGGCTTG
TCAAAGGAATAACAAGAAAATACAAGATCAAAAGCAATCCTCTCACAAAAGACATT
GTTATTAAAATGATTCCGAATGTGTCAAACATGTCTCAATGCACGGGGAGTGTCATG
GAAAACTATAAAACACGATTAAACGGTATCCTAACGCCTATAAAGGGGGCATTAGA
GATTTACAAGAACAACACTCATGACCTTGTCGGTGATGTAAGACTGGCCGGAGTTAT
AATGGCAGGAGTTGCTATTGGAATTGCAACCGCAGCTCAAATTACTGCAGGTGTAGC
ATTATATGAGGCAATGAAAAATGCTGACAACATCAACAAACTCAAAAGCAGCATAG
AATCAACTAATGAAGCTGTTGTTAAGCTTCAAGAGACTGCAGAAAAGACAGTCTAT
GTACTGACCGCTTTGCAGGATTACATTAATACTAACTTGGTACCGACAATTGACAAG
ATAAGCTGCAAACAGACGGAACTCTCATTAGATCTAGCACTATCAAAGTACCTCTCT
GATTTGCTTTTTGTATTTGGTCCCAACCTTCAAGACCCAGTTTCTAATTCAATGACTA
TACAGGCTATATCTCAGGCATTCGGTGGAAATTATGAAACACTGCTAAGAACGTTGG
GTTACGCTACAGAAGACTTTGATGATCTTCTAGAAAGTGACAGCATAACGGGTCAA
ATTATCTACGTTGATTTAAGTGGCTACTACATAATTGTCAGGGTTTATTTTCCTATCC
TGACTGAAATCCAACAGGCCTATATCCAAGAATTGTTGCCAGTGAGCTTTAACAATG
ACAATTCAGAATGGATCAGCATTGTCCCAAATTTCATATTGGTAAGGAACACATTAA
TATCAAATATAGAGATTGGATTTTGCCTAATTACAAAGAGGAGTGTGATCTGCAACC
AAGATTATGCAACACCCATGACAAACAATATGAGGGAATGTTTGACGGGGTCGACT
GAGAAGTGTCCTCGAGAGCTGGTGGTTTCATCACACGTTCCCAGATTTGCACTATCT
AACGGGGTTTTGTTTGCTAATTGCATAAGCGTCACATGCCAGTGTCAAACAACAGGT
AGGGCAATCTCACAGTCAGGAGAACAAACTCTGCTGATGATTGATAACACCACCTG
TCCTACAGCCGTACTCGGTAATGTGATCATCAGCTTGGGAAAATATCTTGGGTCAGT
AAATTATAACTCTGAAGGCATTGCTATTGGTCCTCCTGTCTTTACTGATAAAGTTGAC
ATATCAAGTCAAATATCTAGCATGAATCAGTCCTTACAACAATCTAAGGACTATATC
AAAGAAGCTCAACGACTCCTTGATACTGTTAACCCGTCATTAATAAGCATGTTGTCT
ATGATCATACTGTATGTACTATCAATTGCATCATTGTGTATAGGATTGATTACATTTA
TCAGTTTTATCATTGTTGAGAAAAAAAGAAACACCTATAGCAGATTAGAGGACAGG
AGAGTCAGACCTACAAGTAGTGGGGATCTCTATTACATTGGGTAG

In a further example, to maintain the “rule of 6” an extra stop codon (TAA; bolded and underlined below), can be added to the end of F coding region from HeV, as shown below. The HeV F amino acid sequence encoded by the sequence below is identical to the amino acid sequence of the HeV 2008 Redlands isolate (GenBank: JN255805.1) and protein sequence GenBank: AEQ38070.1. The exemplary rCedV-HeV clones set forth herein use the F coding sequence of the HeV genomic sequence (GenBank: MN062017.1).

Modified HeV F Protein Coding Sequence
(SEQ ID NO: 14)
ATGGCTACACAAGAGGTCAGGCTAAAGTGTTTGCTCTGTGGGATCATAGTTCTGGTT
TTGTCATTAGAAGGGCTAGGGATACTACATTATGAGAAACTTAGTAAGATAGGGCT
GGTTAAAGGTATTACAAGAAAGTACAAGATTAAGAGTAACCCTTTGACCAAGGATA
TTGTGATCAAAATGATCCCTAATGTCTCGAATGTCTCAAAGTGCACCGGGACTGTTA
TGGAGAATTACAAAAGCAGACTCACAGGGATTCTCTCACCAATCAAAGGCGCCATC
GAACTGTACAATAATAACACGCATGACCTAGTTGGTGATGTCAAGCTTGCAGGTGTG
GTGATGGCAGGGATTGCAATCGGGATAGCTACTGCTGCACAAATCACAGCAGGTGT
TGCCTTATATGAGGCAATGAAGAACGCAGACAATATCAATAAACTCAAGAGCAGCA
TAGAGTCTACAAATGAGGCTGTTGTCAAATTACAGGAAACAGCTGAGAAAACAGTC
TACGTCCTTACTGCTCTTCAAGATTACATCAACACTAACCTTGTTCCTACAATAGATC
AAATTAGCTGCAAGCAAACAGAGCTCGCATTAGACTTGGCGTTGTCTAAGTATCTGT
CTGATCTGCTCTTTGTTTTCGGACCTAACTTACAGGATCCAGTCTCTAATTCCATGAC
TATCCAAGCAATATCTCAAGCATTTGGGGGCAATTACGAAACCTTACTGAGAACGCT
TGGTTACGCGACCGAGGACTTCGACGACCTTTTAGAAAGTGATAGCATAACAGGCC
AGATAGTCTATGTAGATCTCAGTAGCTATTACATAATAGTAAGGGTGTATTTTCCCA
TACTAACAGAGATCCAACAGGCTTATGTGCAGGAGTTGCTTCCAGTGAGTTTTAATA
ACGATAATTCAGAATGGATCAGCATTGTCCCGAATTTCGTGCTGATTAGGAACACGC
TGATTTCAAATATAGAAGTCAAGTACTGCTTAATCACCAAGAAAAGTGTGATTTGTA
ATCAGGACTATGCTACACCCATGACGGCTAGCGTGAGAGAATGCTTGACAGGATCC
ACAGATAAGTGCCCAAGGGAGTTAGTAGTCTCATCCCATGTTCCAAGATTTGCCCTC
TCAGGAGGAGTCTTGTTTGCAAATTGTATAAGTGTGACATGTCAGTGTCAGACTACT
GGGAGGGCAATATCTCAATCAGGGGAACAGACACTACTGATGATTGACAATACTAC
CTGCACAACAGTTGTTCTAGGAAACATAATCATAAGCCTTGGAAAATATTTGGGATC
AATAAATTACAATTCTGAGAGCATTGCTGTTGGGCCACCAGTCTATACAGACAAAGT
TGATATCTCAAGTCAGATATCTAGTATGAATCAATCACTACAACAATCTAAGGATTA
CATTAAAGAAGCTCAAAAGATCTTGGACACTGTGAATCCGTCGTTGATAAGTATGCT
ATCAATGATCATCCTTTATGTTTTGTCCATTGCAGCACTGTGCATTGGTCTGATCACT
TTCATAAGCTTTGTAATAGTTGAGAAAAAGAGAGGGAATTACAGCAGGCTAGATGA
TAGGCAAGTGCGACCGGTCAGTAATGGTGATCTGTATTATATTGGAACATAATAA

In a further example, a single nucleotide (T1593A; bolded and underlined below), which does not result in an amino acid change, can be modified to remove an internal PstI site from a HeV G-encoding sequence. The HeV G amino acid sequence encoded by the sequence below is identical to the amino acid sequence of the HeV 2008 Redlands isolate (JN255805.1), and protein sequence GenBank: AEQ38071.1. The exemplary rCedV-HeV clones set forth herein use the G coding sequence derived from the HeV genomic sequence (GenBank: MN062017.1).

Modified HeV G Protein Coding Sequence
(SEQ ID NO: 15)
ATGATGGCTGATTCCAAATTGGTAAGCCTGAACAATAATCTATCTGGTAAAATCAAG
GATCAAGGTAAAGTTATCAAGAATTATTACGGCACAATGGACATCAAGAAAATTAA
CGATGGGTTATTAGATAGTAAGATACTTGGGGCGTTTAACACAGTGATAGCTTTGTT
GGGATCAATCATCATCATTGTGATGAATATCATGATAATTCAAAATTACACCAGAAC
GACTGATAATCAGGCACTAATCAAAGAGTCACTCCAGAGTGTACAGCAACAAATCA
AAGCTTTAACAGACAAAATCGGGACAGAGATAGGCCCCAAAGTCTCACTAATTGAC
ACATCCAGCACCATCACAATTCCTGCTAACATAGGGTTACTGGGATCCAAGATAAGT
CAGTCTACCAGCAGTATTAATGAGAATGTTAACGATAAATGCAAATTTACTCTTCCT
CCTTTAAAGATTCATGAGTGTAATATCTCTTGTCCGAATCCTTTGCCTTTCAGAGAAT
ACCGACCAATCTCACAAGGGGTGAGTGATCTTGTAGGACTGCCGAACCAGATCTGTC
TACAGAAGACAACATCAACAATCTTAAAGCCCAGGCTGATATCCTATACTCTACCAA
TTAATACCAGAGAAGGGGTTTGCATCACTGACCCACTTTTGGCTGTTGATAATGGCT
TCTTCGCCTATAGCCATCTTGAAAAGATCGGATCATGTACTAGAGGAATTGCAAAAC
AAAGGATAATAGGGGTGGGTGAGGTATTGGATAGGGGTGATAAGGTGCCATCAATG
TTTATGACCAATGTTTGGACACCACCCAATCCAAGCACCATCCATCATTGCAGCTCA
ACTTACCATGAAGATTTTTATTACACATTGTGCGCAGTGTCCCATGTGGGAGATCCT
ATCCTTAACAGTACTTCCTGGACAGAGTCACTGTCTCTGATTCGTCTTGCTGTAAGAC
CAAAAAGTGATAGTGGAGACTACAATCAGAAATACATCGCTATAACTAAAGTTGAA
AGAGGGAAGTACGATAAGGTGATGCCTTACGGTCCATCAGGTATCAAGCAAGGGGA
TACATTGTACTTTCCGGCCGTCGGTTTTTTGCCAAGGACCGAATTTCAATATAATGAC
TCTAATTGTCCCATAATTCATTGCAAGTACAGCAAAGCAGAAAACTGTAGGCTTTCA
ATGGGTGTCAACTCCAAAAGTCATTATATTTTGAGATCAGGACTATTGAAGTATAAT
CTATCTCTTGGAGGAGACATCATACTCCAATTTATCGAGATTGCTGACAATAGATTG
ACCATCGGTTCTCCTAGTAAGATATACAATTCCCTAGGTCAACCCGTTTTCTACCAG
GCATCATATTCTTGGGATACGATGATTAAATTAGGCGATGTTGATACCGTTGACCCT
CTAAGAGTACAGTGGAGAAATAACAGTGTGATTTCTAGACCTGGACAGTCACAGTG
TCCTCGATTTAATGTCTGTCCCGAGGTATGCTGGGAAGGGACATATAATGATGCTTT
TCTAATAGACCGGCTAAACTGGGTTAGTGCTGGTGTTTATTTAAACAGTAACCAAAC
AGCAGAGAACCCTGTGTTTGCCGTATTCAAGGATAACGAGATCCTTTACCAAGTTCC
ACTGGCTGAAGATGACACAAATGCACAAAAAACCATCACAGATTGCTTCTTGCTGG
AGAATGTCATATGGTGTATATCACTAGTAGAAATATACGATACAGGAGACAGTGTG
ATAAGGCCAAAACTATTTGCAGTCAAGATACCTGCCCAATGTTCAGAGAGTTGA

In a further example, a single nucleotide (C924A; bolded and underlined below), which does not result in an amino acid change, can be modified to remove an internal AleI site from a MojV F-encoding sequence. Further, to maintain the “rule of 6” an extra stop codon (TAA; bolded and underlined below), can be added to the end of the MojV F coding region.

Modified MojV F Protein Coding Sequence
(SEQ ID NO: 16)
ATGGCACTAAATAAAAATATGTTCAGTTCACTGTTCCTTGGTTATCTATTAGTGTACG
CTACGACTGTTCAGTCTAGTATACACTATGACTCCTTATCTAAGGTCGGTGTCATTAA
GGGTCTGACATACAACTATAAGATCAAGGGTTCGCCATCTACAAAGCTAATGGTGGT
CAAATTGATACCTAACATTGATAGTGTTAAAAACTGTACTCAGAAACAGTATGATGA
ATACAAGAACTTAGTAAGGAAAGCCTTAGAACCGGTCAAAATGGCTATTGACACCA
TGCTCAATAATGTTAAGTCGGGTAATAACAAGTACAGATTTGCAGGTGCAATTATGG
CTGGAGTTGCCCTCGGTGTTGCAACAGCAGCCACTGTTACAGCAGGGATAGCTCTCC
ATAGATCAAATGAAAATGCACAGGCAATTGCAAACATGAAGAGTGCTATTCAAAAT
ACAAATGAGGCAGTAAAGCAATTGCAATTGGCCAATAAACAAACACTAGCTGTGAT
TGACACCATAAGAGGAGAGATCAATAACAATATAATACCCGTTATAAATCAATTGA
GCTGTGACACAATTGGGCTCAGTGTAGGTATAAGACTCACTCAGTACTACTCTGAAA
TAATAACTGCATTTGGGCCAGCTTTGCAGAATCCAGTAAATACAAGGATTACCATTC
AAGCAATATCTAGTGTGTTTAATGGCAACTTTGATGAACTGCTGAAGATTATGGGGT
ATACAAGTGGTGATCTTTATGAAATTCTACATAGTGAATTAATTAGAGGCAACATTA
TAGACGTTGATGTAGATGCAGGATACATAGCTCTAGAAATAGAATTCCCCAATCTAA
CATTGGTACCTAATGCTGTAGTACAGGAGTTAATGCCTATCAGTTATAACATAGACG
GGGATGAGTGGGTAACACTTGTGCCAAGGTTTGTACTTACAAGGACTACACTGTTAT
CAAATATTGATACGAGTAGATGTACAATCACAGATAGTAGTGTCATATGTGACAAC
GACTACGCCTTGCCTATGTCACACGAGCTTATTGGCTGCTTACAGGGAGATACATCT
AAGTGTGCTAGAGAGAAGGTAGTCTCAAGTTATGTCCCTAAATTTGCGTTGTCTGAT
GGGTTAGTGTATGCAAATTGCCTCAATACTATCTGCCGATGTATGGATACAGATACT
CCAATCTCACAAAGTCTCGGAGCCACTGTATCATTACTAGACAACAAGAGGTGTTCA
GTATATCAGGTAGGAGATGTCTTGATTTCTGTCGGATCATATCTAGGAGATGGAGAA
TATAATGCTGATAATGTAGAGCTTGGCCCACCTATAGTTATAGATAAGATTGACATA
GGAAATCAGCTGGCAGGTATTAATCAAACCTTACAAGAGGCAGAAGATTACATTGA
GAAGTCAGAAGAGTTCTTAAAAGGGGTTAACCCTTCAATTATCACTCTTGGTTCCAT
GGTTGTCCTTTATATATTTATGATATTAATAGCCATTGTGTCAGTAATAGCACTAGTA
TTGTCAATTAAATTAACAGTAAAAGGTAACGTGGTAAGGCAGCAGTTCACGTATACT
CAGCATGTTCCTAGCATGGAGAATATCAATTATGTAAGTCATTAATAA

In a further example, to maintain the “rule of 6” an extra stop codon (TGA; bolded and underlined below), can be added to the end of F coding region from GhV, as shown below.

Modified GhV F Protein Coding Sequence
(SEQ ID NO: 17)
ATGAAGAAAAAGACGGACAATCCCACAATATCAAAGAGGGGTCACAACCATTCTCG
AGGAATCAAATCTAGAGCGCTACTCAGAGAGACAGATAATTATTCCAATGGGCTAA
TAGTTGAGAATTTAGTTAGAAACTGTCATCATCCAAGTAAGAACAATCTAAACTATA
CTAAGACACAAAAAAGAGATTCTACAATCCCTTATCGTGTGGAAGAGAGAAAAGGA
CATTATCCAAAGATTAAACATCTTATTGATAAATCTTACAAGCATATAAAAAGAGGG
AAGAGAAGAAATGGTCATAATGGGAACATTATAACTATAATTCTGTTGTTGATTTTA
ATTTTGAAGACACAGATGAGTGAAGGTGCTATCCATTACGAGACTCTAAGTAAGATC
GGATTAATAAAGGGAATCACCAGAGAGTACAAAGTCAAAGGAACTCCGTCAAGTAA
AGACATAGTCATCAAATTGATTCCGAATGTCACCGGTCTTAACAAGTGCACGAACAT
ATCAATGGAAAACTATAAAGAACAACTTGACAAAATACTAATTCCTATTAACAACA
TAATTGAATTGTATGCAAACTCAACTAAATCAGCCCCTGGGAATGCACGTTTTGCTG
GCGTTATAATTGCAGGAGTGGCATTAGGTGTTGCAGCGGCAGCCCAAATAACTGCC
GGCATTGCACTGCATGAAGCTCGACAGAATGCAGAGAGAATTAATCTCTTAAAGGA
TAGCATTTCTGCCACTAACAACGCAGTAGCAGAACTCCAGGAAGCAACTGGTGGAA
TAGTAAATGTCATTACAGGAATGCAAGATTACATCAATACAAATCTAGTCCCGCAGA
TTGACAAACTGCAATGTAGTCAGATCAAAACGGCATTAGACATATCTCTCTCCCAAT
ACTATTCAGAAATATTAACAGTGTTCGGTCCAAACCTTCAAAATCCAGTAACTACTT
CCATGTCAATACAAGCCATATCACAATCCTTTGGGGGAAATATAGATTTGCTCTTAA
ACCTACTAGGTTACACTGCAAACGACTTATTGGATTTGCTCGAAAGTAAAAGTATAA
CAGGCCAAATAACATACATAAATCTTGAACATTACTTCATGGTAATCAGAGTATATT
ATCCTATAATGACAACAATCAGCAATGCTTATGTCCAGGAATTGATCAAAATTAGCT
TCAATGTCGATGGCAGTGAATGGGTATCTCTTGTACCCTCGTATATATTGATTAGAA
ACTCATATCTCTCAAACATAGACATATCAGAATGTCTCATAACCAAAAATTCAGTGA
TATGTCGTCATGACTTTGCAATGCCAATGAGTTACACCTTAAAGGAATGCCTAACTG
GAGACACTGAAAAGTGTCCGAGAGAGGCTGTTGTAACCTCATATGTCCCAAGATTTG
CTATCTCCGGGGGAGTGATTTATGCTAATTGTCTAAGTACAACATGTCAATGCTATC
AAACTGGCAAAGTAATTGCTCAAGACGGCAGCCAAACATTGATGATGATCGATAAT
CAAACATGTTCAATAGTAAGAATTGAAGAAATCCTCATATCAACAGGGAAATATCT
GGGAAGTCAGGAGTACAATACGATGCATGTGTCAGTCGGCAATCCTGTCTTCACTGA
CAAGCTGGACATAACAAGTCAAATTTCCAACATCAACCAATCCATTGAACAATCCA
AATTTTATCTAGATAAGTCTAAGGCTATACTTGACAAGATAAATCTCAACTTAATTG
GCTCTGTACCGATATCAATACTTTTCATAATTGCGATCTTATCATTGATTCTCTCTATT
ATAACTTTTGTGATTGTGATGATAATTGTCAGAAGATATAACAAATACACTCCTCTT
ATAAACTCTGATCCATCCAGTAGGAGGAGTACTATACAGGACGTATATATCATCCCG
AACCCCGGAGAACATTCGATTAGATCAGCTGCTCGATCAATTGACAGAGATCGAGA
TTGATGA

In a further example, two nucleotide changes (T321C to remove an internal SwaI site and C1653A to remove an internal SmaI site; both bolded and underlined below) can be made. These changes do not result in any amino acid changes.

Modified GhV G Protein Coding Sequence
(SEQ ID NO: 18)
ATGCCGCAGAAGACTGTGGAATTCATTAACATGAATTCCCCTCTA
GAAAGAGGGGTCAGCACTCTTTCAGATAAGAAGACCCTCAATCAA
TCTAAAATCACCAAGCAGGGGTATTTTGGGTTAGGATCCCACAGT
GAGAGAAATTGGAAGAAGCAGAAGAATCAAAATGATCATTACATG
ACTGTTTCAACCATGATTCTTGAGATATTAGTTGTCCTGGGCATC
ATGTTTAATCTCATAGTTTTAACTATGGTGTATTATCAGAATGAC
AACATCAATCAAAGGATGGCAGAACTTACAAGCAATATCACAGTC
CTGAACTTAAATCTTAATCAATTGACAAACAAAATTCAAAGAGAA
ATTATTCCTAGGATCACTCTTATTGACACAGCAACCACCATTACA
ATTCCTAGTGCCATTACTTACATATTAGCAACTCTGACAACCAGA
ATCTCGGAATTATTGCCGTCAATCAACCAAAAGTGTGAGTTCAAG
ACACCGACACTTGTCCTGAATGACTGCAGAATAAACTGTACCCCA
CCACTAAACCCGTCTGATGGAGTGAAAATGAGTTCTCTTGCCACT
AACTTGGTTGCACATGGGCCCTCTCCCTGTAGAAACTTTTCATCC
GTACCTACAATTTACTATTATCGGATTCCAGGATTATACAATAGA
ACAGCATTGGACGAAAGATGTATACTAAACCCGAGATTGACAATA
AGCAGTACAAAATTTGCTTATGTCCACTCTGAATATGATAAAAAT
TGCACCAGAGGATTCAAATACTATGAATTGATGACATTTGGAGAA
ATACTGGAGGGTCCGGAAAAAGAACCCAGAATGTTTTCTAGGTCA
TTTTATTCGCCCACAAATGCTGTGAACTATCATTCTTGTACGCCG
ATCGTGACTGTCAATGAAGGATATTTTCTTTGCCTTGAATGCACC
TCCTCAGATCCCTTGTACAAAGCAAATCTATCTAATAGCACATTC
CATTTGGTGATACTGAGGCATAACAAGGATGAGAAAATAGTTTCA
ATGCCTAGCTTTAACCTTTCTACTGATCAAGAGTATGTTCAGATA
ATCCCTGCAGAAGGTGGCGGCACAGCAGAGAGTGGCAATCTTTAC
TTCCCTTGTATTGGAAGGCTCTTACACAAACGAGTCACCCATCCT
TTATGCAAAAAGTCAAATTGTTCGCGAACTGATGATGAATCTTGC
CTGAAAAGTTATTACAATCAAGGGTCGCCTCAGCACCAAGTAGTC
AACTGTCTGATAAGGATCAGAAATGCACAGAGAGATAATCCAACC
TGGGATGTTATCACAGTTGATCTGACTAATACATACCCAGGATCA
AGGAGCAGGATCTTTGGAAGCTTCTCCAAACCGATGCTTTATCAA
TCATCAGTATCATGGCATACTCTTCTTCAGGTAGCAGAGATAACA
GACCTAGATAAGTATCAATTGGACTGGTTGGATACACCCTATATA
TCTCGTCCTGGAGGATCTGAGTGCCCTTTCGGAAATTATTGTCCA
ACGGTATGCTGGGAAGGGACATATAATGATGTCTATAGCTTAACT
CCAAATAACGATCTTTTTGTCACTGTGTATTTGAAGAGTGAACAA
GTTGCAGAGAACCCTTATTTCGCAATCTTCTCACGGGATCAAATC
TTGAAAGAATTCCCTCTTGATGCATGGATAAGCAGTGCACGAACT
ACGACAATATCGTGCTTCATGTTCAACAATGAAATTTGGTGTATA
GCTGCATTAGAGATCACAAGATTGAATGATGACATCATAAGACCA
ATTTATTACTCTTTCTGGCTGCCTACTGATTGCCGGACACCATAT
CCCCACACCGGTAAGATGACCAGGGTTCCCTTGCGCTCCACATAT
AACTACTAA

The present disclosure includes other variations to non-CedV coding sequences for F and G proteins. In some embodiments, a rCedV chimera may comprise a gene encoding a non-CedV F protein, a gene encoding a non-CedV G protein, or both, in which the gene encoding the non-CedV F protein and/or the gene encoding the non-CedV G protein may comprise 1, 2, 3, 4, 5, 6, 7, 8, 9, or 10 or more substitutions, insertions, or deletions, so long as the resulting virus is replication-competent. For instance, the gene encoding the non-CedV F protein may comprise a sequence that is about 90%, about 91%, about 92%, about 93%, about 94%, about 95%, about 96%, about 97%, about 98%, about 99%, or 100% identical to any one of SEQ ID NOs: 3, 5, 6, 9, 11, 13, 14, 16, or 17. Similarly, the gene encoding the non-CedV G protein may comprise a sequence that is about 90%, about 91%, about 92%, about 93%, about 94%, about 95%, about 96%, about 97%, about 98%, about 99%, or 100% identical to any one of SEQ ID NOs: 4, 7, 8, 10, 12, 15, or 18.

In any embodiments of the chimeras described herein, the chimera may further comprise a reporter sequence. For example, the reporter sequence may encode green fluorescent protein (GFP) or luciferase protein (Luc).

As described in more detail below, such recombinant Cedar virus (rCedV) chimeras in which one or both of the F and G envelope glycoprotein genes of CedV are replaced with a corresponding F and/or G envelope glycoprotein from a non-CedV henipavirus may be used to elicit an immune response to a non-CedV henipavirus, e.g., in a vaccine optionally comprising a pharmaceutically acceptable carrier and, further optionally, an adjuvant.

### B. Chimeras Expressing Soluble F and G Proteins

Another aspect of the present disclosure is directed to replication-competent, recombinant Cedar virus (rCedV) chimeras comprising the F and G envelope glycoprotein genes of CedV, and further comprising a coding sequence for one or both of (i) a soluble F envelope glycoprotein (sF) of a non-CedV henipavirus and (ii) a soluble G envelope glycoprotein (sG) of a non-CedV henipavirus. In some embodiments, the non-CedV coding sequences are NiV coding sequences encoding NiV sF and/or NiV sG. In some embodiments, the non-CedV coding sequences are HeV coding sequence encoding HeV sF and/or HeV sG. In some embodiments, the non-CedV coding sequences are MojV coding sequences encoding MojV sF and/or MojV sG. In some embodiments, the non-CedV coding sequences are GhV coding sequences encoding GhV sF and/or GhV sG.

When both non-CedV sF and sG genes are inserted into the rCedV genome, both genes may come from the same non-CedV henipavirus (e.g., HeV, NiV, GhV, or MojV) or each may come from a different non-CedV henipavirus.

Without wishing to be bound by theory, these chimeras may be advantageous because a sF or sG may be superior to that of full-length, membrane-bound protein when used as a vaccine immunogen when administered to subjects. Additionally, chimeras comprising HeV sF and/or sG may induce an immune response that can provide protection against not only HeV, but also NiV-M and NiV-B.

The soluble form of a non-CedV F or G protein can comprise the full ectodomain or an immunogenic fragment thereof. In some embodiments, the sF or sG of the non-CedV henipavirus comprises or consists of the full ectodomain sequence of the protein. In some embodiments, the sF or sG comprise or consist of a fragment of the ectodomain that is about 70%, about 75%, about 80%, about 85%, about 90%, about 91%, about 92%, about 93%, about 94%, about 95%, about 96%, about 97%, about 98%, or about 99% of the length of the full ectodomain sequence of the F or G protein of the given non-CedV henipavirus. In some embodiments there is a GCNtet amino acid sequence inserted to promote tetramerization.

Exemplary sF and sG coding sequences are provided below in Table 2.

TABLE 2
Exemplary soluble F (sF) and G (sG) Sequences

| | SEQ | |
|---|---|---|
| | ID | |
| Name | NO: | Sequence |
| NIV-M | 19 | ATGGTGGTGATCCTGGACAAGAGGTGCTACTGCAACCTGCTGATCCTGATCCTGATGATC |
| SF coding | | AGCGAGTGCAGCGTGGGCATCCTGCACTACGAGAAGCTGTCCAAGATCGGCCTGGTGAAG |
| sequence | | GGCGTGACCAGGAAGTACAAGATCAAGAGCAACCCCCTGACCAAGGACATCGTGATCAAG |
| Genbank: | | ATGATCCCCAACGTGAGCGACATGAGCCAGTGCACCGGCAGCGTGATGGAGAACTACAAG |
| AY816748.1 | | ACCAGGCTGAACGGCATCCTGACCCCCATCAAGGGCGCCCTGGAGATCTACAAGAACAAC |
| | | ACCCACGACCTGGTGGGCGACGTGAGACTGGCCGGCGTGATCATGGCCGGCGTGGCCATC |
| | | GGCATCGCTACAGCCGCCCAGATCACAGCCGGAGTGGCCCTGTACGAGGCCATGAAGAAC |
| | | GCCGACAACATCAACAAGCTGAAGAGCAGCATCGAGAGCACCAACGAGGCCGTGGTGAAG |
| | | CTGCAGGAGACCGCCGAAAAGACCGTGTACGTGCTGACCGCCCTGCAGGACTACATCAAC |
| | | ACCAACCTGGTGCCCACCATCGACAAGATCAGCTGCAAGCAGACCGAGCTGTCCCTGGAC |
| | | CTGGCCCTGAGCAAGTACCTGAGCGACCTGCTGTTCGTGTTCGGCCCCAACCTGCAGGAC |
| | | CCCGTGAGCAACAGCATGACCATCCAGGCCATCAGCCAGGCCTTCGGCGGCAACTACGAG |
| | | ACCCTGCTGAGGACCCTGGGCTACGCCACCGAGGACTTCGACGACCTGCTGGAGAGCGAC |
| | | AGCATCACCGGCCAGATCATCTACGTGGACCTGAGCAGCTACTACATCATCGTGAGGGTG |
| | | TACTTCCCCATCCTGACCGAGATCCAGCAGGCCTACATCCAGGAGCTGCTGCCCGTCAGC |
| | | TTCAACAACGACGACAGCGAGTGGATCAGCATCGTGCCCAACTTCATCCTGGTGCGGAAC |
| | | ACCCTGATCAGCAACATCGAGATCGGCTTTTGCCTGATCACCAAGAGAAGCGTGATCTGC |
| | | AACCAGGACTACGCCACCCCCATGACCAACAACATGAGAGAGTGCCTGACCGGCAGCACC |
| | | GAGAAGTGCCCTCGGGAACTGGTGGTGTCCAGCCACGTGCCCAGGTTCGCCCTGTCCAAC |
| | | GGCGTGCTGTTCGCCAACTGCATCAGCGTGACCTGCCAGTGCCAGACCACCGGCAGGGCC |
| | | ATCTCCCAGAGCGGCGAGCAGACACTGCTGATGATCGACAACACCACCTGCCCCACCGCC |
| | | GTGCTGGGCAACGTGATCATCAGCCTGGGAAAGTACCTGGGCAGCGTGAACTACAACAGC |
| | | GAGGGCATCGCCATCGGCCCTCCCGTGTTCACCGACAAGGTGGACATCAGCAGCCAGATC |
| | | AGCAGCATGAACCAGAGCCTGCAGCAGAGCAAGGATTACATCAAGGAGGCCCAGAGGCTG |
| | | CTGGACACCGTGAACCCCAGCATGAAGCAGATCGAGGACAAGATCGAGGAGATCCTGAGC |
| | | AAGATCTACCACATCGAGAACGAGATCGCCAGGATCAAGAAGCTGATCGGCGAGGCCCCT |
| | | GGCGGC |
| HeV | 20 | ATGGCCACCCAGGAGGTGCGGCTGAAGTGCCTGCTGTGCGGCATCATCGTGCTGGTGCTG |
| sF coding | | TCCCTGGAGGGCCTGGGCATCCTGCACTACGAGAAGCTGTCCAAGATCGGCCTGGTGAAG |
| sequence | | GGCATCACCAGGAAGTACAAGATCAAGAGCAACCCCCTGACCAAGGACATCGTGATCAAG |
| Genbank: | | ATGATCCCCAACGTGAGCAACGTGTCCAAGTGCACCGGCACCGTGATGGAGAACTACAAG |
| AY816747.1 | | AGCAGGCTGACCGGCATCCTGAGCCCCATCAAGGGCGCCATCGAGCTGTACAACAACAAC |
| | | ACCCACGACCTGGTGGGCGACGTGAAGCTGGCCGGCGTGGTGATGGCCGGCATCGCCATC |
| | | GGAATCGCCACAGCCGCCCAGATCACAGCCGGCGTGGCCCTGTACGAGGCCATGAAGAAC |
| | | GCCGACAACATCAACAAGCTGAAGAGCAGCATCGAGAGCACCAACGAGGCCGTGGTGAAG |
| | | CTGCAGGAGACCGCCGAAAAGACCGTGTACGTGCTGACCGCCCTGCAGGACTACATCAAC |
| | | ACCAACCTGGTGCCCACCATCGACCAGATCAGCTGCAAGCAGACCGAGCTGGCCCTGGAC |
| | | CTGGCCCTGAGCAAGTACCTGAGCGACCTGCTGTTCGTGTTCGGCCCCAACCTGCAGGAC |
| | | CCCGTGAGCAACAGCATGACCATCCAGGCCATCAGCCAGGCCTTCGGCGGCAACTACGAG |
| | | ACCCTGCTGAGGACCCTGGGCTACGCCACCGAGGACTTCGACGGCCTGCTGGAGAGCGAC |
| | | AGCATCACCGGCCAGATCGTGTACGTGGACCTGAGCAGCTACTACATCATCGTGAGGGTG |
| | | TACTTCCCCATCCTGACCGAGATCCAGCAGGCCTACGTGCAGGAGCTGCTGCCCGTCAGC |
| | | TTCAACAACGACAACAGCGAGTGGATCAGCATCGTGCCCAACTTCGTGCTGATCAGGAAC |
| | | ACCCTGATCAGCAACATCGAGGTGAAGTACTGCCTGATCACCAAGAAAAGCGTGATCTGC |
| | | AACCAGGACTACGCCACCCCCATGACCGCCAGCGTGAGAGAGTGCCTGACCGGCAGCACC |
| | | GACAAGTGCCCTCGGGAACTGGTGGTGTCCAGCCACGTGCCCAGGTTCGCCCTGAGCGGC |
| | | GGAGTGCTGTTCGCCAACTGCATCAGCGTGACCTGCCAGTGCCAGACCACCGGCAGGGCC |
| | | ATCTCCCAGAGCGGCGAGCAGACACTGCTGATGATCGACAACACCACCTGCACCACCGTG |
| | | GTGCTGGGCAACATCATCATCAGCCTGGGAAAGTACCTGGGCAGCATCAACTACAACTCC |
| | | GAGAGCATCGCCGTGGGACCCCCCGTGTACACCGACAAGGTGGACATCAGCAGCCAGATC |
| | | AGCAGCATGAACCAGAGCCTGCAGCAGAGCAAGGATTACATCAAGGAGGCCCAGAAAATC |
| | | CTGGACACCGTGAACCCCAGCATGAAGCAGATCGAGGACAAGATCGAGGAGATCCTGAGC |
| | | AAGATCTACCACATCGAGAACGAGATCGCCAGGATCAAGAAGCTGATCGGCGAGGCCCCT |
| | | GGCGGC |

The foregoing sF coding sequences (for NiV-M and HeV) are both codon optimized
and contain an optional GCN4t sequence (bold and underlined) to promote
trimerization of the expressed protein.

| | | |
|---|---|---|
| NIV-B | 30 | ATGGCAGTTATACTTAACAAGAGATATTATTCTAATCTCTTAATACTGATTTTGATGATC |
| sF coding | | TCGGAGTGCAGTGTCGGGATTTTGCATTATGAGAAATTGAGTAAGATTGGGCTTGTCAAA |
| sequence | | GGAATAACAAGAAAATACAAGATCAAAAGCAATCCTCTCACAAAAGACATTGTTATTAAA |
| GenBank: | | ATGATTCCGAATGTGTCAAACATGTCTCAATGCACGGGGAGTGTCATGGAAAACTATAAA |
| JN808864.1 | | ACACGATTAAACGGTATCCTAACGCCTATAAAGGGGGCATTAGAGATTTACAAGAACAAC |
| | | ACTCATGACCTTGTCGGTGATGTAAGACTGGCCGGAGTTATAATGGCAGGAGTTGCTATT |
| | | GGAATTGCAACCGCAGCTCAAATTACTGCAGGTGTAGCATTATATGAGGCAATGAAAAAT |
| | | GCTGACAACATCAACAAACTCAAAAGCAGCATAGAATCAACTAATGAAGCTGTTGTTAAG |
| | | CTTCAAGAGACTGCAGAAAAGACAGTCTATGTACTGACCGCTTTGCAGGATTACATTAAT |
| | | ACTAACTTGGTACCGACAATTGACAAGATAAGCTGCAAACAGACGGAACTCTCATTAGAT |
| | | CTAGCACTATCAAAGTACCTCTCTGATTTGCTTTTTGTATTTGGTCCCAACCTTCAAGAC |
| | | CCAGTTTCTAATTCAATGACTATACAGGCTATATCTCAGGCATTCGGTGGAAATTATGAA |
| | | ACACTGCTAAGAACGTTGGGTTACGCTACAGAAGACTTTGATGATCTTCTAGAAAGTGAC |
| | | AGCATAACGGGTCAAATTATCTACGTTGATTTAAGTGGCTACTACATAATTGTCAGGGTT |
| | | TATTTTCCTATCCTGACTGAAATCCAACAGGCCTATATCCAAGAATTGTTGCCAGTGAGC |
| | | TTTAACAATGACAATTCAGAATGGATCAGCATTGTCCCAAATTTCATATTGGTAAGGAAC |
| | | ACATTAATATCAAATATAGAGATTGGATTTTGCCTAATTACAAAGAGGAGTGTGATCTGC |
| | | AACCAAGATTATGCAACACCCATGACAAACAATATGAGGGAATGTTTGACGGGGTCGACT |
| | | GAGAAGTGTCCTCGAGAGCTGGTGGTTTCATCACACGTTCCCAGATTTGCACTATCTAAC |
| | | GGGGTTTTGTTTGCTAATTGCATAAGCGTCACATGCCAGTGTCAAACAACAGGTAGGGCA |
| | | ATCTCACAGTCAGGAGAACAAACTCTGCTGATGATTGATAACACCACCTGTCCTACAGCC |
| | | GTACTCGGTAATGTGATCATCAGCTTGGGAAAATATCTTGGGTCAGTAAATTATAACTCT |
| | | GAAGGCATTGCTATTGGTCCTCCTGTCTTTACTGATAAAGTTGACATATCAAGTCAAATA |
| | | TCTAGCATGAATCAGTCCTTACAACAATCTAAGGACTATATCAAAGAAGCTCAACGACTC |
| | | CTTGATACTGTTAACCCGTCAATGAAGCAGATCGAGGACAAGATCGAGGAGATCCTGAGC |
| | | AAGATCTACCACATCGAGAACGAGATCGCCAGGATCAAGAAGCTGATCGGCGAGGCCCCT |
| | | GGCGGC |

The NiV-B sF coding sequence contains an optional GCN4t sequence (bold and
underlined) to promote trimerization of the expressed protein.

| | | |
|---|---|---|
| NIV-B | 21 | ATGGAAACCGACACTCTGCTGCTGTGGGTCCTGCTGCTGTGGGTCCCTGGCTCAACTGGC |
| sG coding | | GACGCAGCATCAACAGATAATCAGGCCATGATCAAAGATGCATTGCAGAGTATCCAGCAG |
| sequence | | CAGATCAAGGGGCTTGCCGACAAAATTGGCACAGAGATAGGGCCGAAAGTATCACTGATT |
| Genbank: | | GATACATCCAGTACTATCACTATTCCAGCTAATATTGGGCTGTTAGGTTCAAAGATCAGC |
| JN808864.1 | | CAGTCAACTGCAAGTATAAATGAGAATGTGAATGAAAAATGCAAATTTACACTGCCTCCC |
| | | TTGAAAATCCACGAATGTAACATTTCTTGTCCTAACCCACTCCCTTTTAGAGAGTATAAG |
| | | CCGCAGACAGAAGGAGTGAGCAATCTGGTAGGATTACCTAATAATATCTGTCTGCAAAAG |
| | | ACATCTAATCAGATACTGAAACCAAAGCTGATTTCATACACCTTACCCGTAGTCGGTCAA |
| | | AGTGGCACCTGTATCACAGACCCACTGCTGGCTATGGATGAGGGCTACTTTGCATATAGC |
| | | CACCTGGAAAAAATCGGATCATGTTCAAGAGGGGTCTCCAAACAAAGAATAATAGGAGTT |
| | | GGAGAGGTACTAGACAGAGGTGACGAAGTACCTTCTTTGTTTATGACTAACGTCTGGACC |
| | | CCATCAAATCCAAACACCGTTTACCATTGCAGTGCCGTGTACAACAATGAATTCTATTAT |
| | | GTGCTTTGTGCAGTGTCAGTTGTTGGAGACCCTATTCTGAATAGCACCTACTGGTCCGGA |
| | | TCACTAATGATGACTCGTCTAGCTGTAAAACCTAAGAATAATGGTGAGAGTTACAATCAA |
| | | CATCAATTTGCCTTACGGAATATTGAGAAAGGGAAGTATGATAAAGTTATGCCATATGGA |
| | | CCCTCAGGCATCAAACAAGGTGACACCCTGTACTTTCCTGCTGTAGGATTTTTGGTCAGG |
| | | ACAGAGTTCACATACAATGATTCAAATTGTCCCATCGCAGAGTGTCAATACAGCAAACCT |
| | | GAAAACTGCAGGCTATCTATGGGGATTAGACCAAACAGTCATTATATCCTTCGATCTGGA |
| | | CTACTAAAATACAATCTATCGGATGAGGAGAACTCTAAAATTGTATTCATTGAAATATCT |
| | | GATCAAAGACTATCTATTGGATCTCCTAGCAAAATCTATGATTCTTTGGGTCAACCTGTT |
| | | TTCTACCAAGCATCTTTTTCATGGGACACTATGATTAAATTTGGAGATGTCCAAACAGTT |
| | | AACCCTTTAGTTGTAAATTGGCGTGACAACACGGTAATCTCAAGACCTGGGCAATCACAA |
| | | TGCCCTAGATTCAACAAGTGCCCAGAGGTTTGCTGGGAAGGGGTTTATAATGATGCTTTC |
| | | CTGATTGATAGAATCAATTGGATAAGCGCGGGTGTATTCCTTGACAGCAACCAGACCGCA |
| | | GAGAATCCTGTTTTTACTGTATTCAAAGATAATGAAGTACTTTACAGAGCACAACTAGCT |
| | | TCCGAGGACACCAATGCACAAAAAACAATAACTAATTGCTTCCTTTTGAAGAATAAGATC |
| | | TGGTGTATATCACTGGTTGAGATATACGACACAGGAGACAATGTTATAAGACCTAAACTA |
| | | TTCGCAGTTAAGATACCAGAGCAATGTACATAATAA |

The attachment glycoprotein's signal sequence for endoplasmic reticulum
targeting is located at the N terminus of the protein, which also overlaps
with the molecule's transmembrane anchor domain. To create a soluble and
secreted form of NiV-B sG, the Ig κ leader sequence (bold and underlined)
was added to the N-terminus of NiV-B sG. A short linker sequence (GCAGCA)
was inserted between the Ig κ leader sequence and the NiV-B sG sequence
(underlined). To maintain the “rule of 6” an extra stop codon (TAA;
bolded italics) was added to the end of the NiV-B sG coding region.
Both of these modifications are optional.

| | | |
|---|---|---|
| HeV | 22 | ATGGAAACCGACACTCTGCTGCTGTGGGTCCTGCTGCTGTGGGTCCCTGGCTCAACTGGC |
| sG coding | | GACGCAGCAAGAACGACTGATAATCAGGCACTAATCAAAGAGTCACTCCAGAGTGTACAG |
| sequence | | CAACAAATCAAAGCTTTAACAGACAAAATCGGGACAGAGATAGGCCCCAAAGTCTCACTA |
| Genbank: | | ATTGACACATCCAGCACCATCACAATTCCTGCTAACATAGGGTTACTGGGATCCAAGATA |
| MN062017.1 | | AGTCAGTCTACCAGCAGTATTAATGAGAATGTTAACGATAAATGCAAATTTACTCTTCCT |
| | | CCTTTAAAGATTCATGAGTGTAATATCTCTTGTCCGAATCCTTTGCCTTTCAGAGAATAC |
| | | CGACCAATCTCACAAGGGGTGAGTGATCTTGTAGGACTGCCGAACCAGATCTGTCTACAG |
| | | AAGACAACATCAACAATCTTAAAGCCCAGGCTGATATCCTATACTCTACCAATTAATACC |
| | | AGAGAAGGGGTTTGCATCACTGACCCACTTTTGGCTGTTGATAATGGCTTCTTCGCCTAT |
| | | AGCCATCTTGAAAAGATCGGATCATGTACTAGAGGAATTGCAAAACAAAGGATAATAGGG |
| | | GTGGGTGAGGTATTGGATAGGGGTGATAAGGTGCCATCAATGTTTATGACCAATGTTTGG |
| | | ACACCACCCAATCCAAGCACCATCCATCATTGCAGCTCAACTTACCATGAAGATTTTTAT |
| | | TACACATTGTGCGCAGTGTCCCATGTGGGAGATCCTATCCTTAACAGTACTTCCTGGACA |
| | | GAGTCACTGTCTCTGATTCGTCTTGCTGTAAGACCAAAAAGTGATAGTGGAGACTACAAT |
| | | CAGAAATACATCGCTATAACTAAAGTTGAAAGAGGGAAGTACGATAAGGTGATGCCTTAC |
| | | GGTCCATCAGGTATCAAGCAAGGGGATACATTGTACTTTCCGGCCGTCGGTTTTTTGCCA |
| | | AGGACCGAATTTCAATATAATGACTCTAATTGTCCCATAATTCATTGCAAGTACAGCAAA |
| | | GCAGAAAACTGTAGGCTTTCAATGGGTGTCAACTCCAAAAGTCATTATATTTTGAGATCA |
| | | GGACTATTGAAGTATAATCTATCTCTTGGAGGAGACATCATACTCCAATTTATCGAGATT |
| | | GCTGACAATAGATTGACCATCGGTTCTCCTAGTAAGATATACAATTCCCTAGGTCAACCC |
| | | GTTTTCTACCAGGCATCATATTCTTGGGATACGATGATTAAATTAGGCGATGTTGATACC |
| | | GTTGACCCTCTAAGAGTACAGTGGAGAAATAACAGTGTGATTTCTAGACCTGGACAGTCA |
| | | CAGTGTCCTCGATTTAATGTCTGTCCCGAGGTATGCTGGGAAGGGACATATAATGATGCT |
| | | TTTCTAATAGACCGGCTAAACTGGGTTAGTGCTGGTGTTTATTTAAACAGTAACCAAACA |
| | | GCAGAGAACCCTGTGTTTGCCGTATTCAAGGATAACGAGATCCTTTACCAAGTTCCACTG |
| | | GCTGAAGATGACACAAATGCACAAAAAACCATCACAGATTGCTTCTTGCTGGAGAATGTC |
| | | ATATGGTGTATATCACTAGTAGAAATATACGATACAGGAGACAGTGTGATAAGGCCAAAA |
| | | CTATTTGCAGTCAAGATACCTGCCCAATGTTCAGAGAGTTGA |

The attachment glycoprotein's signal sequence for endoplasmic reticulum
targeting is located at the N terminus of the protein, which also overlaps
with the molecule's transmembrane anchor domain. To create a soluble and
secreted form of HeV sG, the Ig κ leader sequence (bold and underlined)
was added to the N-terminus of HeV sG. A short linker sequence (GCAGCA)
was inserted between the Ig κ leader sequence and the NiV-B sG sequence
(underlined). There is a single nucleotide change (T1593A; bold and
underlined) in the above sequence of HeV G, which does not result in an
amino acid change. The HeV sG amino acid sequence is identical to the
amino acid sequence of the HeV 2008 Redlands isolate, protein sequence
GenBank: AEQ38071.1. Both of these modifications are optional.

| | | |
|---|---|---|
| NiV-B | 31 | ATGGAAACCGACACTCTGCTGCTGTGGGTCCTGCTGCTGTGGGTCCCTGGCTCAACTGGC |
| sGtet coding | | GACGTCGACATGAAGCAGATCGAGGACAAGCTGGAGGAGATCGAGAGCAAGCTGAAGAAG |
| sequence | | ATCGAGAACGAGCTGGCCAGGATCAAGAAGGTCGACTACACCTCAACAGATAATCAGGCC |
| Genbank: | | ATGATCAAAGATGCATTGCAGAGTATCCAGCAGCAGATCAAGGGGCTTGCCGACAAAATT |
| JN808864.1 | | GGCACAGAGATAGGGCCGAAAGTATCACTGATTGATACATCCAGTACTATCACTATTCCA |
| | | GCTAATATTGGGCTGTTAGGTTCAAAGATCAGCCAGTCAACTGCAAGTATAAATGAGAAT |
| | | GTGAATGAAAAATGCAAATTTACACTGCCTCCCTTGAAAATCCACGAATGTAACATTTCT |
| | | TGTCCTAACCCACTCCCTTTTAGAGAGTATAAGCCGCAGACAGAAGGAGTGAGCAATCTG |
| | | GTAGGATTACCTAATAATATCTGTCTGCAAAAGACATCTAATCAGATACTGAAACCAAAG |
| | | CTGATTTCATACACCTTACCCGTAGTCGGTCAAAGTGGCACCTGTATCACAGACCCACTG |
| | | CTGGCTATGGATGAGGGCTACTTTGCATATAGCCACCTGGAAAAAATCGGATCATGTTCA |
| | | AGAGGGGTCTCCAAACAAAGAATAATAGGAGTTGGAGAGGTACTAGACAGAGGTGACGAA |
| | | GTACCTTCTTTGTTTATGACTAACGTCTGGACCCCATCAAATCCAAACACCGTTTACCAT |
| | | TGCAGTGCCGTGTACAACAATGAATTCTATTATGTGCTTTGTGCAGTGTCAGTTGTTGGA |
| | | GACCCTATTCTGAATAGCACCTACTGGTCCGGATCACTAATGATGACTCGTCTAGCTGTA |
| | | AAACCTAAGAATAATGGTGAGAGTTACAATCAACATCAATTTGCCTTACGGAATATTGAG |
| | | AAAGGGAAGTATGATAAAGTTATGCCATATGGACCCTCAGGCATCAAACAAGGTGACACC |
| | | CTGTACTTTCCTGCTGTAGGATTTTTGGTCAGGACAGAGTTCACATACAATGATTCAAAT |
| | | TGTCCCATCGCAGAGTGTCAATACAGCAAACCTGAAAACTGCAGGCTATCTATGGGGATT |
| | | AGACCAAACAGTCATTATATCCTTCGATCTGGACTACTAAAATACAATCTATCGGATGAG |
| | | GAGAACTCTAAAATTGTATTCATTGAAATATCTGATCAAAGACTATCTATTGGATCTCCT |
| | | AGCAAAATCTATGATTCTTTGGGTCAACCTGTTTTCTACCAAGCATCTTTTTCATGGGAC |
| | | ACTATGATTAAATTTGGAGATGTCCAAACAGTTAACCCTTTAGTTGTAAATTGGCGTGAC |
| | | AACACGGTAATCTCAAGACCTGGGCAATCACAATGCCCTAGATTCAACAAGTGCCCAGAG |
| | | GTTTGCTGGGAAGGGGTTTATAATGATGCTTTCCTGATTGATAGAATCAATTGGATAAGC |
| | | GCGGGTGTATTCCTTGACAGCAACCAGACCGCAGAGAATCCTGTTTTTACTGTATTCAAA |
| | | GATAATGAAGTACTTTACAGAGCACAACTAGCTTCCGAGGACACCAATGCACAAAAAACA |
| | | ATAACTAATTGCTTCCTTTTGAAGAATAAGATCTGGTGTATATCACTGGTTGAGATATAC |
| | | GACACAGGAGACAATGTTATAAGACCTAAACTATTCGCAGTTAAGATACCAGAGCAATGT |
| | | ACATAA |

This is a sG protein with a tet (tetramerization) peptide sequence.
The attachment glycoprotein's signal sequence for endoplasmic reticulum
targeting is located at the N terminus of the protein, which also overlaps
with the molecule's transmembrane anchor domain. To create a soluble and
secreted form of NiV-B sG, the Ig κ leader sequence (bold and underlined)
was added to the N-terminus of NiV-B sG. The NiV-B sG contains a GCNtet
sequence to promote tetramerization (bold and italic). Short amino acid
linker sequences were included between the Ig κ leader sequence and the
GCNtet sequence (GTCGAC; underlined) and the GCNtet sequence and the
NiV-B sG sequence (GTCGACTACACC (SEQ ID NO: 37); underlined).

| | | |
|---|---|---|
| HeV | 32 | ATGGAAACCGACACTCTGCTGCTGTGGGTCCTGCTGCTGTGGGTCCCTGGCTCAACTGGC |
| sGtet coding | | GACGTCGACATGAAGCAGATCGAGGACAAGCTGGAGGAGATCGAGAGCAAGCTGAAGAAG |
| sequence | | ATCGAGAACGAGCTGGCCAGGATCAAGAAGGTCGACTACACCCGGACCACCGACAACCAG |
| Genbank: | | GCCCTGATCAAAGAGTCCCTGCAGAGCGTCCAGCAGCAGATCAAGGCCCTGACCGACAAG |
| MN062017.1 | | ATCGGCACCGAGATCGGCCCCAAAGTGTCCCTGATCGACACCAGCAGCACCATCACCATC |
| | | CCCGCCAACATCGGGCTGCTGGGCTCCAAGATCAGCCAGAGCACCAGCTCCATCAACGAG |
| | | AACGTGAACGACAAGTGCAAGTTCACCCTGCCCCCCCTGAAGATCCACGAGTGCAACATC |
| | | AGCTGCCCCAACCCCCTGCCCTTCCGGGAGTACCGGCCCATCAGCCAGGGCGTGAGCGAC |
| | | CTGGTGGGCCTGCCCAACCAGATCTGCCTGCAGAAAACCACCTCCACCATCCTGAAGCCC |
| | | CGGCTGATCAGCTACACCCTGCCCATCAACACCCGGGAGGGCGTGTGCATCACCGACCCT |
| | | CTGCTGGCCGTGGACAACGGCTTCTTCGCCTACAGCCACCTGGAAAAGATCGGCAGCTGC |
| | | ACCCGGGGCATTGCCAAGCAGCGGATCATCGGCGTGGGCGAGGTGCTGGACCGGGGCGAC |
| | | AAGGTGCCCAGCATGTTCATGACCAACGTGTGGACCCCCCCCAACCCCAGCACAATCCAC |
| | | CACTGCAGCAGCACCTACCACGAGGACTTCTACTACACCCTGTGCGCCGTGAGCCACGTG |
| | | GGCGACCCCATCCTGAACAGCACCAGCTGGACCGAGAGCCTGAGCCTGATCCGGCTGGCC |
| | | GTGCGGCCCAAGAGCGACAGCGGCGACTACAACCAGAAGTATATCGCCATCACCAAGGTG |
| | | GAGCGGGGCAAGTACGACAAAGTGATGCCCTACGGCCCCAGCGGCATCAAGCAGGGCGAC |
| | | ACACTGTACTTCCCCGCCGTGGGCTTCCTGCCCCGGACCGAGTTCCAGTACAACGACAGC |
| | | AACTGCCCCATCATCCACTGCAAGTACAGCAAGGCCGAGAACTGCAGACTGAGCATGGGC |
| | | GTGAACAGCAAGAGCCACTACATCCTGCGGAGCGGCCTGCTGAAGTACAACCTGTCCCTG |
| | | GGCGGCGACATCATCCTGCAGTTCATCGAGATCGCCGACAACCGGCTGACCATCGGCAGC |
| | | CCCAGCAAGATCTACAACAGCCTGGGCCAGCCCGTGTTCTACCAGGCCAGCTACAGCTGG |
| | | GACACCATGATCAAGCTGGGGGACGTGGACACCGTGGACCCCCTGCGGGTGCAGTGGCGG |
| | | AACAACAGCGTGATCAGCAGACCCGGCCAGAGCCAGTGCCCCCGGTTCAACGTGTGCCCC |
| | | GAAGTGTGCTGGGAGGGCACCTACAACGACGCCTTTCTGATCGACCGGCTGAACTGGGTG |
| | | TCCGCCGGAGTGTACCTGAACTCCAACCAGACCGCCGAGAACCCCGTGTTCGCCGTGTTC |
| | | AAGGACAACGAGATCCTGTACCAGGTGCCCCTGGCCGAGGACGACACCAACGCCCAGAAA |
| | | ACCATCACCGACTGCTTTCTGCTGGAAAACGTGATCTGGTGCATCAGCCTGGTGGAGATC |
| | | TACGACACCGGCGACTCCGTGATCCGGCCCAAGCTGTTTGCCGTGAAGATCCCCGCCCAG |
| | | TGCAGCGAGAGCTGATGA |

This is a sG protein with a tet (tetramerization) peptide sequence.
The attachment glycoprotein's signal sequence for endoplasmic reticulum
targeting is located at the N terminus of the protein, which also overlaps
with the molecule's transmembrane anchor domain. To create a soluble and
secreted form of HeV sG, the Ig κ leader sequence (bold and underlined)
was added to the N-terminus of HeV sG. The HeV sG coding sequence is codon
optimized and also contains a GCNtet sequence to promote tetramerization
(bold and italic). Short linker sequences were included between the Ig κ
leader sequence and the GCNtet sequence (GTCGAC; underlined) and between the
GCNtet sequence and the HeV sG sequence (GTCGACTACACC (SEQ ID NO: 37);
underlined). The HeV sG sequence has the NCBI Reference sequence:
NP_047112.2. To maintain the “rule of 6”, a feature of henipaviruses
to promote replication initiation (Halpin et al., 2004), an extra stop codon
(TGA; bold) was added to the end of the HeV sG coding region.

The present disclosure includes additional variations to non-CedV coding sequences for sF and sG proteins, beyond those illustrated above (e.g., the optional GCN4t sequence on sF proteins, the optional Igκ leader sequence on sG proteins, and optional point mutations shown in Table 2). In some embodiments, a rCedV chimera comprises a gene encoding a non-CedV sF protein, a gene encoding a non-CedV sG protein, or both, in which the gene encoding the non-CedV sF protein and the gene encoding the non-CedV sG protein may comprise 1, 2, 3, 4, 5, 6, 7, 8, 9, or 10 or more substitutions, insertions, or deletions relative to the naturally-occurring sequence, so long as the resulting protein is capable of eliciting an immune response. For instance, the gene encoding the non-CedV sF protein may comprise a sequence that is about 90%, about 91%, about 92%, about 93%, about 94%, about 95%, about 96%, about 97%, about 98%, about 99%, or 100% identical to nucleotides 1-1,461 of SEQ ID NO: 19, nucleotides 1-1,461 of SEQ ID NO: 20, or nucleotides 1-1,461 of SEQ ID NO: 30. In some embodiments, the gene encoding the non-CedV sF protein may comprise a sequence that is about 90%, about 91%, about 92%, about 93%, about 94%, about 95%, about 96%, about 97%, about 98%, about 99%, or 100% identical to any one of SEQ ID NOs: 19, 20, or 30. Similarly, the gene encoding the non-CedV sG protein may comprise a sequence that is about 90%, about 91%, about 92%, about 93%, about 94%, about 95%, about 96%, about 97%, about 98%, about 99%, or 100% identical to nucleotides 64-1,647 of SEQ ID NO: 21 or 31, or nucleotides 64-1,656 of SEQ ID NO: 22 or 32. In some embodiments, the gene encoding the non-CedV sG protein may comprise a sequence that is about 90%, about 91%, about 92%, about 93%, about 94%, about 95%, about 96%, about 97%, about 98%, about 99%, or 100% identical to any one of SEQ ID NOs: 21, 22, 31, or 32.

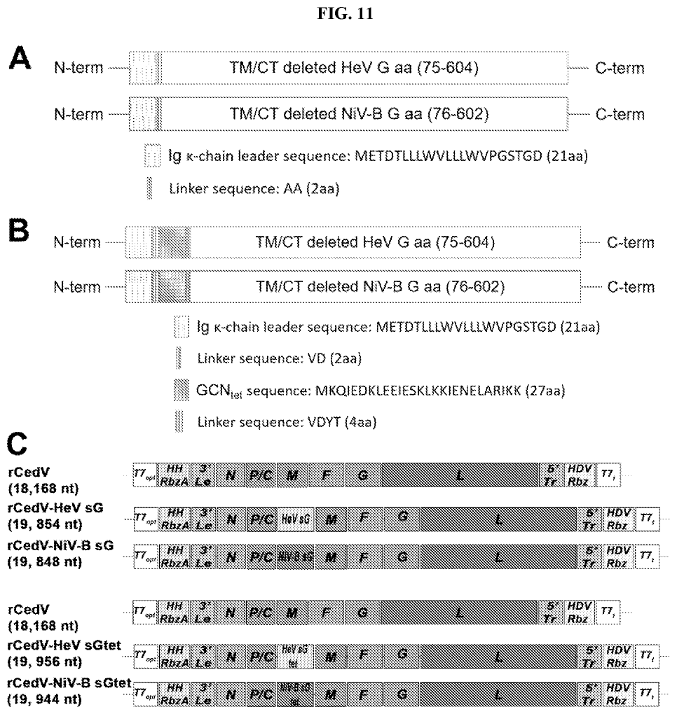

The coding sequences for sF, sG, or both, can be incorporated into the rCedV genome between the P/C gene and M gene, as shown in FIG. 11. Indeed, it was determined that reporter genes (e.g., GFP and Luc) were well-tolerated when inserted between the P/C gene and M gene as well, making this a particularly useful site for insertion. Paramyxoviruses have what is known as a gradient of transcription, whereby more mRNA transcripts are made from the 5′ end (N protein the most) to the 3′ end (L protein the least), and therefore incorporation between P/C and M leads to a high level expression of the non-CedV gene(s) inserted in this location. In this way, the more sG or sF antigen made and secreted from the rCedV chimera, the better the immune response to sG or sF antigen. Nevertheless, the coding sequences for the sF, sG, or both, may be inserted in other locations as well, so long as the virus remains replication-competent.

As described in more detail below, recombinant Cedar virus (rCedV) chimeras that express non-CedV henipavirus sF, sG, or both, may be used to elicit an immune response to a non-CedV henipavirus.

Another aspect of the present disclosure is directed to a method of producing a non-CedV henipavirus sF protein, a non-CedV henipavirus sG protein, or both, using a rCedV as disclosed herein. In some embodiments, the method comprises culturing cells capable of being infected by a henipavirus with a rCedV chimera as described herein comprising a coding sequence for a sF, sG, or both, of a non-CedV henipavirus, and isolating the non-CedV henipavirus sF, sG, or both, from the culture. In some embodiments, the sF and/or sG are NiV (e.g., NiV-M or NiV-B) sF and sG. In some embodiments, the sF and/or sG are HeV sF and sG.

### C. Chimeras Expressing Fusion Proteins

Another aspect of the present disclosure is directed to rCedV chimeras comprising one or both of (i) a gene encoding a henipavirus F envelope protein fusion protein and (ii) a gene encoding a henipavirus G envelope protein fusion, wherein the fusion protein comprises the ectodomain and transmembrane domain of a non-CedV henipavirus F envelope protein or G envelope protein, respectively, fused to the cytoplasmic tail domain of CedV F envelope protein or G envelope protein, respectively. Or the fusion protein comprises the ectodomain of a non-CedV henipavirus F envelope protein or G envelope protein, respectively, fused to the transmembrane domain and cytoplasmic tail domain of CedV F envelope protein or G envelope protein, respectively.

Chimeras comprising these fusion constructs exhibit the predicted interaction of CedV matrix protein (M) (which forms the virus particle) with the internal cytoplasmic tail domains of CedV F and G, and display the functional ectodomains of the non-CedV F and G envelope proteins on the virus particle. Those ectodomains of the non-CedV F and G envelope proteins are anchored by their respective transmembrane domains and the CedV F and G cytoplasmic tail domains, or by the respective transmembrane domains and cytoplasmic tails of the CedV F and G envelope glycoproteins, which allows the ectodomains of the non-CedV F and G proteins to be exposed and serve as antigens, e.g., if the chimera is administered to a subject.

In some instances, particularly with MojV and GhV, expressing the full length sequence of a non-CedV F protein, G protein, or both, in a rCedV chimera (as disclosed herein above) may not result in a replication competent chimera. Chimeras comprising these fusion constructs instead may avoid this problem, and result in replication competent chimeras that effectively display the non-CedV F and/or G envelope protein ectodomains on the virus particle, where they can serve as antigens. Thus, chimeras comprising these fusion constructs may be advantageous for chimeras of certain species of non-CedV henipaviruses, such as MojV or GhV, and may result in the production of more robust and replication-competent rCedV chimeras (which may exhibit better virus production capacity) than chimeras in which a full-length non-CedV F protein or G protein was inserted.

In any embodiments, the fusion proteins may comprise all or a portion of the ectodomain and transmembrane domain of the non-CedV henipavirus. For example, a fusion protein may comprise the full ectodomain or an immunogenic fragment thereof. In some embodiments, the fusion F protein or fusion G protein comprises the full ectodomain sequence of the non-CedV henipavirus. In some embodiments, the fusion F protein or fusion G protein comprises a fragment of the ectodomain that is about 70%, about 75%, about 80%, about 85%, about 90%, about 91%, about 92%, about 93%, about 94%, about 95%, about 96%, about 97%, about 98%, or about 99% of the length of the full ectodomain sequence of the F or G protein of the given non-CedV henipavirus. In some embodiments, the fusion F protein or fusion G protein comprises the full transmembrane domain sequence of the non-CedV henipavirus. In some embodiments, the fusion F protein or fusion G protein comprises a fragment of the transmembrane domain that is about 70%, about 75%, about 80%, about 85%, about 90%, about 91%, about 92%, about 93%, about 94%, about 95%, about 96%, about 97%, about 98%, or about 99% of the length of the full transmembrane domain sequence of the F or G protein of the given non-CedV henipavirus.

The non-CedV henipavirus ectodomain and transmembrane domain sequences can be from any henipavirus, including from any pathogenic henipavirus, including from HeV, NiV, GhV, or MojV. In some embodiments, the non-CedV henipavirus ectodomain and transmembrane domain sequences are MojV ectodomain and transmembrane domain sequences. In some embodiments, the non-CedV henipavirus ectodomain and transmembrane domain sequences are GhV ectodomain and transmembrane domain sequences. In some embodiments, the ectodomains of non-CedV henipavirus F and G glycoproteins are fused to the transmembrane domains and cytoplasmic tail domains of CedV F and G glycoproteins.

In some embodiments, the rCedV chimera comprises F and/or G fusion genes derived from GhV (i.e., a fusion comprising a GhV ectodomain and GhV transmembrane domain with the cytoplasmic domain of CedV). In some embodiments, the rCedV chimera comprises F and G fusion genes derived from GhV (designated herein as chimera rCedV-GhV). In some embodiments, the rCedV chimera comprises a CedV F envelope glycoprotein gene and a GhV G fusion gene. In some embodiments, the rCedV chimera comprises a CedV G envelope glycoprotein gene and a GhV F fusion gene.

In some embodiments, the rCedV chimera comprises F and/or G fusion genes derived from MojV (i.e., a fusion comprising a MojV ectodomain and MojV transmembrane domain with the cytoplasmic domain of CedV). In some embodiments, the rCedV chimera comprises both F and G fusion genes derived from MojV (designated herein as chimera rCedV-MojV). In some embodiments, the rCedV chimera comprises a CedV F envelope glycoprotein gene and a MojV G fusion gene. In some embodiments, the rCedV comprises a CedV G envelope glycoprotein gene and a MojV F fusion gene.

In some embodiments, the rCedV chimera comprises F and/or G fusion genes derived from HeV (i.e., a fusion comprising a HeV ectodomain and HeV transmembrane domain with the cytoplasmic domain of CedV). In some embodiments, the rCedV chimera comprises both F and G fusion genes derived from HeV. In some embodiments, the rCedV chimera comprises a CedV F envelope glycoprotein gene and a HeV G fusion gene. In some embodiments, the rCedV comprises a CedV G envelope glycoprotein gene and a HeV F fusion gene.

In some embodiments, the rCedV chimera comprises F and/or G fusion genes derived from NiV (i.e., a fusion comprising a NiV ectodomain and NiV transmembrane domain with the cytoplasmic domain of CedV). In some embodiments, the rCedV chimera comprises both F and G envelope glycoprotein genes derived from NiV. In some embodiments, the rCedV chimera comprises a CedV F envelope glycoprotein gene and a NiV G fusion gene. In some embodiments, the rCedV comprises a CedV G envelope glycoprotein gene and a NiV F fusion gene.

Exemplary F and G fusion coding sequences are provided below in Table 3.

TABLE 3
Exemplary Fusion F and G Sequences

| | SEQ | |
|---|---|---|
| | ID | |
| Name | NO: | Sequence |
| MojV | 23 | ATGGCACTAAATAAAAATATGTTCAGTTCACTGTTCCTTGGTTATCTATTAGTG |
| Fusion F | | TACGCTACGACTGTTCAGTCTAGTATACACTATGACTCCTTATCTAAGGTCGGT |
| coding seq. | | GTCATTAAGGGTCTGACATACAACTATAAGATCAAGGGTTCGCCATCTACAAAG |
| Genbank: | | CTAATGGTGGTCAAATTGATACCTAACATTGATAGTGTTAAAAACTGTACTCAG |
| KF278639.1 | | AAACAGTATGATGAATACAAGAACTTAGTAAGGAAAGCCTTAGAACCGGTCAAA |
| | | ATGGCTATTGACACCATGCTCAATAATGTTAAGTCGGGTAATAACAAGTACAGA |
| | | TTTGCAGGTGCAATTATGGCTGGAGTTGCCCTCGGTGTTGCAACAGCAGCCACT |
| | | GTTACAGCAGGGATAGCTCTCCATAGATCAAATGAAAATGCACAGGCAATTGCA |
| | | AACATGAAGAGTGCTATTCAAAATACAAATGAGGCAGTAAAGCAATTGCAATTG |
| | | GCCAATAAACAAACACTAGCTGTGATTGACACCATAAGAGGAGAGATCAATAAC |
| | | AATATAATACCCGTTATAAATCAATTGAGCTGTGACACAATTGGGCTCAGTGTA |
| | | GGTATAAGACTCACTCAGTACTACTCTGAAATAATAACTGCATTTGGGCCAGCT |
| | | TTGCAGAATCCAGTAAATACAAGGATTACCATTCAAGCAATATCTAGTGTGTTT |
| | | AATGGCAACTTTGATGAACTGCTGAAGATTATGGGGTATACAAGTGGTGATCTT |
| | | TATGAAATTCTACATAGTGAATTAATTAGAGGCAACATTATAGACGTTGATGTA |
| | | GATGCAGGATACATAGCTCTAGAAATAGAATTCCCCAATCTAACATTGGTACCT |
| | | AATGCTGTAGTACAGGAGTTAATGCCTATCAGTTATAACATAGACGGGGATGAG |
| | | TGGGTAACACTTGTGCCAAGGTTTGTACTTACAAGGACTACACTGTTATCAAAT |
| | | ATTGATACGAGTAGATGTACAATCACAGATAGTAGTGTCATATGTGACAACGAC |
| | | TACGCCTTGCCTATGTCACACGAGCTTATTGGCTGCTTACAGGGAGATACATCT |
| | | AAGTGTGCTAGAGAGAAGGTAGTCTCAAGTTATGTCCCTAAATTTGCGTTGTCT |
| | | GATGGGTTAGTGTATGCAAATTGCCTCAATACTATCTGCCGATGTATGGATACA |
| | | GATACTCCAATCTCACAAAGTCTCGGAGCCACTGTATCATTACTAGACAACAAG |
| | | AGGTGTTCAGTATATCAGGTAGGAGATGTCTTGATTTCTGTCGGATCATATCTA |
| | | GGAGATGGAGAATATAATGCTGATAATGTAGAGCTTGGCCCACCTATAGTTATA |
| | | GATAAGATTGACATAGGAAATCAGCTGGCAGGTATTAATCAAACCTTACAAGAG |
| | | GCAGAAGATTACATTGAGAAGTCAGAAGAGTTCTTAAAAGGGGTTAACCCTTCA |
| | | ATTATCACTCTTGGTTCCATGGTTGTCCTTTATATATTTATGATATTAATAGCC |
| | | ATTGTGTCAGTAATAGCACTAGTATTGTCAATTAAATTAACAGTAAAAAAATAT |
| | | AACAAATTTATAGATGATCCTGATTATTACAATGATTACAAAAGAGAACGTATT |
| | | AATGGCAAAGCCAGTAAGAGTAACAATATATATTATGTAGGTGATTGA |
| GhV | 24 | ATGAAGAAAAAGACGGACAATCCCACAATATCAAAGAGGGGTCACAACCATTCT |
| Fusion F | | CGAGGAATCAAATCTAGAGCGCTACTCAGAGAGACAGATAATTATTCCAATGGG |
| coding seq. | | CTAATAGTTGAGAATTTAGTTAGAAACTGTCATCATCCAAGTAAGAACAATCTA |
| Genbank: | | AACTATACTAAGACACAAAAAAGAGATTCTACAATCCCTTATCGTGTGGAAGAG |
| HQ660129.1 | | AGAAAAGGACATTATCCAAAGATTAAACATCTTATTGATAAATCTTACAAGCAT |
| | | ATAAAAAGAGGGAAGAGAAGAAATGGTCATAATGGGAACATTATAACTATAATT |
| | | CTGTTGTTGATTTTAATTTTGAAGACACAGATGAGTGAAGGTGCTATCCATTAC |
| | | GAGACTCTAAGTAAGATCGGATTAATAAAGGGAATCACCAGAGAGTACAAAGTC |
| | | AAAGGAACTCCGTCAAGTAAAGACATAGTCATCAAATTGATTCCGAATGTCACC |
| | | GGTCTTAACAAGTGCACGAACATATCAATGGAAAACTATAAAGAACAACTTGAC |
| | | AAAATACTAATTCCTATTAACAACATAATTGAATTGTATGCAAACTCAACTAAA |
| | | TCAGCCCCTGGGAATGCACGTTTTGCTGGCGTTATAATTGCAGGAGTGGCATTA |
| | | GGTGTTGCAGCGGCAGCCCAAATAACTGCCGGCATTGCACTGCATGAAGCTCGA |
| | | CAGAATGCAGAGAGAATTAATCTCTTAAAGGATAGCATTTCTGCCACTAACAAC |
| | | GCAGTAGCAGAACTCCAGGAAGCAACTGGTGGAATAGTAAATGTCATTACAGGA |
| | | ATGCAAGATTACATCAATACAAATCTAGTCCCGCAGATTGACAAACTGCAATGT |
| | | AGTCAGATCAAAACGGCATTAGACATATCTCTCTCCCAATACTATTCAGAAATA |
| | | TTAACAGTGTTCGGTCCAAACCTTCAAAATCCAGTAACTACTTCCATGTCAATA |
| | | CAAGCCATATCACAATCCTTTGGGGGAAATATAGATTTGCTCTTAAACCTACTA |
| | | GGTTACACTGCAAACGACTTATTGGATTTGCTCGAAAGTAAAAGTATAACAGGC |
| | | CAAATAACATACATAAATCTTGAACATTACTTCATGGTAATCAGAGTATATTAT |
| | | CCTATAATGACAACAATCAGCAATGCTTATGTCCAGGAATTGATCAAAATTAGC |
| | | TTCAATGTCGATGGCAGTGAATGGGTATCTCTTGTACCCTCGTATATATTGATT |
| | | AGAAACTCATATCTCTCAAACATAGACATATCAGAATGTCTCATAACCAAAAAT |
| | | TCAGTGATATGTCGTCATGACTTTGCAATGCCAATGAGTTACACCTTAAAGGAA |
| | | TGCCTAACTGGAGACACTGAAAAGTGTCCGAGAGAGGCTGTTGTAACCTCATAT |
| | | GTCCCAAGATTTGCTATCTCCGGGGGAGTGATTTATGCTAATTGTCTAAGTACA |
| | | ACATGTCAATGCTATCAAACTGGCAAAGTAATTGCTCAAGACGGCAGCCAAACA |
| | | TTGATGATGATCGATAATCAAACATGTTCAATAGTAAGAATTGAAGAAATCCTC |
| | | ATATCAACAGGGAAATATCTGGGAAGTCAGGAGTACAATACGATGCATGTGTCA |
| | | GTCGGCAATCCTGTCTTCACTGACAAGCTGGACATAACAAGTCAAATTTCCAAC |
| | | ATCAACCAATCCATTGAACAATCCAAATTTTATCTAGATAAGTCTAAGGCTATA |
| | | CTTGACAAGATAAATCTCAACTTAATTGGCTCTGTACCGATATCAATACTTTTC |
| | | ATAATTGCGATCTTATCATTGATTCTCTCTATTATAACTTTTGTGATTGTGATG |
| | | ATAATTAAATATAACAAATTTATAGATGATCCTGATTATTACAATGATTACAAA |
| | | AGAGAACGTATTAATGGCAAAGCCAGTAAGAGTAACAATATATATTATGTAGGT |
| | | GATTGATGA |
| Moj-V | 33 | ATGGCACTAAATAAAAATATGTTCAGTTCACTGTTCCTTGGTTATCTATTAGTG |
| Fusion F | | TACGCTACGACTGTTCAGTCTAGTATACACTATGACTCCTTATCTAAGGTCGGT |
| coding | | GTCATTAAGGGTCTGACATACAACTATAAGATCAAGGGTTCGCCATCTACAAAG |
| sequence, | | CTAATGGTGGTCAAATTGATACCTAACATTGATAGTGTTAAAAACTGTACTCAG |
| GenBank: | | AAACAGTATGATGAATACAAGAACTTAGTAAGGAAAGCCTTAGAACCGGTCAAA |
| KF278639.1 | | ATGGCTATTGACACCATGCTCAATAATGTTAAGTCGGGTAATAACAAGTACAGA |
| | | TTTGCAGGTGCAATTATGGCTGGAGTTGCCCTCGGTGTTGCAACAGCAGCCACT |
| | | GTTACAGCAGGGATAGCTCTCCATAGATCAAATGAAAATGCACAGGCAATTGCA |
| | | AACATGAAGAGTGCTATTCAAAATACAAATGAGGCAGTAAAGCAATTGCAATTG |
| | | GCCAATAAACAAACACTAGCTGTGATTGACACCATAAGAGGAGAGATCAATAAC |
| | | AATATAATACCCGTTATAAATCAATTGAGCTGTGACACAATTGGGCTCAGTGTA |
| | | GGTATAAGACTCACTCAGTACTACTCTGAAATAATAACTGCATTTGGGCCAGCT |
| | | TTGCAGAATCCAGTAAATACAAGGATTACCATTCAAGCAATATCTAGTGTGTTT |
| | | AATGGCAACTTTGATGAACTGCTGAAGATTATGGGGTATACAAGTGGTGATCTT |
| | | TATGAAATTCTACATAGTGAATTAATTAGAGGCAACATTATAGACGTTGATGTA |
| | | GATGCAGGATACATAGCTCTAGAAATAGAATTCCCCAATCTAACATTGGTACCT |
| | | AATGCTGTAGTACAGGAGTTAATGCCTATCAGTTATAACATAGACGGGGATGAG |
| | | TGGGTAACACTTGTGCCAAGGTTTGTACTTACAAGGACTACACTGTTATCAAAT |
| | | ATTGATACGAGTAGATGTACAATCACAGATAGTAGTGTCATATGTGACAACGAC |
| | | TACGCCTTGCCTATGTCACACGAGCTTATTGGCTGCTTACAGGGAGATACATCT |
| | | AAGTGTGCTAGAGAGAAGGTAGTCTCAAGTTATGTCCCTAAATTTGCGTTGTCT |
| | | GATGGGTTAGTGTATGCAAATTGCCTCAATACTATCTGCCGATGTATGGATACA |
| | | GATACTCCAATCTCACAAAGTCTCGGAGCCACTGTATCATTACTAGACAACAAG |
| | | AGGTGTTCAGTATATCAGGTAGGAGATGTCTTGATTTCTGTCGGATCATATCTA |
| | | GGAGATGGAGAATATAATGCTGATAATGTAGAGCTTGGCCCACCTATAGTTATA |
| | | GATAAGATTGACATAGGAAATCAGCTGGCAGGTATTAATCAAACCTTACAAGAG |
| | | GCAGAAGATTACATTGAGAAGTCAGAAGAGTTCTTAAAAGGGGTTAACCCTTCA |
| | | ATTATCACTCCAAGCGTTCAATTGTTTCTAATAATAATATCAGTCCTCTCATTT |
| | | ATTATATTATTGATTATCATAGTATACTTGTACTGTAAATCAAAACATTCATAT |
| | | AAATATAACAAATTTATAGATGATCCTGATTATTACAATGATTACAAAAGAGAA |
| | | CGTATTAATGGCAAAGCCAGTAAGAGTAACAATATATATTATGTAGGTGATTAA |
| | | TAA |
| GhV | 35 | ATGAAGAAAAAGACGGACAATCCCACAATATCAAAGAGGGGTCACAACCATTCT |
| Fusion F | | CGAGGAATCAAATCTAGAGCGCTACTCAGAGAGACAGATAATTATTCCAATGGG |
| coding | | CTAATAGTTGAGAATTTAGTTAGAAACTGTCATCATCCAAGTAAGAACAATCTA |
| sequence | | AACTATACTAAGACACAAAAAAGAGATTCTACAATCCCTTATCGTGTGGAAGAG |
| GenBank: | | AGAAAAGGACATTATCCAAAGATTAAACATCTTATTGATAAATCTTACAAGCAT |
| HQ660129.1 | | ATAAAAAGAGGGAAGAGAAGAAATGGTCATAATGGGAACATTATAACTATAATT |
| | | CTGTTGTTGATTTTAATTTTGAAGACACAGATGAGTGAAGGTGCTATCCATTAC |
| | | GAGACTCTAAGTAAGATCGGATTAATAAAGGGAATCACCAGAGAGTACAAAGTC |
| | | AAAGGAACTCCGTCAAGTAAAGACATAGTCATCAAATTGATTCCGAATGTCACC |
| | | GGTCTTAACAAGTGCACGAACATATCAATGGAAAACTATAAAGAACAACTTGAC |
| | | AAAATACTAATTCCTATTAACAACATAATTGAATTGTATGCAAACTCAACTAAA |
| | | TCAGCCCCTGGGAATGCACGTTTTGCTGGCGTTATAATTGCAGGAGTGGCATTA |
| | | GGTGTTGCAGCGGCAGCCCAAATAACTGCCGGCATTGCACTGCATGAAGCTCGA |
| | | CAGAATGCAGAGAGAATTAATCTCTTAAAGGATAGCATTTCTGCCACTAACAAC |
| | | GCAGTAGCAGAACTCCAGGAAGCAACTGGTGGAATAGTAAATGTCATTACAGGA |
| | | ATGCAAGATTACATCAATACAAATCTAGTCCCGCAGATTGACAAACTGCAATGT |
| | | AGTCAGATCAAAACGGCATTAGACATATCTCTCTCCCAATACTATTCAGAAATA |
| | | TTAACAGTGTTCGGTCCAAACCTTCAAAATCCAGTAACTACTTCCATGTCAATA |
| | | CAAGCCATATCACAATCCTTTGGGGGAAATATAGATTTGCTCTTAAACCTACTA |
| | | GGTTACACTGCAAACGACTTATTGGATTTGCTCGAAAGTAAAAGTATAACAGGC |
| | | CAAATAACATACATAAATCTTGAACATTACTTCATGGTAATCAGAGTATATTAT |
| | | CCTATAATGACAACAATCAGCAATGCTTATGTCCAGGAATTGATCAAAATTAGC |
| | | TTCAATGTCGATGGCAGTGAATGGGTATCTCTTGTACCCTCGTATATATTGATT |
| | | AGAAACTCATATCTCTCAAACATAGACATATCAGAATGTCTCATAACCAAAAAT |
| | | TCAGTGATATGTCGTCATGACTTTGCAATGCCAATGAGTTACACCTTAAAGGAA |
| | | TGCCTAACTGGAGACACTGAAAAGTGTCCGAGAGAGGCTGTTGTAACCTCATAT |
| | | GTCCCAAGATTTGCTATCTCCGGGGGAGTGATTTATGCTAATTGTCTAAGTACA |
| | | ACATGTCAATGCTATCAAACTGGCAAAGTAATTGCTCAAGACGGCAGCCAAACA |
| | | TTGATGATGATCGATAATCAAACATGTTCAATAGTAAGAATTGAAGAAATCCTC |
| | | ATATCAACAGGGAAATATCTGGGAAGTCAGGAGTACAATACGATGCATGTGTCA |
| | | GTCGGCAATCCTGTCTTCACTGACAAGCTGGACATAACAAGTCAAATTTCCAAC |
| | | ATCAACCAATCCATTGAACAATCCAAATTTTATCTAGATAAGTCTAAGGCTATA |
| | | CTTGACAAGATAAATCTCAACTTAATTGGCCCAAGCGTTCAATTGTTTCTAATA |
| | | ATAATATCAGTCCTCTCATTTATTATATTATTGATTATCATAGTATACTTGTAC |
| | | TGTAAATCAAAACATTCATATAAATATAACAAATTTATAGATGATCCTGATTAT |
| | | TACAATGATTACAAAAGAGAACGTATTAATGGCAAAGCCAGTAAGAGTAACAAT |
| | | ATATATTATGTAGGTGATTGATGA |

The cytoplasmic tail of CedV F protein coding sequence is bolded and underlined. The predicted
transmembrane domain of the non-CedV F protein coding sequences is underlined.
A single nucleotide (C924A; bold and underlined), which did not result in an amino acid change,
was modified to remove an internal AleI site in the MojV fusion F protein.
To maintain the “rule of 6” an extra TGA or TAA was added at the end of CedV F cytoplasmic
tail (bold italics) of the GhV fusion F protein.

| | | |
|---|---|---|
| MojV | 25 | ATGCTTTCTCAGCTCCAAAAAAATTACTTAGACAACTCAAACCAACAAGGTGAT |
| Fusion G | | AAAATGAACAACCCAGATAAGAAATTAAGTGTCAACTTCAACCCTTTAGAATTA |
| coding seq. | | GATAAAGGTCAAAAAGATCTCAATAAGTCTTATTATGTTAAAAACAAGAATTAT |
| Genbank: | | AACGTTTCAAATCTATTAAATGAAAGTAGTGGTAATAAGGTATTCATATTGATG |
| KF278639.1 | | AATACACTTCTGATACTGACAGGTGCTATTATTACAATAACACTAAATATCACC |
| | | AACCTGACAGCGGCTAAAAGTCAACAGAATATGCTGAAAATAATCCAAGATGAC |
| | | GTGAATGCCAAATTAGAAATGTTCGTGAATCTTGATCAATTGGTGAAGGGTGAA |
| | | ATTAAGCCAAAAGTGTCACTCATAAATACAGCAGTGAGCGTCAGCATACCCGGT |
| | | CAGATCTCAAACCTCCAGACCAAATTCCTGCAAAAATATGTTTACTTAGAAGAA |
| | | TCTATTACTAAGCAGTGCACTTGCAACCCTTTATCTGGGATATTTCCAACATCA |
| | | GGCCCAACCTACCCTCCAACTGATAAACCAGACGATGATACCACAGATGATGAC |
| | | AAAGTGGACACCACGATTAAGCCTATTGAGTACCCCAAGCCGGATGGGTGCAAT |
| | | AGAACTGGCGACCATTTCACGATGGAGCCCGGAGCTAACTTTTATACTGTCCCT |
| | | AACCTAGGACCGGCAAGTTCTAATTCTGACGAGTGTTACACAAACCCCTCTTTT |
| | | TCAATTGGGTCCTCCATCTATATGTTTTCTCAAGAGATTAGAAAAACGGACTGC |
| | | ACAGCAGGAGAGATATTATCAATTCAGATCGTCTTAGGCCGAATAGTAGACAAG |
| | | GGTCAGCAGGGTCCTCAAGCATCACCCTTATTAGTATGGGCCGTCCCAAATCCA |
| | | AAGATCATAAACTCGTGTGCTGTCGCAGCTGGAGACGAGATGGGATGGGTGTTA |
| | | TGCTCAGTGACATTAACTGCAGCATCAGGGGAGCCCATACCTCACATGTTTGAT |
| | | GGGTTCTGGTTGTATAAGTTAGAACCTGACACCGAAGTTGTATCCTATAGAATC |
| | | ACAGGCTATGCTTATCTCTTAGATAAACAATATGACTCTGTCTTTATAGGTAAG |
| | | GGCGGTGGTATTCAGAAAGGTAACGATCTATACTTTCAGATGTATGGATTGTCC |
| | | AGAAATAGGCAAAGTTTTAAGGCACTGTGTGAACATGGATCATGCCTCGGCACT |
| | | GGAGGTGGAGGGTATCAAGTGTTGTGTGACAGGGCTGTGATGTCTTTCGGGAGT |
| | | GAAGAATCACTAATTACAAATGCATATCTGAAGGTGAATGATCTGGCAAGTGGG |
| | | AAACCTGTGATAATAGGACAGACATTTCCGCCCTCAGATTCTTATAAAGGCTCA |
| | | AATGGTCGGATGTACACTATAGGTGATAAATATGGTCTGTATCTTGCTCCGTCA |
| | | TCCTGGAACAGATATCTTAGATTTGGGATAACACCAGATATTTCTGTAAGATCA |
| | | ACAACCTGGTTGAAAAGTCAAGATCCGATAATGAAGATTTTGTCAACATGCACG |
| | | AACACTGATAGAGATATGTGTCCTGAAATTTGCAATACTAGAGGTTATCAAGAT |
| | | ATTTTTCCATTGTCGGAGGATTCAGAGTATTATACATACATAGGCATAACTCCT |
| | | AATAATGGTGGAACTAAAAACTTTGTGGCCGTACGTGACTCAGATGGTCATATA |
| | | GCATCCATTGATATTTTACAAAATTATTATAGTATCACCTCAGCTACTATAAGC |
| | | TGCTTCATGTACAAAGATGAGATTTGGTGTATTGCAATCACAGAAGGGAAAAAA |
| | | CAGAAAGACAATCCTCAACGGATATATGCACATTCTTACAAAATTAGGCAAATG |
| | | TGTTATAATATGAAGTCTGCCACAGTGACTGTGGGTAATGCCAAAAATATCACA |
| | | ATCAGGAGGTATTAA |
| GhV | 26 | ATGCTTTCTCAGCTCCAAAAAAATTACTTAGACAACTCAAACCAACAAGGTGAT |
| Fusion G | | AAAATGAACAACCCAGATAAGAAATTAAGTGTCAACTTCAACCCTTTAGAATTA |
| coding seq. | | GATAAAGGTCAAAAAGATCTCAATAAGTCTTATTATGTTAAAAACAAGAATTAT |
| Genbank: | | AACGTTTCAAATCTATTAAATGAAAGTCAAAATGATCATTACATGACTGTTTCA |
| HQ660129.1 | | ACCATGATTCTTGAGATATTAGTTGTCCTGGGCATCATGTTTAATCTCATAGTT |
| | | TTAACTATGGTGTATTATCAGAATGACAACATCAATCAAAGGATGGCAGAACTT |
| | | ACAAGCAATATCACAGTCCTGAACTTAAATCTTAATCAATTGACAAACAAAATT |
| | | CAAAGAGAAATTATTCCTAGGATCACTCTTATTGACACAGCAACCACCATTACA |
| | | ATTCCTAGTGCCATTACTTACATATTAGCAACTCTGACAACCAGAATCTCGGAA |
| | | TTATTGCCGTCAATCAACCAAAAGTGTGAGTTCAAGACACCGACACTTGTCCTG |
| | | AATGACTGCAGAATAAACTGTACCCCACCACTAAACCCGTCTGATGGAGTGAAA |
| | | ATGAGTTCTCTTGCCACTAACTTGGTTGCACATGGGCCCTCTCCCTGTAGAAAC |
| | | TTTTCATCCGTACCTACAATTTACTATTATCGGATTCCAGGATTATACAATAGA |
| | | ACAGCATTGGACGAAAGATGTATACTAAACCCGAGATTGACAATAAGCAGTACA |
| | | AAATTTGCTTATGTCCACTCTGAATATGATAAAAATTGCACCAGAGGATTCAAA |
| | | TACTATGAATTGATGACATTTGGAGAAATACTGGAGGGTCCGGAAAAAGAACCC |
| | | AGAATGTTTTCTAGGTCATTTTATTCGCCCACAAATGCTGTGAACTATCATTCT |
| | | TGTACGCCGATCGTGACTGTCAATGAAGGATATTTTCTTTGCCTTGAATGCACC |
| | | TCCTCAGATCCCTTGTACAAAGCAAATCTATCTAATAGCACATTCCATTTGGTG |
| | | ATACTGAGGCATAACAAGGATGAGAAAATAGTTTCAATGCCTAGCTTTAACCTT |
| | | TCTACTGATCAAGAGTATGTTCAGATAATCCCTGCAGAAGGTGGCGGCACAGCA |
| | | GAGAGTGGCAATCTTTACTTCCCTTGTATTGGAAGGCTCTTACACAAACGAGTC |
| | | ACCCATCCTTTATGCAAAAAGTCAAATTGTTCGCGAACTGATGATGAATCTTGC |
| | | CTGAAAAGTTATTACAATCAAGGGTCGCCTCAGCACCAAGTAGTCAACTGTCTG |
| | | ATAAGGATCAGAAATGCACAGAGAGATAATCCAACCTGGGATGTTATCACAGTT |
| | | GATCTGACTAATACATACCCAGGATCAAGGAGCAGGATCTTTGGAAGCTTCTCC |
| | | AAACCGATGCTTTATCAATCATCAGTATCATGGCATACTCTTCTTCAGGTAGCA |
| | | GAGATAACAGACCTAGATAAGTATCAATTGGACTGGTTGGATACACCCTATATA |
| | | TCTCGTCCTGGAGGATCTGAGTGCCCTTTCGGAAATTATTGTCCAACGGTATGC |
| | | TGGGAAGGGACATATAATGATGTCTATAGCTTAACTCCAAATAACGATCTTTTT |
| | | GTCACTGTGTATTTGAAGAGTGAACAAGTTGCAGAGAACCCTTATTTCGCAATC |
| | | TTCTCACGGGATCAAATCTTGAAAGAATTCCCTCTTGATGCATGGATAAGCAGT |
| | | GCACGAACTACGACAATATCGTGCTTCATGTTCAACAATGAAATTTGGTGTATA |
| | | GCTGCATTAGAGATCACAAGATTGAATGATGACATCATAAGACCAATTTATTAC |
| | | TCTTTCTGGCTGCCTACTGATTGCCGGACACCATATCCCCACACCGGTAAGATG |
| | | ACCAGGGTTCCCTTGCGCTCCACATATAACTACTAA |
| Moj-V | 34 | ATGCTTTCTCAGCTCCAAAAAAATTACTTAGACAACTCAAACCAACAAGGTGAT |
| Fusion G | | AAAATGAACAACCCAGATAAGAAATTAAGTGTCAACTTCAACCCTTTAGAATTA |
| coding | | GATAAAGGTCAAAAAGATCTCAATAAGTCTTATTATGTTAAAAACAAGAATTAT |
| sequence, | | AACGTTTCAAATCTATTAAATGAAAGTCTGCACGATATCAAGTTTTGTATTTAT |
| GenBank: | | TGTATATTCTCACTGCTAATTATCATTACAATAATCAATATAATCACAATATCA |
| KF278639.1 | | ATTGTTATAACTCGTCTGAAAGTACAACAGAATATGCTGAAAATAATCCAAGAT |
| | | GACGTGAATGCCAAATTAGAAATGTTCGTGAATCTTGATCAATTGGTGAAGGGT |
| | | GAAATTAAGCCAAAAGTGTCACTCATAAATACAGCAGTGAGCGTCAGCATACCC |
| | | GGTCAGATCTCAAACCTCCAGACCAAATTCCTGCAAAAATATGTTTACTTAGAA |
| | | GAATCTATTACTAAGCAGTGCACTTGCAACCCTTTATCTGGGATATTTCCAACA |
| | | TCAGGCCCAACCTACCCTCCAACTGATAAACCAGACGATGATACCACAGATGAT |
| | | GACAAAGTGGACACCACGATTAAGCCTATTGAGTACCCCAAGCCGGATGGGTGC |
| | | AATAGAACTGGCGACCATTTCACGATGGAGCCCGGAGCTAACTTTTATACTGTC |
| | | CCTAACCTAGGACCGGCAAGTTCTAATTCTGACGAGTGTTACACAAACCCCTCT |
| | | TTTTCAATTGGGTCCTCCATCTATATGTTTTCTCAAGAGATTAGAAAAACGGAC |
| | | TGCACAGCAGGAGAGATATTATCAATTCAGATCGTCTTAGGCCGAATAGTAGAC |
| | | AAGGGTCAGCAGGGTCCTCAAGCATCACCCTTATTAGTATGGGCCGTCCCAAAT |
| | | CCAAAGATCATAAACTCGTGTGCTGTCGCAGCTGGAGACGAGATGGGATGGGTG |
| | | TTATGCTCAGTGACATTAACTGCAGCATCAGGGGAGCCCATACCTCACATGTTT |
| | | GATGGGTTCTGGTTGTATAAGTTAGAACCTGACACCGAAGTTGTATCCTATAGA |
| | | ATCACAGGCTATGCTTATCTCTTAGATAAACAATATGACTCTGTCTTTATAGGT |
| | | AAGGGCGGTGGTATTCAGAAAGGTAACGATCTATACTTTCAGATGTATGGATTG |
| | | TCCAGAAATAGGCAAAGTTTTAAGGCACTGTGTGAACATGGATCATGCCTCGGC |
| | | ACTGGAGGTGGAGGGTATCAAGTGTTGTGTGACAGGGCTGTGATGTCTTTCGGG |
| | | AGTGAAGAATCACTAATTACAAATGCATATCTGAAGGTGAATGATCTGGCAAGT |
| | | GGGAAACCTGTGATAATAGGACAGACATTTCCGCCCTCAGATTCTTATAAAGGC |
| | | TCAAATGGTCGGATGTACACTATAGGTGATAAATATGGTCTGTATCTTGCTCCG |
| | | TCATCCTGGAACAGATATCTTAGATTTGGGATAACACCAGATATTTCTGTAAGA |
| | | TCAACAACCTGGTTGAAAAGTCAAGATCCGATAATGAAGATTTTGTCAACATGC |
| | | ACGAACACTGATAGAGATATGTGTCCTGAAATTTGCAATACTAGAGGTTATCAA |
| | | GATATTTTTCCATTGTCGGAGGATTCAGAGTATTATACATACATAGGCATAACT |
| | | CCTAATAATGGTGGAACTAAAAACTTTGTGGCCGTACGTGACTCAGATGGTCAT |
| | | ATAGCATCCATTGATATTTTACAAAATTATTATAGTATCACCTCAGCTACTATA |
| | | AGCTGCTTCATGTACAAAGATGAGATTTGGTGTATTGCAATCACAGAAGGGAAA |
| | | AAACAGAAAGACAATCCTCAACGGATATATGCACATTCTTACAAAATTAGGCAA |
| | | ATGTGTTATAATATGAAGTCTGCCACAGTGACTGTGGGTAATGCCAAAAATATC |
| | | ACAATCAGGAGGTATTAA |
| GhV | 36 | ATGCTTTCTCAGCTCCAAAAAAATTACTTAGACAACTCAAACCAACAAGGTGAT |
| Fusion G | | AAAATGAACAACCCAGATAAGAAATTAAGTGTCAACTTCAACCCTTTAGAATTA |
| coding | | GATAAAGGTCAAAAAGATCTCAATAAGTCTTATTATGTTAAAAACAAGAATTAT |
| sequence | | AACGTTTCAAATCTATTAAATGAAAGTCTGCACGATATCAAGTTTTGTATTTAT |
| GenBank: | | TGTATATTCTCACTGCTAATTATCATTACAATAATCAATATAATCACAATATCA |
| HQ660129.1 | | ATTGTTATAACTCGTCTGAAAGTAAATGACAACATCAATCAAAGGATGGCAGAA |
| | | CTTACAAGCAATATCACAGTCCTGAACTTAAATCTTAATCAATTGACAAACAAA |
| | | ATTCAAAGAGAAATTATTCCTAGGATCACTCTTATTGACACAGCAACCACCATT |
| | | ACAATTCCTAGTGCCATTACTTACATATTAGCAACTCTGACAACCAGAATCTCG |
| | | GAATTATTGCCGTCAATCAACCAAAAGTGTGAGTTCAAGACACCGACACTTGTC |
| | | CTGAATGACTGCAGAATAAACTGTACCCCACCACTAAACCCGTCTGATGGAGTG |
| | | AAAATGAGTTCTCTTGCCACTAACTTGGTTGCACATGGGCCCTCTCCCTGTAGA |
| | | AACTTTTCATCCGTACCTACAATTTACTATTATCGGATTCCAGGATTATACAAT |
| | | AGAACAGCATTGGACGAAAGATGTATACTAAACCCGAGATTGACAATAAGCAGT |
| | | ACAAAATTTGCTTATGTCCACTCTGAATATGATAAAAATTGCACCAGAGGATTC |
| | | AAATACTATGAATTGATGACATTTGGAGAAATACTGGAGGGTCCGGAAAAAGAA |
| | | CCCAGAATGTTTTCTAGGTCATTTTATTCGCCCACAAATGCTGTGAACTATCAT |
| | | TCTTGTACGCCGATCGTGACTGTCAATGAAGGATATTTTCTTTGCCTTGAATGC |
| | | ACCTCCTCAGATCCCTTGTACAAAGCAAATCTATCTAATAGCACATTCCATTTG |
| | | GTGATACTGAGGCATAACAAGGATGAGAAAATAGTTTCAATGCCTAGCTTTAAC |
| | | CTTTCTACTGATCAAGAGTATGTTCAGATAATCCCTGCAGAAGGTGGCGGCACA |
| | | GCAGAGAGTGGCAATCTTTACTTCCCTTGTATTGGAAGGCTCTTACACAAACGA |
| | | GTCACCCATCCTTTATGCAAAAAGTCAAATTGTTCGCGAACTGATGATGAATCT |
| | | TGCCTGAAAAGTTATTACAATCAAGGGTCGCCTCAGCACCAAGTAGTCAACTGT |
| | | CTGATAAGGATCAGAAATGCACAGAGAGATAATCCAACCTGGGATGTTATCACA |
| | | GTTGATCTGACTAATACATACCCAGGATCAAGGAGCAGGATCTTTGGAAGCTTC |
| | | TCCAAACCGATGCTTTATCAATCATCAGTATCATGGCATACTCTTCTTCAGGTA |
| | | GCAGAGATAACAGACCTAGATAAGTATCAATTGGACTGGTTGGATACACCCTAT |
| | | ATATCTCGTCCTGGAGGATCTGAGTGCCCTTTCGGAAATTATTGTCCAACGGTA |
| | | TGCTGGGAAGGGACATATAATGATGTCTATAGCTTAACTCCAAATAACGATCTT |
| | | TTTGTCACTGTGTATTTGAAGAGTGAACAAGTTGCAGAGAACCCTTATTTCGCA |
| | | ATCTTCTCACGGGATCAAATCTTGAAAGAATTCCCTCTTGATGCATGGATAAGC |
| | | AGTGCACGAACTACGACAATATCGTGCTTCATGTTCAACAATGAAATTTGGTGT |
| | | ATAGCTGCATTAGAGATCACAAGATTGAATGATGACATCATAAGACCAATTTAT |
| | | TACTCTTTCTGGCTGCCTACTGATTGCCGGACACCATATCCCCACACCGGTAAG |
| | | ATGACCAGGGTTCCCTTGCGCTCCACATATAACTACTAA |

The cytoplasmic tail of CedV G protein coding sequence is bolded and underlined.
The predicted transmembrane domain of the non-CedV G protein coding sequences is underlined.
Two nucleotide changes were made in SEQ ID NOs: 26 and 36 (T321C to remove an internal
SwaI site and C1653A to remove an internal SmaI site; bold and underlined), which did not
result in an amino acid changes, were made in the GhV fusion G coding sequence.

As is evident from Table 3, Paramyxoviridae F proteins are type I membrane glycoproteins, which means the cytoplasmic domain is at the 3′ end of the sequence. In contrast, Paramyxoviridae G proteins are type II membrane glycoproteins, which means the cytoplasmic domain is at the 5′ end of the sequence.

The present disclosure includes additional variations to non-CedV coding sequences of the F and G proteins, beyond the optional point mutations shown in Table 3. In some embodiments, a rCedV chimera comprises a gene encoding a fusion F protein, a gene encoding fusion G protein, or both, in which the gene encoding the fusion F protein and the gene encoding the fusion G protein may comprise 1, 2, 3, 4, 5, 6, 7, 8, 9, or 10 or more substitutions, insertions, or deletions relative to the naturally-occurring sequence, so long as the resulting protein is capable of eliciting an immune response and the virus remains replication-competent. For instance, the gene encoding the fusion F protein may comprise a sequence that is about 90%, about 91%, about 92%, about 93%, about 94%, about 95%, about 96%, about 97%, about 98%, about 99%, or 100% identical to any one of SEQ ID NOs: 23, 24, 33, or 35. Similarly, the gene encoding the fusion G protein may comprise a sequence that is about 90%, about 91%, about 92%, about 93%, about 94%, about 95%, about 96%, about 97%, about 98%, about 99%, or 100% identical to any one of SEQ ID NOs: 25, 26, 34, Or 36.

As described in more detail below, such recombinant Cedar virus (rCedV) chimeras, in which one or both of the F and G envelope glycoprotein genes of CedV are replaced with a corresponding F envelope glycoprotein fusion protein and/or G envelope glycoprotein fusion protein, comprising an ectodomain with or without a transmembrane domain from a non-CedV henipavirus, may be used to elicit an immune response to a non-CedV henipavirus.

### III. Vaccine Compositions

Because CedV is naturally attenuated and non-pathogenic, the disclosed rCedV chimeras can be used as vaccines to elicit an immune response or establish immune protection against pathogenic strains of henipavirus.

In one aspect, a vaccine composition as disclosed herein comprises a recombinant Cedar virus (rCedV) chimera as disclosed herein, wherein one or both of the F and G envelope glycoprotein genes of CedV are replaced with one or both of the F and G envelope glycoprotein genes, respectively, of a non-CedV henipavirus, as described in more detail above.

In another aspect, a vaccine composition as disclosed herein comprises a rCedV chimera as disclosed herein comprising the F and G envelope glycoprotein genes of CedV, and further comprising a coding sequence for one or both of (i) a soluble F envelope glycoprotein (sF) of a non-CedV henipavirus and (ii) a soluble G envelope glycoprotein (sG) of a non-CedV henipavirus, as described in more detail above.

In another aspect, a vaccine composition as disclosed herein comprises a rCedV chimera as disclosed herein comprising one or both of a gene encoding a fusion protein of a henipavirus F envelope protein and a gene encoding a fusion protein of a henipavirus G envelope protein, wherein the fusion protein comprises the ectodomain and transmembrane domain of a non-CedV henipavirus F envelope protein or a non-CedV henipavirus G envelope protein fused to the cytoplasmic tail domain of the CedV F envelope protein or CedV G envelope protein, respectively, or the fusion protein comprises the ectodomain of a non-CedV henipavirus F envelope protein or a non-CedV henipavirus G envelope protein fused to the transmembrane domain and cytoplasmic tail domain of the CedV F envelope protein or CedV G envelope protein, respectively, as described in more detail above.

In another aspect, a vaccine composition as disclosed herein may comprise or further comprise (in addition to a chimera as described herein) a non-CedV sF, a non-CedV sG, or both. The non-CedV henipavirus can be selected from HeV, NiV, GhV, or MojV or any other pathogenic henipavirus. In some embodiments, the sF and/or sG is HeV or NiV (e.g., NiV-B or NiV-M).

The immune response elicited by immunization with a chimera or vaccine as disclosed herein is expected to induce production of antibodies that bind henipavirus and provide broad spectrum immune protection against henipaviruses. For example, as noted above, a chimera displaying a non-CedV ectodomain of one species of henipavirus, such as HeV, may induce a protective immune response against other species of henipavirus, such as NiV-M and NiV-B.

In accordance with any embodiments, a vaccine as disclosed herein may comprise a pharmaceutically acceptable carrier suitable for the intended subject and route of administration. Pharmaceutically acceptable carriers for various dosage forms and routes of administration are known in the art. For example, solvents, solubilizing agents, suspending agents, isotonicity agents, buffers, and soothing agents for liquid preparations are known. In some embodiments, the disclosed vaccine compositions include one or more additional components, such as one or more preservatives, antioxidants, and the like.

In accordance with any embodiments, a vaccine as disclosed herein may be formulated for injection and administered parenterally, such as intravenously, intramuscularly, subcutaneously, or intradermally. Thus, a vaccine as disclosed herein may be formulated for intravenous injection or infusion. Alternatively, with any embodiments, a vaccine as disclosed herein may be formulated for administration by inhalation. Vaccine compositions disclosed herein can be formulated according to standard methods (see, for example, Remington's Pharmaceutical Science, Mark Publishing Company, Easton, USA).

In accordance with any vaccine composition embodiments described herein, a vaccine composition as described herein optionally may further comprise an adjuvant. An adjuvant is an ingredient used in some vaccines that helps create a stronger immune response in people receiving the vaccine. Adjuvants typically help the body produce an immune response strong enough to protect the person from the disease he or she is being vaccinated against. Adjuvants suitable for use in vaccine compositions are known in the art, including adjuvants suitable for use in henipavirus vaccine compositions. Any such adjuvants can be used in the vaccine compositions described herein.

Any of the vaccine compositions disclosed herein can be used for treating, reducing the risk of, and/or preventing a henipavirus infection. Optimal doses and routes of administration may vary. The administration methods can be properly selected according to the patient's age, weight, and condition.

The disclosed vaccines may be formulated to be administered alone or concurrently with another therapeutic agent for treating henipavirus (i.e., an antiviral agent). The vaccines may be formulated to be administered in sequence with another therapeutic agent or concurrently with another therapeutic agent. For example, the vaccine may be administered either before or after the subject has received a regimen of an antiviral therapy. The disclosed vaccines may be administered as a single dose or an initial dose followed by one or more booster doses.

### IV. Methods and Uses

### A. Treatments, Reductions of Risk, and Prevention

The vaccines and chimeras disclosed herein can be used for treating henipavirus infections, reducing the risk of henipavirus infections, or preventing henipavirus infections. Accordingly, in another aspect, the present disclosure provides uses of the chimeras and vaccine compositions described herein in methods of treating, reducing the risk of, or preventing a pathogenic henipavirus (e.g., HeV, NiV, GhV, or MojV) infection in a subject in need thereof, comprising administering to the subject an effective amount of a chimera or vaccine composition as described herein.

Administration may be via an injection. The injection may be given, for example, intravenously, intramuscularly, subcutaneously, or intradermally. Alternatively, administration may be via an inhalation.

The targeted henipavirus may be any known, presumed, or suspected pathogenic henipavirus, such as HeV, NiV, GhV, or MojV. In some embodiments, the target henipavirus is HeV. In some embodiments, the target henipavirus is NiV (e.g., NiV-B or NiV-M).

The subject may be any subject in need of treatment of, reduction of risk of, or prevention of henipavirus infection. In some embodiments, the subject is a mammalian subject. In some embodiments, the subject is a human subject. In some embodiments, the subject is a non-human mammal. In some embodiments, the subject is a livestock mammal.

Another aspect of the present disclosure is directed to uses of the chimeras and vaccine compositions described herein in methods of inducing an immune response against a pathogenic henipavirus in a subject, comprising administering to the subject an effective amount of a chimera or vaccine composition as described herein.

Dosage regimens for the disclosed methods and uses can be adjusted to provide the optimum desired response. For example, in some embodiments, a single bolus of a vaccine or rCedV may be administered, while in some embodiments, several doses may be administered over time, or the dose may be proportionally reduced or increased as indicated by the situation. In some embodiments, a subject may be administered more than one distinct rCedV, such as two or three or more distinct rCedV disclosed herein.

### B. Assay Reagents

Another aspect of the present disclosure is directed to uses of the chimeras described herein as assay reagents. The replication competent rCedV chimeras described herein can be used under BSL-2 containment (as opposed to the BSL-4 containment required for pathogenic henipaviruses), making them ideal for use in studying henipaviruses and agents being developed to target them. For example, the rCedV chimeras described herein can be used as the virus component of a virus neutralization assay to assess and characterize virus-neutralizing antibodies or antibody responses, such as for studies of vaccines against NiV or HeV. The rCedV based chimeras disclosed herein also may be used to study henipavirus entry mechanisms and in entry receptor studies, and in entry inhibitor antiviral drug discovery studies of other henipaviruses.

The following examples are given to illustrate the present disclosure. It should be understood that the invention is not to be limited to the specific conditions or details described in these examples.

### EXAMPLES

### Example 1—Rescue of Replication-Competent rCedV Chimeras

### Rescue of rCedV Stock

In order to rescue replication-competent rCedV chimeras, such as rCedV-NiV-B, rCedV-HeV, rCedV-GhV, rCedV-MojV, or other yet to be described henipaviruses, a stock preparation of each rCedV chimera antigenome plasmid was prepared and sequenced.

BSR-T7/5 cells that stably express T7 RNA polymerase were transfected with four plasmids: first; with the individual rCedV antigenome clone, and three support plasmids expressing CedV nucleoprotein (N), phosphoprotein (P), and polymerase protein (L) required for encapsidation and replication. Four to five days post transfection, GFP expression (if included in the genome construction) or syncytia formation were monitored. After seven days, the supernatant was passaged to fresh Vero E6 cells to amplify rescued virus. Alternatively, six hours post-transfection, the media was changed, and Vero E6 cells were co-cultured with transfected BSR-T7/5 cells. GFP expression and syncytia were monitored, and after seven days supernatant was passaged to fresh Vero E6 cells, which was monitored for syncytia and GFP expression. When maximal GFP expression and syncytia was observed, supernatants were purified by centrifugation and stored or used for experimentation. For virus stock storage, rCedVs are stored at −80° C. as single use aliquots. The virus was removed and thawed and diluted for infection immediately prior to use.

### Construction of Genetic Cassettes

Novel genetic cassettes with various intergenic regions for the cloning and manipulations of the full-length rCedV antigenome clones were individually designed and constructed to accommodate additional reporter genes, such as green fluorescent protein (GFP) or luciferase protein (Luc). The CedV intergenic regions between each given F and G henipavirus gene cassette were retained, while also retaining the flanking intergenic sequences on the ends of each cassette to that of CedV.

The F and G gene cassettes of either NiV-B or HeV were individually designed to have CedV F start and stop untranslated intergenic regions flanking the NiV-B or HeV F coding region and the CedV G start and stop untranslated intergenic regions flanking the NiV-B or HeV G coding region. To maintain the “rule-of-six” (a feature employed by the henipaviruses to promote replication initiation (38)), additional modifications to the NiV-B and HeV F coding regions were made. Specifically, for the NiV-B F coding region, the last three nucleotides (ACG) from the NiV-B F coding region were not included in the chimera design, as deletion of this amino acid (Threonine) has been shown not to interfere with endocytosis, trafficking or fusion (39) and therefore would not interfere with the proper maturation and biological activity of the NiV-B F glycoprotein and a rCedV chimera rescue. For the HeV F coding region, an additional TAA (stop codon) sequence was added to the end of the HeV F coding region, which would not impact protein expression or functionality. These custom designed genetic cassettes were synthesized and various rCedV whole genome clones (the chimeras) encoding the F and G envelope glycoproteins of HeV and NiV-B, both with and without reporter genes, were used to rescue replication competent viruses.

In addition, an optimized full-length antigenome clone of rCedV for the reverse genetics system was designed to enhance recombinant virus rescue efficiency. This was achieved by modifying the 5-prime end of the antigenome clone by inserting additional nucleotides GGGAGA to the minimal T7 RNA polymerase promoter (T7 min) to create an optimized T7 RNA polymerase promoter (T7opt) followed by a self-cleaving autocatalytic hammerhead ribozyme A (HHRbzA) sequence.

### Construction of rCedV Chimeric Genome Clones

A T7 polymerase promoter (T7 min) sequence containing additional nucleotides GGGAGA (T7ₒₚₜ) (19) preceding a hammerhead ribozyme A (HHRbzA) sequence was synthesized (Genscript; NJ, USA) and enzymatically inserted before the 3′Le (3′ Leader) sequence of the pOLTV5-rCedV antigenome plasmid. The F and G glycoprotein open reading frames from NiV-B 2010 Faridpur isolate (F: NCBI accession number AEZ01396.1 and G: NCBI accession number AEZ013971.1) and HeV 2008 Redlands isolate (F: NCBI accession number AEQ38070.1 and G: NCBI accession number AEQ38071.1) (Table 4) were synthesized separately (Genscript; NJ, USA). The NiV-B F and G open reading frames are from the NiV-B 2010 Faridpur isolate GenBank: JN808864.1, with protein sequences GenBank: AEZ01396.1 and GenBank: AEZ013971.1, for NiV-B F and G glycoproteins, respectively. The HeV F and G open reading frames are derived from the HeV genome GenBank: MN062017.1. The HeV F and G amino acid sequences are identical to the F and G amino acid sequences of the HeV 2008 Redlands isolate (GenBank: JN255805.1) and protein sequences GenBank: AEQ38070.1 and GenBank: AEQ38071.1, for HeV F and G glycoproteins, respectively.

TABLE 4
Genbank accession numbers of envelope glycoproteins from NiV-B
and HeV isolates inserted in rCedV antigenome plasmid.

| Henipavirus | Isolate | Protein | Genbank Accession # |
|---|---|---|---|
| NiV-B | 2010 Faridpur | F | AEZ01396.1 |
| | | G | AEZ01397.1 |
| HeV | 2008 Redlands | F | AEQ38070.1 |
| | | G | AEQ38071.1 |

The synthesized gene cassettes were inserted by standard molecular techniques into the newly generated pOLTV5-rCedV antigenome plasmid. A modified turbo Green Fluorescent Protein (GFP) gene or a Firefly Luciferase protein (Luc) gene as previously described (5) was enzymatically inserted between the CedV P and M genes of the newly generated pOLTV5-rCedV antigenome plasmid. All cloning was performed with Escherichia coli Stbl2 cells (Invitrogen; CA, USA). All plasmids were sequenced to obtain at least 2-fold coverage.

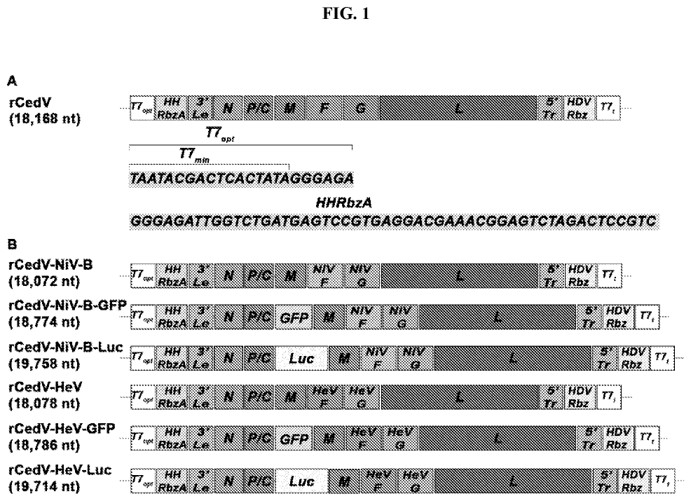

FIGS. 1A-B show the optimized rCedV chimera antigenomes prepared in comparison to rCedV. FIG. 1A shows sequences of the T7 minimal promoter (T7ₘᵢₙ) with additional GGGAGA nucleotides to generate the T7 optimal promoter (T7ₒₚₜ) and the Hammerhead Ribozyme A (HHRbzA) in the context of the rCedV antigenome plasmid. The T7ₒₚₜ promoter and HHRbzA sequences were inserted before the 3′ Leader (3′ Le) sequence of the rCedV antigenome plasmid. The long arrows indicate regions of self-cleavage. Unique restriction sites MluI and SphI used to construct the rCedV chimeric plasmids are shown. FIG. 1B shows the genomes and the lengths of the generated chimeras. CedV F and G glycoproteins were enzymatically replaced with NiV-B or HeV fusion (F) and attachment (G) open reading frames to yield rCedV-NiV-B or rCedV-HeV, respectively. A modified turbo Green Fluorescent Protein (GFP) gene or Firefly Luciferase protein (Luc) gene was inserted between CedV P and M genes to generate rCedV-NiV-B-GFP and rCedV-HeV-GFP, or rCedV-NiV-B-Luc and rCedV-HeV-Luc, respectively. Abbreviations: T7ₘᵢₙ, T7 minimal promoter; T7ₒₚₜ, T7 optimal promoter; HHRbzA, Hammerhead Ribozyme A; 3′Le, 3′ Leader; 5′Tr, 5′ Trailer; HDVRbz, hepatitis delta virus ribozyme; T7ₜ, T7 terminator.

### Rescue of rCedV Chimeras

BSR-T7/5 cells in a 12-well plate (2.5×10⁵ cells/well) were co-transfected with pCMV-CedV helper plasmids pCMV-CedV-N (1.25 μg), pCMV-CedV-P (0.8 μg) and pCMV-CedV-L (0.4 μg) together with each of the rCedV chimera antigenome constructs (3.5 μg) using TransIT-LT1 transfection reagent (Mirus Bio; WI, USA) according to the manufacturer's recommendations. After 4-5 days, transfected cells were microscopically observed for syncytia formation and expression of GFP. Supernatants from successful rescue wells were collected and passaged onto naïve Vero E6 cells in a T-75 flask to prepare a master stock of each of the rCedV chimeras. When maximal syncytia or GFP expression was observed (˜2-3 days), viral supernatants were collected, clarified by centrifugation at 948×g for 10 mins to pellet cell debris, and transferred to screw cap tubes as single use aliquots stored at −80° C. Viral stocks were titrated by plaque assay (5, 20). Briefly, a ten-fold serial dilution of the virus stock was prepared in DMEM-10, 200 μl of which was applied to pre-seeded Vero E6 cells in duplicate (5×10⁵ cells/well) in a 12-well plate and incubated for 1 hour at 37° C., 5% C02. A 2 ml overlay of a 1:1 mix of DMEM-5 with 2% carboxymethylcellulose sodium salt (medium viscosity) (Sigma-Aldrich; MO, USA) was applied to all wells and incubated for 4 days at 37° C., 5% C02. Cells were fixed with 4% Formaldehyde in diH2O for 1 hour at room temperature and stained with 0.5% crystal violet in 80% methanol for 15 mins at room temperature. The stain was removed and washed with diH2O and plaques were counted and expressed as plaque forming units (PFU)/ml. All rCedV chimera virus stocks were deep-sequenced.

### Western Blot Analysis

Vero E6 cells in a 6-well plate were infected at a density of 1×10⁶ cells with each of the rCedV-NiV-B chimeras, rCedV-HeV chimeras or rCedV viruses, at a multiplicity of infection (MOI) of 0.01. In addition, Vero E6 cells were transfected with pcDNA3.1-CMV-NiV-F, pcDNA3.1-CMV-NiV-G, pcDNA3.1-CMV-HeV-F or pcDNA3.1-CMV-HeV-G in a 1:3 ratio (F:G). At 24 hours post infection (hpi), cells were collected and lysed with 1×RIPA (Radioimmunoprecipitation assay) buffer containing a protein inhibitor cocktail. Total protein (30 μg) was separated on a 4-12% Bis-Tris gel (Thermo Fisher Scientific) and transferred to a nitrocellulose membrane (Thermo Fisher Scientific). The membranes were blocked in 5% milk in 1×PBS with 0.1% Tween-20 at room temperature. NiV and HeV cross reactive murine monoclonal antibodies (mAbs) specific to G glycoprotein (mAb 48D3) and F glycoprotein (mAb 5G7), polyclonal rabbit sera to CedV-N(CSIRO, Australia), and j-actin (Thermo Fisher Scientific) were used and subsequently probed with a corresponding secondary HRP-coupled antibody and visualized by autoradiography.

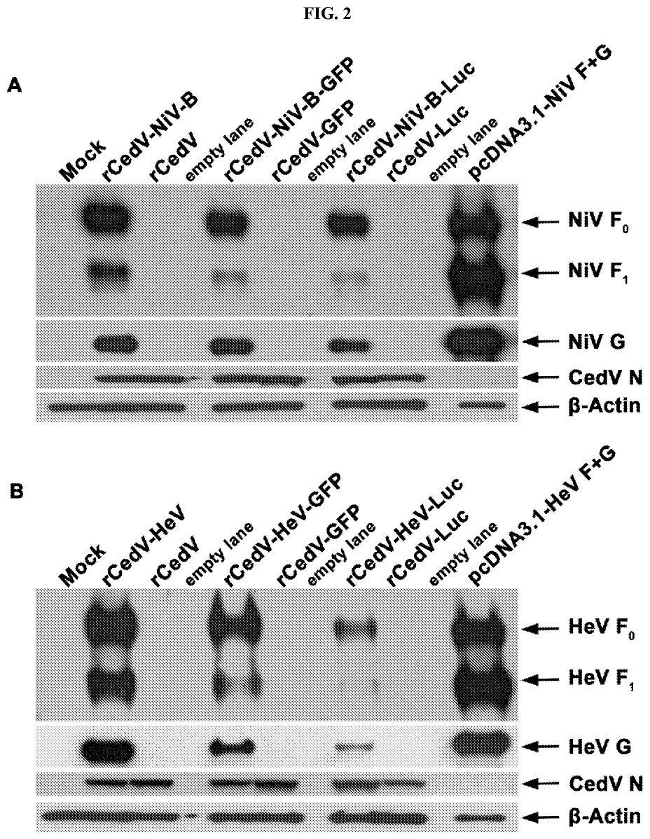

rCedV chimeras for HeV and NiV-B without reporter genes, and with the GFP or Luc reporter genes, are shown in FIGS. 2A-B. Vero E6 cells were uninfected (Mock) or infected at a multiplicity of infection (MOI) of 0.01 with either rCedV-NiV-B, rCedV-NiV-B-GFP, rCedV-NiV-B-Luc (A), rCedV-HeV, rCedV-HeV-GFP, rCedV-HeV-Luc (B), rCedV, rCedV-GFP or rCedV-Luc. As a reference, cells were co-transfected with a total of 2 μg of pcDNA3.1-NiV F+G or pcDNA3.1-HeV F+G. All cells were harvested at 24 hours post infection (hpi), lysates prepared and total protein (˜30 μg) resolved by electrophoresis (SDS-PAGE). The subsequent membrane was probed with HeV and NiV cross-reactive mAbs against F glycoprotein (mAb 5G7) and G glycoprotein (mAb 48D3), polyclonal rabbit serum to CedV-N and β-actin. Representative images from two independent experiments are shown. This confirms the identity, by western blot detection, of the NiV-B and HeV species of the F and G glycoproteins being expressed by the rCedV chimeras: rCedV-NiV-B, rCedV-NiV-B-GFP, and rCedV-NiV-B-Luc (FIG. 2A) and rCedV-HeV, rCedV-HeV-GFP, and rCedV-HeV-Luc (FIG. 2B).

### Cell-Cell Fusion Assay

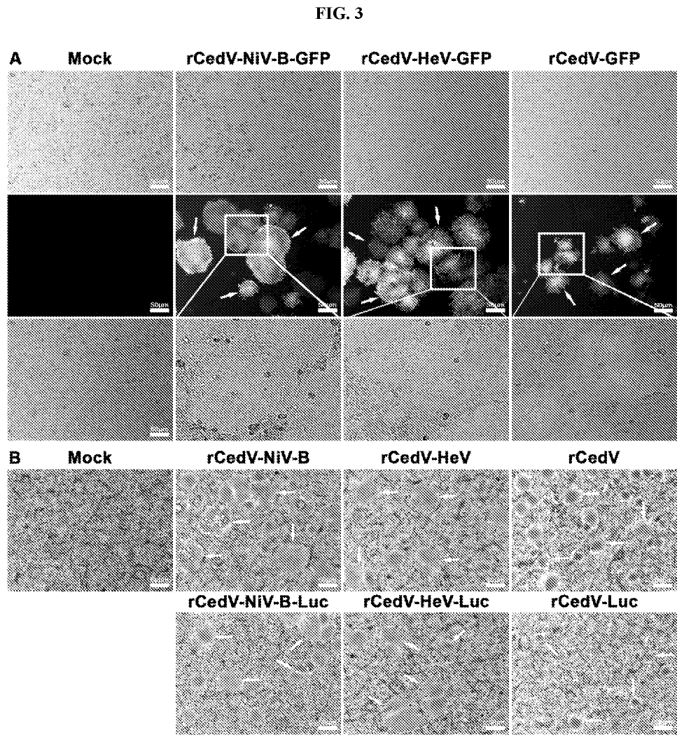

Cell-cell fusion mediated by rCedV chimeras was demonstrated by syncytia formation (giant cells) by rCedV chimeras as shown in FIG. 3. The rCedV chimeras bearing NiV-B or HeV envelope glycoproteins form syncytia in infected Vero E6 cells. Vero E6 cells were uninfected (Mock) or infected with either rCedV-NiV-B, rCedV-NiV-B-GFP, rCedV-NiV-B-Luc, rCedV-HeV, rCedV-HeV-GFP, rCedV-HeV-Luc, rCedV, rCedV-GFP or rCedV-Luc at a multiplicity of infection (MOI) of 0.01. All images were taken 24 hpi. (FIG. 3A) Cells infected with GFP expressing viruses. Transmitted light (top row), fluorescence (middle row) and merged (bottom row) images are shown. The respective zoomed in fluorescence images (3rd row) are regions in the boxes. (FIG. 3B) Cells infected with non-reporter or Luc expressing rCedV chimeras were fixed, stained and then imaged for syncytia. The images taken with transmitted light are shown. Images were captured with a Zeiss Axio Observer A1 inverted microscope using a 5× objective. Arrows indicate giant multinucleated cells (syncytia). Representative images from three independent experiments are shown. Scale bar, 50 μm.

### Example 2—Replication Kinetics of rCedV Chimeras

Vero E6 cells seeded at a density of 2×10⁴ cells/well in a 96-well cell culture plate were infected at a MOI of 0.01 for 1 hour at 37° C., 5% CO₂. Viral overlay was removed and fresh medium added to all wells. Supernatants were collected at 0, 8, 24, 48 and 72 hpi and stored at −80° C. until ready to analyze. Viral titers (PFU/ml) were determined by plaque assay. Intracellular luciferase activity was determined with the Steady-Glo® Luciferase Assay System (Promega; Madison, WI) in a 1:1 mixture with cell culture medium. After a 10 minute incubation at room temperature, the homogenate was transferred to a white opaque 96-well cell culture plate, Nunc™ F96 MicroWell™ White Polystyrene Plate (ThermoFisher Scientific) and read using the GloMax® —Multi Detection System (Promega).

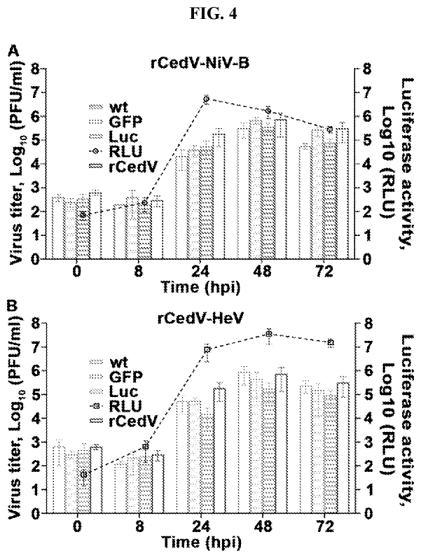

The various rCedVs have similar infection and replication kinetics. FIGS. 4A-B show a comparison of progeny virus production over time by determining infectious virus titers (PFU/ml) from supernatants harvested at the indicated time points from Vero E6 cells infected at a multiplicity of infection (MOI) of 0.01 with the rCedV-NiV-B (clear leftmost bar), rCedV-NiV-B-GFP (dotted bar), rCedV-NiV-B-Luc (striped bar) (A), rCedV-HeV (clear leftmost bar), rCedV-HeV-GFP (dotted bar) or rCedV-HeV-Luc (striped bar) (B). As a reference Vero E6 cells were also infected with rCedV (black rightmost bars) (A and B). Normalized relative light units (RLU) for CedV-NiV-B-Luc (A) and rCedV-HeV-Luc (B) infected cells are represented on the right Y-axes as dashed lines. The data represent mean±standard deviation from three independent experiments. Virus titers and luciferase activity levels at 0 hpi indicate the lower limit of detection for the plaque assay and the luminometer, respectively. Statistical analysis was performed in GraphPad Prism 9 by two-way ANOVA followed by Tukey's post hoc test (α=0.05).

### Example 3—Ephrin Receptor Tropism

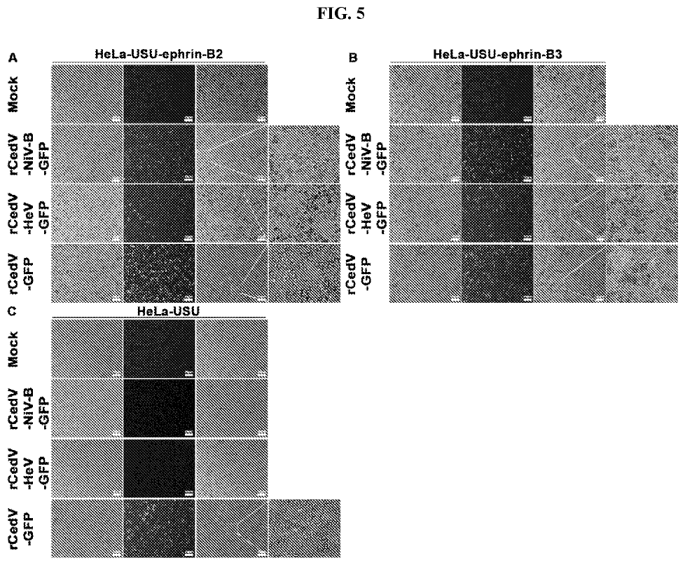

To evaluate and confirm the entry and infection ephrin receptor tropism of the rCedV chimeras for HeV and NiV-B, infection and syncytia formation assays were conducted. A representative experiment with the GFP encoding rCedV chimeras for HeV and NiV-B is shown in FIGS. 5A-C.

Previously, HeLa-USU cells, which lack expression of ephrin-B2 and ephrin-B3 were used to generate stable cell lines expressing ephrin-B2 (HeLa-USU-ephrin-B2) and ephrin-B3 (HeLa-USU-ephrin-B3) (5). HeLa-USU, HeLa-USU-ephrin-B2 and HeLa-USU-ephrin-B3 cell lines were seeded at a density of 2.5×105 cells/well in a 12-well cell culture plate. When confluent, cell culture medium was removed and cells were left uninfected (Mock) or infected at a multiplicity of infection (MOI) of 0.5 with rCedV-NiV-B-GFP, rCedV-HeV-GFP or rCedV-GFP. Infected cells were imaged for fluorescence and syncytia at 24 hpi. In each panel, transmitted light (1st column), fluorescence (2nd column) and merged (3rd column) images are shown. Zoomed in regions are from the boxes. Images were captured with a Zeiss Axio Observer A1 inverted microscope using a 5× objective. Representative images from two independent experiments are shown. Scale bar, 50 μm.

Of the B class ephrin ligands, CedV utilizes ephrin-B2 and not ephrin-B3; whereas NiV-B and HeV utilize ephrin-B2 and ephrin-B3 (4). Thus, the rCedV-HeV-GFP and rCedV-NiV-B-GFP have the same entry receptor tropism as authentic NiV-B and HeV.

### Example 4—Neutralization Tests

The utility of these new rCedV chimeras were tested as viable surrogate viruses for the conduct of authentic NiV-B and HeV neutralization tests. The GFP-encoding rCedV chimeras for NiV-B and HeV were evaluated as tools for determining antibody neutralization activities in both the standard Plaque reduction neutralization test (PRNT) and a new Fluorescent reduction neutralization test (FRNT) utilizing the GFP reporter gene activity. Well-characterized HeV and NiV cross-reactive neutralizing mAbs (human mAb m102.4, humanized mAb h5B3.1, and murine mAbs 12B2 and 1F5 (21, 23-25), and also polyclonal anti-NiV G nonhuman primate sera from animals immunized with recombinant NiV-M and NiV-B sG glycoprotein (Auro Vaccines, LLC) were assayed using the rCedV-NiV-B-GFP and rCedV-HeV-GFP chimeric viruses; these mAbs were also directly compared to authentic NiV-B and HeV in side-by-side virus neutralization tests in the BSL-4 facility at the Galveston National Laboratory (GNL), University of Texas Medical Branch (UTMB), Galveston, TX.

### Plaque Reduction Neutralization Test (PRNT)

Vero 76 cells were seeded at a density of 6×10⁵ cells/well in a 6-well plate and incubated overnight at 37° C., 5% CO₂. The mAbs, serially diluted 3-fold in DMEM-10 to final concentrations ranging from 10 μg/ml to 0.0046 μg/ml were incubated with a target concentration of 100 PFU of rCedV-NiV-B-GFP or rCedV-HeV-GFP for 1 hour at 37° C., 5% C02. Each of the virus-mAb mixtures (400 μl/well) was added to duplicate wells of the pre-seeded 6-well plate. Following a 1-hour incubation at 37° C., 5% CO₂, the wells were overlaid with 1:1 mix of 0.8% agarose/DMEM-10 and incubated for 4 days. Neutral red solution was added to each well and incubated for 24 hours at which time plaques were counted. Neutralization percent (%) was calculated based on PFU for each virus without antibody. The 50% inhibitory concentration (IC₅₀) was determined as the antibody concentration at which there was a 50% reduction in plaque counts versus untreated control wells. The IC₅₀ values were calculated by non-linear regression curve fit with variable slope with GraphPad prism (GraphPad Software Inc.). The limit of detection for this assay was 50 PFU.

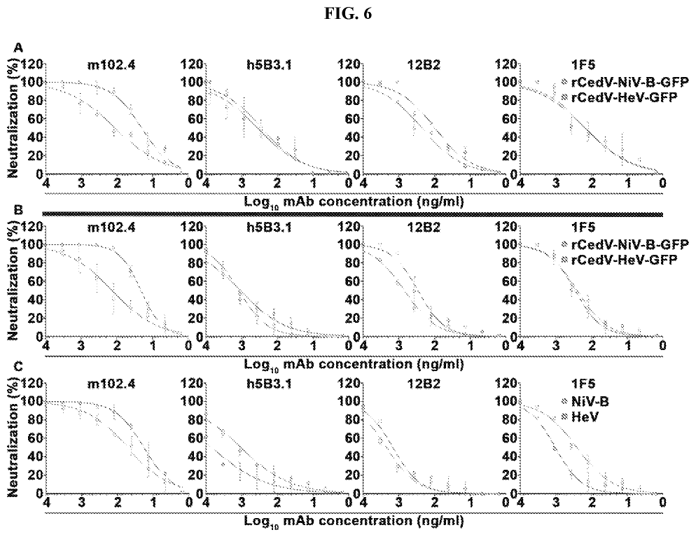

FIGS. 6A-C show the virus neutralization (PRNT) activities of a neutralizing cross-reactive mAb (m102.4) that targets the G glycoprotein of NiV and HeV, and three neutralizing cross-reactive mAbs (h5B3.1, 12B2 and 1F5) that target the F glycoprotein of NiV and HeV, against the GFP-encoding rCedV chimeras rCedV-NiV-B-GFP and rCedV-HeV-GFP (FIGS. 6A and 6B) in comparison to authentic NiV-B and HeV (FIG. 6C). The neutralization activity by these mAbs (neutralization inhibitory concentrations for 50% inhibition in comparison to no mAb (IC₅₀)) against the rCedV chimeras are highly comparable to the neutralization profiles against authentic NiV-B and HeV. The limit of detection for this assay was 50 PFU. Data are plotted as non-linear regression curve fit with variable slope using GraphPad prism and are representative of a single experiment performed in duplicate. Grey circles and lines represent rCedV-NiV-B-GFP or NiV-B and light grey squares and lines represent rCedV-HeV-GFP or HeV.

Table 5 tabulates the IC₅₀ calculations by PRNT for the rCedV-NiV-B-GFP and rCedV-HeV-GFP conducted at BSL-2; and also by PRNT assay carried out in BSL-4 of the rCedV-NiV-B-GFP and rCedV-HeV-GFP chimeras and authentic NiV-B and HeV side-by-side in the same assay.

TABLE 5
IC₅₀ doses of HeV and NiV cross-reactive specific monoclonal antibodies against rCedV-NiV-B-GFP
and rCedV-HeV-GFP infection in Vero 76 cells by plaque reduction neutralization test (PRNT).

| Monoclonal | IC₅₀ (95% CI) (ng/ml) | | | | | |
|---|---|---|---|---|---|---|
| antibody | BSL-2 | BSL-4 | | BSL-2 | BSL-4 | |
| (mAb) | rCedV-NiV-B-GFP | rCedV-NiV-B-GFP | NiV-B | rCedV-HeV-GFP | rCedV-HeV-GFP | HeV |
| m102.4 | 20.30 | 21.20 | 18.36 | 112.9 | 137.0 | 52.41 |
| | (16.58-24.99) | (18.80-23.89) | (15.21-22.17) | (82.82-154.1) | (89.09-208.8) | (39.32-70.07) |
| h5B3.1 | 274.8 | 1,122 | 7,101 | 363.5 | 1,202 | 1,064 |
| | (185.9-403.7) | (813.6-1,548) | (4,323-15,087) | (241.6-546.3) | (975.2-1,481) | (827.4-1,372) |
| 12B2 | 130.0 | 291.9 | 1,467 | 502.3 | 700.1 | 2,202 |
| | (97.10-174.0) | (219.9-381.5) | (1,098-1,925) | (377.4-658.3) | (570.0-857.0) | (1,692-2,846) |
| 1F5 | 153.8 | 289.4 | 1,036 | 140.6 | 253.2 | 259.8 |
| | (107.0-219.4) | (229.9-360.9) | (812.3-1,298) | (83.29-232.0) | (200.5-318.8) | (213.2-315.8) |

Note:
All IC₅₀ values are calculated by a nonlinear fit model and are shown with 95% confidence intervals (95% CI). BSL-2 studies are representative of two independent experiments each performed in duplicate and BSL-4 studies are from a single experiment performed in duplicate.

Together, these results show that the rCedV-NiV-B-GFP or rCedV-HeV-GFP chimeras respond to mAb neutralization in a highly similar fashion to that of authentic NiV-B and HeV in the context of a standard virus PRNT and are a remarkably ideal surrogate virus assay system that does not need to be used under BSL-4 containment.

Correlation analysis of plaque reduction neutralization test (PRNT) neutralization values from the data in FIG. 6A-C is shown in FIG. 7A-H. Pearson correlation analysis of PRNT neutralization (%) values of rCedV-NiV-B-GFP versus NiV-B (A, C, E, G) and of rCedV-HeV-GFP versus HeV (B, D, F, H) with mAbs m102.4, h5B3.1, 12B2 or 1F5. The Pearson correlation coefficient ‘r’, p-value (two-tailed), linear regression line (solid lines) and 95% confidence intervals (dashed lines) are represented. Pearson's r≥0.8 and p value <0.05 indicate a strong significant positive correlation. Table 6 summarizes the correlation analysis of rCedV chimeric virus BSL-2 PRNT versus NiV-B and HeV BSL-4 PRNT.

TABLE 6
Correlation analysis of rCedV chimeric virus BSL-2 PRNT versus NiV-B and HeV BSL-4 PRNT.

| | Monoclonal | Pearson's correlation | Coefficient of | Significance | 95% confidence |
|---|---|---|---|---|---|
| Virus | antibody (mAb) | coefficient (r) | determination (R²) | (p) | interval (CI) |
| rCedV-NiV-B-GFP | m102.4 | 0.9949 | 0.9898 | <0.0001 | 0.9750-0.990 |
| vs NiV-B | h5B3.1 | 0.8624 | 0.7437 | 0.0028 | 0.4640-0.9706 |
| | 12B2 | 0.8363 | 0.6994 | 0.0050 | 0.3873-0.9647 |
| | 1F5 | 0.9239 | 0.8536 | 0.0004 | 0.6722-0.9842 |
| rCedV-HeV-GFP | m102.4 | 0.9771 | 0.9547 | <0.0001 | 0.8914-0.9953 |
| vs HeV | h5B3.1 | 0.9548 | 0.9117 | <0.0001 | 0.7945-0.9907 |
| | 12B2 | 0.8863 | 0.7855 | 0.0015 | 0.5400-0.9760 |
| | 1F5 | 0.9898 | 0.9796 | <0.0001 | 0.9503-0.9979 |

Note:
Correlation analysis was performed with the neutralization values from FIGS. 6A and 6C.

### Fluorescent Reduction Neutralization Test (FRNT)

To highlight the further utility of the GFP-encoding rCedV chimeras rCedV-NiV-B-GFP and rCedV-HeV-GFP as surrogate viruses for authentic NiV-B and HeV, a new more rapid and quantitative neutralization assay measuring GFP fluorescence was established and used to conduct virus neutralization tests by fluorescence quantification.

Vero 76 cells were seeded at a density of 2×10⁴ cells/well in black-walled clear bottom 96-well plates (Corning Life Sciences; NY, USA) and incubated overnight at 37° C., 5% C02. The mAbs were 3-fold serially diluted in DMEM-10 to final concentrations ranging from 10 μg/ml to 0.0046 μg/ml or monkey sera were 3-fold serially diluted in DMEM-10 starting with a 1:200 dilution. An equal volume of DMEM-10 containing rCedV-NiV-B-GFP or rCedV-HeV-GFP was added to each dilution for a final concentration of 2000 PFU (MOI: 0.05) and incubated for 2 hours at 37° C., 5% C02. Each of the virus-mAb mixtures (90 μl/well) was added to the pre-seeded Vero 76 cells in triplicate and incubated for an additional 24 hours at 37° C., 5% C02. The plate was then fixed with 4% formaldehyde in 1×PBS for 20 minutes at room temperature and then washed extensively with diH₂O. The fixed plate was then scanned using the CTL S6 analyzer (Cellular Technology Limited; OH, USA). Fluorescent foci were counted using the CTL Basic Count™ feature. The 50% inhibitory concentration (IC₅₀) was determined as the antibody concentration at which there was a 50% reduction in fluorescent foci versus untreated control wells. The IC₅₀ values were calculated by non-linear regression curve fit with variable slope with GraphPad prism (GraphPad Software Inc.). The limit of detection for this assay was 50 fluorescent foci.

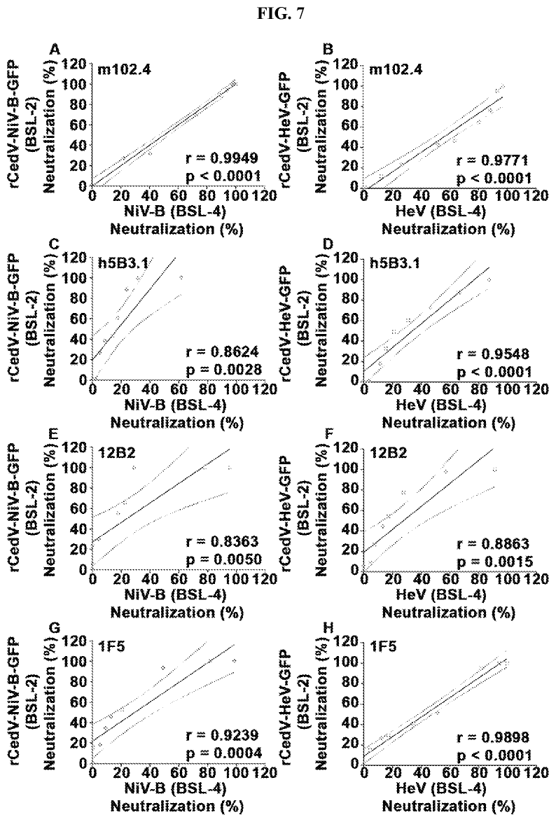

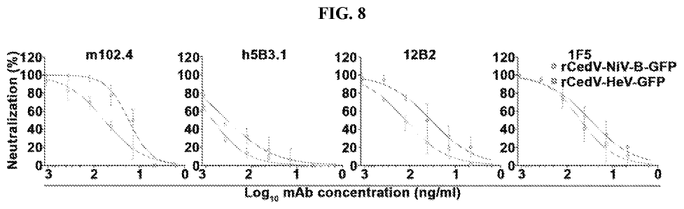

FIG. 8 show the results of the virus neutralization (FRNT) activities of a neutralizing cross-reactive mAb (m102.4) that targets the G glycoprotein of NiV and HeV, and three neutralizing cross-reactive mAbs (h5B3.1, 12B2 and 1F5) that target the F glycoprotein of NiV and HeV, against the GFP-encoding rCedV chimeras rCedV-NiV-B-GFP and rCedV-HeV-GFP.

In FIG. 8, mAbs serially diluted 3-fold to final concentrations ranging from 10 μg/ml to 0.0046 μg/ml and a target concentration of 2000 PFU (MOI. 0.05) of rCedV-NiV-B-GFP (left) or rCedV-HeV-GFP (right) were incubated for 2 hours at 37° C., 5% C02. Virus-mAb mixture was added to triplicate wells of black-walled 96-well plates containing pre-seeded Vero 76 cells. After 24 hours, plates were fixed with 4% formaldehyde in 1×PBS for 20 minutes. The fixed plates were scanned using the CTL S6 analyzer. Fluorescent foci were counted using the CTL Basic Count™ feature. Each mAb was tested in triplicate with 3-fold dilutions of each monoclonal antibody. The limit of detection for this assay was 50 fluorescent foci.

FIG. 8 shows seven point dose response curves for m102.4, h5B3.1, 12B2 and 1F5 monoclonal antibodies against rCedV-NiV-B-GFP and rCedV-HeV-GFP. Neutralization percent (%) was calculated based on fluorescent foci for each virus without mAb. The data represent mean±standard deviation from three independent experiments each performed in triplicate. Data are plotted as non-linear regression curve fit with variable slope using GraphPad prism and are representative of three independent experiments performed in triplicate. The limit of detection for this assay was 50 fluorescent foci. The gray circles and lines represent rCedV-NiV-B-GFP and light gray squares and lines represent rCedV-HeV-GFP.

The neutralization profiles of the mAbs by FRNT (Table 7) are remarkably comparable to the IC₅₀ values calculated by PRNT assays for both authentic NiV-B and HeV and rCedV-NiV-B-GFP and rCedV-HeV-GFP previously determined (shown in FIG. 6 and Table 5).

TABLE 7
IC₅₀ doses of HeV and NiV cross-reactive specific
monoclonal antibodies against rCedV-NiV-B-GFP
and rCedV-HeV-GFP infection in Vero 76 cells by
fluorescent reduction neutralization test (FRNT).

| | IC₅₀ (95% CI) (ng/ml) | |
|---|---|---|
| Monoclonal antibody (mAb) | rCedV-NiV-B-GFP | rCedV-HeV-GFP |
| m102.4 | 16.91 (14.72-19.45) | 58.12 (49.27-68.70) |
| h5B3.1 | 333.0 (255.5-439.9) | 700.2 (620.0-798.8) |
| 12B2 | 34.07 (24.88-46.48) | 124.5 (98.17-157.2) |
| 1F5 | 28.97 (22.86-36.65) | 50.16 (40.95-61.07) |

Note:
All IC₅₀ values are calculated by a nonlinear fit model from three independent experiments each performed in triplicate and are shown with 95% confidence intervals (95% CI).

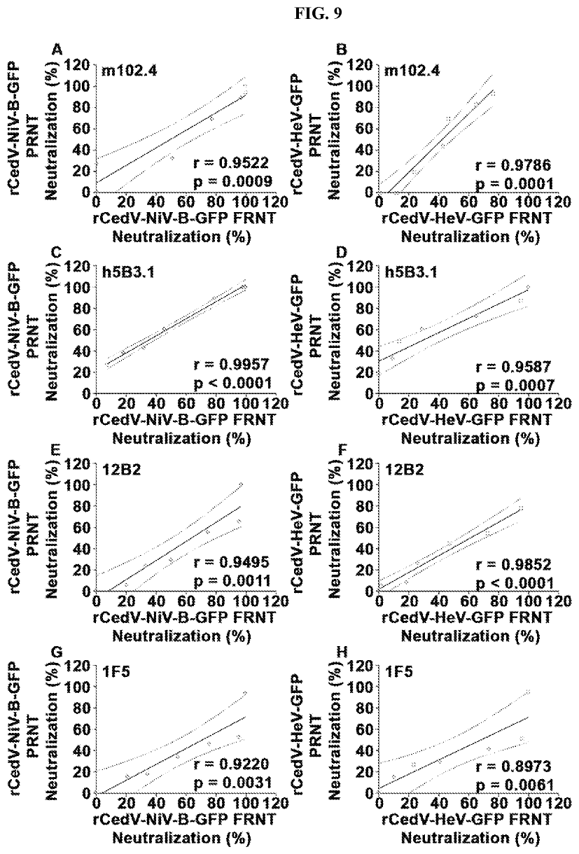

Correlation analysis of neutralization assays using the GFP expressing rCedV chimeric viruses. FIG. 9A-H shows the Pearson correlation analysis of neutralization (%) values from plaque reduction neutralization tests (PRNTs) (y-axes) (data from FIG. 6A) and fluorescence reduction neutralization tests (FRNTs) (x-axes) (data from FIG. 8) performed with rCedV-NiV-B-GFP (A, C, E, G) and with rCedV-HeV-GFP (B, D, F, H) with mAbs m102.4, h5B3.1, 12B2 or 1F5. The Pearson correlation coefficient ‘r’, p-value (two-tailed), linear regression line (solid lines) and 95% confidence intervals (dashed lines) are represented. Pearson's r≥0.8 and p value <0.05 indicate a strong significant positive correlation. Table 8: summarizes the correlation analysis of rCedV chimeric GFP viruses by PRNT versus FRNT.

TABLE 8
Correlation analysis of rCedV chimeric PRNT versus FRNT.

| | Monoclonal | Pearson's correlation | Coefficient of | Significance | 95% confidence |
|---|---|---|---|---|---|
| Virus | antibody (mAb) | coefficient (r) | determination (R²) | (p) | interval |
| rCedV-NiV-B-GFP | m102.4 | 0.9522 | 0.9067 | 0.0009 | 0.7038-0.9931 |
| vs NiV-B | h5B3.1 | 0.9957 | 0.9914 | <0.0001 | 0.9698-0.9994 |
| | 12B2 | 0.9495 | 0.9016 | 0.0011 | 0.6894-0.9927 |
| | 1F5 | 0.9220 | 0.8501 | 0.0031 | 0.5527-0.9886 |
| rCedV-HeV-GFP | m102.4 | 0.9786 | 0.9576 | 0.0001 | 0.8573-0.9970 |
| vs HeV | h5B3.1 | 0.9587 | 0.9191 | 0.0007 | 0.7397-0.9941 |
| | 12B2 | 0.9852 | 0.9707 | <0.0001 | 0.8997-0.9979 |
| | 1F5 | 0.8973 | 0.8051 | 0.0061 | 0.4448-0.9849 |

Note:
Correlation analysis was performed with the neutralization values from FIGS. 6A and 8.

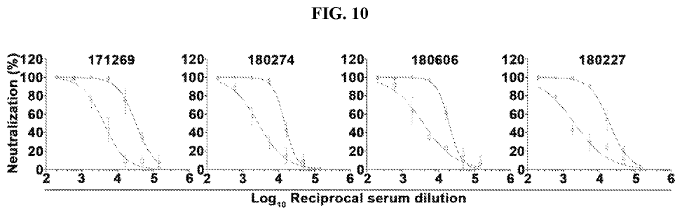

The GFP-encoding chimeras were then tested by FRNT using anti-NiV G glycoprotein polyclonal antisera. FIG. 10 shows the results of the virus neutralization (FRNT) activities of the sera from four Rhesus macaques that were immunized in a prime boost strategy at 3 weeks apart with a 0.2 mg/ml total of an equal mixture of NiV-B and NiV-M sG proteins AI 3+ Aluminum hydroxide suspension. On day 42, sera from 4 subjects was collected and stored at −80° C. Sera was tested for neutralization activity against the GFP-encoding rCedV chimeras rCedV-NiV-B-GFP and rCedV-HeV-GFP.

In FIG. 10, sera serially diluted 3-fold to final dilutions of 1:200 to 1:437, 400 and a final concentration of 2000 PFU (MOI: 0.05) of rCedV-NiV-B-GFP or rCedV-HeV-GFP were incubated for 2 hours at 37° C., 5% CO₂. Virus-sera mixture were added to triplicate wells of black-walled 96-well plates containing pre-seeded Vero 76 cells. After 24 hours, plates were fixed with 4% formaldehyde in 1×PBS for 20 minutes. The fixed plates were scanned using the CTL S6 analyzer. Fluorescent foci were counted using the CTL Basic Count™ feature. Each serum sample was tested in triplicate with 3-fold dilutions of each serum sample. The limit of detection for this assay was 50 fluorescent foci.

In FIG. 10, Neutralization percent (%) was calculated based on fluorescent foci for each virus without serum. The seven point dose response curves of sera collected on day 42 post immunization against rCedV-NiV-B-GFP and rCedV-HeV-GFP are shown. The data represent mean±standard deviation from two independent experiments each performed in triplicate. Data are plotted as non-linear regression curve fit with variable slope. The limit of detection for this assay was 50 fluorescent foci. Animal ID numbers are 171269, 180274, 180606 and 180227. Grey circles and lines represent rCedV-NiV-B-GFP and light grey squares and lines represent rCedV-HeV-GFP.

Here again, rCedV-NiV-B-GFP and rCedV-HeV-GFP performed well as surrogate virus assay systems for measuring the neutralization potency of all four subject sera samples. The sera cross-neutralized the viruses and the sera was more potent against the homologous surrogate virus (rCedV-NiV-B-GFP) because the sera was generated by NiV sG glycoprotein immunization. Table 9 summarizes the IC₅₀ titers of each of the sera samples.

TABLE 9
IC₅₀ doses of NiV-M and NiV-B sG immunized Rhesus macaque sera
against rCedV-NiV-B-GFP and rCedV-HeV-GFP infection in Vero
76 cells by fluorescent reduction neutralization test (FRNT).

| | IC₅₀ (95% CI) (Serum titer) | |
|---|---|---|
| Animal ID | rCedV-NiV-B-GFP | rCedV-HeV-GFP |
| 171269 | 1:32,147 (1:29,414-1:35,182) | 1:4,157 (1:3,711-1:4,658) |
| 180274 | 1:14,860 (1:14,018-1:15,761) | 1:2,704 (1:2,375-1:3,082) |
| 180606 | 1:19,480 (1:18,181-1:20,948) | 1:3,739 (1:3,094-1:4,542) |
| 180227 | 1:19,408 (1:17,817-1:21,158) | 1:2,048 (1:1,668-1:2,539) |

Note:
All IC₅₀ values are calculated by a nonlinear fit model from two independent experiments each performed in triplicate and are shown with 95% confidence intervals (95% CI).

Taken together, these results show this panel of rCedV chimeras can serve as a highly useful toolset for the study of otherwise highly pathogenic henipaviruses such as NiV and HeV that can be used outside of BSL-4 containment. The GFP-encoding chimeras can also be used in quantitative fluorescence based FRNT assays that can be conducted much faster than the standard PRNT assay. Based on the growth kinetics of all chimeras, the Luc-encoding chimeras and the non-reporter gene containing chimeras would be expected to perform similarly to the GFP-encoding chimeras. In addition, these data provide evidence that rCedV can be used to generate a chimera with any henipavirus G and F envelope glycoproteins or functional combinations of any G or F envelope glycoproteins of any other henipavirus, including GhV or MojV, which are both expected to be pathogenic viruses based on their genome sequences, and which is currently being constructed, or of any henipavirus species, strains or mutants.

### Example 5—Vaccine Platform

Another aspect of the present disclosure relates to vaccine compositions comprising rCedV chimeras for the treatment of, reduction of risk of, or prevention of infection by pathogenic henipaviruses.

Wild-type (non-chimeric) rCedV has been shown to be non-pathogenic in non-human primates (8 subjects infected with 5×10⁵ TCID₅₀ of rCedV split equally by intranasal and intratracheal administration) (Geisbert, T. W. and Broder, C. C., Unpublished), adding to the data in-hand that rCedV has been shown to be non-pathogenic in three species (ferrets, hamsters, and African green monkeys (AGMs)), all of which are acutely susceptible to HeV and NiV disease. Back-challenge of previously rCedV-infected AGMs with NiV-B (n=4) or HeV (n=4) resulted in death or severe disease showing that prior rCedV infection offers no cross-protection activity against pathogenic henipaviruses.

Two additional studies carried out in hamsters also showed that infection with rCedV provides no cross-protection against a NiV-B back-challenge. In contrast, initial infection of hamsters with the rCedV-NiV-B chimera described herein provided protection against NiV-B back-challenge (4/4 in one experiment), and initial infection with the rCedV-HeV chimera described herein provided protection against NiV-B back-challenge (8/8 in one experiment).

These data provide evidence that a rCedV chimera as described herein can be used to elicit a protective immune response against NiV-B by prior infection by administering to a subject a rCedV chimera expressing either the NiV-B or HeV G and F glycoproteins, and are indicative of vaccine platform success.

### Example 6—rCedV Chimera Expressing Soluble G (sG) Glycoproteins

rCedV chimeras have been developed that comprise the coding sequences for wild-type rCedV F and G glycoproteins, as well as sequences encoding soluble G (sG) from other henipaviruses, including HeV and NiV-B.

Shown in FIG. 11 is a schematic representation of the rCedV virus chimeric antigenome plasmids encoding soluble G glycoproteins (sG) of HeV or NiV-B either as sG or sGtet versions. (A) The sG of HeV and NiV with the Igκ-chain leader sequence (speckled) and the location on the N-terminal end of HeV and NiV-B sG ectodomain sequence are diagramed. (B) The sGtet of HeV and NiV with the Igκ-chain leader sequence (speckled), the GCNtet peptide domain (hatched), linker domains (striped), and the location on the N-terminal end of HeV and NiV-B sGtet ectodomain sequence are diagramed. (C) The antigenomes and lengths of the chimeras are schematically diagrammed as rCedV, rCedV-HeV sGtet and rCedV-NiV-B sGtet. T7ₘᵢₙ, T7 minimal promoter; T7ₒₚₜ, T7 optimal promoter; HHRbzA, Hammerhead Ribozyme A; 3′Le, 3′ Leader; 5′Tr, 5′ Trailer; HDVRbz, hepatitis delta virus ribozyme; T7t, T7 terminator.

The rCedV-HeV sG chimera virus was rescued using the virus rescue protocol and rCedV-HeV sG stocks were amplified and titered as in subsection “Rescue of rCedV Chimeras” in Example 1 above.

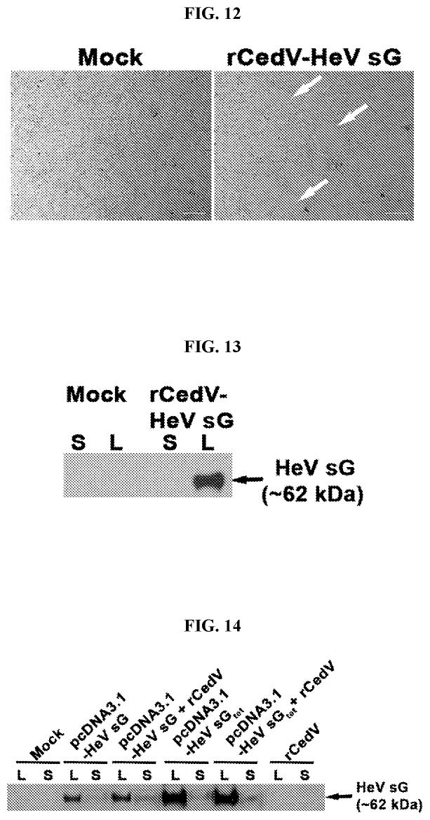

FIG. 12 shows syncytia induced by rCedV expressing HeV sG glycoprotein. Vero E6 cells were uninfected (Mock) or infected with rCedV-HeV sG at a multiplicity of infection (MOI) of 0.01. Images were taken 24 hours post infection (hpi). Images were captured with a Zeiss Axio Observer A1 inverted microscope using a 5× objective. Arrows indicate giant multinucleated cells (syncytia). Scale bar, 50 μm.

FIG. 13 shows western blot data demonstrating intracellular expression of the HeV sG glycoprotein in cells infected with rCedV-HeV sG. Vero E6 cells were uninfected (Mock) or infected with rCedV-HeV sG, at a multiplicity of infection (MOI) of 0.01. At 24 hrs post infection (hpi), supernatants and cells were collected. All cells were lysed with 1×RIPA (Radioimmunoprecipitation assay) buffer containing a protein inhibitor cocktail. Total protein (L) (30 μg) and 30 μl of supernatant (S) were separated on a 4-12% Bis-Tris gel and transferred to a nitrocellulose membrane. The membrane was blocked in 5% milk in 1×PBS with 0.1% Tween-20 at room temperature. Cross reactive murine monoclonal antibody (mAb), (48D3), specific to NiV and HeV G glycoproteins was used as the primary antibody and subsequently probed with a corresponding anti-mouse secondary HRP-coupled antibody. A band corresponding to HeV sG (˜62 kDa) was observed only in the lysates of infected cells. This data provides proof of concept that heterologous HeV sG glycoprotein (a vaccine antigen) can be expressed and produced from a HeV sG encoding rCedV, and this rCedV-HeV sG could serve as another version of a live-attenuated vaccine for HeV and NiV.

FIG. 14 shows rCedV does not interfere with expression of HeV sG glycoproteins as determined by western blot. The first generation of rCedV-HeV sG chimera did not appear to produce high levels of secreted sG while high levels of sG was detected in cell lysates. Here, duplicate wells of confluent Vero E6 cells were untreated or transfected with 1 μg of either pcDNA3.1-HeV sG or pcDNA3.1-HeV sGtet, a second generation sG that maintains a native-like tetrameric (tet) structure (4, 22). At 4 hrs post transfection, 1 set of cells was infected with rCedV at a multiplicity of infection (MOI) of 0.01 and the other set remained uninfected. At 24 hrs post infection (hpi), supernatants and cells were collected. All cells were lysed with 1×RIPA (Radioimmunoprecipitation assay) buffer containing a protein inhibitor cocktail. Total protein (L) (20 μl) and 20 μl of supernatant (S) were separated on a 4-12% Bis-Tris gel and transferred to a nitrocellulose membrane. The membrane was blocked in 5% milk in 1×PBS with 0.1% Tween-20 at room temperature then probed with HeV sG polyclonal rabbit serum followed by a corresponding anti-rabbit secondary HRP-coupled antibody. A band corresponding to HeV sG (˜62 kDa) was observed in the lysates (L) and supernatants (S) (boxes), of all samples except in the untreated and rCedV only infected cells. These data show that sG and newer versions of tetrameric sG (sGtet) can be expressed and secreted in the context of replicating rCedV, and second generation versions of rCedV-HeV sGtet or rCedV-NiV sGtet viruses could also serve as live-attenuated vaccines for HeV and NiV.

### Example 7—rCedV Chimera Expressing Fusion F or G Surface Glycoproteins

rCedV chimeras are created that express chimeric henipavirus F or G envelope glycoproteins, wherein the ectodomain and transmembrane domain of a non-CedV henipavirus (such as NiV, HeV, GhV and MojV) F or G is fused to the cytoplasmic tail domain of CedV F or G, or the ectodomain of a non-CedV henipavirus (such as NiV, HeV, GhV and MojV) F or G is fused to the transmembrane domain and cytoplasmic tail domain of CedV F or G.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims. The claims are intended to cover the components and steps in any sequence which is effective to meet the objectives there intended unless the context specifically indicates the contrary.

TABLE 9
CedV Genome and rCedV Genome incorporating modification that
add or remove restriction enzyme sites

| | SEQ | |
|---|---|---|
| | ID | |
| Virus | NO: | Sequence |
| CedV | 1 | ACCAGACAAAGGAAGTCTAGTCTCCGGATTAAATCATATTCGTATGATTAATCTTAGGAT |
| Isolate | | CCCGGTATCTAGAATCTGGATCTGGATTCGGTTTAATTGAATTGCGATCGTTTATAAATT |
| CG1a | | AGAAAGGAGATTTACTACTCAAAATGTCTGACATTTTCAATGAGACTCAATCATTTAGAA |
| Genbank: | | ACTATCAGTCCAACTTAGGCAGAGATGGCAGGGCCAGTGCAGCAACGACTACTTTGACAA |
| JQ001776.1 | | CTAAAGTGAGGATCTTTGTTCCAGCGAATAATAATCCAAACCTCAGATGGCGTTTAACAC |
| | | TATTCTTGATGGATGTCGTGAGGTCACCTGCCTCCGCAGAGTCTATGAAAGTGGGTGCTG |
| | | GGATATCCTTGGTATCTATGTATGCTGAAAAACCCGGGGCTCTTGTGAGAGCATTATTGA |
| | | ATGACCCAGATGTTGAAGCGATAATCATAGATGTTTATGGCTTTGATGAAGGTATTCCTA |
| | | TAATGGAACGAAGAGGTGATAAAGCTACAGATGACATGGATTCCCTAAGAAAGATTGTTA |
| | | AAGCTGCACATGATTTCAGCAGAGGAAGGAGTTTATTTGTTGATCAAAGGGTCCAGGATA |
| | | TTGTTATGTCAGATATGGGGTCATTTGTGAATGCTATTACTTCCATAGAGACGCAGATAT |
| | | GGATTTTGATCGCAAAGGCTGTAACTGCCCCAGATACAGCAGAAGAGAGCGAAGGAAGAA |
| | | GATGGGCAAAATATGTTCAGCAAAAGAGGGTTAATCCTTTGTTCTTGATTTCTCCACAAT |
| | | GGATCAATGACATGAGATCCCTGATTGCGGCAAGTCTTTCGCTTCGTAAATTCATGGTTG |
| | | AACTACTGATGGAAGCTAAGAAAGGACGGGGGACAAAAGGAAGAATAATGGAGATTGTAT |
| | | CCGATATCGGAAATTACGTTGAAGAGACAGGAATGGCAGGGTTCTTCGCTACAATAAAGT |
| | | TCGGTCTTGAGACCAAATTCCCTGCTTTGGCACTTAATGAGCTCCAGAGTGACTTGAACA |
| | | CAATGAAAAGTCTCATGATACTGTACAGAAGCATAGGACCAAAGGCCCCCTTTATGGTGT |
| | | TGTTGGAAGATTCAATTCAGACCAAATTTGCTCCAGGAAGCTATCCACTTCTTTGGAGTT |
| | | TTGCGATGGGTGTAGGCACAACTATTGACAGAGCTATGGGTGCCTTGAACATTAACAGAA |
| | | GTTATCTTGAACCTGTCTATTTTAGGCTAGGGCAACAATCAGCTAAACATCAAGCAGGAA |
| | | ATGTTGACAAAGAAATGGCAGAAAAGTTAGGATTGACAGAAGACCAGATCGTGCACCTAT |
| | | CAGCTAATGTGAAGGATGCAAGTCAAGGTAGAGATGACAATCAAATCAACATCCGAGAAG |
| | | GGAAGTTCACAAATGTTGTTGATGACATCCAGGATCATGCCCAGAGTTCCTCTGAGGATT |
| | | ACAATCCTAGTAAAAAGAGTTTCTCAATATTGACGAGCATCACATCCACCGTAGATAGTG |
| | | CTGACAGTAGGTCTGCAATGAATGAGTCAATGACAACAACATCCTTGCTGAAATTGAGAC |
| | | AGAGGCTGGCAGAGAAGAAAGGAGACTCCAAGAACAGTCAAGACACACCTCCAAAACCAC |
| | | CCAGAGCAAAAGATCAACCCACTGATGAGGTCTCCTTCATGGATTCCAATATATGATCAG |
| | | AATGATGGTTAAAATCAACCAACTAAGGGCGCGTAGAGTACCTTCAGATAGAACACTACA |
| | | TTAATCGGGTGAAACAATAGATTTATGGGTTTGGTGCTTAATTTTTATTTAATCTTACTT |
| | | GCAAAACAGGCAGCTGCTACACTCGTAACCACTCCTCACAGTAAGGGCAACACGGGTCAT |
| | | AGAACTTATGCCTATAGATTACCTCTATCTGTATATCTAGCTATGATTAAAATGTATACT |
| | | TCTGCTGACCGGTTTTCTAGCAACAGTCCACATTATTACTTTATGGGTATTTTTTAATCA |
| | | ACCTTTTATAATCAAATATATTACAAAAAACTTAGGATCCAAGTGGTCCAAACTTTTTTT |
| | | GATCAAGAGTCATATTGGCTACTTTAGGAGGACACTTTAAACACAAATTGTTACAAGAGG |
| | | ATATTCATCAGATGGACAAACTACAATTGATTGAAGATGGCCTCTCTACTATCAATTTTA |
| | | TACAGGAAAATAAGGAAAAATTACAGCATTCTTACGGAAGATCCTCCATCAGAGAGCCAC |
| | | CCACAAGTGTCAGGGTTGAAGAGTGGGAGAAATTTATTCGAAAGATCGCTTCTGGACCTG |
| | | AACAAGTTCAAGGGGGAGGATCTGAGACTGAGATCACAGGCGATAATGGAGATAGAGGCA |
| | | ATTTTACCAATCCTGATCAGGGAGGCGGAGTCACAGGACAATTCGAAGAAAGGTATCAAA |
| | | AATGGGGGTCACAAGATTCAGAATTACAACTGGACCCAATGGTTGTACACGATTTCTTCT |
| | | ATGACGAGAGAAGGGAGAATCCCGACAATGGAAAATATGACCGCAGCTCTAAAAAACGGG |
| | | ATAATATCAGAGAAGGAACACGACAGGATAAGTACAATAATCAGTCTACTGATGAATTAC |
| | | TGTCCTGCCTACAACCATCTTCTAAGAACGATGTCATCAAGAATGAAAGTACATCAGTGT |
| | | CAAATTTGCATGTTACAGGAAATAAACTGAATCCTGACGCAAAACCCTTTGAACCCACCT |
| | | CCCAGTCGAAAGAGCACCCAACCACCACACAGCACAACAAAAATGACCATCAGACCGATG |
| | | ATGATTATAAGAATAGAAGATCCAGTGAAAACAATGTGATCTCTGATCATGCCACCACAA |
| | | TGGAAGACAACAACAATTTTATCCCGGCGACCAAAAGAAAGAATGCATTGAGCGAACCCA |
| | | TATACGTCCAGGTATTGCCCTCAAACACAGAGGGTTTCTCGGGAAAAGATTATCCACTCC |
| | | TCAAGGACAACTCTGTCAAGAAGCGTGCAGAGCCAGTCATCCTAGAAACTGCCAACCACC |
| | | CTGCAGGCTCTGCCGACCAAGACACAAATCAGATTGAAGAAAACATGCAGTTCAACCTTC |
| | | CAAAACTGCTCACAGAAGATACAGACGATGAACCAGAGGATAACAATGATTCCATGCCTC |
| | | TTGAGGAAGACATTAGAGAGATCGGTTCCATGCTAAAAGATGGAACCAAAGATATCAAGA |
| | | CAAGGATGAATGAGATAGATGACGCAATCAAGAAGATAAATAAGAAATCAAAAAATAGAA |
| | | GTCTGGATCTAGAATCAGACGGTAAAGATCAGGGGAGAAGAGATCCATCAGTAGACCTCG |
| | | GGATTAAAAAAAGAAAGGAAGGGCTAAAGGCCGCAATGCAAAAGACAAAAGAGCAATTGT |
| | | CTATAAAAGTGGAGAGAGAGATTGGATTGAACGACAGGATATGTCAAAATTCGAAGATGA |
| | | GTACAGAAAAGAAATTGATATATGCTGGGATGGAAATGGAGTATGGACAAACGAGTACTG |
| | | GGTCAGGAGGTCCACAAGGATCAAAGGATGGGACTTCTGATGATGTCCAGGTAGACGAAG |
| | | ACTACGATGAAGGGGAAGACTATGAGGCTATGCCGTCAGATAGGTTTTATACAACATTAT |
| | | CAGGTGAACAAAAGGATAGATTTGATCTAGATGCTAACCAAATGTCTCAGTATGACCTCG |
| | | AGGCCCAGGTGGATGAATTAACCAGAATGAATCTCATACTCTATTCTAGATTAGAAACTA |
| | | CTAATAAGTTGCTTATTGACATATTAGATCTAGCTAAAGAAATGCCAAAGTTAGTTAGAA |
| | | AAGTGGATAATCTTGAGAGACAGATGGGTAACTTGAATATGTTAACCTCTACCCTTGAGG |
| | | GTCACCTATCTTCTGTAATGATTATGATACCCGGTAAGGATAAGAGCGAAAAGGAAATCC |
| | | CTAAAAATCCGGACCTGAGACCAATACTGGGGAGAAGCAACACGTCGTTAACTGATGTTA |
| | | TCGACCTAGACCATTACCCTGATAAAGGCTCCAAAGGTATCAAACCAAGTGGATCTGGAG |
| | | ACAGACAGTACATCGGCTCTCTAGAGAGCAAATTTTCTATAAATGATGAGTACAATTTTG |
| | | CTCCATACCCTATCAGGGACGAACTCCTATTGCCAGGTTTAAGAGATGACAAAACCAATG |
| | | CTTCATCGTTCATCCCAGATGACACGGACAGGTCTCCAATGGTGCTCAAAATAATAATTC |
| | | GACAGAACATCCATGATGAAGAAGTGAAGGATGAGCTACTGTCCATACTAGAACAACATA |
| | | ACACTGTGGAGGAATTGAATGAAATATGGAATACTGTGAATGATTACCTCGATGGCAACA |
| | | TCTGATTAACAGATATTGAGATTGATCCTATTCTAAACAAGTAATCTCTGATAATGATAG |
| | | TATGGAATAAGAATACTAATCACACTATTGTACTCTTGTAGAATCTTAACGAGTGTCTAA |
| | | TGTCAGATTTTAGCAACACATACTAATAACTTGTAATCCATTTCTCCTTATTCCATTTAA |
| | | TCTCACATTAGAAAAAACTTAGGATCCCAGATTTGCAAAGTCAAAACGGGATCTACTATC |
| | | AGGTGTTGGAGCTAACAATAGCGGAGTCTGCATAACAAATAGCGTTCAAAGAAGTTTGAA |
| | | AACCATCATAGAATATGGATCCGTCAGATTTGAGGAGGATTATAATGGAGGATGATAAGA |
| | | GTCTGGTCAACAATGATGATAGTACAGAAACTGATTTTCTCGAGAAAACTTGGAGAGAAG |
| | | GGAGTAAGATTGACAAGATCACACCAGAGGTTGATGAAAACGGGAATATGGTCCCCAAGT |
| | | ACGTTGTCTTCAACCCGGGGAAAAATGAGAGGAAAACATCCGGATATCAATATATGATTT |
| | | GTTATGGTTTCATTGAGGATGGACCTATCAATGGCTCACCAAGAGTCAAAGGTAATATCA |
| | | GAACCACCGCTTCTTTTCCTTTGGGTGTTGGAAAAACTTACTCGTCTCCAGAAGAGATCT |
| | | TACAAGAGCTGACAACACTCAAGATCACTGTCAGAAGGACAGCCGGATCAAATGAGAAGT |
| | | TGGTGTATGGAATAACAGGGCCTTTAAATCACCTTTACCCGTGGTATAAAGTTTTGACAG |
| | | GTGGCTCCATTTTTAGTGCGGTGAAGGTCTGTAGGAATGTGGATCAAATACTATTAGACA |
| | | GACCCCAAATACTTAGAGTATTCTTTCTAAGTATAACTAAATTAACAGATAAAGGTGTGT |
| | | ATATGATACCCAAAAGTGTTCTCGACTTCAGATCGGATAATTCGATGGCCTTCAATCTGC |
| | | TTGTGTATCTCAAGATAGACACTGACATCACCAAAGCAGGCATCAGAGGGATTGTCAACA |
| | | AAGAAGGGGAGAGGATAACGTCATTCATGTTACACATCGGTAACTTTACAAGAAGAGGAG |
| | | GAAAACATTACTCAGTGGAGTATTGCAAAAGGAAAATTGACAAAATGAAGCTCACATTCG |
| | | CCTTAGGCACTATAGGCGGTCTAAGCTTACATATCAGGATCGATGGAAGGATAAGTAAAA |
| | | GGCTCCAAGCACAAGTTGGCTTTCAGAGAAACATTTGCTACTCACTAATGGACACAAACC |
| | | CATGGTTGAATAAATTAACGTGGAACAATAGTTGTGAAATACACAAAGTCACCGCTGTCA |
| | | TTCAGCCATCTGTGCCAAAGGACTTCATGTTGTATGAGGACATCTTAATAGATAATACAG |
| | | GCAAGATCTTAAAATAAAGTAGGAGAGTCAGTCATTACCCAGTATATTGAATACTAATGA |
| | | CAACTTTATTAATCCAATTCTATCTCCAGTTACTAGAATTTCTAAAACAATTCTACTGCT |
| | | CAGCAACGCATCTCAAACATTGTGATCTTCAATTATGATCGACGCATTGTAATCTATATA |
| | | GCTTTTAGTTCATGAAATACTAAAAAGGGCTTAATCTTGTAAGTTCTCAGCAAATACTCC |
| | | AATGCAAAAGAGCGCCTCAACATCTCAAGCAGCACCAAAATAAACCACAATCAATGTGCA |
| | | ACAAGAGCAATCGTCTAAAGTGTGAAAACCAAAATCACAGATCAGAAAGGGCACATATTT |
| | | CAGTCCTGTAAAAATACCAAGTGGGATTAATAAAAGAGGATCAATCCTTATCATTTTAAG |
| | | AAAAACTTAGGATCCCAGAGATCCTAAAGAGCCAATTCCTTTATATTTTGATCTTGAAGG |
| | | GCTAGAAGTCAGGCTGAAACACAGAGGTGGAGGAACACAGGAACTAAAATTGATGAAATC |
| | | AACCTTAGCTCAACATCTAATCAATCAAGCTTAAGTCATCCTAATACTGTATACAACCAG |
| | | CAGCGTAGAGAGTGGATTTGATTTCGGCACCCTTGCGAAGTGAAGGCTATTACTGCCTGT |
| | | CCTTTCAATCAGAAAATTACATTTACCCATAAAGTAATCTCAACATGTCTAACAAGAGGA |
| | | CAACAGTATTGATCATAATAAGCTATACGTTATTTTATTTGAATAATGCAGCAATTGTAG |
| | | GGTTTGATTTTGATAAATTGAATAAAATAGGTGTGGTGCAAGGGAGAGTCCTAAATTATA |
| | | AAATTAAAGGAGATCCAATGACAAAAGACCTTGTCTTGAAATTTATCCCTAACATAGTGA |
| | | ATATCACTGAATGTGTGAGAGAGCCCTTGAGTAGGTACAATGAGACCGTGAGGAGATTGC |
| | | TTTTACCTATACACAACATGCTTGGGTTATACTTGAATAACACAAATGCTAAAATGACTG |
| | | GGTTGATGATCGCGGGTGTGATCATGGGTGGGATAGCAATAGGTATAGCCACAGCAGCTC |
| | | AGATCACAGCAGGTTTTGCTCTTTATGAGGCAAAAAAGAACACAGAAAATATTCAGAAAT |
| | | TAACAGACAGCATCATGAAAACACAGGACTCGATTGATAAACTTACTGACAGTGTGGGGA |
| | | CAAGCATACTTATATTGAATAAGCTACAGACATACATCAACAATCAACTGGTACCAAATC |
| | | TAGAGCTTCTATCCTGCCGACAAAACAAAATTGAGTTTGATCTAATGTTAACCAAGTATT |
| | | TGGTGGATCTTATGACTGTTATTGGTCCTAATATCAATAATCCTGTTAATAAAGATATGA |
| | | CTATTCAATCTTTGTCACTTCTTTTTGATGGCAATTATGATATAATGATGTCAGAACTTG |
| | | GTTATACACCTCAGGATTTCTTAGATTTGATAGAGAGTAAGAGTATAACAGGGCAAATAA |
| | | TTTATGTTGATATGGAAAACTTGTACGTTGTGATCAGGACATATCTACCTACCCTAATTG |
| | | AAGTACCTGATGCCCAAATATATGAGTTCAACAAAATAACTATGAGTAGCAATGGAGGAG |
| | | AATACTTGTCAACCATACCTAATTTCATATTAATAAGAGGTAATTATATGTCTAATATAG |
| | | ATGTTGCAACATGTTATATGACCAAAGCAAGCGTAATTTGTAATCAAGATTATTCACTCC |
| | | CGATGAGCCAAAACTTAAGAAGCTGTTATCAAGGTGAGACAGAATACTGTCCTGTTGAGG |
| | | CAGTCATCGCGTCACACTCTCCAAGATTTGCTCTTACAAATGGAGTTATTTTCGCCAATT |
| | | GTATAAATACAATTTGTAGGTGTCAAGACAATGGTAAGACTATCACTCAAAACATAAACC |
| | | AATTCGTAAGCATGATCGACAACAGTACTTGTAATGATGTCATGGTAGATAAGTTTACTA |
| | | TCAAGGTAGGAAAATATATGGGGAGAAAAGATATCAATAATATTAATATCCAGATAGGAC |
| | | CGCAGATCATAATTGATAAGGTTGACTTGTCTAATGAAATAAACAAGATGAATCAATCTT |
| | | TAAAAGATAGTATTTTCTACCTGAGAGAAGCCAAGAGAATTTTAGACTCAGTAAATATCA |
| | | GTCTTATATCTCCAAGCGTTCAATTGTTTCTAATAATAATATCAGTCCTCTCATTTATTA |
| | | TATTATTGATTATCATAGTATACTTGTACTGTAAATCAAAACATTCATATAAATATAACA |
| | | AATTTATAGATGATCCTGATTATTACAATGATTACAAAAGAGAACGTATTAATGGCAAAG |
| | | CCAGTAAGAGTAACAATATATATTATGTAGGTGATTAACAATCGATAATCTAAAGGATTA |
| | | CCTCACTATCACTACCAAGGTAACTTCCATGTAAGATCGGACCTTCCCCGAAGACATTAA |
| | | ATAAAACTTAGGATCCCAGAGTATCCCTCTAAGTGATCCTTCTAGATTGGTTACTGATAT |
| | | ATATACATATTTATCCTCTTTCCGTCGTTGTTTATTGATCATTAATAATGCTTTCTCAGC |
| | | TCCAAAAAAATTACTTAGACAACTCAAACCAACAAGGTGATAAAATGAACAACCCAGATA |
| | | AGAAATTAAGTGTCAACTTCAACCCTTTAGAATTAGATAAAGGTCAAAAAGATCTCAATA |
| | | AGTCTTATTATGTTAAAAACAAGAATTATAACGTTTCAAATCTATTAAATGAAAGTCTGC |
| | | ACGATATCAAGTTTTGTATTTATTGTATATTCTCACTGCTAATTATCATTACAATAATCA |
| | | ATATAATCACAATATCAATTGTTATAACTCGTCTGAAAGTACATGAAGAGAATAATGGCA |
| | | TGGAATCTCCTAATTTACAATCTATTCAAGATAGTCTCTCATCTCTTACTAACATGATCA |
| | | ATACAGAGATAACTCCTAGAATAGGGATTTTAGTTACAGCCACTTCTGTTACTCTCTCTT |
| | | CATCTATCAATTATGTCGGGACTAAGACAAATCAACTGGTCAATGAATTAAAAGATTATA |
| | | TAACCAAAAGTTGTGGCTTTAAGGTCCCTGAATTAAAGTTACATGAATGCAACATAAGTT |
| | | GTGCTGATCCAAAAATTAGCAAATCTGCAATGTACAGCACCAATGCCTATGCCGAGCTTG |
| | | CTGGTCCACCTAAGATATTTTGTAAAAGTGTATCCAAAGACCCCGACTTTAGACTGAAGC |
| | | AGATAGATTATGTAATACCAGTGCAGCAAGATCGGTCTATTTGTATGAACAACCCTTTAT |
| | | TGGATATTTCTGATGGGTTTTTTACCTACATACATTATGAAGGAATAAATAGCTGTAAAA |
| | | AATCAGATTCATTTAAAGTGCTGCTGTCACATGGTGAAATAGTTGACAGGGGTGATTATC |
| | | GACCATCATTATATCTATTATCAAGTCATTACCATCCTTATTCAATGCAGGTAATAAACT |
| | | GTGTACCTGTGACTTGTAACCAGTCATCCTTTGTATTCTGTCATATCTCCAACAACACTA |
| | | AAACATTGGACAATTCAGATTACTCGTCAGACGAGTACTACATAACATATTTCAATGGCA |
| | | TAGATCGTCCCAAAACCAAGAAGATTCCCATTAACAATATGACAGCAGACAATCGTTATA |
| | | TCCATTTTACATTCTCAGGTGGGGGAGGTGTATGTTTAGGTGAAGAATTTATTATTCCTG |
| | | TTACCACAGTCATCAATACTGATGTATTCACGCATGATTATTGTGAGAGTTTCAACTGTT |
| | | CAGTCCAAACCGGTAAAAGTCTAAAGGAGATATGCTCTGAGTCATTAAGATCTCCAACGA |
| | | ACTCATCGCGATACAATTTAAACGGAATCATGATTATAAGTCAAAACAACATGACAGATT |
| | | TTAAGATTCAGTTGAATGGTATAACTTATAACAAACTGTCATTCGGAAGTCCTGGAAGAC |
| | | TGAGCAAGACACTGGGCCAGGTCCTTTATTACCAATCTTCAATGAGTTGGGATACTTATC |
| | | TAAAGGCAGGATTTGTCGAGAAATGGAAACCCTTTACCCCGAATTGGATGAACAATACTG |
| | | TGATATCCAGACCTAACCAAGGTAATTGTCCAAGGTATCATAAATGCCCCGAGATATGTT |
| | | ATGGAGGGACATACAATGATATTGCTCCTTTAGATCTAGGAAAAGACATGTATGTTAGCG |
| | | TTATTCTAGATTCAGATCAGCTTGCAGAGAATCCAGAGATTACAGTATTTAACTCTACTA |
| | | CTATACTTTATAAGGAGAGAGTATCCAAAGATGAACTAAACACAAGAAGTACTACAACGA |
| | | GCTGTTTTCTTTTCCTAGATGAACCTTGGTGTATATCAGTATTAGAAACAAACAGATTTA |
| | | ACGGCAAATCTATTAGGCCCGAGATTTATTCATACAAAATTCCTAAGTATTGTTAATTTG |
| | | ATGAGCTTATTCCTCATACTTCAATCAAATTTAATATAACTAATATCAAATTGTTGCACT |
| | | CAGCTATTATTAAAACTGGATCATCAGACAATAAAGATGTATACAAAGATATATCGAAGA |
| | | GGGTATTAAAGAAAACTTAGGATCCCAGATCCTTCAATAAGGCAGAGCCTTGATTGTATC |
| | | AGCGTCATTTACAATTGAATCTCAATTAACAACACTGATTAATAACTTAAGCAGAATACT |
| | | CCTATTACAGTGTTTAATTGACTTAATTTTAATTGAGGATTTTATAATCCTATAATTGGA |
| | | GCAGATCTAAACTCTCACCGATTCAGTTCTAATCCTTTATTAACTAAAGAACAAATTCTA |
| | | AATAATTGGATGACGTCACAGGAGACAAGCTGGAAACAATTTAGTTAGAAGGAAGAAACC |
| | | TTTTACCAGATATGGAAAGTGACTTTGATATATCTGTTAGCGACGTACTGTACCCAGAAT |
| | | GTCATTTGGACAGTCCTATAGTCGGCGGTAAGCTCATTACTTCTCTTGAGTATGCGAATT |
| | | TGACTCATAACCAACCTCATGAAGATCAGACATTGCTGACTAATATAAATGTCAATAAAA |
| | | AGAAGAAGATAAAAAGTCCTCTAATATCCCAACAATCTTTATTTGGAAATGAGGTTAATA |
| | | AGGAGATTTTCGATCTTAAAAATTATTACCATGTCCCCTATCCAGAATGTAACAGAGATT |
| | | TATTCTTAATCTCTGATGACAAAATAGCATTCAAACTCAGTAAAATCATGGATAATTCTA |
| | | ATAAACTGTTTGATGGTTTAGAGAGGAAACTGAGTCGCTTAATTTCGAATGTAGATAATC |
| | | AACTATTAAATGCAACCTCTCTTCATAATAATTCTGAGATGGATCGGAAGGGAAAAGAAC |
| | | ATCCTTGCTTCCCAGAAAAGAGCACAATTGATGATGTAAGACAGCAGAGACAGACACGAG |
| | | ATTTTCCAAAGAATTCAACTAGAGAGGGAAGATCTCCAAAACACCCTGATGCCGGTCCTA |
| | | CACCTGAAAACAGTGCCAAAAACGATTTGCATAGAGACAACACAGACAATATGCCAACAG |
| | | GCCATAGTTCGACATCTATGAAAAAACCTAAAATATCTGGAGAAGAATATCTTAGTATGT |
| | | GGCTAGACTCAGAGGATTTGGGTTCTAAACGAATTTCTGCACAATTAGGGAAGGATGTAT |
| | | CATGTAAAGGCCATCTGCACACGACAGAAGACAAACCGATAATAGTTCCTGACACTCGAT |
| | | ATATCCAAAATCATGAATCTAATAACGATATTTTCCCCAAAAAAGAGAAAAAATTCTGCA |
| | | AACTTCCACCGTCATCGGATAATTTAACCAAAATCATGGTGAATTCAAAATGGTACAATC |
| | | CTTTCCTTTTTTGGTTTACTGTCAAGACTGAACTTAGAGCCTGCCAGAAGGAGAACTACA |
| | | AAAGGAAAAACAGAAAATTGGGAATTATCACATCGATTAAAGGTTCATGCTATAAGTTGA |
| | | TACTCAACCAGAATCTAGTAGCAATATTCGAGGAAGACAGCAGTGGATACTCAGATCATA |
| | | AAAAAAGAAAAAAACGATGCTACTATCTAACTCCCGAAATGGTCCTTATGTTCTCCGATG |
| | | TAACTGAAGGAAGATTGATGATTGATGTTGCAATGAGATTTGACAAAAAGTACAAAACTC |
| | | TAGAGAAAAAGGCTTTGAAATTATGGTTTCTTATAGACGAGTTATTTCCTTCTATGGGAA |
| | | ATAGAGTGTATAATATTATATCCATGCTTGAGCCTTTGACTCTCGCGATATTACAGGTTA |
| | | AGGATGAGTCAAGGTTGTTGAGAGGTGCATTCATGCATCATTGTTTAGGTGACCTCTTCG |
| | | AAGAACTTCGAGAGTCCAAGAACTACCCGGAAGATGAGATCAAGAGATTTGCCAACGACC |
| | | TAATAAATGTCATGACCTGTCGGGACATTCATTTAGTAGCAGAATTCTTCTCATTCTTTA |
| | | GGACTTTCGGACATCCAATATTGAACGCTCAAACTGCAGCCAGGAAAGTTAGAGAGTACA |
| | | TGTTAGCAGATAAAATCCTTGAGTACGAACCTATCATGAAAGGTCATGCGATTTTCTGTG |
| | | CTATAATCATAAATGGATTTAGAGATAGACATGGAGGAGTTTGGCCTCCTCTTGATCTTC |
| | | CAAAACATTGTTCAAAGAACATAATATCTCTCAAAAATACAGGTGAAGGGGTAACTTATG |
| | | AAGTAGCAATAAACAATTGGAGATCATTTGTCGGGTTAAAGTTCAAATGTTTTATGGGTC |
| | | TCAATTTAGACAATGATCTCAGCATGTACATGAAAGATAAAGCATTATCACCTTTAAGGG |
| | | ATCTTTGGGATTCAATCTATTCACGTGAAGTAATGTCCTACCAACCACCTAGAAACAAAA |
| | | AATCAAGAAGATTGGTTGAGGTTTTCGTTGATGATCAGGACTTTGATCCCGTTGATATGA |
| | | TAAATTATGTTCTGACCGGAGAATATCTCAGAGATGATGATTTCAATGCTTCTTATAGTT |
| | | TAAAAGAGAAAGAGACCAAACAAGTTGGCAGGTTGTTTGCTAAGATGACTTATAAAATGA |
| | | GGGCCTGTCAAGTTATTGCTGAGAATTTAATTGCACATGGGATTGGGAGATATTTCCATG |
| | | AAAACGGGATGGTTAAGGATGAGCATGAGCTCAGCAAATCACTGTTTCAATTGTCTATAT |
| | | CAGGAATACCAAGAGGGAACAAAAACAACAAATCGACGAACGACACAATCCACGAAAGCA |
| | | AGATCGAGAATAACCATTCCTTTAAAAACATCCAGAATCGATCATTTCGAAAGACGGATA |
| | | ACCCATACAATAGATTTAACATTGATAACCCAACTTTCTTATCCCCAAACTGTAACCCCA |
| | | AGTATAACCGTAAGAATTCAGAGACAATAGGTATATTCTCTCGTGCAGAAACCAAAAGCA |
| | | TGATTAGAGAACAGAAAAGTCACAGAGAAGTCAAAATAAATAAGCTAGATATCGGCAGTG |
| | | ATAATGAAGAGCAAGGAAAAGAGATAGATGCCGCCAAGTACAAAATCACGGACAACCCAA |
| | | ATCCACACATAAATCCTCAAGATCAACCCGGAATCTGTCAAGAAGACAAAGGCAAAGAAG |
| | | GAGCAAAGTCAGATCTCACAGAAGGCATGAGTTTTCTGGAGATGCACACACTCTTTAACC |
| | | CGAGTAAGAGCGATATCAGAACAAATCTCGAATTGGAAAAGAGTTCACTTTCAAACCCTG |
| | | GATTTATATCACAAAAAGAGAAAAGAGGCAAAACTTATAATGAATCCCATTCACTGGGAA |
| | | AGTTCTCTAAAGAGGATGAAGAAAGATACGATGTCATCAGTGCATTCCTGACAACAGATT |
| | | TACGAAAATTCTGCTTAAATTGGAGACATGAATCAATCGGCATTTTTGCAAGAAGGATGG |
| | | ACGAAATCTATGGTTTGCCTGGTTTCTTTAATTGGATGCACAGAAGACTAGAGCGATCTG |
| | | TGTTATATGTTGCGGACCCTCATTGCCCGCCGTCTATCAATGAACATATCGATCTAAACG |
| | | ATTCACCCGAAAGAGACATATTTATACATCATCCGAAAGGGGGTATAGAAGGATACAGCC |
| | | AAAAACTGTGGACAATAGCGACTATCCCTTTTCTATTCCTCAGTGCTCATGAGACAAACA |
| | | CCCGGATAGCGGCAGTTGTACAAGGTGACAATCAATCAATTGCAATTACACATAAGGTCC |
| | | ACCCTCATTTGCCTTACAAAATGAAGAAAGAACTCTCTGCAATGCAGGCAAAAAAATATT |
| | | TTTCAAGGTTACGGCACAACATGAAGGCATTAGGGCATGAATTGAAGGCGACCGAGACTA |
| | | TCATTAGTACTCATTTCTTCATTTATTCCAAGAAAATCCACTATGACGGGGCTGTTTTAT |
| | | CACAATCTCTGAAATCAATGGCAAGGTGTGTATTTTGGTCAGAAACCCTTGTTGATGAAA |
| | | CTAGAGCAGCATGCAGTAATATCAGCACAACAATTGCAAAGGCTATTGAGAATGGTTATA |
| | | GCAGGAGATCTGGCTATCTGATAAATGTTCTTAAAACCATCCAACAAATTAATATATCAT |
| | | TGAGTTTTAATATAAATGAATGCATGACAGATGACATAATCAGACCGTTTAGAGATAATC |
| | | CAAACTGGATCAAACATGCCGCATTAATCCCCGCCAGCTTGGGAGGACTCAACTATATGA |
| | | ACATGTCTCGATTGTATGTGAGGAATATAGGGGATCCAGTCACAGCATCGATAGCAGATG |
| | | TTAAGAGAATGATTCTCGGTGGTGTACTACCCATTGGAATACTCCACAATATCATGTTGC |
| | | AAGAACCCGGTGATGCCACTTATTTGGACTGGTGTAGTGATCCATACTCCATCAACCTAA |
| | | AGCAGACTCAAAGTATCACAAAAGTTATAAAGAACATAACGGCAAGAGTGATACTAAGGA |
| | | ATTCGGTCAATCCACTGCTCAAAGGTCTATTTCATGAAGGTGCTTATGAGGAGGACACTG |
| | | AATTAGCAACATTCATTTTGGACAGGAGAGTCATCTTACCACGAGTCGGTCACGAGATCT |
| | | TAAACAACTCCATCACAGGAGCAAGAGAAGAGATCTCGGGCTTACTGGATACCACAAAAG |
| | | GATTGATAAGAATTGGCATAGCAAAGGGAGGATTAACTCAGAGAACATTATCTCGAATTT |
| | | CCAATTATGATTATGAACAATTTTTGAACCTAATGAATATGTTGAAGAACAAAGAACAAA |
| | | ACAGTGTCATTTCCCTGTCAGCTTGCTCTGTTGACTTTGCTATAGCTTTAAGAAGCAGGA |
| | | TGTGGAGGAAATTGGCAAAAGGAAGATTAATATATGGTTTAGAAGTCCCTGATCCAATAG |
| | | AAGCAATGATTGGCTTTCTCATTCTTGGGAGTGAAAATTGTCTACTCTGTGATTCAGGAA |
| | | GCAAAAACTATACCTGGTTTTTCATACCAAAGGATGTACAGTTGGATAAGATTGATAAAG |
| | | ATCACGCATCAATAAGGGTACCCTATGTCGGATCAACTACCGAAGAAAGATCAGAGATAA |
| | | AGTTAGGATCCGTGAAAAATCCAAGCAAATCCCTGAAATCTGCTATAAGACTCGCAACTG |
| | | TGTACACTTGGGCATTTGGCACAAGTGATGCTGAATGGTGGGAGGCTTGGTACTTGTCTA |
| | | ATCAACGAGCAAATATACCCTTAGATGTTCTCAAAACGATAACACCTATATCTACTTCAA |
| | | CGAATATTGCTCATAGATTACGAGACCGATCAACACAGGTTAAATACGCCAGTACATCTC |
| | | TTAACAGAGTATCGCGGCATGTAACAATTAGTAACGATAACATGAATTTTGAATTTGACG |
| | | GGGTTAAAATGGATACCAACTTGATTTATCAACAAGTCATGCTGTTAGGGCTTTCATGCT |
| | | TGGAGAGTTTATTCCGAAATAGGAAAATGACAAATAGTTACAATATCGTGTACCATTTAC |
| | | ACGTTCAAGAACATTGTTGTGTAAAGGCTCTGAATGATTTACCTTATACACCGTCAACAC |
| | | ATCCAGTGCCAAATTATACAGAAGTTAGAGATAATAGGTTAATTTACGATCCTCAACCTA |
| | | TATTAGAATTTGATGAGCTAAGATTAGCAATTCAGCAAACAAAGAAAGTAGATTTGGAAT |
| | | TTTCATTGTGGGATACAAAAGAACTTCATGAGAATTTAGCTCAAAGTTTAGCGATTACAG |
| | | TAACGGATATTATGACAAAATCTGATAAAGATCATATTAAAGACCAAAGAAGTATAGATG |
| | | TTGATGATAATATTAAGACACTAATAACTGAGTTTTTATTAGTAGACCCTGAAATGTTTG |
| | | CCGTAAATTTAGGATTGCATATATCAATAAAATGGTCATTTGATATTCACTTTAAAAGAC |
| | | CAAGAGGACGCTATAGCATGATAGAATACTTGACTGATCTTTTGGATAATACTTCTTCTC |
| | | ATGTTTATCGAATCCTTACTAATGTATTATCTCATCCCAGAGTTATGAGAAAATTCACTA |
| | | ATGCCGGGCTACTAGTACCGAAATACGGTCCCTACCTTACAAGTCAAGATTTCAAAAAGA |
| | | TGGCGGTAGATTTCATAATAACAGCGTATACCACATTTTTGACCAATTGGTGTAATAATA |
| | | ACAAGTTTTCAATTCTAATACCTGAACAAGACCCTGATATACTTGAATTAAGAAAAGACA |
| | | TCACTCATGCAAGGCATTTATGTATGATCTCGGATCTTTACTGCTACTCTTTCAAGCAAC |
| | | CTTGGATAAAGGAGCTTACACCACAAGAGAAGATCTGCGTCATGGAGGACTTCATAGCCA |
| | | ATTGTGTTGCTAATGATCAAACAAGTGCGGGCTGGAACATAACGCCCTTAAGAGTTTACA |
| | | ATCTCCCTGCATCGACCACATACATCAGGAGAGGGATAATAAAACAATTAAGAATCCGTC |
| | | AAAGCAATGAGCCTATTGATCTGGAAGATATTAGGATTGGTCAGAACCCCGATTTTGTGA |
| | | ATAAACCTATTGAGTTTTGTAGCAGTGAATTCGGTATCACAATTTATAACCTTGAAGAAA |
| | | TTCTTCAATCAAATGTGCATCTCAGTGTAAATATGAACATTGACTCCTCAACAAGTAACA |
| | | ATACTGAAAATCATTTATTTAGAAGGGTAGGCTTGAACTCTACTTCATCTTATAAAGCAC |
| | | TATCTTTAACACCTGTTATTAAAAGATATCATCAACAGAACACTAATAGGCTGTTTATAG |
| | | GAGAAGGATCAGGGTCTATGATGTATCTTTACCAGAAAACCTTGGGGGAGACAATATGCT |
| | | TCTTTAATTCGGGAGTTCAGTACAATGAGGATCTGGGTCAAAGGGAACAATCATTATACC |
| | | CGAGTGAATACAGTATCTGTGAACAAGGAGTAAAAAAAGAAAACCCTCTCACCGGGCATG |
| | | TTATACCACTATTCAATGGAAGACCAGAAACCACATGGGTAGGCAATGATGATTCTTTCA |
| | | AGTATATATTGGAACATACTATAAATAGAGACATCGGGCTTGTTCACTCCGATATGGAAA |
| | | CAGGAATAGGGAAGGATAATTATACTATCTTAAATGAACATGCACATCTTATAGCACTGA |
| | | GCCTTACAGTAATGATTGATGATGGAATCTTGGTGTCTAAGGTAGCTTATGCCCCTGGGT |
| | | TTTGCATCTCTTCATTATTGAATATGTACCGGACATTTTTTTCATTAGTTCTATGTGCGT |
| | | TTCCACCGTATAGCAATTTTGAATCAACTGAATTTTACCTGATTTGCTTGCAAAAAAGTA |
| | | TACCCGGACCTATCACACCAGCTAGAGCCATCCAACAAACGACGAAGCAATCTAGAGAAG |
| | | AGGATAATAGTATAACTAATAATATCCTCAAAATCAAAAATCTTGTTCAGAAAGAATTTA |
| | | TCAAAACAGTAAAGAAAAAATACGAAATCCATCCTTCGTTTAACTGTCCTATCAACTTCA |
| | | CAAAGGATGATAAATATTTAATGAGTGTTGGGTTTCAAGCCAATGGTCCTGATATGATAC |
| | | GTAAAGAGACGGGCTATGACATAGGTAGCAATGTAGAGAATCTCCGAGATGTCTTAATCA |
| | | AGTTGTTTGCAGATGCAGTCACCTTCTATGATGATGTCACAAATAAAAAGAACTTTTTAA |
| | | ATCCTTATCCAGTCTACACAAGAACTCAGTATAAAATTCTGATGGATAAAATATGCAAGA |
| | | AAGTCACCTTATACACCTTAATCATATCATGTAAAGGATCCAATCAATATTGCTGGGAAA |
| | | TTAAATCCCAAATAAGAAAGCATTGTCTCATACTTGATTTGAAAAGTAAGGTTTTTACAA |
| | | AACTTATTCCAAAGGGATTAAGAGAAAGGGGTGACTCAAAAGGGATGAAGAGCATATGGT |
| | | TCACTAAACTAACCAGTCAAGAGGTGAAAAGATGGTGGAAGATGATATCTTACATCGTGA |
| | | TAATAAGCAATCCATAACCACATCCAACTTGTCAGTTAAACACTTAAATCACAATAAACT |
| | | TGTCATCAGATTAAAGAAAACTTATAATTCCCTTTTTTAGGT |
| rCedV | 2 | ACCAGAAAAAGGAAGTCTAGTCTCCGGATTAAATCATATTCGTATGATTAATCTTAGGAT |
| Derived | | CCCGGTATCTAGAATCTGGATCTGGATTCGGTTTAATTGAATTGCGATCGTTTATAAATT |
| from isolate | | AGAAAGGAGATTTACTACTCAAAATGTCTGACATTTTCAATGAGACTCAATCATTTAGAA |
| CG1a | | ACTATCAGTCCAACTTAGGCAGAGATGGCAGGGCCAGTGCAGCAACGACTACTTTGACAA |
| Genbank: | | CTAAAGTGAGGATCTTTGTTCCAGCGAATAATAATCCAAACCTCAGATGGCGTTTAACAC |
| JQ001776.1 | | TATTCTTGATGGATGTCGTGAGGTCACCTGCCTCCGCAGAGTCTATGAAAGTGGGTGCTG |
| Mods. | | GGATATCCTTGGTATCTATGTATGCTGAAAAACCAGGGGCTCTTGTGAGAGCATTATTGA |
| shown in | | ATGACCCAGATGTTGAAGCGATAATCATAGATGTTTATGGCTTTGATGAAGGTATTCCTA |
| bold & | | TAATGGAACGAAGAGGTGATAAAGCTACAGATGACATGGATTCCCTAAGAAAGATTGTTA |
| underlined | | AAGCTGCACATGATTTCAGCAGAGGAAGGAGTTTATTTGTTGATCAAAGGGTCCAGGATA |
| F and G | | TTGTTATGTCAGATATGGGGTCATTTGTGAATGCTATTACTTCCATAGAGACGCAGATAT |
| coding | | GGATTTTGATCGCAAAGGCTGTAACTGCCCCAGATACAGCAGAAGAGAGCGAAGGAAGAA |
| regions | | GATGGGCAAAATATGTTCAGCAAAAGAGGGTTAATCCTTTGTTCTTGATTTCTCCACAAT |
| underlined | | GGATCAATGACATGAGATCCCTGATTGCGGCAAGTCTTTCGCTTCGTAAATTCATGGTTG |
| | | AACTACTGATGGAAGCTAAGAAAGGACGGGGGACAAAAGGAAGAATAATGGAGATTGTAT |
| | | CCGATATCGGAAATTACGTTGAAGAGACAGGAATGGCAGGGTTCTTCGCTACAATAAAGT |
| | | TCGGTCTTGAGACCAAATTCCCTGCTTTGGCACTTAATGAGCTCCAGAGTGACTTGAACA |
| | | CAATGAAAAGTCTCATGATACTGTACAGAAGCATAGGACCAAAGGCCCCCTTTATGGTGT |
| | | TGTTGGAAGATTCAATTCAGACCAAATTTGCTCCAGGAAGCTATCCACTTCTTTGGAGTT |
| | | TTGCGATGGGTGTAGGCACAACTATTGACAGAGCTATGGGTGCCTTGAACATTAACAGAA |
| | | GTTATCTTGAACCTGTCTATTTTAGGCTAGGGCAACAATCAGCTAAACATCAAGCAGGAA |
| | | ATGTTGACAAAGAAATGGCAGAAAAGTTAGGATTGACAGAAGACCAGATCGTGCACCTAT |
| | | CAGCTAATGTGAAGGATGCAAGTCAAGGTAGAGATGACAATCAAATCAACATCCGAGAAG |
| | | GGAAGTTCACAAATGTTGTTGATGACATCCAGGATCATGCCCAGAGTTCCTCTGAGGATT |
| | | ACAATCCTAGTAAAAAGAGTTTCTCAATATTGACGAGCATCACATCCACCGTAGATAGTG |
| | | CTGACAGTAGGTCTGCAATGAATGAGTCAATGACAACAACATCCTTGCTGAAATTGAGAC |
| | | AGAGGCTGGCAGAGAAGAAAGGAGACTCCAAGAACAGTCAAGACACACCTCCAAAACCAC |
| | | CCAGAGCAAAAGATCAACCCACTGATGAGGTCTCCTTCATGGATTCCAATATATGATCAG |
| | | AATGATGGTTAAAATCAACCAACTAAGGGCGCGTAGAGTACCTTCAGATAGAACACTACA |
| | | TTAATCGGGTGAAACAATAGATTTATGGGTTTGGTGCTTAATTTTTATTTAATCTTACTT |
| | | GCAAAACAGGCAGCTGCTACACTCGTAACCACTCCTCACAGTAAGGGCAACACGGGTCAT |
| | | AGAACTTATGCCTATAGATTACCTCTATCTGTATATCTAGCTATGATTAAAATGTATACT |
| | | TCTGCTGACCGGTTTTCTAGCAACAGTCCACATTATTACTTTATGGGTATTTTTTAATCA |
| | | ACCTTTTATAATCAAATATATTACAAAAAACTTAGGATCCAAGTGGTCCAAACTTTTTTT |
| | | GATCAAGAGTCATATTGGCTACTTTAGGAGGACACTTTAAACACAAATTGTTACAAGAGG |
| | | ATATTCATCAGATGGACAAACTACAATTGATTGAAGATGGCCTCTCTACTATCAATTTTA |
| | | TACAGGAAAATAAGGAAAAATTACAGCATTCTTACGGAAGATCCTCCATCAGAGAGCCAC |
| | | CCACAAGTGTCAGGGTTGAAGAGTGGGAGAAATTTATTCGAAAGATCGCTTCTGGACCTG |
| | | AACAAGTTCAAGGGGGAGGATCTGAGACTGAGATCACAGGCGATAATGGAGATAGAGGCA |
| | | ATTTTACCAATCCTGATCAGGGAGGCGGAGTCACAGGACAATTCGAAGAAAGGTATCAAA |
| | | AATGGGGGTCACAAGATTCAGAATTACAACTGGACCCAATGGTTGTACACGATTTCTTCT |
| | | ATGACGAGAGAAGGGAGAATCCCGACAATGGAAAATATGACCGCAGCTCTAAAAAACGGG |
| | | ATAATATCAGAGAAGGAACACGACAGGATAAGTACAATAATCAGTCTACTGATGAATTAC |
| | | TGTCCTGCCTACAACCATCTTCTAAGAACGATGTCATCAAGAATGAAAGTACATCAGTGT |
| | | CAAATTTGCATGTTACAGGAAATAAACTGAATCCTGACGCAAAACCCTTTGAACCCACCT |
| | | CCCAGTCGAAAGAGCACCCAACCACCACACAGCACAACAAAAATGACCATCAGACCGATG |
| | | ATGATTATAAGAATAGAAGATCCAGTGAAAACAATGTGATCTCTGATCATGCCACCACAA |
| | | TGGAAGACAACAACAATTTTATCCCGGCGACCAAAAGAAAGAATGCATTGAGCGAACCCA |
| | | TATACGTCCAGGTATTGCCCTCAAACACAGAGGGTTTCTCGGGAAAAGATTATCCACTCC |
| | | TCAAGGACAACTCTGTCAAGAAGCGTGCAGAGCCAGTCATCCTAGAAACTGCCAACCACC |
| | | CTGCAGGCTCTGCCGACCAAGACACAAATCAGATTGAAGAAAACATGCAGTTCAACCTTC |
| | | CAAAACTGCTCACAGAAGATACAGACGATGAACCAGAGGATAACAATGATTCCATGCCTC |
| | | TTGAGGAAGACATTAGAGAGATCGGTTCCATGCTAAAAGATGGAACCAAAGATATCAAGA |
| | | CAAGGATGAATGAGATAGATGACGCAATCAAGAAGATAAATAAGAAATCAAAAAATAGAA |
| | | GTCTGGATCTAGAATCAGACGGTAAAGATCAGGGGAGAAGAGATCCATCAGTAGACCTCG |
| | | GGATTAAAAAAAGAAAGGAAGGGCTAAAGGCCGCAATGCAAAAGACAAAAGAGCAATTGT |
| | | CTATAAAAGTGGAGAGAGAGATTGGATTGAACGACAGGATATGTCAAAATTCGAAGATGA |
| | | GTACAGAAAAGAAATTGATATATGCTGGGATGGAAATGGAGTATGGACAAACGAGTACTG |
| | | GGTCAGGAGGTCCACAAGGATCAAAGGATGGGACTTCTGATGATGTCCAGGTAGACGAAG |
| | | ACTACGATGAAGGGGAAGACTATGAGGCTATGCCGTCAGATAGGTTTTATACAACATTAT |
| | | CAGGTGAACAAAAGGATAGATTTGATCTAGATGCTAACCAAATGTCTCAGTATGACCTCG |
| | | AGGCCCAGGTGGATGAATTAACCAGAATGAATCTCATACTCTATTCTAGATTAGAAACTA |
| | | CTAATAAGTTGCTTATTGACATATTAGATCTAGCTAAAGAAATGCCAAAGTTAGTTAGAA |
| | | AAGTGGATAATCTTGAGAGACAGATGGGTAACTTGAATATGTTAACCTCTACCCTTGAGG |
| | | GTCACCTATCTTCTGTAATGATTATGATACCCGGTAAGGATAAGAGCGAAAAGGAAATCC |
| | | CTAAAAATCCGGACCTGAGACCAATACTGGGGAGAAGCAACACGTCGTTAACTGATGTTA |
| | | TCGACCTAGACCATTACCCTGATAAAGGCTCCAAAGGTATCAAACCAAGTGGATCTGGAG |
| | | ACAGACAGTACATCGGCTCTCTAGAGAGCAAATTTTCTATAAATGATGAGTACAATTTTG |
| | | CTCCATACCCTATCAGGGACGAACTCCTATTGCCAGGTTTAAGAGATGACAAAACCAATG |
| | | CTTCATCGTTCATCCCAGATGACACGGACAGGTCTCCAATGGTGCTCAAAATAATAATTC |
| | | GACAGAACATCCATGATGAAGAAGTGAAGGATGAGCTACTGTCCATACTAGAACAACATA |
| | | ACACTGTGGAGGAATTGAATGAAATATGGAATACTGTGAATGATTACCTCGATGGCAACA |
| | | TCTGATTAACAGATATTGAGATTGATCCTATTCTAAACAAGTAATCTCTGATAATGATAG |
| | | TATGGAATAAGAATACTAATCACACTATTGTACTCTTGTAGAATCTTAACGAGTGTCTAA |
| | | TGTCAGATTTTAGCAACACATACTAATAACTTGTAATCCATTTCTCCTTATTCCATTTAA |
| | | TCTCACATTAGAAAAAACTTAGGATCCCAGACGCGTATTTGCAAAGTCAAAACGGGATCT |
| | | ACTATCAGGTGTTGGAGCTAACAATAGCGGAGTCTGCATAACAAATAGCGTTCAAAGAAG |
| | | TTTGAAAACCATCATAGAATATGGATCCGTCAGATTTGAGGAGGATTATAATGGAGGATG |
| | | ATAAGAGTCTGGTCAACAATGATGATAGTACAGAAACTGATTTTCTCGAGAAAACTTGGA |
| | | GAGAAGGGAGTAAGATTGACAAGATCACACCAGAGGTTGATGAAAACGGGAATATGGTCC |
| | | CCAAGTACGTTGTCTTCAACCCAGGGAAAAATGAGAGGAAAACATCCGGATATCAATATA |
| | | TGATTTGTTATGGTTTCATTGAGGATGGACCTATCAATGGCTCACCAAGAGTCAAAGGTA |
| | | ATATCAGAACCACCGCTTCTTTTCCTTTGGGTGTTGGAAAAACTTACTCGTCTCCAGAAG |
| | | AGATCTTACAAGAGCTGACAACACTCAAGATCACTGTCAGAAGGACAGCCGGATCAAATG |
| | | AGAAGTTGGTGTATGGAATAACAGGGCCTTTAAATCACCTTTACCCGTGGTATAAAGTTT |
| | | TGACAGGTGGCTCCATTTTTAGTGCGGTGAAGGTCTGTAGGAATGTGGATCAAATACTAT |
| | | TAGACAGACCCCAAATACTTAGAGTATTCTTTCTAAGTATAACTAAATTAACAGATAAAG |
| | | GTGTGTATATGATACCCAAAAGTGTTCTCGACTTCAGATCGGATAATTCGATGGCCTTCA |
| | | ATCTGCTTGTGTATCTCAAGATAGACACTGACATCACCAAAGCAGGCATCAGAGGGATTG |
| | | TCAACAAAGAAGGGGAGAGGATAACGTCATTCATGTTACACATCGGTAACTTTACAAGAA |
| | | GAGGAGGAAAACATTACTCAGTGGAGTATTGCAAAAGGAAAATTGACAAAATGAAGCTCA |
| | | CATTCGCCTTAGGCACTATAGGCGGTCTAAGCTTACATATCAGGATCGATGGAAGGATAA |
| | | GTAAAAGGCTCCAAGCACAAGTTGGCTTTCAGAGAAACATTTGCTACTCACTAATGGACA |
| | | CAAACCCATGGTTGAATAAATTAACGTGGAACAATAGTTGTGAAATACACAAAGTCACCG |
| | | CTGTCATTCAGCCATCTGTGCCAAAGGACTTCATGTTGTATGAGGACATCTTAATAGATA |
| | | ATACAGGCAAGATCTTAAAATAAAGTAGGAGAGTCAGTCATTACCCAGTATATTGAATAC |
| | | TAATGACAACTTTATTAATCCAATTCTATCTCCAGTTACTAGAATTTCTAAAACAATTCT |
| | | ACTGCTCAGCAACGCATCTCAAACATTGTGATCTTCAATTATGATCGACGCATTGTAATC |
| | | TATATAGCTTTTAGTTCATGAAATACTAAAAAGGGCTTAATCTTGTAAGTTCTCAGCAAA |
| | | TACTCCAATGCAAAAGAGCGCCTCAACATCTCAAGCAGCACCAAAATAAACCACAATCAA |
| | | TGTGCAACAAGAGCAATCGTCTAAAGTGTGAAAACCAAAATCACAGATCAGAAAGGGCAC |
| | | ATATTTCAGTCCTGTAAAAATACCAAGTGGGATTAATAAAAGAGGATCAATCCTTATCAT |
| | | TTTAAGAAAAACTTAGGATCCCAGAGATCCTAAAGAGCCAATTCCTTTATATTTTGATCT |
| | | TGAAGGGCTAGAAGTCAGGCTGAAACACAGAGGTGGAGGAACACAGGAACTAAAATTGAT |
| | | GAAATCAACCTTAGCTCAACATCTAATCAATCAAGCTTAAGTCATCCTAATACTGTATAC |
| | | AACCAGCAGCGTAGAGAGTGGATTTGATTTCGGCACCCTTGCGAAGTGAAGGCTATTACT |
| | | GCCTGTCCTTTCAATCAGAAAATTACATTTACCCATAAAGTAATCTCAACATGTCTAACA |
| | | AGAGGACAACAGTATTGATCATAATAAGCTATACGTTATTTTATTTGAATAATGCAGCAA |
| | | TTGTAGGGTTTGATTTTGATAAATTGAATAAAATAGGTGTGGTGCAAGGGAGAGTCCTAA |
| | | ATTATAAAATTAAAGGAGATCCAATGACAAAAGACCTTGTCTTGAAATTTATCCCTAACA |
| | | TAGTGAATATCACTGAATGTGTGAGAGAGCCCTTGAGTAGGTACAATGAGACCGTGAGGA |
| | | GATTGCTTTTACCTATACACAACATGCTTGGGTTATACTTGAATAACACAAATGCTAAAA |
| | | TGACTGGGTTGATGATCGCGGGTGTGATCATGGGTGGGATAGCAATAGGTATAGCCACAG |
| | | CAGCTCAGATCACAGCAGGTTTTGCTCTTTATGAGGCAAAAAAGAACACAGAAAATATTC |
| | | AGAAATTAACAGACAGCATCATGAAAACACAGGACTCGATTGATAAACTTACTGACAGTG |
| | | TGGGGACAAGCATACTTATATTGAATAAGCTACAGACATACATCAACAATCAACTGGTAC |
| | | CAAATCTAGAGCTTCTATCCTGCCGACAAAACAAAATTGAGTTTGATCTAATGTTAACCA |
| | | AGTATTTGGTGGATCTTATGACTGTTATTGGTCCTAATATCAATAATCCTGTTAATAAAG |
| | | ATATGACTATTCAATCTTTGTCACTTCTTTTTGATGGCAATTATGATATAATGATGTCAG |
| | | AACTTGGTTATACACCTCAGGATTTCTTAGATTTGATAGAGAGTAAGAGTATAACAGGGC |
| | | AAATAATTTATGTTGATATGGAAAACTTGTACGTTGTGATCAGGACATATCTACCTACCC |
| | | TAATTGAAGTACCTGATGCCCAAATATATGAGTTCAACAAAATAACTATGAGTAGCAATG |
| | | GAGGAGAATACTTGTCAACCATACCTAATTTCATATTAATAAGAGGTAATTATATGTCTA |
| | | ATATAGATGTTGCAACATGTTATATGACCAAAGCAAGCGTAATTTGTAATCAAGATTATT |
| | | CACTCCCGATGAGCCAAAACTTAAGAAGCTGTTATCAAGGTGAGACAGAATACTGTCCTG |
| | | TTGAGGCAGTCATCGCGTCACACTCTCCAAGATTTGCTCTTACAAATGGAGTTATTTTCG |
| | | CCAATTGTATAAATACAATTTGTAGGTGTCAAGACAATGGTAAGACTATCACTCAAAACA |
| | | TAAACCAATTCGTAAGCATGATCGACAACAGTACTTGTAATGATGTCATGGTAGATAAGT |
| | | TTACTATCAAGGTAGGAAAATATATGGGGAGAAAAGATATCAATAATATTAATATCCAGA |
| | | TAGGACCGCAGATCATAATTGATAAGGTTGACTTGTCTAATGAAATAAACAAGATGAATC |
| | | AATCTTTAAAAGATAGTATTTTCTACCTGAGAGAAGCCAAGAGAATTTTAGACTCAGTAA |
| | | ATATCAGTCTTATATCTCCAAGCGTTCAATTGTTTCTAATAATAATATCAGTCCTCTCAT |
| | | TTATTATATTATTGATTATCATAGTATACTTGTACTGTAAATCAAAACATTCATATAAAT |
| | | ATAACAAATTTATAGATGATCCTGATTATTACAATGATTACAAAAGAGAACGTATTAATG |
| | | GCAAAGCCAGTAAGAGTAACAATATATATTATGTAGGTGATTAACAATCGATAATCTAAA |
| | | GGATTACCTCACTATCACTACCAAGGTAACTTCCATGTAAGATCGGACCTTCCCCGAAGA |
| | | CATTAAATAAAACTTAGGATCCCAGAGTATCCCTCTAAGTGATCCTTCTAGATTGGTTAC |
| | | TGATATATATACATATTTATCCTCTTTCCGTCGTTGTTTATTGATCATTAATAATGCTTT |
| | | CTCAGCTCCAAAAAAATTACTTAGACAACTCAAACCAACAAGGTGATAAAATGAACAACC |
| | | CAGATAAGAAATTAAGTGTCAACTTCAACCCTTTAGAATTAGATAAAGGTCAAAAAGATC |
| | | TCAATAAGTCTTATTATGTTAAAAACAAGAATTATAACGTTTCAAATCTATTAAATGAAA |
| | | GTCTGCACGATATCAAGTTTTGTATTTATTGTATATTCTCACTGCTAATTATCATTACAA |
| | | TAATCAATATAATCACAATATCAATTGTTATAACTCGTCTGAAAGTACATGAAGAGAATA |
| | | ATGGCATGGAATCTCCTAATTTACAATCTATTCAAGATAGTCTCTCATCTCTTACTAACA |
| | | TGATCAATACAGAGATAACTCCTAGAATAGGGATTTTAGTTACAGCCACTTCTGTTACTC |
| | | TCTCTTCATCTATCAATTATGTCGGGACTAAGACAAATCAACTGGTCAATGAATTAAAAG |
| | | ATTATATAACCAAAAGTTGTGGCTTTAAGGTCCCTGAATTAAAGTTACATGAATGCAACA |
| | | TAAGTTGTGCTGATCCAAAAATTAGCAAATCTGCAATGTACAGCACCAATGCCTATGCCG |
| | | AGCTTGCTGGTCCACCTAAGATATTTTGTAAAAGTGTATCCAAAGACCCCGACTTTAGAC |
| | | TGAAGCAGATAGATTATGTAATACCAGTGCAGCAAGATCGGTCTATTTGTATGAACAACC |
| | | CTTTATTGGATATTTCTGATGGGTTTTTTACCTACATACATTATGAAGGAATAAATAGCT |
| | | GTAAAAAATCAGATTCATTTAAAGTGCTGCTGTCACATGGTGAAATAGTTGACAGGGGTG |
| | | ATTATCGACCATCATTATATCTATTATCAAGTCATTACCATCCTTATTCAATGCAGGTAA |
| | | TAAACTGTGTACCTGTGACTTGTAACCAGTCATCCTTTGTATTCTGTCATATCTCCAACA |
| | | ACACTAAAACATTGGACAATTCAGATTACTCGTCAGACGAGTACTACATAACATATTTCA |
| | | ATGGCATAGATCGTCCCAAAACCAAGAAGATTCCCATTAACAATATGACAGCAGACAATC |
| | | GTTATATCCATTTTACATTCTCAGGTGGGGGAGGTGTATGTTTAGGTGAAGAATTTATTA |
| | | TTCCTGTTACCACAGTCATCAATACTGATGTATTCACGCATGATTATTGTGAGAGTTTCA |
| | | ACTGTTCAGTCCAAACCGGTAAAAGTCTAAAGGAGATATGCTCTGAGTCATTAAGATCTC |
| | | CAACGAACTCATCGCGATACAATTTAAACGGAATCATGATTATAAGTCAAAACAACATGA |
| | | CAGATTTTAAGATTCAGTTGAATGGTATAACTTATAACAAACTGTCATTCGGAAGTCCTG |
| | | GAAGACTGAGCAAGACACTGGGCCAGGTCCTTTATTACCAATCTTCAATGAGTTGGGATA |
| | | CTTATCTAAAGGCAGGATTTGTCGAGAAATGGAAACCCTTTACCCCGAATTGGATGAACA |
| | | ATACTGTGATATCCAGACCTAACCAAGGTAATTGTCCAAGGTATCATAAATGCCCCGAGA |
| | | TATGTTATGGAGGGACATACAATGATATTGCTCCTTTAGATCTAGGAAAAGACATGTATG |
| | | TTAGCGTTATTCTAGATTCAGATCAGCTTGCAGAGAATCCAGAGATTACAGTATTTAACT |
| | | CTACTACTATACTTTATAAGGAGAGAGTATCCAAAGATGAACTAAACACAAGAAGTACTA |
| | | CAACGAGCTGTTTTCTTTTCCTAGATGAACCTTGGTGTATATCAGTATTAGAAACAAACA |
| | | GATTTAACGGCAAATCTATTAGGCCCGAGATTTATTCATACAAAATTCCTAAGTATTGTT |
| | | AATTTGATGAGCTTATTCCTCATACTTCAATCAAATTTAATATAACTAATATCAAATTGT |
| | | TGCACTCAGCTATTATTAAAACTGGATCATCAGACAATAAAGATGTATACAAAGATATAT |
| | | CGAAGAGGGTATTAAAGAAAACTTAGGATCCCAGATCCTTCAATAAGGCAGAGCCTTGAT |
| | | TGTATCAGCGTCATTTACAATTGAATCTCAATTAACAACACTGATTAATAACTTAAGCAG |
| | | AATACTCCTATTACAGTGTTTAATTGACTTAATTTTAATTGAGGATTTTATAATCCTATA |
| | | ATTGGAGCAGATCTAAACTCTCACCGATTCAGTTCTAATCCTTTATTAACTAAAGAACAA |
| | | ATTCTAAATAATTGGATGACGTCACAGGAGACAAGCTGGAAACAATTTAGTTAGAAGGAA |
| | | GAAACCTTTTACCAGATATGGAAAGTGACTTTGATATATCTGTTAGCGACGTACTGTACC |
| | | CAGAATGTCATTTGGACAGTCCTATAGTCGGCGGTAAGCTCATTACTTCTCTTGAGTATG |
| | | CGAATTTGACTCATAACCAACCTCATGAAGATCAGACATTGCTGACTAATATAAATGTCA |
| | | ATAAAAAGAAGAAGATAAAAAGTCCTCTAATATCCCAACAATCTTTATTTGGAAATGAGG |
| | | TTAATAAGGAGATTTTCGATCTTAAAAATTATTACCATGTCCCCTATCCAGAATGTAACA |
| | | GAGATTTATTCTTAATCTCTGATGACAAAATAGCATTCAAACTCAGTAAAATCATGGATA |
| | | ATTCTAATAAACTGTTTGATGGTTTAGAGAGGAAACTGAGTCGCTTAATTTCGAATGTAG |
| | | ATAATCAACTATTAAATGCAACCTCTCTTCATAATAATTCTGAGATGGATCGGAAGGGAA |
| | | AAGAACATCCTTGCTTCCCAGAAAAGAGCACAATTGATGATGTAAGACAGCAGAGACAGA |
| | | CACGAGATTTTCCAAAGAATTCAACTAGAGAGGGAAGATCTCCAAAACACCCTGATGCCG |
| | | GTCCTACACCTGAAAACAGTGCCAAAAACGATTTGCATAGAGACAACACAGACAATATGC |
| | | CAACAGGCCATAGTTCGACATCTATGAAAAAACCTAAAATATCTGGAGAAGAATATCTTA |
| | | GTATGTGGCTAGACTCAGAGGATTTGGGTTCTAAACGAATTTCTGCACAATTAGGGAAGG |
| | | ATGTATCATGTAAAGGCCATCTGCACACGACAGAAGACAAACCGATAATAGTTCCTGACA |
| | | CTCGATATATCCAAAATCATGAATCTAATAACGATATTTTCCCCAAAAAAGAGAAAAAAT |
| | | TCTGCAAACTTCCACCGTCATCGGATAATTTAACCAAAATCATGGTGAATTCAAAATGGT |
| | | ACAATCCTTTCCTTTTTTGGTTTACTGTCAAGACTGAACTTAGAGCCTGCCAGAAGGAGA |
| | | ACTACAAAAGGAAAAACAGAAAATTGGGAATTATCACATCGATTAAAGGTTCATGCTATA |
| | | AGTTGATACTCAACCAGAATCTAGTAGCAATATTCGAGGAAGACAGCAGTGGATACTCAG |
| | | ATCATAAAAAAAGAAAAAAACGATGCTACTATCTAACTCCCGAAATGGTCCTTATGTTCT |
| | | CCGATGTAACTGAAGGAAGATTGATGATTGATGTTGCAATGAGATTTGACAAAAAGTACA |
| | | AAACTCTAGAGAAAAAGGCTTTGAAATTATGGTTTCTTATAGACGAGTTATTTCCTTCTA |
| | | TGGGAAATAGAGTGTATAATATTATATCCATGCTTGAGCCTTTGACTCTCGCGATATTAC |
| | | AGGTTAAGGATGAGTCAAGGTTGTTGAGAGGTGCATTCATGCATCATTGTTTAGGTGACC |
| | | TCTTCGAAGAACTTCGAGAGTCCAAGAACTACCCGGAAGATGAGATCAAGAGATTTGCCA |
| | | ACGACCTAATAAATGTCATGACCTGTCGGGACATTCATTTAGTAGCAGAATTCTTCTCAT |
| | | TCTTTAGGACTTTCGGACATCCAATATTGAACGCTCAAACTGCAGCCAGGAAAGTTAGAG |
| | | AGTACATGTTAGCAGATAAAATCCTTGAGTACGAACCTATCATGAAAGGTCATGCGATTT |
| | | TCTGTGCTATAATCATAAATGGATTTAGAGATAGACATGGAGGAGTTTGGCCTCCTCTTG |
| | | ATCTTCCAAAACATTGTTCAAAGAACATAATATCTCTCAAAAATACAGGTGAAGGGGTAA |
| | | CTTATGAAGTAGCAATAAACAATTGGAGATCATTTGTCGGGTTAAAGTTCAAATGTTTTA |
| | | TGGGTCTCAATTTAGACAATGATCTCAGCATGTACATGAAAGATAAAGCATTATCACCTT |
| | | TAAGGGATCTTTGGGATTCAATCTATTCACGTGAAGTAATGTCCTACCAACCACCTAGAA |
| | | ACAAAAAATCAAGAAGATTGGTTGAGGTTTTCGTTGATGATCAGGACTTTGATCCCGTTG |
| | | ATATGATAAATTATGTTCTGACCGGAGAATATCTCAGAGATGATGATTTCAATGCTTCTT |
| | | ATAGTTTAAAAGAGAAAGAGACCAAACAAGTTGGCAGGTTGTTTGCTAAGATGACTTATA |
| | | AAATGAGGGCCTGTCAAGTTATTGCTGAGAATTTAATTGCACATGGGATTGGGAGATATT |
| | | TCCATGAAAACGGGATGGTTAAGGATGAGCATGAGCTCAGCAAATCACTGTTTCAATTGT |
| | | CTATATCAGGAATACCAAGAGGGAACAAAAACAACAAATCGACGAACGACACAATCCACG |
| | | AAAGCAAGATCGAGAATAACCATTCCTTTAAAAACATCCAGAATCGATCATTTCGAAAGA |
| | | CGGATAACCCATACAATAGATTTAACATTGATAACCCAACTTTCTTATCCCCAAACTGTA |
| | | ACCCCAAGTATAACCGTAAGAATTCAGAGACAATAGGTATATTCTCTCGTGCAGAAACCA |
| | | AAAGCATGATTAGAGAACAGAAAAGTCACAGAGAAGTCAAAATAAATAAGCTAGATATCG |
| | | GCAGTGATAATGAAGAGCAAGGAAAAGAGATAGATGCCGCCAAGTACAAAATCACGGACA |
| | | ACCCAAATCCACACATAAATCCTCAAGATCAACCCGGAATCTGTCAAGAAGACAAAGGCA |
| | | AAGAAGGAGCAAAGTCAGATCTCACAGAAGGCATGAGTTTTCTGGAGATGCACACACTCT |
| | | TTAACCCGAGTAAGAGCGATATCAGAACAAATCTCGAATTGGAAAAGAGTTCACTTTCAA |
| | | ACCCTGGATTTATATCACAAAAAGAGAAAAGAGGCAAAACTTATAATGAATCCCATTCAC |
| | | TGGGAAAGTTCTCTAAAGAGGATGAAGAAAGATACGATGTCATCAGTGCATTCCTGACAA |
| | | CAGATTTACGAAAATTCTGCTTAAATTGGAGACATGAATCAATCGGCATTTTTGCAAGAA |
| | | GGATGGACGAAATCTATGGTTTGCCTGGTTTCTTTAATTGGATGCACAGAAGACTAGAGC |
| | | GATCTGTGTTATATGTTGCGGACCCTCATTGCCCGCCGTCTATCAATGAACATATCGATC |
| | | TAAACGATTCACCCGAAAGAGACATATTTATACATCATCCGAAAGGGGGTATAGAAGGAT |
| | | ACAGCCAAAAACTGTGGACAATAGCGACTATCCCTTTTCTATTCCTCAGTGCTCATGAGA |
| | | CAAACACCCGGATAGCGGCAGTTGTACAAGGTGACAATCAATCAATTGCAATTACACATA |
| | | AGGTCCACCCTCATTTGCCTTACAAAATGAAGAAAGAACTCTCTGCAATGCAGGCAAAAA |
| | | AATATTTTTCAAGGTTACGGCACAACATGAAGGCATTAGGGCATGAATTGAAGGCGACCG |
| | | AGACTATCATTAGTACTCATTTCTTCATTTATTCCAAGAAAATCCACTATGACGGGGCTG |
| | | TTTTATCACAATCTCTGAAATCAATGGCAAGGTGTGTATTTTGGTCAGAAACCCTTGTTG |
| | | ATGAAACTAGAGCAGCATGCAGTAATATCAGCACAACAATTGCAAAGGCTATTGAGAATG |
| | | GTTATAGCAGGAGATCTGGCTATCTGATAAATGTTCTTAAAACCATCCAACAAATTAATA |
| | | TATCATTGAGTTTTAATATAAATGAATGCATGACAGATGACATAATCAGACCGTTTAGAG |
| | | ATAATCCAAACTGGATCAAACATGCCGCATTAATCCCCGCCAGCTTGGGAGGACTCAACT |
| | | ATATGAACATGTCTCGATTGTATGTGAGGAATATAGGGGATCCAGTCACAGCATCGATAG |
| | | CAGATGTTAAGAGAATGATTCTCGGTGGTGTACTACCCATTGGAATACTCCACAATATCA |
| | | TGTTGCAAGAACCCGGTGATGCCACTTATTTGGACTGGTGTAGTGATCCATACTCCATCA |
| | | ACCTAAAGCAGACTCAAAGTATCACAAAAGTTATAAAGAACATAACGGCAAGAGTGATAC |
| | | TAAGGAATTCGGTCAATCCACTGCTCAAAGGTCTATTTCATGAAGGTGCTTATGAGGAGG |
| | | ACACTGAATTAGCAACATTCATTTTGGACAGGAGAGTCATCTTACCACGAGTCGGTCACG |
| | | AGATCTTAAACAACTCCATCACAGGAGCAAGAGAAGAGATCTCGGGCTTACTGGATACCA |
| | | CAAAAGGATTGATAAGAATTGGCATAGCAAAGGGAGGATTAACTCAGAGAACATTATCTC |
| | | GAATTTCCAATTATGATTATGAACAATTTTTGAACCTAATGAATATGTTGAAGAACAAAG |
| | | AACAAAACAGTGTCATTTCCCTGTCAGCTTGCTCTGTTGACTTTGCTATAGCTTTAAGAA |
| | | GCAGGATGTGGAGGAAATTGGCAAAAGGAAGATTAATATATGGTTTAGAAGTCCCTGATC |
| | | CAATAGAAGCAATGATTGGCTTTCTCATTCTTGGGAGTGAAAATTGTCTACTCTGTGATT |
| | | CAGGAAGCAAAAACTATACCTGGTTTTTCATACCAAAGGATGTACAGTTGGATAAGATTG |
| | | ATAAAGATCACGCATCAATAAGGGTACCCTATGTCGGATCAACTACCGAAGAAAGATCAG |
| | | AGATAAAGTTAGGATCCGTGAAAAATCCAAGCAAATCCCTGAAATCTGCTATAAGACTCG |
| | | CAACTGTGTACACTTGGGCATTTGGCACAAGTGATGCTGAATGGTGGGAGGCTTGGTACT |
| | | TGTCTAATCAACGAGCAAATATACCCTTAGATGTTCTCAAAACGATAACACCTATATCTA |
| | | CTTCAACGAATATTGCTCATAGATTACGAGACCGATCAACACAGGTTAAATACGCCAGTA |
| | | CATCTCTTAACAGAGTATCGCGGCATGTAACAATTAGTAACGATAACATGAATTTTGAAT |
| | | TTGACGGGGTTAAAATGGATACCAACTTGATTTATCAACAAGTCATGCTGTTAGGGCTTT |
| | | CATGCTTGGAGAGTTTATTCCGAAATAGGAAAATGACAAATAGTTACAATATCGTGTACC |
| | | ATTTACACGTTCAAGAACATTGTTGTGTAAAGGCTCTGAATGATTTACCTTATACACCGT |
| | | CAACACATCCAGTGCCAAATTATACAGAAGTTAGAGATAATAGGTTAATTTACGATCCTC |
| | | AACCTATATTAGAATTTGATGAGCTAAGATTAGCAATTCAGCAAACAAAGAAAGTAGATT |
| | | TGGAATTTTCATTGTGGGATACAAAAGAACTTCATGAGAATTTAGCTCAAAGTTTAGCGA |
| | | TTACAGTAACGGATATTATGACAAAATCTGATAAAGATCATATTAAAGACCAAAGAAGTA |
| | | TAGATGTTGATGATAATATTAAGACACTAATAACTGAGTTTTTATTAGTAGACCCTGAAA |
| | | TGTTTGCCGTAAATTTAGGATTGCATATATCAATAAAATGGTCATTTGATATTCACTTTA |
| | | AAAGACCAAGAGGACGCTATAGCATGATAGAATACTTGACTGATCTTTTGGATAATACTT |
| | | CTTCTCATGTTTATCGAATCCTTACTAATGTATTATCTCATCCCAGAGTTATGAGAAAAT |
| | | TCACTAATGCCGGGCTACTAGTACCGAAATACGGTCCCTACCTTACAAGTCAAGATTTCA |
| | | AAAAGATGGCGGTAGATTTCATAATAACAGCGTATACCACATTTTTGACCAATTGGTGTA |
| | | ATAATAACAAGTTTTCAATTCTAATACCTGAACAAGACCCTGATATACTTGAATTAAGAA |
| | | AAGACATCACTCATGCAAGGCATTTATGTATGATCTCGGATCTTTACTGCTACTCTTTCA |
| | | AGCAACCTTGGATAAAGGAGCTTACACCACAAGAGAAGATCTGCGTCATGGAGGACTTCA |
| | | TAGCCAATTGTGTTGCTAATGATCAAACAAGTGCGGGCTGGAACATAACGCCCTTAAGAG |
| | | TTTACAATCTCCCTGCATCGACCACATACATCAGGAGAGGGATAATAAAACAATTAAGAA |
| | | TCCGTCAAAGCAATGAGCCTATTGATCTGGAAGATATTAGGATTGGTCAGAACCCCGATT |
| | | TTGTGAATAAACCTATTGAGTTTTGTAGCAGTGAATTCGGTATCACAATTTATAACCTTG |
| | | AAGAAATTCTTCAATCAAATGTGCATCTCAGTGTAAATATGAACATTGACTCCTCAACAA |
| | | GTAACAATACTGAAAATCATTTATTTAGAAGGGTAGGCTTGAACTCTACTTCATCTTATA |
| | | AAGCACTATCTTTAACACCTGTTATTAAAAGATATCATCAACAGAACACTAATAGGCTGT |
| | | TTATAGGAGAAGGATCAGGGTCTATGATGTATCTTTACCAGAAAACCTTGGGGGAGACAA |
| | | TATGCTTCTTTAATTCGGGAGTTCAGTACAATGAGGATCTGGGTCAAAGGGAACAATCAT |
| | | TATACCCGAGTGAATACAGTATCTGTGAACAAGGAGTAAAAAAAGAAAACCCTCTCACCG |
| | | GGCATGTTATACCACTATTCAATGGAAGACCAGAAACCACATGGGTAGGCAATGATGATT |
| | | CTTTCAAGTATATATTGGAACATACTATAAATAGAGACATCGGGCTTGTTCACTCCGATA |
| | | TGGAAACAGGAATAGGGAAGGATAATTATACTATCTTAAATGAACATGCACATCTTATAG |
| | | CACTGAGCCTTACAGTAATGATTGATGATGGAATCTTGGTGTCTAAGGTAGCTTATGCCC |
| | | CTGGGTTTTGCATCTCTTCATTATTGAATATGTACCGGACATTTTTTTCATTAGTTCTAT |
| | | GTGCGTTTCCACCGTATAGCAATTTTGAATCAACTGAATTTTACCTGATTTGCTTGCAAA |
| | | AAAGTATACCCGGACCTATCACACCAGCTAGAGCCATCCAACAAACGACGAAGCAATCTA |
| | | GAGAAGAGGATAATAGTATAACTAATAATATCCTCAAAATCAAAAATCTTGTTCAGAAAG |
| | | AATTTATCAAAACAGTAAAGAAAAAATACGAAATCCATCCTTCGTTTAACTGTCCTATCA |
| | | ACTTCACAAAGGATGATAAATATTTAATGAGTGTTGGGTTTCAAGCCAATGGTCCTGATA |
| | | TGATACGTAAAGAGACGGGCTATGACATAGGTAGCAATGTAGAGAATCTCCGAGATGTCT |
| | | TAATCAAGTTGTTTGCAGATGCAGTCACCTTCTATGATGATGTCACAAATAAAAAGAACT |
| | | TTTTAAATCCTTATCCAGTCTACACAAGAACTCAGTATAAAATTCTGATGGATAAAATAT |
| | | GCAAGAAAGTCACCTTATACACCTTAATCATATCATGTAAAGGATCCAATCAATATTGCT |
| | | GGGAAATTAAATCCCAAATAAGAAAGCATTGTCTCATACTTGATTTGAAAAGTAAGGTTT |
| | | TTACAAAACTTATTCCAAAGGGATTAAGAGAAAGGGGTGACTCAAAAGGGATGAAGAGCA |
| | | TATGGTTCACTAAACTAACCAGTCAAGAGGTGAAAAGATGGTGGAAGATGATATCTTACA |
| | | TCGTGATAATAAGCAATCCATAACCACATCCAACTTGTCAGTTAAACACTTAAATCACAA |
| | | TAAACTTGTCATCAGATTAAAGAAAACTTATAATTCCCTTTTTTAGGT |

### REFERENCES

1. Marsh G A, de Jong C, Barr J A, Tachedjian M, Smith C, Middleton D, Yu M, Todd S, Foord A J, Haring V, Payne J, Robinson R, Broz I, Crameri G, Field H E, Wang L F. 2012. Cedar virus: a novel Henipavirus isolated from Australian bats. PLoS Pathog 8:e1002836.
2. Eaton B T, Broder C C, Middleton D, Wang L F. 2006. Hendra and Nipah viruses: different and dangerous. Nat Rev Microbiol 4:23-35.
3. Anonymous. 2020. Biosafety in Microbiological and Biomedical Laboratories (BMBL) 6th Edition. Services USDoHaH, US Government Printing Office, Washington. https://www.cdc.gov/labs/pdf/CDC-BiosafetyMicrobiologicalBiomedicalLaboratories-2020-P.pdf.
4. Laing E D, Navaratnarajah C K, Cheliout Da Silva S, Petzing S R, Xu Y, Sterling S L, Marsh G A, Wang L F, Amaya M, Nikolov D B, Cattaneo R, Broder C C, Xu K. 2019. Structural and functional analyses reveal promiscuous and species specific use of ephrin receptors by Cedar virus. Proc Natl Acad Sci USA 116:20707-20715.
5. Laing E D, Amaya M, Navaratnarajah C K, Feng Y R, Cattaneo R, Wang L F, Broder C C. 2018. Rescue and characterization of recombinant cedar virus, a non-pathogenic Henipavirus species. Virol J 15:56.
6. Wang L-F, Mackenzie J S, Broder C C. 2013. Henipaviruses, p 1070-1085. In Knipe D M, Howley P M (ed), Fields Virology, vol 1. Lippincott Williams & Wilkins, Philadelphia.
7. Geisbert T W, Feldmann H, Broder C C. 2012. Animal Challenge Models of Henipavirus Infection and Pathogenesis. Curr Top Microbiol Immunol 359:153-77.
8. Sharma V, Kaushik S, Kumar R, Yadav J P, Kaushik S. 2019. Emerging trends of Nipah virus: A review. Rev Med Virol 29:e2010.
9. Drexler J F, Corman V M, Gloza-Rausch F, Seebens A, Annan A, Ipsen A, Kruppa T, Muller M A, Kalko E K, Adu-Sarkodie Y, Oppong S, Drosten C. 2009. Henipavirus RNA in African bats. PLoS One 4:e6367.
10. Wu Z, Yang L, Yang F, Ren X, Jiang J, Dong J, Sun L, Zhu Y, Zhou H, Jin Q. 2014. Novel Henipa-like virus, Mojiang Paramyxovirus, in rats, China, 2012. Emerg Infect Dis 20:1064-6.
11. Pelissier R, Iampietro M, Horvat B. 2019. Recent advances in the understanding of Nipah virus immunopathogenesis and anti-viral approaches. F1000Res 8.
12. Park M S, Shaw M L, Munoz-Jordan J, Cros J F, Nakaya T, Bouvier N, Palese P, Garcia-Sastre A, Basler C F. 2003. Newcastle disease virus (NDV)-based assay demonstrates interferon-antagonist activity for the NDV V protein and the Nipah virus V, W, and C proteins. J Virol 77:1501-11.
13. Virtue E R, Marsh G A, Wang L F. 2011. Interferon signaling remains functional during henipavirus infection of human cell lines. J Virol 85:4031-4.
14. Yoneda M, Guillaume V, Sato H, Fujita K, Georges-Courbot M C, Ikeda F, Omi M, Muto-Terao Y, Wild T F, Kai C. 2010. The nonstructural proteins of Nipah virus play a key role in pathogenicity in experimentally infected animals. PLoS One 5:e12709.
15. Satterfield B A, Borisevich V, Foster S L, Rodriguez S E, Cross R W, Fenton K A, Agans K N, Basler C F, Geisbert T W, Mire C E. 2019. Antagonism of STAT1 by Nipah virus P gene products modulates disease course but not lethal outcome in the ferret model. Sci Rep 9:16710.
16. Satterfield B A, Cross R W, Fenton K A, Agans K N, Basler C F, Geisbert T W, Mire C E. 2015. The immunomodulating V and W proteins of Nipah virus determine disease course. Nat Commun 6:7483.
17. Satterfield B A, Cross R W, Fenton K A, Borisevich V, Agans K N, Deer D J, Graber J, Basler C F, Geisbert T W, Mire C E. 2016. Nipah Virus C and W Proteins Contribute to Respiratory Disease in Ferrets. J Virol 90:6326-43.
18. Schountz T, Campbell C, Wagner K, Rovnak J, Martellaro C, DeBuysscher B L, Feldmann H, Prescott J. 2019. Differential Innate Immune Responses Elicited by Nipah Virus and Cedar Virus Correlate with Disparate In Vivo Pathogenesis in Hamsters. Viruses 11.
19. Yun T, Park A, Hill T E, Pernet O, Beaty S M, Juelich T L, Smith J K, Zhang L, Wang Y E, Vigant F, Gao J, Wu P, Lee B, Freiberg A N. 2015. Efficient Reverse Genetics Reveals Genetic Determinants of Budding and Fusogenic Differences between Nipah and Hendra Viruses and Enables Real-Time Monitoring of Viral Spread in Small Animal Models of Henipavirus Infection. J Virol 89:1242-53.
20. Weingartl H M, Berhane Y, Caswell J L, Loosmore S, Audonnet J C, Roth J A, Czub M. 2006. Recombinant nipah virus vaccines protect pigs against challenge. J Virol 80:7929-38.
21. Dang H V, Cross R W, Borisevich V, Bornholdt Z A, West B R, Chan Y P, Mire C E, Da Silva S C, Dimitrov A S, Yan L, Amaya M, Navaratnarajah C K, Zeitlin L, Geisbert T W, Broder C C, Veesler D. 2021. Broadly neutralizing antibody cocktails targeting Nipah virus and Hendra virus fusion glycoproteins. Nat Struct Mol Biol. 28(5):426-434.
22. Cheliout Da Silva S, Yan L, Dang H V, Xu K, Epstein J H, Veesler D, Broder C C. 2021. Functional analysis of the fusion and attachment glycoproteins of Mojiang Henipavirus. Viruses 13(3):517.
23. Mire C E, Chan Y P, Borisevich V, Cross R W, Yan L, Agans K N, Dang H V, Veesler D, Fenton K A, Geisbert T W, Broder C C. 2020. A Cross-Reactive Humanized Monoclonal Antibody Targeting Fusion Glycoprotein Function Protects Ferrets Against Lethal Nipah Virus and Hendra Virus Infection. J Infect Dis 221:S471-S479.
24. Dang H V, Chan Y-P, Park Y-J, Snijder J, Da Silva S C, Vu B, Yan L, Feng Y-R, Rockx B, Geisbert T W, Mire C E, Broder C C, Veesler D. 2019. An antibody against the F glycoprotein inhibits Nipah and Hendra virus infections. Nature Structural & Molecular Biology 26:980-987.
25. Xu K, Rockx B, Xie Y, Debuysscher B L, Fusco D L, Zhu Z, Chan Y P, Xu Y, Luu T, Cer R Z, Feldmann H, Mokashi V, Dimitrov D S, Bishop-Lilly K A, Broder C C, Nikolov D B. 2013. Crystal structure of the hendra virus attachment g glycoprotein bound to a potent cross-reactive neutralizing human monoclonal antibody. PLoS Pathog 9:e1003684.
26. Kaku Y, Noguchi A, Marsh G A, Barr J A, Okutani A, Hotta K, Bazartseren B, Fukushi S, Broder C C, Yamada A, Inoue S, Wang L F. 2012. Second generation of pseudotype-based serum neutralization assay for Nipah virus antibodies: sensitive and high-throughput analysis utilizing secreted alkaline phosphatase. J Virol Methods 179:226-32.
27. Kaku Y, Noguchi A, Marsh G A, McEachern J A, Okutani A, Hotta K, Bazartseren B, Fukushi S, Broder C C, Yamada A, Inoue S, Wang L F. 2009. A neutralization test for specific detection of Nipah virus antibodies using pseudotyped vesicular stomatitis virus expressing green fluorescent protein. J Virol Methods 160:7-13.
28. Tamin A, Harcourt B H, Lo M K, Roth J A, Wolf M C, Lee B, Weingartl H, Audonnet J C, Bellini W J, Rota P A. 2009. Development of a neutralization assay for Nipah virus using pseudotype particles. J Virol Methods 160:1-6.
29. van den Pol A N, Mao G, Chattopadhyay A, Rose J K, Davis J N. 2017. Chikungunya, Influenza, Nipah, and Semliki Forest Chimeric Viruses with Vesicular Stomatitis Virus: Actions in the Brain. J Virol 91.
30. Khetawat D, Broder C C. 2010. A functional henipavirus envelope glycoprotein pseudotyped lentivirus assay system. Virol J 7:312.
31. Chattopadhyay A, Rose J K. 2011. Complementing defective viruses that express separate paramyxovirus glycoproteins provide a new vaccine vector approach. J Virol 85:2004-11.
32. Prescott J, DeBuysscher B L, Feldmann F, Gardner D J, Haddock E, Martellaro C, Scott D, Feldmann H. 2015. Single-dose live-attenuated vesicular stomatitis virus-based vaccine protects African green monkeys from Nipah virus disease. Vaccine 33:2823-9.
33. Lo M K, Bird B H, Chattopadhyay A, Drew C P, Martin B E, Coleman J D, Rose J K, Nichol S T, Spiropoulou C F. 2014. Single-dose replication-defective VSV-based Nipah virus vaccines provide protection from lethal challenge in Syrian hamsters. Antiviral Res 101:26-9.
34. DeBuysscher B L, Scott D, Marzi A, Prescott J, Feldmann H. 2014. Single-dose live-attenuated Nipah virus vaccines confer complete protection by eliciting antibodies directed against surface glycoproteins. Vaccine 32:2637-44.
35. Mire C E, Versteeg K M, Cross R W, Agans K N, Fenton K A, Whitt M A, Geisbert T W. 2013. Single injection recombinant vesicular stomatitis virus vaccines protect ferrets against lethal Nipah virus disease. Virol J 10:353.
36. Mire C E, Geisbert J B, Agans K N, Versteeg K M, Deer D J, Satterfield B A, Fenton K A, Geisbert T W. 2019. Use of Single-Injection Recombinant Vesicular Stomatitis Virus Vaccine to Protect Nonhuman Primates Against Lethal Nipah Virus Disease. Emerg Infect Dis 25:1144-1152.
37. Amaya M, Broder C C. 2020. Vaccines to Emerging Viruses: Nipah and Hendra. Annu Rev Virol 7:447-473.
38. Halpin K, Bankamp B, Harcourt B H, Bellini W J, Rota P A. 2004. Nipah virus conforms to the rule of six in a minigenome replication assay. J Gen Virol 85:701-7.
39. Weis M, Maisner A. 2015. Nipah virus fusion protein: Importance of the cytoplasmic tail for endosomal trafficking and bioactivity. Eur J Cell Biol 94:316-22.
40. Middleton D, Pallister J, Klein R, Feng Y R, Haining J, Arkinstall R, Frazer L, Huang J A, Edwards N, Wareing M, Elhay M, Hashmi Z, Bingham J, Yamada M, Johnson D, White J, Foord A, Heine H G, Marsh G A, Broder C C, Wang L F. 2014. Hendra virus vaccine, a one health approach to protecting horse, human, and environmental health. Emerg Infect Dis 20:372-9.
41. Steffen D L, Xu K, Nikolov D B, Broder C C. 2012. Henipavirus mediated membrane fusion, virus entry and targeted therapeutics. Viruses. 4(2):280-308.
42. Navaratnarajah C K, Generous A R, Yousaf I, Cattaneo R. 2020. Receptor-mediated cell entry of paramyxoviruses: Mechanisms, and consequences for tropism and pathogenesis. J Biol Chem. 28; 295(9):2771-2786.
43. Bishop K A, Stantchev T S, Hickey A C, Khetawat D, Bossart K N, et al. 2007. Identification of Hendra virus G glycoprotein residues that are critical for receptor binding. J. Virol. 81:5893-901.
44. Bonaparte M I, Dimitrov A S, Bossart K N, Crameri G, Mungall B A, et al. 2005. Ephrin-B2 ligand is a functional receptor for Hendra virus and Nipah virus. PNAS 102:10652-57.
45. Negrete O A, Levroney E L, Aguilar H C, Bertolotti-Ciarlet A, Nazarian R, et al. 2005. EphrinB2 is the entry receptor for Nipah virus, an emergent deadly paramyxovirus. Nature 436:401-5.
46. Negrete O A, Wolf M C, Aguilar H C, Enterlein S, Wang W, et al. 2006. Two key residues in ephrinB3 are critical for its use as an alternative receptor for Nipah virus. PLOS Pathog. 2:e7.
47. Drescher U. 2002. Eph family functions from an evolutionary perspective. Curr. Opin. Genet. Dev. 12:397-402.

1. A replication-competent recombinant Cedar virus (rCedV) chimera wherein one or both of the F and G envelope glycoprotein genes of CedV are replaced with one or both of the F and G envelope glycoprotein genes, respectively, of a non-CedV henipavirus.

2. The rCedV chimera of claim 1, wherein the non-CedV henipavirus is selected from the group consisting of Hendra virus (HeV), Nipah virus (NiV), Ghana virus (GhV), and Mojiang virus (MojV).

3. The rCedV chimera of claim 1, wherein the non-CedV henipavirus is selected from the Malaysian strain of NiV (NiV-M) and the Bangladesh strain of NiV (NiV-B).

4. The rCedV chimera of claim 2, comprising the F and G envelope glycoprotein genes of HeV (rCedV-HeV).

5. The rCedV chimera of claim 2, comprising the F and G envelope glycoprotein genes of NiV (rCedV-NiV).

6. The rCedV chimera of claim 3, comprising the F and G envelope glycoprotein genes of NiV-M (rCedV-NiV-M).

7. The rCedV chimera of claim 3, comprising the F and G envelope glycoprotein genes of NiV-B (rCedV-NiV-B).

8. The rCedV chimera of claim 1, further comprising a reporter sequence.

9. The rCedV of claim 8, wherein the reporter sequence encodes green fluorescent protein (GFP) or luciferase protein (Luc).

10. (canceled)

11. A replication-competent recombinant Cedar virus (rCedV) chimera, comprising the F and G envelope glycoprotein genes of CedV, further comprising a coding sequence for one or both of (i) a soluble F envelope glycoprotein (sF) of a non-CedV henipavirus, (ii) a soluble G envelope glycoprotein (sG) of a non-CedV henipavirus.

12. The rCedV chimera of claim 11, wherein the sF coding sequence, the sG coding sequence, or both, are from NiV.

13. The rCedV chimera of claim 11, wherein the sF coding sequence, the sG coding sequence, or both, are from HeV.

14. A replication-competent recombinant Cedar virus (rCedV) chimera, comprising one or both of (i) a gene encoding a henipavirus F envelope protein fusion protein, and (ii) a gene encoding a henipavirus G envelope protein fusion protein, wherein the fusion protein comprises the ectodomain and transmembrane domain of a non-CedV henipavirus F envelope protein or G envelope protein, respectively, fused to the cytoplasmic tail domain of CedV F envelope protein or G envelope protein, respectively, or the fusion protein comprises the ectodomain of a non-CedV henipavirus F envelope protein or G envelope protein, respectively, fused to the transmembrane domain and cytoplasmic tail domain of CedV F envelope protein or G envelope protein, respectively.

15. The rCedV chimera of claim 14, wherein the non-CedV henipavirus is selected from the group consisting of HeV, NiV, GhV, and MojV.

16. (canceled)

17. The rCedV chimera of claim 14, wherein the non-CedV henipavirus is the Malaysian strain of NiV (NiV-M) or the Bangladesh strain of NiV (NiV-B).

18. A vaccine composition, comprising a rCedV chimera according to claim 1 and a pharmaceutically acceptable carrier.

19. The vaccine composition of claim 18, further comprising an adjuvant.

20. The vaccine composition of claim 18, further comprising one or both of (i) a soluble F envelope glycoprotein (sF) of a non-CedV henipavirus, (ii) a soluble G envelope glycoprotein (sG) of a non-CedV henipavirus.

21. The vaccine composition of claim 20, wherein the non-CedV henipavirus sG and sF comprise NiV sG and sF or HeV sG and sF.

22. (canceled)

23. A method of treating, reducing the risk of, or preventing henipavirus infection in a subject in need thereof, comprising administering to the subject an effective amount of the vaccine composition of claim 18.

24-30. (canceled)
